(19) United States
(12) Patent Application Publication
(10) Pub. No.: US 2018/0203017 A1
(43) Pub. Date: Jul. 19, 2018

(54) PROTEIN-PROTEIN INTERACTION DETECTION SYSTEMS AND METHODS OF USE THEREOF

(71) Applicant: The Board of Trustees of the Leland Stanford Junior University, Stanford, CA (US)

(72) Inventors: Alice Y. Ting, Stanford, CA (US); Wenjing Wang, Stanford, CA (US)

(21) Appl. No.: 15/855,638

(22) Filed: Dec. 27, 2017

Related U.S. Application Data
(60) Provisional application No. 62/440,825, filed on Dec. 30, 2016.
(60) Provisional application No. 62/523,609, filed on Jun. 22, 2017.

(51) Int. Cl. G01N 33/68; G01N 33/50; G01N 33/542; C12N 15/86

(52) U.S. Cl. CPC G01N 33/6845; G01N 33/5032; C07K 2319/03; C12N 15/86; C07K 2319/02; G01N 33/542

(57) ABSTRACT

The present disclosure provides polypeptides, nucleic acids, polypeptide systems, and nucleic acid systems for detecting protein-protein interactions. The polypeptides, nucleic acids, and systems are useful for detecting protein-protein interactions. The present disclosure also provides such methods.

### CROSS-REFERENCE

This application claims the benefit of U.S. Provisional Patent Application No. 62/440,825, filed Dec. 30, 2016, and U.S. Provisional Patent Application No. 62/523,609, filed Jun. 22, 2017, which applications are incorporated herein by reference in their entirety.

### INTRODUCTION

Systems for detecting protein-protein interactions are currently available, and include, e.g., the TANGO™ system (see, e.g., Barnea et al. (2008) Proc. Natl. Acad. Sci. USA 105:64); and the split ubiquitin system (see, e.g., Petschnigg et al. (2014) Nat. Methods 11:585). However, disadvantages of current systems include lack of temporal control, low sensitivity, the requirement for long stimulation periods (e.g., 4 hours or more), and low signal-to-noise ratios.

There is a need in the art for improved systems for detecting protein-protein interactions.

### SUMMARY

The present disclosure provides polypeptides, nucleic acids, polypeptide systems, and nucleic acid systems for detecting protein-protein interactions. The polypeptides, nucleic acids, and systems are useful for detecting protein-protein interactions. The present disclosure also provides such methods.

### BRIEF DESCRIPTION OF THE DRAWINGS

FIG. 1 is a schematic depiction of the requirement for two input signals for functioning of a system of the present disclosure.

FIG. 2 presents a comparison of a protein-protein interaction (PPI) detection system of the present disclosure to the TANGO system.

FIG. 3 is a schematic depiction of an example of a PPI detection system of the present disclosure.

FIG. 4 depicts PPI detection using a PPI detection system as schematically depicted in FIG. 3.

FIG. 5 is a schematic depiction of an example of a PPI detection system of the present disclosure.

FIG. 6 is a workflow diagram for use of a PPI detection system as schematically depicted in FIG. 5.

FIG. 7 and FIG. 8 depict PPI detection using a PPI detection system as schematically depicted in FIG. 5.

FIG. 9 is a schematic depiction of an example of a PPI detection system of the present disclosure.

FIG. 10 depicts PPI detection using a PPI detection system as schematically depicted in FIG. 9.

FIG. 11A-11G provide amino acid sequences of LOV domains of light-activated polypeptides.

FIG. 12A-12D provide amino acid sequences of tobacco etch virus (TEV) protease.

FIG. 13 provides the amino acid sequence of a Streptomyces pyogenes Cas9 polypeptide.

FIG. 14 provides the amino acid sequence of a Staphylococcus aureus Cas9 polypeptide.

FIG. 15 provides amino acid sequences of various depolarizing opsins.

FIG. 16 provides amino acid sequences of various hyperpolarizing opsins.

FIG. 17A-17B provide amino acid sequences of a PPI detection system of the present disclosure.

FIG. 18A-18B provide amino acid sequences of a PPI detection system of the present disclosure.

FIG. 19A-19C provide amino acid sequences (FIGS. 19A and 19B) and nucleotide sequences (FIG. 19C) of a PPI detection system of the present disclosure.

FIG. 20A-20B provide amino acid sequences of a PPI detection system of the present disclosure.

FIG. 21A-21F depict design of FLARE-PPI to light- and agonist-dependent detection of β2-adrenergic receptor (β2-AR)-arrestin2 interaction.

FIG. 22A-22B depict agonist-dependent detection of β2-adrenergic receptor (β2-AR)-arrestin2 interaction.

FIG. 23 depicts Western blot quantification of cleavage extent.

FIG. 24 depicts agonist-dependent detection of β2-adrenergic receptor (β2-AR)-arrestin2 interaction in various light conditions.

FIG. 25 depicts FLARE with 3 different TEV protease cleavable linkers (TEV protease cleavage site; TEVcs).

FIG. 26A-26D depict light gating of FLARE-PPI in the dynamic analysis of GPCR-arrestin2 interactions.

FIG. 27A-27D depict application of FLARE-PPI to a variety of PPIs.

FIG. 28A-28B depict coupling of FLARE to genetic selections.

FIG. 29A-29D depict the effect of various LOV domains on FLARE-PPI.

FIG. 30A-30C depict comparisons of FLARE-PPI to TANGO and iTango.

### DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

“Operably linked” refers to a juxtaposition wherein the components so described are in a relationship permitting them to function in their intended manner. For instance, a promoter is operably linked to a coding region of a nucleic acid if the promoter affects transcription or expression of the coding region of a nucleic acid.

A “vector” or “expression vector” is a replicon, such as plasmid, phage, virus, or cosmid, to which another DNA segment, i.e. an “insert”, may be attached so as to bring about the replication of the attached segment in a cell.

“Heterologous,” as used herein, refers to a nucleotide or polypeptide sequence that is not found in the native (e.g., naturally-occurring) nucleic acid or protein, respectively.

As used herein, the term “affinity” refers to the equilibrium constant for the reversible binding of two agents (e.g., a protease and a polypeptide comprising a protease cleavage site) and is expressed as Km. Km is the concentration of peptide at which the catalytic rate of proteolytic cleavage is half of Vmax (maximal catalytic rate). Km is often used in the literature as an approximation of affinity when speaking about enzyme-substrate interactions.

The term “binding” refers to a direct association between two molecules (e.g., two polypeptide members of a protein interaction pair), due to, for example, covalent, electrostatic, hydrophobic, and ionic and/or hydrogen-bond interactions, including interactions such as salt bridges and water bridges. “Specific binding” refers to binding with an affinity of at least about 10⁻⁷ M or greater, e.g., 5×10⁻⁷ M, 10⁻⁸ M, 5×10⁻⁸ M, and greater. “Non-specific binding” refers to binding with an affinity of less than about 10⁻⁷ M, e.g., binding with an affinity of 10⁻⁶ M, 10⁻⁵ M, 10⁻⁴ M, etc. In some cases, e.g., in instances of transient protein-protein interactions, “specific binding” can be lower than 10⁻⁷ M; e.g., specific binding can be binding with an affinity of at least 10⁻⁵ M or greater, e.g., 10⁻⁵ M, 10⁻⁶ M, or 10⁻⁷ M. Binding affinities can depend on the chemical environment, e.g. the pH value, the ionic strength, the presence of co-factors, etc. In the context of the present disclosure, the term “protein-protein interaction” can refer to protein-protein interactions occurring under physiological conditions, i.e. in a living cell.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

As used herein, the term “bait protein” refers to a protein which is used to investigate an interaction with another protein. As used herein, the term “prey protein” refers to a protein which is a potential interaction partner of the “bait protein” and becomes a target which is investigated, analyzed, or detected. As used herein, the term “candidate interaction regulator” refers to an agent that promotes, induces, suppresses, or inhibits the interaction between a “bait protein” and a “prey protein”. A “protein interaction pair” (also referred to herein as a “protein-protein interaction pair”) comprises a prey protein (also referred to herein as a second polypeptide member of a protein interaction pair) and a bait protein (also referred to herein as a first polypeptide member of a protein interaction pair).

An “isolated” polypeptide or an “isolated” nucleic acid is one that has been identified and separated and/or recovered from a component of its natural environment. Contaminant components of its natural environment are materials that would interfere with use of the polypeptide or nucleic acid, and may include enzymes, hormones, and other proteinaceous or nonproteinaceous solutes. In some embodiments, the polypeptide or nucleic acid will be purified to greater than 80%, greater than 85%, greater than 90%, greater than 95%, or greater than 98%, by weight.

The term “genetic modification” refers to a permanent or transient genetic change induced in a cell following introduction into the cell of a heterologous nucleic acid (e.g., a nucleic acid exogenous to the cell). Genetic change (“modification”) can be accomplished by incorporation of the heterologous nucleic acid into the genome of the host cell, or by transient or stable maintenance of the heterologous nucleic acid as an extrachromosomal element. Where the cell is a eukaryotic cell, a permanent genetic change can be achieved by introduction of the nucleic acid into the genome of the cell. Suitable methods of genetic modification include viral infection, transfection, conjugation, protoplast fusion, electroporation, particle gun technology, calcium phosphate precipitation, direct microinjection, use of a CRISPR/Cas9 system, and the like.

A “host cell,” as used herein, denotes an in vivo or in vitro eukaryotic cell, or a cell from a multicellular organism (e.g., a cell line) cultured as a unicellular entity, which eukaryotic cells can be, or have been, used as recipients for a nucleic acid (e.g., an expression vector that comprises a nucleotide sequence encoding a PPI detection system of the present disclosure; an expression vector that comprises a nucleotide sequence encoding a component of a PPI detection system of the present disclosure; or any other nucleic acid or expression vector described herein), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a genetically modified eukaryotic host cell is genetically modified by virtue of introduction into a suitable eukaryotic host cell of a heterologous nucleic acid, e.g., an exogenous nucleic acid that is foreign to the eukaryotic host cell, or a recombinant nucleic acid that is not normally found in the eukaryotic host cell, where such nucleic acids and expression vectors are described herein.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a transcription factor” includes a plurality of such transcription factors and reference to “the proteolytically cleavable linker” includes reference to one or more proteolytically cleavable linkers and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides polypeptides, nucleic acids, polypeptide systems, and nucleic acid systems for detecting protein-protein interactions. The polypeptides, nucleic acids, and systems are useful for detecting protein-protein interactions. The present disclosure also provides such methods.

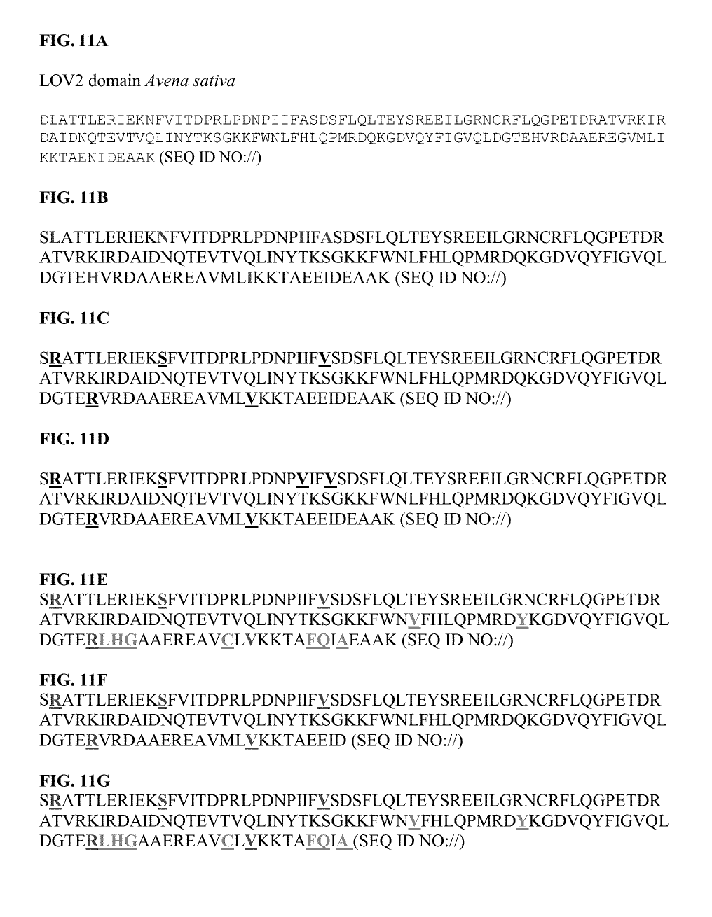

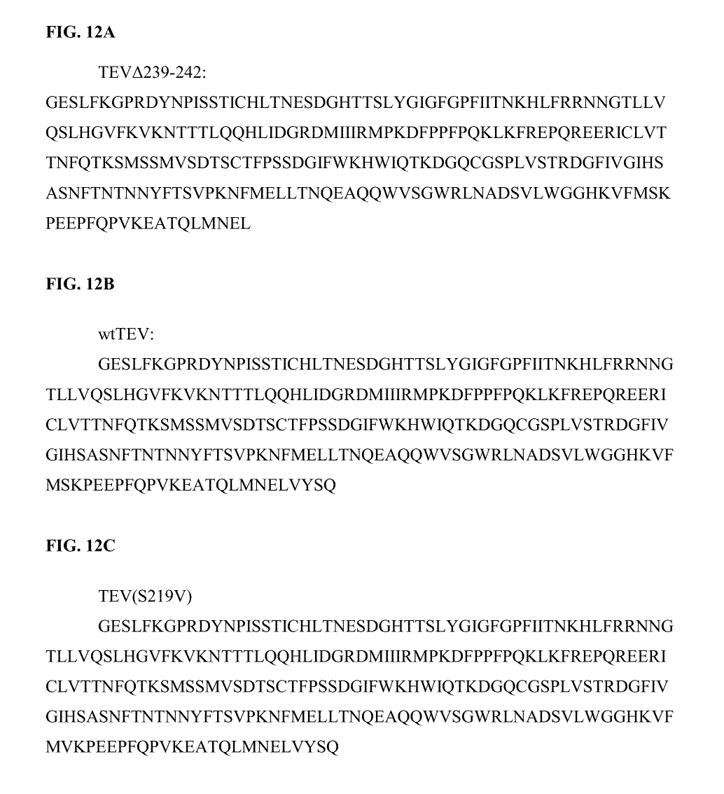

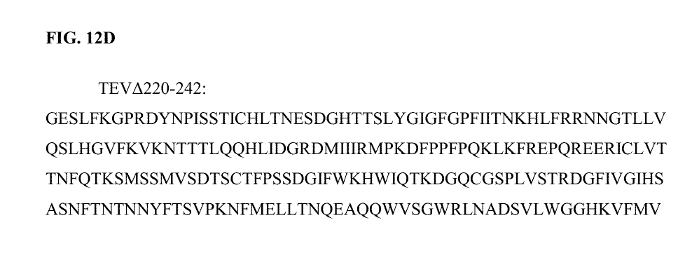

A protein-protein interaction (PPI) detection system of the present disclosure comprises two polypeptide chains (or one or more nucleic acids comprising nucleotide sequences encoding the two polypeptide chains), where the first polypeptide chain is a first fusion polypeptide that comprises, in order from amino terminus (N-terminus) to carboxyl terminus (C-terminus): i) a tethering domain (e.g., a transmembrane domain or other tethering domain); ii) a first member of a protein interaction pair; iii) a LOV-domain light-activated polypeptide comprising an amino acid sequence having at least 80% amino acid sequence identity to the amino acid sequence depicted in any one of FIG. 11A-11G; iv) a proteolytically cleavable linker; and v) a polypeptide of interest; and where the second polypeptide chain is a second fusion polypeptide that comprises, in order from N-terminus to C-terminus: i) a second member of the protein interaction pair; and ii) a protease that cleaves the proteolytically cleavable linker. In some cases, instead of a polypeptide of interest, a PPI detection system of the present disclosure provides an insertion site in a nucleic acid encoding a PPI system of the present disclosure, where a nucleic acid encoding a polypeptide of interest can be inserted into the insertion site. In some cases, e.g., where the polypeptide of interest is a transcription factor, a PPI detection system of the present disclosure further comprises a nucleic acid comprising: a) a promoter that is activated or repressed by the transcription factor; and b) a nucleotide sequence that is operably linked to the promoter, and that encodes a polypeptide or a nucleic acid gene product. For example, a polypeptide gene product can be a polypeptide that provides a detectable signal, that induces transcription of a further nucleic acid, or that provides a function that modulates an activity of a cell.

A PPI detection system of the present disclosure is an “AND” gate, and requires two signals in order for the first fusion polypeptide and the second fusion polypeptide to be brought into proximity to one another in a cell and for the polypeptide of interest to be released from the first fusion polypeptide. One signal is blue light, which activates the LOV domain polypeptide such that the proteolytically cleavable linker, which is sequestered by the LOV domain polypeptide in the absence of blue light, to become accessible to the protease. The second signal is the protein-protein interaction, which can be induced by an agent or effect, or is always on. In some cases, the second signal is an agent or effect that induces the first and second members of the protein interaction pair to bind to one another; in other cases, the second signal is an agent or effect that inhibits or reduces binding of the first and second members of the protein interaction pair to bind to one another. In some cases, the polypeptide of interest is a transcription factor that, when released from the first fusion polypeptide by action of the protease on the proteolytically cleavable linker, enters the nucleus of the cell and induces transcription of a gene product that produces a detectable signal. For example, in some cases, the gene product is a fluorescent polypeptide. When the cell is exposed to the two requisite signals, the fluorescent polypeptide is produced.

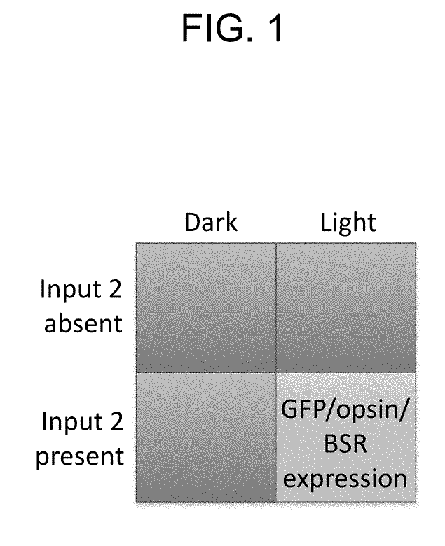

A PPI detection system of the present disclosure, when present in a cell, provides a high signal-to-noise (S/N) ratio. As depicted in schematically in FIG. 1, in the absence of light of an activating wavelength (e.g., blue light), and in the absence of an agent or effect that induces the first and second members of the protein interaction pair to bind to one another, the first fusion polypeptide and the second polypeptide do not substantially bind to one another, because the first and second members of the protein interaction pair do not substantially bind to one another in the absence of the agent or effect. Furthermore, even if the first fusion polypeptide and the second fusion polypeptide were to bind to one another, since the LOV light-activated polypeptide cages the proteolytically cleavable linker in the absence of light of an activating wavelength, the proteolytically cleavable linker is not accessible to the protease. Thus, two signals are required for: 1) binding of the first and second members of the protein-interaction pair; and 2) cleavage of the proteolytically cleavable linker by the protease.

A PPI detection system of the present disclosure, when present in a cell, provides a signal-to-noise ratio of at least 3:1, at least 4:1, at least 5:1, at least 6:1, at least 7:1, at least 8:1, at least 9:1, at least 10:1, from 10:1 to 15:1, from 15:1 to 20:1, or more than 20:1 (e.g., from 20:1 to 50:1, from 50:1 to 100:1, from 100:1 to 150:1, or more than 150:1); i.e., the signal produced when the cell is exposed to light of an activating wavelength (e.g., blue light) and to a second signal (a “binding inducing signal”) that induces binding of the first and second polypeptide members of a protein interaction pair to one another is at least 2-fold, at lease 3-fold, at least 4-fold, at least 5-fold, at least 6-fold, at least 7-fold, at least 8-fold, at least 9-fold, at least 10-fold, at least 15-fold, at least 20-fold, or more than 20-fold (e.g., more than 25-fold, more than 50-fold, more than 75-fold, more than 100-fold, more than 125-fold, or more than 150-fold), higher than the signal produced by the cell when the cell is: i) not exposed to either light of an activating wavelength or to a binding inducing signal; ii) exposed to light of an activating wavelength, but not to a binding inducing signal; or iii) exposed to binding inducing signal, but not to light of an activating wavelength.

A PPI detection system of the present disclosure, when present in a cell, can be activated within less than one hour upon exposure to a first and a second stimulus; e.g., a PPI detection system of the present disclosure, when present in a cell, can be activated within 60 minutes, within 45 minutes, within 30 minutes, within 15 minutes, within 10 minutes, within 5 minutes, within 1 minute, within 50 seconds, within 45 seconds, within 30 seconds, within 15 seconds, within 5 seconds, or within less than 1 second, following exposure to a first and a second stimulus (e.g., following exposure to blue light and an agent that induces protein-protein interaction).

A PPI detection system of the present disclosure, when present in a cell, can provide for temporal information regarding a PPI. Thus, a method of the present disclosure can be carried out over time.

A PPI detection system of the present disclosure is useful for: 1) controlling an activity of a cell in response to a signal that induces PPI; 2) identifying, from a library of unknown proteins, a protein that interacts with a known protein; 3) identifying an agent that inhibits a PPI; 4) identifying an agent that induces PPI; 5) identifying, from a library of variants of a known protein, a protein that interacts with a given protein; 6) identifying an agent that modulates a PPI; 7) identifying, from a library of variants of a known protein, a protein that does not interact with a given protein; 8) providing a rapid light (or ligand) gated protein expression system; 9) identifying a third gene that modulates the known PPI; 10) identifying mutations of a known protein interaction pair that strengthens or weakens the PPI; and the like.

### PPI Detection Systems

System 1.

The present disclosure provides a nucleic acid system (“System 1”) comprising: A) a first nucleic acid comprising, in order from 5′ to 3′: a) a nucleotide sequence encoding a first, light-activated fusion polypeptide comprising, in order from amino terminus to carboxyl terminus: i) a transmembrane domain (or other tethering domain); ii) a first member of a protein interaction pair; iii) a LOV-domain light-activated polypeptide comprising an amino acid sequence having at least 80% amino acid sequence identity to the amino acid sequence depicted in any one of FIG. 11A-11G; and iv) a proteolytically cleavable linker; and b) an insertion site for a nucleic acid comprising a nucleotide sequence encoding a polypeptide of interest; and B) a second nucleic acid comprising a nucleotide sequence encoding a second fusion polypeptide comprising: i) a second member of the protein interaction pair; and ii) a protease that cleaves the proteolytically cleavable linker, wherein the first member of the protein interaction pair and the second member of the protein interaction pair bind to one another in the presence of an agent.

In some cases, the insertion site is a multiple cloning site. For example, the insertion site can comprise multiple (e.g., 2, 3, 4, or more) restriction endonuclease cleavage sites. The insertion site can comprise a restriction endonuclease cleavage site; in such a case, a nucleic acid comprising a nucleotide sequence encoding a polypeptide of interest can comprise, at its 5′ and 3′ ends, nucleotide sequences (e.g., complementary overhangs) that anneal with the ends created by restriction endonuclease cleavage.

The insertion site is within 10 nucleotides (nt), within 9 nt, within 8 nt, within 7 nt, within 6 nt, within 5 nt, within 4 nt, within 3 nt, within 2 nt, or 1 nt, of the 3′ end of the nucleotide sequence encoding the first (light-activated) fusion polypeptide. The insertion site is positioned relative to the nucleotide sequence encoding the first fusion polypeptide such that, after insertion of a nucleic acid comprising a nucleotide sequence encoding a polypeptide of interest, and after transcription and translation, a fusion polypeptide comprising: i) a transmembrane domain; ii) a first polypeptide member of a protein-interaction pair; iii) a LOV-domain light-activated polypeptide comprising an amino acid sequence having at least 80% amino acid sequence identity to the amino acid sequence depicted in any one of FIG. 11A-11G; iv) a proteolytically cleavable linker; and v) the polypeptide of interest, is produced.

System 2.

The present disclosure provides a nucleic acid system (“System 2”) comprising: a) a first nucleic acid comprising a nucleotide sequence encoding a first fusion polypeptide comprising, in order from amino terminus to carboxyl terminus: i) a transmembrane domain (or other tethering domain); ii) a first member of a protein interaction pair; iii) a LOV light-activated polypeptide comprising an amino acid sequence having at least 80% amino acid sequence identity to the amino acid sequence depicted in any one of FIG. 11A-11G; iv) a proteolytically cleavable linker; and v) a polypeptide of interest; and b) a second nucleic acid comprising a nucleotide sequence encoding a second fusion polypeptide comprising: i) a second member of the protein interaction pair; and ii) a protease that cleaves the proteolytically cleavable linker, wherein the first member of the protein interaction pair and the second member of the protein interaction pair bind to one another in the presence of a binding-inducing agent.

A transmembrane domain, a polypeptide member of a protein interaction pair, a LOV-domain light-activated polypeptide, a proteolytically cleavable linker, and a protease, that can be encoded by a nucleotide sequence included in one or more embodiments of System 1 or System 2, are described below.

### System Components

The present disclosure provides components of a system of the present disclosure, e.g., components of System 1 and System 2.

For example, the present disclosure provides a nucleic acid comprising: a) a nucleotide sequence encoding a first (light-activated) fusion polypeptide comprising, in order from amino terminus to carboxyl terminus: i) a transmembrane domain; ii) first polypeptide member of a protein interaction pair; iii) a LOV-domain light-activated polypeptide comprising an amino acid sequence having at least 80% amino acid sequence identity to the amino acid sequence depicted in any one of FIG. 11A-11G; and iv) a proteolytically cleavable linker; and b) an insertion site for a nucleic acid comprising a nucleotide sequence encoding a polypeptide of interest. In some cases, the nucleotide sequence encoding the first fusion polypeptide is operably linked to a promoter. Suitable promoters are described below. In some cases, the nucleic acid is present in a recombinant expression vector, e.g., a recombinant viral vector. Suitable vectors are described below. The present disclosure provides a genetically modified host cell that is genetically modified with the nucleic acid. The present disclosure provides a genetically modified host cell that is genetically modified with the recombinant expression vector. Suitable host cells are described below.

As another example, the present disclosure provides a nucleic acid comprising a nucleotide sequence encoding a fusion polypeptide comprising: i) second polypeptide member of a protein interaction pair; and ii) a protease. In some cases, the nucleotide sequence encoding the fusion polypeptide is operably linked to a promoter. Suitable promoters are described below. In some cases, the nucleic acid is present in a recombinant expression vector, e.g., a recombinant viral vector. Suitable vectors are described below. The present disclosure provides a genetically modified host cell that is genetically modified with the nucleic acid. The present disclosure provides a genetically modified host cell that is genetically modified with the recombinant expression vector. Suitable host cells are described below.

As another example, the present disclosure provides a nucleic acid comprising a nucleotide sequence encoding a first (light-activated) fusion polypeptide comprising, in order from amino terminus to carboxyl terminus: i) a transmembrane domain; ii) a first polypeptide member of a protein interaction pair; iii) a LOV-domain light-activated polypeptide comprising an amino acid sequence having at least 80% amino acid sequence identity to the amino acid sequence depicted in any one of FIG. 11A-11G; iv) a proteolytically cleavable linker; and v) a polypeptide of interest. In some cases, the nucleotide sequence encoding the first fusion polypeptide is operably linked to a promoter. Suitable promoters are described below. In some cases, the nucleic acid is present in a recombinant expression vector, e.g., a recombinant viral vector. Suitable vectors are described below. The present disclosure provides a genetically modified host cell that is genetically modified with the nucleic acid. The present disclosure provides a genetically modified host cell that is genetically modified with the recombinant expression vector. Suitable host cells are described below.

As another example, the present disclosure provides a nucleic acid comprising a nucleotide sequence encoding a first (light-activated) fusion polypeptide comprising, in order from amino terminus to carboxyl terminus: i) a first polypeptide member of a protein interaction pair, where the first polypeptide member of a protein interaction pair is a membrane polypeptide (e.g., comprises a transmembrane domain); iii) a LOV-domain light-activated polypeptide comprising an amino acid sequence having at least 80% amino acid sequence identity to the amino acid sequence depicted in any one of FIG. 11A-11G; iv) a proteolytically cleavable linker; and v) a polypeptide of interest. In some cases, the nucleotide sequence encoding the first fusion polypeptide is operably linked to a promoter. Suitable promoters are described below. In some cases, the nucleic acid is present in a recombinant expression vector, e.g., a recombinant viral vector. Suitable vectors are described below. The present disclosure provides a genetically modified host cell that is genetically modified with the nucleic acid. The present disclosure provides a genetically modified host cell that is genetically modified with the recombinant expression vector. Suitable host cells are described below.

### Transmembrane Domain

Any of a variety of transmembrane domains (polypeptides) can be used in the first fusion polypeptide of the present disclosure. A suitable transmembrane domain is any polypeptide that is thermodynamically stable in a membrane, e.g., a eukaryotic cell membrane such as a mammalian cell membrane. Suitable transmembrane domains include a single alpha helix, a transmembrane beta barrel, or any other structure.

A “mammalian cell membrane” includes the membrane of a membrane-bound organelle (e.g., the nucleus, a mitochondrion, a lysosome, the endoplasmic reticulum, the Golgi apparatus, a vacuole, a chloroplast); and the plasma membrane. Thus, a suitable transmembrane domain is in some cases a transmembrane domain that provides for insertion into the plasma membrane. In some cases, a suitable transmembrane domain provides for insertion into a chloroplast membrane. In some cases, a suitable transmembrane domain provides for insertion into a mitochondrial membrane. In some cases, a suitable transmembrane domain provides for insertion into a lysosome.

A suitable transmembrane domain can have a length of from about 10 to 50 amino acids, e.g., from about 10 amino acids to about 40 amino acids, from about 20 amino acids to about 40 amino acids, from about 15 amino acids to about 25 amino acids, e.g., from about 10 amino acids to about 15 amino acids, from about 15 amino acids to about 20 amino acids, from about 20 amino acids to about 25 amino acids, from about 25 amino acids to about 30 amino acids, from about 30 amino acids to about 35 amino acids, from about 35 amino acids to about 40 amino acids, from about 40 amino acids to about 45 amino acids, or from about 45 amino acids to about 50 amino acids.

Suitable transmembrane (TM) domains include, e.g., a Syne homology nuclear TM domain; a CD4 TM domain; a CD8 TM domain; a KASH protein TM domain; a neurexin3b TM domain; a Notch receptor polypeptide TM domain; etc.

For example, a CD4 TM domain can comprise the amino acid sequence MALIVLGGVAGLLLFIGLGIFF (SEQ ID NO://); a CD8 TM domain can comprise the amino acid sequence IYIWAPLAGTCGVLLLSLVIT (SEQ ID NO://); a neurexin3b TM domain can comprise the amino acid sequence GMVVGIVAAAALCILILLYAM (SEQ ID NO://); a Notch receptor polypeptide TM domain can comprise the amino acid sequence FMYVAAAAFVLLFFVGCGVLL (SEQ ID NO://).

### Alternative Tethers

In some cases, in place of a transmembrane domain, first fusion polypeptide comprises a polypeptide that tethers the first fusion polypeptide to actin. A suitable actin-binding polypeptide includes, e.g., filamin, spectrin, transgelin, fimbrin, villin, fascin, formin, tensin, tropomodulin, gelsolin, and actin-binding fragments thereof.

In some cases, in place of a transmembrane domain, the first fusion polypeptide comprises a polypeptide that excludes first fusion polypeptide from the nucleus. Such a polypeptide can be a nuclear exclusion signal (NES) or nuclear export signal. Suitable NES polypeptides include, e.g., MVKELQEIRL (SEQ ID NO://); MTASALARMEV (SEQ ID NO://); LALKLAGLDI (SEQ ID NO://); LQKKLEELEL (SEQ ID NO://); LESNLRELQI (SEQ ID NO://); LCQAFSDVLI (SEQ ID NO://); MVKELQEIRLEP (SEQ ID NO://); LQKKLEELELA (SEQ ID NO://); LALKLAGLDIN (SEQ ID NO://); LQLPPLERLTLD (SEQ ID NO://); LQKKLEELELE (SEQ ID NO://); MTKKFGTLTI (SEQ ID NO://); LAEMLEDLHI (SEQ ID NO://); LDQQFAGLDL (SEQ ID NO://); LCQAFSDVIL (SEQ ID NO://); LPVLENLTL (SEQ ID NO://); and IQQQLGQLTLENLQML (SEQ ID NO://).

Another suitable protein is an estrogen receptor protein. For example, an estrogen receptor protein can comprise an amino acid sequence having at least 85%, at least 90%, at least 95%, at least 98%, at least 99%, or 100%, amino acid sequence identity to the following amino acid sequence: PSAGDMRAANLWPSPLMIKRSKKNSLALSLTADQMVSALLDAEPPILYSEYDPTRPFSEASMMG LLTNLADRELVHMINWAKRVPGFVDLTLHDQVHLLECAWLEILMIGLVWRSMEHPVKLLFAPN LLLDRNQGKCVEGMVEIFDMLLATSSRFRMMNLQGEEFVCLKSIILLNSGVYTFLSSTLKSLEEK DHIHRVLDKITDTLIHLMAKAGLTLQQQHQRLAQLLLILSHIRHMSNKGMEHLYSMKCKNVVP LYDLLLEAADAHRLHAPTSRGGASVEETDQSHLATAGSTSSHSLQKYYITGEAEGFPATA; where the amino acid sequence is a MyoD-ERT2 fusion polypeptide, comprising the ligand-binding domain of estrogen receptor (amino acids 203-440), a basic domain in helix-loop-helix proteins of the MYOD family (amino acids 1-114).

### Binding Inducing Agents

In some cases, the first and the second polypeptides of the protein interaction pair bind to one another in the presence of a small molecule agent. In some cases, the first and the second polypeptides of the protein interaction pair bind to one another in the presence of light of an activating wavelength. In some cases, the first and the second polypeptides of the protein interaction pair bind to one another in the presence of a hormone. In some cases, the first and the second polypeptides of the protein interaction pair bind to one another in the presence of an ion. In some cases, the first and the second polypeptides of the protein interaction pair bind to one another in the presence of a peptide that comprises a portion that binds to the first polypeptide and a portion that binds to the second polypeptide. In some cases, the first and the second polypeptides of the protein interaction pair bind to one another in the presence of a chemical. In some cases, the first and the second polypeptides of the protein interaction pair bind to one another in the presence of a ligand. In some cases, the first and the second polypeptides of the protein interaction pair bind to one another in the presence of a stimulant. In some cases, the first and the second polypeptides of the protein interaction pair bind to one another in the presence of a certain temperature or temperature range. In some cases, the first and the second polypeptides of the protein interaction pair bind to one another in the presence of light of a wavelength that is different from the wavelength(s) of light that activate the LOV domain polypeptide. In some cases, the first and the second polypeptides of the protein interaction pair bind to one another in the presence of a certain pH, or a certain pH range. In some cases, the first and the second polypeptides of the protein interaction pair bind to one another upon exposure of a cell harboring a PPI system of the present disclosure to: i) a ligand; ii) another cell; iii) a cytokine; iv) a chemokine; v) a neurotransmitter; etc.

### Protein Interaction Pairs

In some cases, the first and the second members of protein interaction pair are naturally-occurring polypeptides. In some cases, one or both of the first and the second members of protein interaction pair is a non-naturally-occurring polypeptide, e.g., a recombinant polypeptide made in the laboratory, or mutated compared to a naturally-occurring polypeptide. In some cases, the first member of the protein interaction pair is an N-terminal portion of a polypeptide; and the second member of the protein interaction pair is a C-terminal portion of the polypeptide. In some cases, the first member of the protein interaction pair is a known protein; and the second member of the protein interaction pair is an unknown protein, e.g., a member of a library of proteins. In some cases, the first member of the protein interaction pair is a first known protein that binds to a second known protein, and the second member of the protein interaction pair is a variant of the second known protein.

In some cases, the first or the second member of the protein interaction pair is a protein interaction domain (e.g., the first or the second member of the protein interaction pair is not a full-length protein, but instead is a portion of a full-length protein). Protein interaction domains include, but are not limited to, e.g., a 14-3-3 domain (e.g., as present in PDB (RCSB Protein Data Bank available online at www(dot)rcsb(dot)org) structure 2B05), an Actin-Depolymerizing Factor (ADF) domain (e.g., as present in PDB structure 1CFY), an ANK domain (e.g., as present in PDB structure 1SW6), an ANTH (AP180 N-Terminal Homology) domain (e.g., as present in PDB structure 5AHV), an Armadillo (ARM) domain (e.g., as present in PDB structure 1BK6), a BAR (Bin/Amphiphysin/Rvs) domain (e.g., as present in PDB structure 1I4D), a BEACH (beige and CHS) domain (e.g., as present in PDB structure 1MI1), a BH (Bcl-2 Homology) domains (BH1, BH2, BH3 and BH4) (e.g., as present in PDB structure 1BXL), a Baculovirus IAP Repeat (BIR) domain (e.g., as present in PDB structure 1G73), a BRCT (BRCA1 C-terminal) domain (e.g., as present in PDB structure 1T29), a bromodomain (e.g., as present in PDB structure 1E6I), a BTB (BR-C, ttk and bab) domain (e.g., as present in PDB structure 1R2B), a C1 domain (e.g., as present in PDB structure 1PTQ), a C2 domain (e.g., as present in PDB structure 1A25), a Caspase recruitment domains (CARDs) (e.g., as present in PDB structure 1CWW), a Coiled-coils (CC) domain (e.g., as present in PDB structure 1QEY), a CALM (Clathrin Assembly Lymphoid Myeloid) domain (e.g., as present in PDB structure 1HFA), a calponin homology (CH) domain (e.g., as present in PDB structure 1BKR), a Chromatin Organization Modifier (Chromo) domain (e.g., as present in PDB structure 1KNA), a CUE domain (e.g., as present in PDB structure 1OTR), a Death domains (DD) (e.g., as present in PDB structure 1FAD), a death-effector domain (DED) (e.g., as present in PDB structure 1A1W), a Disheveled, EGL-10 and Pleckstrin (DEP) domain (e.g., as present in PDB structure 1FSH), a Db1 homology (DH) domain (e.g., as present in PDB structure 1FOE), an EF-hand (EFh) domain (e.g., as present in PDB structure 2PMY), an Eps15-Homology (EH) domain (e.g., as present in PDB structure 1EH2), an epsin NH2-terminal homology (ENTH) domain (e.g., as present in PDB structure 1EDU), an Ena/Vasp Homology domain 1 (EVH1) (e.g., as present in PDB structure 1QC6), a F-box domain (e.g., as present in PDB structure 1FS1), a FERM (Band 4.1, Ezrin, Radixin, Moesin) domain (e.g., as present in PDB structure 1GC6), a FF domain (e.g., as present in PDB structure 1UZC), a Formin Homology-2 (FH2) domain (e.g., as present in PDB structure 1UX4), a Forkhead-Associated (FHA) domain (e.g., as present in PDB structure 1G6G), a FYVE (Fab-1, YGL023, Vps27, and EEA1) domain (e.g., as present in PDB structure 1VFY), a GAT (GGA and Tom1) domain (e.g., as present in PDB structure 1O3X), a gelsolin homology domain (GEL) (e.g., as present in PDB structure 1H1V), a GLUE (GRAM-like ubiquitin-binding in EAP45) domain (e.g., as present in PDB structure 2CAY), a GRAM (from glucosyltransferases, Rab-like GTPase activators and myotubularins) domain (e.g., as present in PDB structure 1LW3), a GRIP domain (e.g., as present in PDB structure 1UPT), a glycine-tyrosine-phenylalanine (GYF) domain (e.g., as present in PDB structure 1GYF), a HEAT (Huntington, Elongation Factor 3, PR65/A, TOR) domain (e.g., as present in PDB structure 1IBR), a Homologous to the E6-AP Carboxyl Terminus (HECT) domain (e.g., as present in PDB structure 1C4Z), an IQ domain (e.g., as present in PDB structure 1N2D), a LIM (Lin-1, Isl-1, and Mec-3) domain (e.g., as present in PDB structure 1QLI), a Leucine-Rich Repeats (LRR) domain (e.g., as present in PDB structure 1YRG), a Malignant brain tumor (MBT) domain (e.g., as present in PDB structure 1OYX), a MH1 (Mad homology 1) domain (e.g., as present in PDB structure 1OZJ), a MH2 (Mad homology 2) domain (e.g., as present in PDB structure 1DEV), a MIU (Motif Interacting with Ubiquitin) domain (e.g., as present in PDB structure 2C7M), a NZF (Np14 zinc finger) domain (e.g., as present in PDB structure 1Q5W), a PAS (Per-ARNT-Sim) domain (e.g., as present in PDB structure 1P97), a Phox and Beml (PB 1) domain (e.g., as present in PDB structure 1IPG), a PDZ (postsynaptic density 95, PSD-85; discs large, D1g; zonula occludens-1, ZO-1) domain (e.g., as present in PDB structure 1BE9), a Pleckstrin-homology (PH) domain (e.g., as present in PDB structure 1MAI), a Polo-Box domain (e.g., as present in PDB structure 1Q4K), a Phosphotyrosine binding (PTB) domain (e.g., as present in PDB structure 1SHC), a Pumilio/Puf (PUF) domain (e.g., as present in PDB structure 1M8W), a PWWP domain (e.g., as present in PDB structure 1KHC), a Phox homology (PX) domain (e.g., as present in PDB structure 1H6H), a RGS (Regulator of G protein Signaling) domain (e.g., as present in PDB structure 1AGR), a RING domain (e.g., as present in PDB structure 1FBV), a SAM (Sterile Alpha Motif) domain (e.g., as present in PDB structure 1B0X), a Shadow Chromo (SC) Domain (e.g., as present in PDB structure 1E0B), a Src-homology 2 (SH2) domain (e.g., as present in PDB structure 1SHB), a Src-homology 3 (SH3) domain (e.g., as present in PDB structure 3SEM), a SOCS (supressors of cytokine signaling) domain (e.g., as present in PDB structure 1VCB), a SPRY domain (e.g., as present in PDB structure 2AFJ), a steroidogenic acute regulatory protein (StAR) related lipid transfer (START) domain (e.g., as present in PDB structure 1EM2), a SWIRM domain (e.g., as present in PDB structure 2AQF), a Toll/Il-1 Receptor (TIR) domain (e.g., as present in PDB structure 1FYV), a tetratricopeptide repeat (TPR) domain (e.g., as present in PDB structure 1ELW), a TRAF (Tumor Necrosis Factor (TNF) receptor-associated factors) domain (e.g., as present in PDB structure 1F3V), a tSNARE (SNARE (soluble NSF attachment protein (SNAP) receptor) domain (e.g., as present in PDB structure 1SFC), a Tubby domain (e.g., as present in PDB structure 1I7E), a TUDOR domain (e.g., as present in PDB structure 2GFA), an ubiquitin-associated (UBA) domain (e.g., as present in PDB structure 1IFY), an UEV (Ubiquitin E2 variant) domain (e.g., as present in PDB structure 1S1Q), an ubiquitin-interacting motif (UIM) domain (e.g., as present in PDB structure 1Q0W), a VHL domain (e.g., as present in PDB structure 1LM8), a VHS (Vps27p, Hrs and STAM) domain (e.g., as present in PDB structure 1ELK), a WD40 domain (e.g., as present in PDB structure 1NEX), a WW domain (e.g., as present in PDB structure 1I6C), and the like.

### Protein Interaction Pairs; First Member is Known, Second Member is Unknown

In some cases, the first member of a protein interaction pair is a known protein; and the second member of the protein interaction pair is an unknown protein. For example, in some cases, the first member of a protein interaction pair (which first member may be referred to as a “bait” protein) is a known polypeptide; and the second member of the protein interaction pair (which second member may be referred to as a “prey” protein) is a member of a library of proteins (e.g., a plurality of proteins) of unknown amino acid sequence and/or function.

The known protein can be any of a variety of proteins, where such proteins include membrane proteins, receptors, enzymes, cytoskeletal proteins, regulatory proteins, transcription factors, and the like.

The unknown protein can be a member of a protein library, where the protein library can have from 10 to 10⁹ protein members, e.g., from 10 proteins to 10² proteins, from 10² proteins to 10³ proteins, from 10³ proteins to 10⁴ proteins, from 10⁴ proteins to 10⁵ proteins, from 10⁵ proteins to 10⁶ proteins, from 10⁶ proteins to 10⁷ proteins, from 10⁷ proteins to 10⁸ proteins, or from 10⁸ proteins to 10⁹ proteins. In some cases, the library has more than 10⁹ proteins.

The library can be a library of proteins from a particular organism. For example, a library can be a library of proteins of, e.g., Bacteria (e.g., Eubacteria); Archaebacteria; Protista; Fungi; Plantae; and Animalia. A library can be a library of proteins of plant-like members of the kingdom Protista, including, but not limited to, algae (e.g., green algae, red algae, glaucophytes, cyanobacteria); fungus-like members of Protista, e.g., slime molds, water molds, etc.; animal-like members of Protista, e.g., flagellates (e.g., Euglena), amoeboids (e.g., amoeba), sporozoans (e.g, Apicomplexa, Myxozoa, Microsporidia), and ciliates (e.g., Paramecium). A library can be a library of proteins of the kingdom Fungi, including, but not limited to, members of any of the phyla: Basidiomycota (club fungi; e.g., members of Agaricus, Amanita, Boletus, Cantherellus, etc.); Ascomycota (sac fungi, including, e.g., Saccharomyces); Mycophycophyta (lichens); Zygomycota (conjugation fungi); and Deuteromycota. A library can be a library of proteins of a member of the kingdom Plantae, including, but not limited to, members of any of the following divisions: Bryophyta (e.g., mosses), Anthocerotophyta (e.g., hornworts), Hepaticophyta (e.g., liverworts), Lycophyta (e.g., club mosses), Sphenophyta (e.g., horsetails), Psilophyta (e.g., whisk ferns), Ophioglossophyta, Pterophyta (e.g., ferns), Cycadophyta, Gingkophyta, Pinophyta, Gnetophyta, and Magnoliophyta (e.g., flowering plants). A library can be a library of proteins of a member of the kingdom Animalia, including, but not limited to, members of any of the following phyla: Porifera (sponges); Placozoa; Orthonectida (parasites of marine invertebrates); Rhombozoa; Cnidaria (corals, anemones, jellyfish, sea pens, sea pansies, sea wasps); Ctenophora (comb jellies); Platyhelminthes (flatworms); Nemertina (ribbon worms); Ngathostomulida (jawed worms)p Gastrotricha; Rotifera; Priapulida; Kinorhyncha; Loricifera; Acanthocephala; Entoprocta; Nemotoda; Nematomorpha; Cycliophora; Mollusca (mollusks); Sipuncula (peanut worms); Annelida (segmented worms); Tardigrada (water bears); Onychophora (velvet worms); Arthropoda (including the subphyla: Chelicerata, Myriapoda, Hexapoda, and Crustacea, where the Chelicerata include, e.g., arachnids, Merostomata, and Pycnogonida, where the Myriapoda include, e.g., Chilopoda (centipedes), Diplopoda (millipedes), Paropoda, and Symphyla, where the Hexapoda include insects, and where the Crustacea include shrimp, krill, barnacles, etc.; Phoronida; Ectoprocta (moss animals); Brachiopoda; Echinodermata (e.g. starfish, sea daisies, feather stars, sea urchins, sea cucumbers, brittle stars, brittle baskets, etc.); Chaetognatha (arrow worms); Hemichordata (acorn worms); and Chordata. Suitable members of Chordata include any member of the following subphyla: Urochordata (sea squirts; including Ascidiacea, Thaliacea, and Larvacea); Cephalochordata (lancelets); Myxini (hagfish); and Vertebrata, where members of Vertebrata include, e.g., members of Petromyzontida (lampreys), Chondrichthyces (cartilaginous fish), Actinopterygii (ray-finned fish), Actinista (coelocanths), Dipnoi (lungfish), Reptilia (reptiles, e.g., snakes, alligators, crocodiles, lizards, etc.), Aves (birds); and Mammalian (mammals). A library can be a library of proteins of any monocotyledon and cells of any dicotyledon.

A library can be a library of proteins of a diseased cell or organism. For example, a protein library can be a library of proteins from a cancer cell, from a muscle cell comprising a defect in a muscle protein, and the like. A library can be a library of proteins of a healthy cell or organism.

A library can be a library of proteins of a cell or organism that has been exposed to any of a variety of stimuli, stresses, etc.

In some cases, any one of the aforementioned libraries is barcoded. In instances where barcode identification and/or quantification is performed by sequencing, including e.g., Next Generation Sequencing methods, conventional considerations for barcodes detected by sequencing will be applied. In some instances, commercially available barcodes and/or kits containing barcodes and/or barcode adapters may be used or modified for use in the methods described herein, including e.g., those barcodes and/or barcode adapter kits commercially available from suppliers such as but not limited to, e.g., New England Biolabs (Ipswich, Mass.), Illumina, Inc. (Hayward, Calif.), Life Technologies, Inc. (Grand Island, N.Y.), Bioo Scientific Corporation (Austin, Tex.), and the like, or may be custom manufactured, e.g., as available from e.g., Integrated DNA Technologies, Inc. (Coralville, Iowa).

Barcode length will vary and will depend upon the complexity of the library and the barcode detection method utilized. As nucleic acid barcodes (e.g., DNA barcodes) are well-known, design, synthesis and use of nucleic acid barcodes is within the skill of the ordinary relevant artisan.

### Protein Interaction Pairs; First Member is a Known Protein, Second Member is a Variant of a Reference Protein

In some cases, the first member of a protein interaction pair is a known protein; and the second member of the protein interaction pair is a variant of a reference protein (e.g., a variant of a naturally-occurring protein; a known protein; etc.). For example, in some cases, the first member of the protein interaction pair is a first known protein that binds to a second known protein, and the second member of the protein interaction pair is a variant of the second known protein. For example, in some cases, the first member of a protein interaction pair (which first member may be referred to as a “bait” protein) is a known polypeptide; and the second member of the protein interaction pair comprises one or more amino acid changes (e.g., substitutions, insertions, deletions, etc.) relative to a reference protein.

In some cases, the second member of the protein interaction pair is a member of a library of proteins (“variant proteins”), each of which contains a single amino acid substitution relative to a reference protein, where the reference protein that is known to interact with the first member of the protein interaction pair. The variant protein library can have from 10 to 10⁹ protein members, e.g., from 10 proteins to 10² proteins, from 10² proteins to 10³ proteins, from 10³ proteins to 10⁴ proteins, from 10⁴ proteins to 10⁵ proteins, from 10⁵ proteins to 10⁶ proteins, from 10⁶ proteins to 10⁷ proteins, from 10⁷ proteins to 10⁸ proteins, or from 10⁸ proteins to 10⁹ proteins. In some cases, the library has more than 10⁹ proteins.

In some cases, a single amino acid in a variant protein is mutated relative to the reference protein.

In some cases, the single amino acid is mutated to a different coded amino acid; for example, a library can comprise variant proteins, each of which contains substitution of a single amino acid to a different coded amino acid. For example, a protein variant library can comprise: a first member comprising a first substitution of amino acid X of the reference protein; a second member comprising a second substitution of amino acid X of the reference protein; a third member comprising a third substitution of amino acid X of the reference protein; etc., such that the library comprises all possible substitutions of amino acid X of the reference protein.

In other cases, a library of variant proteins comprises members each of which comprises a single amino acid substitution in a different amino acid of the reference protein. For example, where a reference protein comprises 200 amino acids, a library of variant proteins can comprise a first member comprising a substitution of amino acid 1 of the reference protein; a second member comprising a substitution of amino acid 2 of the reference protein; a third member comprising a substitution of amino acid 3 of the reference protein; etc., such that variants of each of the 200 amino acids is represented in the library.

The variant protein library can comprise members each of which comprises a different amino acid substitution in a different amino acid of the reference protein. For example, where a reference protein comprises 200 amino acids, a library of variant proteins can comprise: A) a first member comprising a first substitution of amino acid 1 of the reference protein; a second member comprising a second substitution of amino acid 1 of the reference protein; etc., up to a 19^{th} member comprising a 19^{th} substitution of amino acid 1 of the reference protein, such that the library comprises all possible substitutions of amino acid 1 of the reference protein; B) a 20th member comprising a first substitution of amino acid 2 of the reference protein; a 21st member comprising a second substitution of amino acid 2 of the reference protein; etc., such that the library comprises all possible substitutions of amino acid 2 of the reference protein; etc., such that the variant protein library contains individual members, where, for each amino acid of the reference protein, the library comprises a plurality of members each of which comprises a single amino acid substitution covering all possible substitutions (e.g., all coded amino acids) of each amino acid in the reference protein. Such a library could include, e.g., 3800 members (200 amino acid positions×19 amino acids).

As another example, in some cases, the second member of the protein interaction pair is a member of a library of proteins, each of which contains from 2 to 5 amino acid substitutions substitution relative to a reference protein that is known to interact with the first member of the protein interaction pair. In some cases, the from 2 to 5 amino acid substitutions are random. In some cases, the from 2 to 5 amino acid substitutions are in defined locations of a reference protein.

As another example, in some cases, the second member of the protein interaction pair is a member of a library of proteins, each of which contains an insertion (e.g., an insertion of 1, 2, 3, 4, 5, 6, 7, 8, 9, or 10 amino acids) at a different site relative to a reference protein that is known to interact with the first member of the protein interaction pair.

In some cases, any one of the aforementioned libraries is barcoded. In instances where barcode identification and/or quantification is performed by sequencing, including e.g., Next Generation Sequencing methods, conventional considerations for barcodes detected by sequencing will be applied. In some instances, commercially available barcodes and/or kits containing barcodes and/or barcode adapters may be used or modified for use in the methods described herein, including e.g., those barcodes and/or barcode adapter kits commercially available from suppliers such as but not limited to, e.g., New England Biolabs (Ipswich, Mass.), Illumina, Inc. (Hayward, Calif.), Life Technologies, Inc. (Grand Island, N.Y.), Bioo Scientific Corporation (Austin, Tex.), and the like, or may be custom manufactured, e.g., as available from e.g., Integrated DNA Technologies, Inc. (Coralville, Iowa).

Barcode length will vary and will depend upon the complexity of the library and the barcode detection method utilized. As nucleic acid barcodes (e.g., DNA barcodes) are well-known, design, synthesis and use of nucleic acid barcodes is within the skill of the ordinary relevant artisan.

Protein Interaction Pairs; Known Protein Interaction Pairs

In some cases, the first and the second members of the protein interaction pair are polypeptides that are known to interact with one another in the presence of a binding-inducing agent.

Examples of known protein interaction polypeptides include, but are not limited to:

a) FK506 binding protein (FKBP) and FKBP;

b) FKBP and calcineurin catalytic subunit A (CnA);

c) FKBP and cyclophilin;

d) FKBP and FKBP-rapamycin associated protein (FRB);

e) gyrase B (GyrB) and GyrB;

f) dihydrofolate reductase (DHFR) and DHFR;

g) DmrB and DmrB;

h) PYL and ABI;

i) Cry2 and CIB1;

j) GAI and GID1;

k) mineralcorticoid receptor (MR) ligand-binding domain (LBD) and an SRC1-2 peptide;

l) a PPAR-γ LBD and an SRC1 peptide;

m) an androgen receptor LBF and an SRC3-1 peptide;

n) a PPAR-γ LBD and an SRC3 peptide;

o) an MR LBD and a PGC1a peptide;

p) an MR LBD and a TRAP220-1 peptide;

q) a progesterone receptor LBD and an NCoR peptide;

r) an estrogen receptor-β LBD and an NR0B1 peptide;

s) a PPAR-γ LBD and a TIF2 peptide;

t) an ERα LBD and a CoRNR box peptide;

u) an ERα LBD and an abV peptide;

v) a G protein-coupled receptor (GPCR) and a G protein;

w) a GPCR and a beta-arrestin polypeptide;

x) an epidermal growth factor receptor (EGFR) and Src/Shc/Grb2;

y) calmodulin and calmodulin binding polypeptide; and

z) troponin C and troponin I.

FKBP/FRB Protein Interaction Pair

In some cases, a first or a second polypeptide of a protein interaction pair is an FKBP. In some cases, a suitable FKBP comprises an amino acid sequence having at least about 75%, at least about 80%, at least about 85%, at least about 90%, at least about 95%, at least about 98%, or 100% amino acid sequence identity to the following amino acid sequence:

(SEQ ID NO: //)
MGVQVETISPGDGRTFPKRGQTCVVHYTGMLEDGKKFDSSRDRNKPFKFM
LGKQEVIRGWEEGVAQMSVGQRAKLTISPDYAYGATGHPGIIPPHATLVF
DVELLKLE.

FKBP and Calcineurin Catalytic Subunit A (CnA) Protein Interaction Pair

In some cases, a first or a second polypeptide of a protein interaction pair is a calcineurin catalytic subunit A polypeptide (also known as PPP3CA; CALN; CALNA; CALNA1; CCN1; CNA1; PPP2B; CAM-PRP catalytic subunit; calcineurin A alpha; calmodulin-dependent calcineurin A subunit alpha isoform; protein phosphatase 2B, catalytic subunit, alpha isoform; etc.). For example, a suitable calcineurin catalytic subunit A polypeptide can comprise an amino acid sequence having at least about 75%, at least about 80%, at least about 85%, at least about 90%, at least about 95%, at least about 98%, or 100% amino acid sequence identity to the following amino acid sequence (PP2Ac domain):

(SEQ ID NO: //)
LEESVALRIITEGASILRQEKNLLDIDAPVTVCGDIHGQFFDLMKLFEVG
GSPANTRYLFLGDYVDRGYFSIECVLYLWALKILYPKTLFLLRGNHECRH
LTEYFTFKQECKIKYSERVYDACMDAFDCLPLAALMNQQFLCVHGGLSPE
INTLDDIRKLDRFKEPPAYGPMCDILWSDPLEDFGNEKTQEHFTHNTVRG
CSYFYSYPAVCEFLQHNNLLSILRAHEAQDAGYRMYRKSQTTGFPSLITI
FSAPNYLDVYNNKAAVLKYENNVMNIRQFNCSPHPYWLPNFM.

FKBP/Cyclophilin Protein Interaction Pair

In some cases, a first or a second polypeptide of a protein interaction pair is a cyclophilin polypeptide (also known cyclophilin A, PPIA, CYPA, CYPH, PPIase A, etc.). For example, a suitable cyclophilin polypeptide can comprise an amino acid sequence having at least about 75%, at least about 80%, at least about 85%, at least about 90%, at least about 95%, at least about 98%, or 100% amino acid sequence identity to the following amino acid sequence:

(SEQ ID NO: //)
MVNPTVFFDIAVDGEPLGRVSFELFADKVPKTAENFRALSTGEKGFGYKG
SCFHRIIPGFMCQGGDFTRHNGTGGKSIYGEKFEDENFILKHTGPGILSM
ANAGPNTNGSQFFICTAKTEWLDGKHVVFGKVKEGMNIVEAMERFGSRNG
KTSKKITIADCGQLE.

FKBP/MTOR Protein Interaction Pair

In some cases, a first or a second polypeptide of a protein interaction pair is a MTOR polypeptide (also known as FKBP-rapamycin associated protein; FK506 binding protein 12-rapamycin associated protein 1; FK506 binding protein 12-rapamycin associated protein 2; FK506-binding protein 12-rapamycin complex-associated protein 1; FRAP; FRAP1; FRAP2; RAFT1; and RAPT1). For example, a suitable MTOR polypeptide can comprise an amino acid sequence having at least about 75%, at least about 80%, at least about 85%, at least about 90%, at least about 95%, at least about 98%, or 100% amino acid sequence identity to the following amino acid sequence (also known as “Frb”: Fkbp-Rapamycin Binding Domain):

(SEQ ID NO: //)
MILWHEMWHEGLEEASRLYFGERNVKGMFEVLEPLHAMMERGPQTLKETS
FNQAYGRDLMEAQEWCRKYMKSGNVKDLLQAWDLYYHVFRRISK.

GyrB/GyrB Protein Interaction Pair

In some cases, a first and a second polypeptide of a protein interaction pair is a GyrB polypeptide (also known as DNA gyrase subunit B). For example, a suitable GyrB polypeptide can comprise an amino acid sequence having at least about 75%, at least about 80%, at least about 85%, at least about 90%, at least about 95%, at least about 98%, or 100% amino acid sequence identity to a contiguous stretch of from about 100 amino acids to about 200 amino acids (aa), from about 200 aa to about 300 aa, from about 300 aa to about 400 aa, from about 400 aa to about 500 aa, from about 500 aa to about 600 aa, from about 600 aa to about 700 aa, or from about 700 aa to about 800 aa, of the following GyrB amino acid sequence from Escherichia coli (or to the DNA gyrase subunit B sequence from any organism):

MSNSYDSSSIKVLKGLDAVRKRPGMYIGDTDDGTGLHHMVFEVVDNAIDEALAGHCKE IIVTIHADNSVSVQDDGRGIPTGIHPEEGVSAAEVIMTVLHAGGKFDDNSYKVSGGLHGVGVSV VNALSQKLELVIQREGKIHRQIYEHGVPQAPLAVTGETEKTGTMVRFWPSLETFTNVTEFEYEIL AKRLRELSFLNSGVSIRLRDKRDGKEDHFHYEGGIKAFVEYLNKNKTPIHPNIFYFSTEKDGIGVE VALQWNDGFQENIYCFTNNIPQRDGGTHLAGFRAAMTRTLNAYMDKEGYSKKAKVSATGDD AREGLIAVVSVKVPDPKFSSQTKDKLVSSEVKSAVEQQMNELLAEYLLENPTDAKIVVGKIIDA ARAREAARRAREMTRRKGALDLAGLPGKLADCQERDPALSELYLVEGDSAGGSAKQGRNRKN QAILPLKGKILNVEKARFDKMLSSQEVATLITALGCGIGRDEYNPDKLRYHSIIIMTDADVDGSHI RTLLLTFFYRQMPEIVERGHVYIAQPPLYKVKKGKQEQYIKDDEAMDQYQISIALDGATLHTNA SAPALAGEALEKLVSEYNATQKMINRMERRYPKAMLKELIYQPTLTEADLSDEQTVTRWVNAL VSELNDKEQHGSQWKFDVHTNAEQNLFEPIVRVRTHGVDTDYPLDHEFITGGEYRRICTLGEKL RGLLEEDAFIERGERRQPVASFEQALDWLVKESRRGLSIQRYKGLGEMNPEQLWETTMDPESRR MLRVTVKDAIAADQLFTTLMGDAVEPRRAFIEENALKAANIDI (SEQ ID NO://). In some cases, a suitable GyrB polypeptide comprises an amino acid sequence having at least about 75%, at least about 80%, at least about 85%, at least about 90%, at least about 95%, at least about 98%, or 100% amino acid sequence identity to amino acids 1-220 of the above-listed GyrB amino acid sequence from Escherichia coli.

DHFR/DYR Protein Interaction Pair

In some cases, a first polypeptide or a second polypeptide of a protein interaction pair is a DHFR polypeptide (also known as dihydrofolate reductase, DHFRP1, and DYR). For example, a suitable DHFR polypeptide can comprise an amino acid sequence having at least about 75%, at least about 80%, at least about 85%, at least about 90%, at least about 95%, at least about 98%, or 100% amino acid sequence identity to the following amino acid sequence:

(SEQ ID NO: //)
MVGSLNCIVAVSQNMGIGKNGDLPWPPLRNEFRYFQRMTTTSSVEGKQNL
VIMGKKTWFSIPEKNRPLKGRINLVLSRELKEPPQGAHFLSRSLDDALKL
TEQPELANKVDMVWIVGGSSVYKEAMNHPGHLKLFVTRIMQDFESDTFFP
EIDLEKYKLLPEYPGVLSDVQEEKGIKYKFEVYEKND.

DmrB/DmrB Protein Interaction Pair

In some cases, a first and a second polypeptide of a protein interaction pair is a DmrB polypeptide. For example, a suitable DmrB polypeptide can comprise an amino acid sequence having at least about 75%, at least about 80%, at least about 85%, at least about 90%, at least about 95%, at least about 98%, or 100% amino acid sequence identity to the following amino acid sequence: MASRGVQVETISPGDGRTFPKRGQTCVVHYTGMLEDGKKVDSSRDRNKPFKFMLGKQEVIRG WEEGVAQMSVGQRAKLTISPDYAYGATGHPGIIPPHATLVFDVELLKLE (SEQ ID NO://).

### PYL/ABI Protein Interaction Pair

In some cases, a first polypeptide or a second polypeptide of a protein interaction pair is a PYL polypeptide (also known as abscisic acid receptor and as RCAR). For example a suitable PYL polypeptide can be derived from proteins such as those of Arabidopsis thaliana: PYR1, RCAR1(PYL9), PYL1, PYL2, PYL3, PYL4, PYL5, PYL6, PYL7, PYL8 (RCAR3), PYL10, PYL11, PYL12, PYL13. For example, a suitable PYL polypeptide can comprise an amino acid sequence having at least about 75%, at least about 80%, at least about 85%, at least about 90%, at least about 95%, at least about 98%, or 100% amino acid sequence identity to any one of the following amino acid sequences:

PYL10:
(SEQ ID NO: //)
MNGDETKKVESEYIKKHHRHELVESQCSSTLVKHIKAPLHLVWSIVRRFD
EPQKYKPFISRCVVQGKKLEVGSVREVDLKSGLPATKSTEVLEILDDNEH
ILGIRIVGGDHRLKNYSSTISLHSETIDGKTGTLAIESFVVDVPEGNTKE
ETCFFVEALIQCNLNSLADVTERLQAESMEKKI;
PYL11:
(SEQ ID NO: /)
METSQKYHTCGSTLVQTIDAPLSLVWSILRRFDNPQAYKQFVKTCNLSSG
DGGEGSVREVTVVSGLPAEFSRERLDELDDESHVMMISIIGGDHRLVNYR
SKTMAFVAADTEEKTVVVESYVVDVPEGNSEEETTSFADTIVGFNLKSLA
KLSERVAHLKL;
PYL12:
(SEQ ID NO: //)
MKTSQEQHVCGSTVVQTINAPLPLVWSILRRFDNPKTFKHFVKTCKLRSG
DGGEGSVREVTVVSDLPASFSLERLDELDDESHVMVISIIGGDHRLVNYQ
SKTTVFVAAEEEKTVVVESYVVDVPEGNTEEETTLFADTIVGCNLRSLAK
LSEKMMELT;
PYL13:
(SEQ ID NO: //)
MESSKQKRCRSSVVETIEAPLPLVWSILRSFDKPQAYQRFVKSCTMRSGG
GGGKGGEGKGSVRDVTLVSGFPADFSTERLEELDDESHVMVVSIIGGNHR
LVNYKSKTKVVASPEDMAKKTVVVESYVVDVPEGTSEEDTIFFVDNIIRY
NLTSLAKLTKKMMK;
PYL1:
(SEQ ID NO: //)
MANSESSSSPVNEEENSQRISTLHHQTMPSDLTQDEFTQLSQSIAEFHTY
QLGNGRCSSLLAQRIHAPPETVWSVVRRFDRPQIYKHFIKSCNVSEDFEM
RVGCTRDVNVISGLPANTSRERLDLLDDDRRVTGFSITGGEHRLRNYKSV
TTVHRFEKEEEEERIWTVVLESYVVDVPEGNSEEDTRLFADTVIRLNLQK
LASITEAMNRNNNNNNSSQVR;
PYL2:
(SEQ ID NO: //)
MSSSPAVKGLTDEEQKTLEPVIKTYHQFEPDPTTCTSLITQRIHAPASVV
WPLIRRFDNPERYKHFVKRCRLISGDGDVGSVREVTVISGLPASTSTERL
EFVDDDHRVLSFRVVGGEHRLKNYKSVTSVNEFLNQDSGKVYTVVLESYT
VDIPEGNTEEDTKMFVDTVVKLNLQKLGVAATSAPMHDDE;
PYL3:
(SEQ ID NO: //)
MNLAPIHDPSSSSTTTTSSSTPYGLTKDEFSTLDSIIRTHHTFPRSPNTC
TSLIAHRVDAPAHAIWRFVRDFANPNKYKHFIKSCTIRVNGNGIKEIKVG
TIREVSVVSGLPASTSVEILEVLDEEKRILSFRVLGGEHRLNNYRSVTSV
NEFVVLEKDKKKRVYSVVLESYIVDIPQGNTEEDTRMFVDTVVKSNLQNL
AVISTASPT;
PYL4:
(SEQ ID NO: //)
MLAVHRPSSAVSDGDSVQIPMMIASFQKRFPSLSRDSTAARFHTHEVGPN
QCCSAVIQEISAPISTVWSVVRRFDNPQAYKHFLKSCSVIGGDGDNVGSL
RQVHVVSGLPAASSTERLDILDDERHVISFSVVGGDHRLSNYRSVTTLHP
SPISGTVVVESYVVDVPPGNTKEETCDFVDVIVRCNLQSLAKIAENTAAE
SKKKMSL;
PYL5:
(SEQ ID NO: //)
MRSPVQLQHGSDATNGFHTLQPHDQTDGPIKRVCLTRGMHVPEHVAMHHT
HDVGPDQCCSSVVQMIHAPPESVWALVRRFDNPKVYKNFIRQCRIVQGDG
LHVGDLREVMVVSGLPAVSSTERLEILDEERHVISFSVVGGDHRLKNYRS
VTTLHASDDEGTVVVESYIVDVPPGNTEEETLSFVDTIVRCNLQSLARST
NRQ;
PYL6:
(SEQ ID NO: //)
MPTSIQFQRSSTAAEAANATVRNYPHHHQKQVQKVSLTRGMADVPEHVEL
SHTHVVGPSQCFSVVVQDVEAPVSTVWSILSRFEHPQAYKHFVKSCHVVI
GDGREVGSVREVRVVSGLPAAFSLERLEIMDDDRHVISFSVVGGDHRLMN
YKSVTTVHESEEDSDGKKRTRVVESYVVDVPAGNDKEETCSFADTIVRCN
LQSLAKLAENTSKFS;
PYL7:
(SEQ ID NO: //)
MEMIGGDDTDTEMYGALVTAQSLRLRHLHHCRENQCTSVLVKYIQAPVHL
VWSLVRRFDQPQKYKPFISRCTVNGDPEIGCLREVNVKSGLPATTSTERL
EQLDDEEHILGINIIGGDHRLKNYSSILTVHPEMIDGRSGTMVMESFVVD
VPQGNTKDDTCYFVESLIKCNLKSLACVSERLAAQDITNSIATFCNASNG
YREKNHTETNL;
PYL8:
(SEQ ID NO: //)
MEANGIENLTNPNQEREFIRRHHKHELVDNQCSSTLVKHINAPVHIVWSL
VRRFDQPQKYKPFISRCVVKGNMEIGTVREVDVKSGLPATRSTERLELLD
DNEHILSIRIVGGDHRLKNYSSIISLHPETIEGRIGTLVIESFVVDVPEG
NTKDETCYFVEALIKCNLKSLADISERLAVQDTTESRV;
PYL9:
Client Rel. S174/56
(SEQ ID NO: //)
MMDGVEGGTAMYGGLETVQYVRTHHQHLCRENQCTSALVKHIKAPLHLVW
SLVRRFDQPQKYKPFVSRCTVIGDPEIGSLREVNVKSGLPATTSTERLEL
LDDEEHILGIKIIGGDHRLKNYSSILTVHPEIIEGRAGTMVIESFVVDVP
QGNTKDETCYFVEALIRCNLKSLADVSERLASQDITQ;
and
PYR1:
(SEQ ID NO: //)
MPSELTPEERSELKNSIAEFHTYQLDPGSCSSLHAQRIHAPPELVWSIVR
RFDKPQTYKHFIKSCSVEQNFEMRVGCTRDVIVISGLPANTSTERLDILD
DERRVTGFSIIGGEHRLTNYKSVTTVHRFEKENRIWTVVLESYVVDMPEG
NSEDDTRMFADTVVKLNLQKLATVAEAMARNSGDGSGSQVT.

In some cases, a first polypeptide or a second polypeptide of a protein interaction pair is an ABI polypeptide (also known as Abscisic Acid-Insensitive). For example, a ABI polypeptide can be an ABI polypeptide of Arabidopsis thaliana: ABI1 (Also known as ABSCISIC ACID-INSENSITIVE 1, Protein phosphatase 2C 56, AtPP2C56, P2C56, and PP2C ABI1) and/or ABI2 (also known as P2C77, Protein phosphatase 2C 77, AtPP2C77, ABSCISIC ACID-INSENSITIVE 2, Protein phosphatase 2C ABI2, and PP2C ABI2). For example, a suitable ABI polypeptide can comprise an amino acid sequence having at least about 75%, at least about 80%, at least about 85%, at least about 90%, at least about 95%, at least about 98%, or 100% amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, from about 150 aa to about 160 aa, from about 160 aa to about 170 aa, from about 170 aa to about 180 aa, from about 180 aa to about 190 aa, or from about 190 aa to about 200 aa of any one of the following amino acid sequences:

ABI1:
(SEQ ID NO: //)
MEEVSPAIAGPFRPFSETQMDFTGIRLGKGYCNNQYSNQDSENGDLMVSL
PETSSCSVSGSHGSESRKVLISRINSPNLNMKESAAADIVVVDISAGDEI
NGSDITSEKKMISRTESRSLFEFKSVPLYGFTSICGRRPEMEDAVSTIPR
FLQSSSGSMLDGRFDPQSAAHFFGVYDGHGGSQVANYCRERMHLALAEEI
AKEKPMLCDGDTWLEKWKKALFNSFLRVDSEIESVAPETVGSTSVVAVVF
PSHIFVANCGDSRAVLCRGKTALPLSVDHKPDREDEAARIEAAGGKVIQW
NGARVFGVLAMSRSIGDRYLKPSIIPDPEVTAVKRVKEDDCLILASDGVW
DVMTDEEACEMARKRILLWHKKNAVAGDASLLADERRKEGKDPAAMSAAE
YLSKLAIQRGSKDNISVVVVDLKPRRKLKSKPLN;
and
ABI2:
(SEQ ID NO: //)
MDEVSPAVAVPFRPFTDPHAGLRGYCNGESRVTLPESSCSGDGAMKDSSF
EINTRQDSLTSSSSAMAGVDISAGDEINGSDEFDPRSMNQSEKKVLSRTE
SRSLFEFKCVPLYGVTSICGRRPEMEDSVSTIPRFLQVSSSSLLDGRVTN
GFNPHLSAHFFGVYDGHGGSVANYCRERMHLALTEEIVKEKPEFCDGDTW
QEKWKKALFNSFMRVDSEIETVAHAPETVGSTSVVAVVFPTHIFVANCGD
SRAVLCRGKTPLALSVDHKPDRDDEAARIEAAGGKVIRWNGARVFGVLAM
SRSIGDRYLKPSVIPDPEVTSVRRVKEDDCLILASDGLWDVMTNEEVCDL
ARKRILLWHKKNAMAGEALLPAEKRGEGKDPAAMSAAEYLSKMALQKGSK
DNISVVVVDLKGIRKFKSKSLN.

GAI and GID1 Protein Interaction Pair

In some cases, a first polypeptide or a second polypeptide of a protein interaction pair is a GAI polypeptide (also known as Gibberellic Acid Insensitive, and DELLA protein GAI). For example, a suitable GAI polypeptide can comprise an amino acid sequence having at least about 75%, at least about 80%, at least about 85%, at least about 90%, at least about 95%, at least about 98%, or 100% amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, from about 150 aa to about 160 aa, from about 160 aa to about 170 aa, from about 170 aa to about 180 aa, from about 180 aa to about 190 aa, or from about 190 aa to about 200 aa of the following amino acid sequence:

(SEQ ID NO: //)
MKRDHHHHHHQDKKTMMMNEEDDGNGMDELLAVLGYKVRSSEMADVAQKL
EQLEVMMSNVQEDDLSQLATETVHYNPAELYTWLDSMLTDLNPPSSNAEY
DLKAIPGDAILNQFAIDSASSSNQGGGGDTYTTNKRLKCSNGVVETTTAT
AESTRHVVLVDSQENGVRLVHALLACAEAVQKENLTVAEALVKQIGFLAV
SQIGAMRKVATYFAEALARRIYRLSPSQSPIDHSLSDTLQMHFYETCPYL
KFAHFTANQAILEAFQGKKRVHVIDFSMSQGLQWPALMQALALRPGGPPV
FRLTGIGPPAPDNFDYLHEVGCKLAHLAEAIHVEFEYRGFVANTLADLDA
SMLELRPSEIESVAVNSVFELHKLLGRPGAIDKVLGVVNQIKPEIFTVVE
QESNHNSPIFLDRFTESLHYYSTLFDSLEGVPSGQDKVMSEVYLGKQICN
VVACDGPDRVERHETLSQWRNRFGSAGFAAAHIGSNAFKQASMLLALFNG
GEGYRVEESDGCLMLGWHTRPLIATSAWKLSTN.

In some cases, a first polypeptide or a second polypeptide of a protein interaction pair is a GID1 polypeptide. In some cases, a suitable GID1 polypeptide is derived from a GID1 Arabidopsis thaliana protein (also known as Gibberellin receptor GID1). For example, a suitable GID1 polypeptide can comprise an amino acid sequence having at least about 75%, at least about 80%, at least about 85%, at least about 90%, at least about 95%, at least about 98%, or 100% amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, from about 150 aa to about 160 aa, from about 160 aa to about 170 aa, from about 170 aa to about 180 aa, from about 180 aa to about 190 aa, or from about 190 aa to about 200 aa of any one of the following amino acid sequences:

GID1A:
(SEQ ID NO: //)
MAASDEVNLIESRTVVPLNTWVLISNFKVAYNILRRPDGTFNRHLA
EYLDRKVTANANPVDGVFSFDVLIDRRINLLSRVYRPAYADQEQPP
SILDLEKPVDGDIVPVILFFHGGSFAHSSANSAIYDTLCRRLVGLC
KCVVVSVNYRRAPENPYPCAYDDGWIALNWVNSRSWLKSKKDSKVH
IFLAGDSSGGNIAHNVALRAGESGIDVLGNILLNPMFGGNERTESE
KSLDGKYFVTVRDRDWYWKAFLPEGEDREHPACNPFSPRGKSLEGV
SFPKSLVVVAGLDLIRDWQLAYAEGLKKAGQEVKLMHLEKATVGFY
LLPNNNHFHNVMDEISAFVNAEC;
GID1B:
(SEQ ID NO: //)
MAGGNEVNLNECKRIVPLNTWVLISNFKLAYKVLRRPDGSFNRDLA
EFLDRKVPANSFPLDGVFSFDHVDSTTNLLTRIYQPASLLHQTRHG
TLELTKPLSTTEIVPVLIFFHGGSFTHSSANSAIYDTFCRRLVTIC
GVVVVSVDYRRSPEHRYPCAYDDGWNALNWVKSRVWLQSGKDSNVY
VYLAGDSSGGNIAHNVAVRATNEGVKVLGNILLHPMFGGQERTQSE
KTLDGKYFVTIQDRDWYWRAYLPEGEDRDHPACNPFGPRGQSLKGV
NFPKSLVVVAGLDLVQDWQLAYVDGLKKTGLEVNLLYLKQATIGFY
FLPNNDHFHCLMEELNKFVHSIEDSQSKSSPVLLTP;
and
GID1C:
(SEQ ID NO: //)
MAGSEEVNLIESKTVVPLNTWVLISNFKLAYNLLRRPDGTFNRHLA
EFLDRKVPANANPVNGVFSFDVIIDRQTNLLSRVYRPADAGTSPSI
TDLQNPVDGEIVPVIVFFHGGSFAHSSANSAIYDTLCRRLVGLCGA
VVVSVNYRRAPENRYPCAYDDGWAVLKWVNSSSWLRSKKDSKVRIF
LAGDSSGGNIVHNVAVRAVESRIDVLGNILLNPMFGGTERTESEKR
LDGKYFVTVRDRDWYWRAFLPEGEDREHPACSPFGPRSKSLEGLSF
PKSLVVVAGLDLIQDWQLKYAEGLKKAGQEVKLLYLEQATIGFYLL
PNNNHFHTVMDEIAAFVNAECQ.

In some cases, a first polypeptide or a second polypeptide of a protein interaction pair is a Cry2 polypeptide (also known as cryptochrome 2). For example, a suitable Cry2 polypeptide can comprise an amino acid sequence having at least about 75%, at least about 80%, at least about 85%, at least about 90%, at least about 95%, at least about 98%, or 100% amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, from about 150 aa to about 160 aa, from about 160 aa to about 170 aa, from about 170 aa to about 180 aa, from about 180 aa to about 190 aa, or from about 190 aa to about 200 aa of the following amino acid sequence:

Cry2 (Arabidopsis thaliana)
(SEQ ID NO: //)
MKMDKKTIVWFRRDLRIEDNPALAAAAHEGSVFPVFIWCPEEEGQF
YPGRASRWWMKQSLAHLSQSLKALGSDLTLIKTHNTISAILDCIRV
TGATKVVFNHLYDPVSLVRDHTVKEKLVERGISVQSYNGDLLYEPW
EIYCEKGKPFTSFNSYWKKCLDMSIESVMLPPPWRLMPITAAAEAI
WACSIEELGLENEAEKPSNALLTRAWSPGWSNADKLLNEFIEKQLI
DYAKNSKKVVGNSTSLLSPYLHFGEISVRHVFQCARMKQIIWARDK
NSEGEESADLFLRGIGLREYSRYICFNFPFTHEQSLLSHLRFFPWD
ADVDKFKAWRQGRTGYPLVDAGMRELWATGWMHNRIRVIVSSFAVK
FLLLPWKWGMKYFWDTLLDADLECDILGWQYISGSIPDGHELDRLD
NPALQGAKYDPEGEYIRQWLPELARLPTEWIHHPWDAPLTVLKASG
VELGTNYAKPIVDIDTARELLAKAISRTREAQIMIGAAPDEIVADS
FEALGANTIKEPGLCPSVSSNDQQVPSAVRYNGSKRVKPEEEEERD
MKKSRGFDERELFSTAESSSSSSVFFVSQSCSLASEGKNLEGIQDS
SDQITTSLGKNGCK.

In some cases, a cryptochrome-2 polypeptide comprises only the conserved photoresponsive region (phytolyase homology domain) of the cryptochrome-2 protein; this polypeptide is referred to as “CRY2 PHR.” In some cases, a CRY2 PHR polypeptide is the first member of the protein interaction pair; and a full-length calcium and integrin-binding protein 1 (C1B1) polypeptide is the second member of the protein interaction pair.

In some cases, a first polypeptide or a second polypeptide of a protein interaction pair is a CIB1 polypeptide (also known as transcription factor bHLH63). For example, a suitable CIB1 polypeptide can comprise an amino acid sequence having at least about 75%, at least about 80%, at least about 85%, at least about 90%, at least about 95%, at least about 98%, or 100% amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, from about 150 aa to about 160 aa, from about 160 aa to about 170 aa, from about 170 aa to about 180 aa, from about 180 aa to about 190 aa, or from about 190 aa to about 200 aa of the following amino acid sequence:

(SEQ ID NO: //)
MNGAIGGDLLLNFPDMSVLERQRAHLKYLNPTFDSPLAGFFADSSM
ITGGEMDSYLSTAGLNLPMMYGETTVEGDSRLSISPETTLGTGNFK
KRKFDTETKDCNEKKKKMTMNRDDLVEEGEEEKSKITEQNNGSTKS
IKKMKHKAKKEENNFSNDSSKVTKELEKTDYIHVRARRGQATDSHS
IAERVRREKISERMKFLQDLVPGCDKITGKAGMLDEIINYVQSLQR
QIEFLSMKLAIVNPRPDFDMDDIFAKEVASTPMTVVPSPEMVLSGY
SHEMVHSGYSSEMVNSGYLHVNPMQQVNTSSDPLSCFNNGEAPSMW
DSHVQNLYGNLGV.

Nuclear Hormone Receptor/Co-Regulator Peptide Protein Interaction Pairs

In some cases, the first polypeptide of a protein interaction pair is any naturally occurring or engineered derivative of any nuclear receptor, thyroid hormone receptor, retinoic acid receptor, estrogen receptor, estrogen-related receptor, glucocorticoid receptor, progesterone receptor, or androgen receptor; and the second polypeptide of the protein interaction pair is a nuclear hormone-binding polypeptide. In some cases, the ligand-binding domain of a nuclear hormone receptor is used. A ligand-binding domain of a nuclear hormone receptor can be from any of a variety of nuclear hormone receptors, including, but not limited to, ERα, ERβ, PR, AR, GR, MR, RARα, RARβ, RARγ, TRα, TRβ, VDR, EcR, RXRα, RXRβ, RXRγ, PPARα, PPARβ, PPARγ, LXRα, LXRβ, FXR, PXR, SXR, CAR, SF-1, LRH-1, DAX-1, SHP, TLX, PNR, NGF1-Bα, NGF1-Bβ, NGF1-Bγ, RORα, RORβ, RORγ, ERRα, ERRβ, ERRγ, GCNF, TR2/4, HNF-4, COUP-TFα, COUP-TFβ and COUP-TFγ.

Abbreviations for nuclear hormone receptors are as follows. ER: Estrogen Receptor; PR: Progesterone Receptor; AR: Androgen Receptor; GR: Glucocorticoid Receptor; MR: Mineralocorticoid Receptor; RAR: Retinoic Acid Receptor; TRα, β: Thyroid Receptor; VDR: Vitamin D3 Receptor; EcR: Ecdysone Receptor; RXR: Retinoic Acid X Receptor; PPAR: Peroxisome Proliferator Activated Receptor; LXR: Liver X Receptor; FXR: Farnesoid X Receptor; PXR/SXR: Pregnane X Receptor/Steroid and Xenobiotic Receptor; CAR: Constitutive Adrostrane Receptor; SF-1: Steroidogenic Factor 1; DAX-1: Dosage sensitive sex reversal-adrenal hypoplasia congenital critical region on the X chromosome, gene 1; LRH-1: Liver Receptor Homolog 1; SHP: Small Heterodimer Partner; TLX: Tailless Gene; PNR: Photoreceptor-Specific Nuclear Receptor; NGF1-B: Nerve Growth Factor; ROR: RAR related orphan receptor; ERR: Estrogen Related Receptor; GCNF: Germ Cell Nuclear Factor; TR2/4: Testicular Receptor; HNF-4: Hepatocyte Nuclear Factor; COUP-TF: Chicken Ovalbumin Upstream Promoter, Transcription Factor.

A nuclear hormone receptor, or a ligand-binding domain of a nuclear hormone receptor, may be obtained from a steroid/thyroid hormone nuclear receptor selected from the group consisting of thyroid hormone receptor α (TRα), thyroid receptor 1 (c-erbA-1), thyroid hormone receptor β (TRβ), retinoic acid receptor α (RARα), retinoic acid receptor β (RARβ, HAP), retinoic acid receptor γ (RARγ), retinoic acid receptor gamma-like (RARD), peroxisome proliferator-activated receptor α (PPARα), peroxisome proliferator-activated receptor β (PPARβ), peroxisome proliferator-activated δ (PPARdelta, NUC-1), peroxisome proliferator-activator related receptor (FFAR), peroxisome proliferator-activated receptor γ (PPARγ), orphan receptor encoded by non-encoding strand of thyroid hormone receptor α (REVERBα), v-erb A related receptor (EAR-1), v-erb related receptor (EAR-IA), γ), orphan receptor encoded by non-encoding strand of thyroid hormone receptor β (REVERBβ), v-erb related receptor (EAR-1β), orphan nuclear receptor BD73 (BD73), rev-erbA-related receptor (RVR), zinc finger protein 126 (HZF2), ecdysone-inducible protein E75 (E75), ecdysone-inducible protein E78 (E78), Drosophila receptor 78 (DR-78), retinoid-related orphan receptor α (RORα), retinoid Z receptor α (RZRα), retinoid related orphan receptor β (RORβ), retinoid Z receptor β (RZRβ), retinoid-related orphan receptor γ (RORγ), retinoid Z receptor γ (RZRγ), retinoid-related orphan receptor (TOR), hormone receptor 3 (HR-3), Drosophila hormone receptor 3 (DHR-3), Manduca hormone receptor (MHR-3), Galleria hormone receptor 3 (GHR-3), C. elegans nuclear receptor 3 (CNR-3), Choristoneura hormone receptor 3 (CHR-3), C. elegans nuclear receptor 14 (CNR-14), ecdysone receptor (ECR), ubiquitous receptor (UR), orphan nuclear receptor (OR-1), NER-1, receptor-interacting protein 15 (RIP-15), liver X receptor β (LXRβ), steroid hormone receptor like protein (RLD-1), liver X receptor (LXR), liver X receptor α (LXRα), farnesoid X receptor (FXR), receptor-interacting protein 14 (RIP-14), HRR-1, vitamin D receptor (VDR), orphan nuclear receptor (ONR-1), pregnane X receptor (PXR), steroid and xenobiotic receptor (SXR), benzoate X receptor (BXR), nuclear receptor (MB-67), constitutive androstane receptor 1 (CAR-1), constitutive androstane receptor α (CARα), constitutive androstane receptor 2 (CAR-2), constitutive androstane receptor β (CARβ), Drosophila hormone receptor 96 (DHR-96), nuclear hormone receptor 1 (NHR-1), hepatocyte nuclear factor 4 (HNF-4), hepatocyte nuclear factor 4G (HNF-4G), hepatocyte nuclear factor 4B (HNF-4B), hepatocyte nuclear factor 4D (HNF-4D, DHNF-4), retinoid X receptor α (RXRα), retinoid X receptor β (RXRβ), H-2 region II binding protein (H-2RIIBP), nuclear receptor co-regulator-1 (RCoR-1), retinoid X receptor γ (RXRγ), Ultraspiracle (USP), 2C1 nuclear receptor, chorion factor 1 (CF-1), testicular receptor 2 (TR-2), testicular receptor 2-11 (TR2-11), testicular receptor 4 (TR4), TAK-1, Drosophila hormone receptor (DHR78), Tailless (TLL), tailless homolog (TLX), XTLL, chicken ovalbumin upstream promoter transcription factor I (COUP-TFI), chicken ovalbumin upstream promoter transcription factor A (COUP-TFA), EAR-3, SVP-44, chicken ovalbumin upstream promoter transcription factor II (COUP-TFII), chicken ovalbumin upstream promoter transcription factor B (COUP-TFB), ARP-1, SVI O, SVP, chicken ovalbumin upstream promoter transcription factor III (COUP-TFIII), chicken ovalbumin upstream promoter transcription factor G (COUP-TFG), SVP-46, EAR-2, estrogen receptor α (ERα), estrogen receptor β (ERβ), estrogen related receptor 1 (ERR1), estrogen related receptor α (ERRα), estrogen related receptor 2 (ERR2), estrogen related receptor β (ERRβ), glucocorticoid receptor (GR), mineralocorticoid receptor (MR), progesterone receptor (PR), androgen receptor (AR), nerve growth factor induced gene B (NGFI-B), nuclear receptor similar to Nur-77 (TRS), N10, orphan receptor (NUR-77), Human early response gene (NAK-1), Nun related factor 1 (NURR-1), a human immediate-early response gene (NOT), regenerating liver nuclear receptor 1 (RNR-1), hematopoietic zinc finger 3 (HZF-3), Nur rekated protein-1 (TINOR), Nuclear orphan receptor 1 (NOR-1), NOR1 related receptor (MINOR), Drosophila hormone receptor 38 (DHR-38), C. elegans nuclear receptor 8 (CNR-8), C48D5, steroidogenic factor 1 (SF1), endozepine-like peptide (ELP), fushi tarazu factor 1 (FTZ-F1), adrenal 4 binding protein (AD4BP), liver receptor homolog (LRH-1), Ftz-F1-related orphan receptor A (xFFrA), Ftz-F1-related orphan receptor B (xFFrB), nuclear receptor related to LRH-1 (FFLR), nuclear receptor related to LRH-1 (PHR), fetoprotein transcription factor (FTF), germ cell nuclear factor (GCNFM), retinoid receptor-related testis-associated receptor (RTR), knirps (KNI), knirps related (KNRL), Embryonic gonad (EGON), Drosophila gene for ligand dependent nuclear receptor (EAGLE), nuclear receptor similar to trithorax (ODR7), Trithorax, dosage sensitive sex reversal adrenal hypoplasia congenita critical region chromosome X gene (DAX-1), adrenal hypoplasia congenita and hypogonadotropic hypogonadism (AHCH), and short heterodimer partner (SHP).

In some cases, a co-activator peptide comprises the amino acid sequence LXXLL, where X is any amino acid. In some cases, a co-activator peptide comprises the amino acid sequence FXXLF, where X is any amino acid.

For example, the first or the second member of a protein interaction pair can be a mineralcorticoid receptor, e.g., a ligand-binding domain (LBD) of a mineralocorticoid receptor (MR). The LBD of a MR can comprise an amino acid sequence having at least 85%, at least 90%, at least 95%, at least 98%, at least 99%, or 100%, amino acid sequence identity to the following amino acid sequence: EEQPQ QQQPPPPPPP PQSPEEGTTY IAPAKEPSVN TALVPQLSTI SRALTPSPVM VLENIEPEIV YAGYDSSKPD TAENLLSTLN RLAGKQMIQV VKWAKVLPGF KNLPLEDQIT LIQYSWMCLS SFALSWRSYK HTNSQFLYFA PDLVFNEEKM HQSAMYELCQ GMHQISLQFV RLQLTFEEYT IMKVLLLLST IPKDGLKSQA AFEEMRTNYI KELRKMVTKC PNNSGQSWQR FYQLTKLLDS MHDLVSDLLE FCFYTFRESH ALKVEFPAML VEIISDQLPK VESGNAKPLY FHRK (SEQ ID NO://); and the other member of the protein interaction pair can be a co-regulator peptide comprising the amino acid sequence SLTARHKILHRLLQEGSPSDI (SEQ ID NO://), QEAEEPSLLKKLLLAPANTQL (SEQ ID NO://), or SKVSQNPILTSLLQITGNGGS (SEQ ID NO://).

As another example, the first or the second member of a protein interaction pair can be an androgen receptor (AR), e.g., an LBD of an AR. The LBD of an AR can comprise an amino acid sequence having at least 85%, at least 90%, at least 95%, at least 98%, at least 99%, or 100%, amino acid sequence identity to the following amino acid sequence:

D NNQPDSFAAL LSSLNELGER
QLVHVVKWAK ALPGFRNLHV DDQMAVIQYS WMGLMVFAMG
WRSFTNVNSR MLYFAPDLVF NEYRMHKSRM YSQCVRMRHL
SQEFGWLQIT PQEFLCMKAL LLFSIIPVDG LKNQKFFDEL
RMNYIKELDR IIACKRKNPT SCSRRFYQLT KLLDSVQPIA
RELHQFTFDL LIKSHMVSVD FPEMMAEIIS VQVPKILSGK
VKPIYFHTQ;

and the other member of the protein interaction pair can be a co-regulator peptide comprising the amino acid sequence ESKGHKKLLQLLTCSSDDR (SEQ ID NO://).

As another example, the first or the second member of a protein interaction pair can be a progesterone receptor (PR), e.g., an LBD of a PR. The LBD of a PR can comprise an amino acid sequence having at least 85%, at least 90%, at least 95%, at least 98%, at least 99%, or 100%, amino acid sequence identity to the following amino acid sequence: and the other member of the protein interaction pair can be a co-regulator peptide comprising the amino acid sequence GQD IQLIPPLINL LMSIEPDVIY AGHDNTKPDT SSSLLTSLNQ DLILNEQRMK ESSFYSLCLT MWQIPQEFVK LQVSQEEFLC MKVLLLLNTI PLEGLRSQTQ FEEMRSSYIR ELIKAIGLRQ KGVVSSSQRF YQLTKLLDNL HDLVKQLHLY CLNTFIQSRA LSVEFPEMMS EVIAAQLPKI LAGMVKPLLF HKK (SEQ ID NO://); and the other member of the protein interaction pair can be a co-regulator peptide comprising the amino acid sequence GHSFADPASNLGLEDIIRKALMGSF (SEQ ID NO://).

Suitable co-regulator peptides include, but are not limited to, Steroid Receptor Coactivator (SRC)-1, SRC-2, SRC-3, TRAP220-1, TRAP220-2, NR0B1, NRIP1, CoRNR box, αβV, TIF1, TIF2, EA2, TA1, EAB1, SRC1-1, SRC1-2, SRC1-3, SRC1-4a, SRC1-4b, GRIP1-1, GRIP1-2, GRIP1-3, AIB1-1, AIB1-2, AIB1-3, PGC1a, PGC1b, PRC, ASC2-1, ASC2-2, CBP-1, CBP-2, P300, CIA, ARA70-1, ARA70-2, NSD1, SMAP, Tip60, ERAP140, Nix1, LCoR, CoRNR1 (N-CoR), CoRNR2, SMRT, RIP140-C, RIP140-1, RIP140-2, RIP140-3, RIP140-4, RIP140-5, RIP140-6, RIP140-7, RIP140-8, RIP140-9, PRIC285-1, PRIC285-2, PRIC285-3, PRIC285-4, and PRIC285-5.

In some cases, a suitable co-regulator peptide comprises an LXXLL motif, where X is any amino acid; where the co-regulator peptide has a length of from 8 amino acids to 50 amino acids, e.g., from 8 amino acids to 10 amino acids, from 10 amino acids to 12 amino acids, from 12 amino acids to 15 amino acids, from 15 amino acids to 20 amino acids, from 20 amino acids to 25 amino acids, from 25 amino acids to 30 amino acids, from 30 amino acids to 35 amino acids, from 35 amino acids to 40 amino acids, from 40 amino acids to 45 amino acids, or from 45 amino acids to 50 amino acids.

Non-limiting examples of suitable co-regulator peptides are as follows:

SRC1:
(SEQ ID NO: //)
CPSSHSSLTERHKILHRLLQEGSPS;
SRC1-2:
(SEQ ID NO: //)
SLTARHKILHRLLQEGSPSDI;
SRC3-1:
(SEQ ID NO: //)
ESKGHKKLLQLLTCSSDDR;
SRC3:
(SEQ ID NO: //)
PKKENNALLRYLLDRDDPSDV;
PGC-1:
(SEQ ID NO: //)
AEEPSLLKKLLLAPANT;
PGC1a:
(SEQ ID NO: //)
QEAEEPSLLKKLLLAPANTQL;
TRAP220-1:
(SEQ ID NO: //)
SKVSQNPILTSLLQITGNGGS;
NCoR (2051-2075):
(SEQ ID NO: //)
GHSFADPASNLGLEDIIRKALMGSF;
NR0B1:
(SEQ ID NO: //)
PRQGSILYSMLTSAKQT;
NRIP1:
(SEQ ID NO: //)
AANNSLLLHLLKSQTIP;
TIF2:
(SEQ ID NO: //)
PKKKENALLRYLLDKDDTKDI;
CoRNR Box:
(SEQ ID NO: //)
DAFQLRQLILRGLQDD;
abV:
(SEQ ID NO: //)
SPGSREWFKDMLS;
TRAP220-2:
(SEQ ID NO: //)
GNTKNHPMLMNLLKDNPAQDF;
EA2:
(SEQ ID NO: //)
SSKGVLWRMLAEPVSR;
TA1:
(SEQ ID NO: //)
SRTLQLDWGTLYWSR;
EAB1:
(SEQ ID NO: //)
SSNHQSSRLIELLSR;
SRC2:
(SEQ ID NO: //)
LKEKHKILHRLLQDSSSPV;
SRC1-3:
(SEQ ID NO: //)
QAQQKSLLQQLLTE;
SRC1-1:
(SEQ ID NO: //)
KYSQTSHK LVQLL TTTAEQQL;
SRC1-2:
(SEQ ID NO: //)
SLTARHKI LHRLL QEGSPSDI;
SRC1-3:
(SEQ ID NO: //)
KESKDHQL LRYLL DKDEKDLR;
SRC1-4a:
(SEQ ID NO: //)
PQAQQKSL LQQLL TE;
SRC1-4b:
(SEQ ID NO: //)
PQAQQKSL RQQLL TE;
GRIP1-1:
(SEQ ID NO: //)
HDSKGQTK LLQLL TTKSDQME;
GRIP1-2:
(SEQ ID NO: //)
SLKEKHKI LHRLL QDSSSPVD;
GRIP1-3:
(SEQ ID NO: //)
PKKKENAL LRYLL DKDDTKDI;
AIB1-1:
(SEQ ID NO: //)
LESKGHKK LLQLL TCSSDDRG;
AIB1-2:
(SEQ ID NO: //)
LLQEKHRI LHKLL QNGNSPAE;
AIB1-3:
(SEQ ID NO: //)
KKKENNAL LRYLL DRDDPSDA;
PGC1a:
(SEQ ID NO: //)
QEAEEPSL LKKLL LAPANTQL;
PGC1b:
(SEQ ID NO: //)
PEVDELSL LQKLL LATSYPTS;
PRC:
(SEQ ID NO: //)
VSPREGSS LHKLL TLSRTPPE;
TRAP220-1:
(SEQ ID NO: //)
SKVSQNPI LTSLL QITGNGGS;
TRAP220-2:
(SEQ ID NO: //)
GNTKNHPM LMNLL KDNPAQDF;
ASC2-1:
(SEQ ID NO: //)
DVTLTSPL LVNLL QSDISAGH;
ASC2-2:
(SEQ ID NO: //)
AMREAPTS LSQLL DNSGAPNV;
CBP-1:
(SEQ ID NO: //)
DAASKHKQ LSELL RGGSGSSI;
CBP-2:
(SEQ ID NO: //)
KRKLIQQQ LVLLL HAHKCQRR;
P300:
(SEQ ID NO: //)
DAASKHKQ LSELL RSGSSPNL;
CIA:
(SEQ ID NO: //)
GHPPAIQS LINLL ADNRYLTA;
ARA70-1:
(SEQ ID NO: //)
TLQQQAQQ LYSLL GQFNCLTH;
ARA70-2:
(SEQ ID NO: //)
GSRETSEK FKLLF QSYNVNDW;
TIF1:
(SEQ ID NO: //)
NANYPRSI LTSLL LNSSQSST;
NSD1:
(SEQ ID NO: //)
IPIEPDYK FSTLL MMLKDMHD;
SMAP:
(SEQ ID NO: //)
ATPPPSPL LSELL KKGSLLPT;
Tip60:
(SEQ ID NO: //)
VDGHERAM LKRLL RIDSKCLH;
ERAP140:
(SEQ ID NO: //)
HEDLDKVK LIEYY LTKNKEGP;
Nix1:
(SEQ ID NO: //)
ESPEFCLG LQTLL SLKCCIDL;
LCoR:
(SEQ ID NO: //)
AATTQNPV LSKLL MADQDSPL;
CoRNR1 (N-CoR):
(SEQ ID NO: //)
MGQVPRTHRLITLADH ICQII TQDFARNQV;
CoRNR2 (N-CoR):
(SEQ ID NO: //)
NLG LEDII RKALMG;
CoRNR1 (SMRT):
(SEQ ID NO: //)
APGVKGHQRVVTLAQH ISEVI TQDTYRHHPQQLSAPLPAP;
CoRNR2 (SMRT):
(SEQ ID NO: //)
NMG LEAII RKALMG;
RIP140-C:
(SEQ ID NO: //)
RLTKTNPI LYYML QKGGNSVA;
RIP140-1:
(SEQ ID NO: //)
QDSIVLTY LEGLL MHQAAGGS;
RIP140-2:
(SEQ ID NO: //)
KGKQDSTL LASLL QSFSSRLQ;
RIP140-3:
(SEQ ID NO: //)
CYGVASSH LKTLL KKSKVKDQ;
RIP140-4:
(SEQ ID NO: //)
KPSVACSQ LALLL SSEAHLQQ;
RIP140-5:
(SEQ ID NO: //)
KQAANNSL LLHLL KSQTIPKP;
RIP140-6:
(SEQ ID NO: //)
NSHQKVTL LQLLL GHKNEENV;
RIP140-7:
(SEQ ID NO: //)
NLLERRTV LQLLL GNPTKGRV;
RIP140-8:
(SEQ ID NO: //)
FSFSKNGL LSRLL RQNQDSYL;
RIP140-9:
(SEQ ID NO: //)
RESKSFNV LKQLL LSENCVRD;
PRIC285-1:
(SEQ ID NO: //)
ELNADDAI LRELL DESQKVMV;
PRIC285-2:
(SEQ ID NO: //)
YENLPPAA LRKLL RAEPERYR;
PRIC285-3:
(SEQ ID NO: //)
MAFAGDEV LVQLL SGDKAPEG;
PRIC285-4:
(SEQ ID NO: //)
SCCYLCIR LEGLL APTASPRP;
and
PRIC285-5:
(SEQ ID NO: //)
PSNKSVDV LAGLL LRRMELKP.

### Calcium Binding Protein Pairs

In some cases, a calcium-binding protein pair comprises calmodulin and a calmodulin-binding protein.

A suitable calmodulin polypeptide can comprise an amino acid sequence having at least 80%, at least 85%, at least 90%, at least 95%, at least 98%, at least 99%, or 100%, amino acid sequence identity to the following calmodulin amino acid sequence:

(SEQ ID NO: //)
GESLFKGPRDYNPISSTICHLTNESDGHTTSLYGIGFGPFIITNKHLFRR
NNGTLLVQSLHGVFKVKNTTTLQQHLIDGRDMIIIRMPKDFPPFPQKLKF
REPQREERICLVTTNFQTKSMSSMVSDTSCTFPSSDGIFWKHWIQTKDGQ
CGSPLVSTRDGFIVGIHSASNFTNTNNYFTSVPKNFMELLTNQEAQQWVS
GWRLNADSVLWGGHKVFMV.

A suitable calmodulin-binding polypeptide can comprise the following amino acid sequence: NARRKLAGAILFTMLATRNFS (SEQ ID NO://); and has a length of from 21 amino acids to about 25 amino acids. In some cases, two copies of a calmodulin-binding polypeptide are present in a PPI detection system of the present disclosure. In some cases, the two copies are in tandem, with no intervening linker. In some cases, the two copies are in tandem and are separated by a linker (e.g., a linker of from 2 to 5, 5 to 10, or 10 to 15 amino acids).

A suitable calmodulin-binding polypeptide binds a calmodulin polypeptide under conditions of high Ca2⁺ concentration. For example, a suitable calmodulin-binding polypeptide binds a calmodulin polypeptide when the concentration of Ca2⁺ is greater than 100 nM, greater than 150 nM, greater than 200 nM, greater than 250 nM, greater than 300 nM, greater than 350 nM, greater than 400 nM, greater than 500 nM, or greater than 750 nM.

A suitable calmodulin-binding polypeptide does not substantially bind a calmodulin polypeptide under conditions of low Ca2⁺ concentration. For example, a suitable calmodulin-binding polypeptide does not substantially bind a calmodulin polypeptide when the intracellular Ca2⁺ concentration is less than about 300 nM, less than about 250 nM, less than about 200 nM, less than about 110 nM, less than about 105 nM, or less than about 100 nM.

A calmodulin-binding polypeptide can have a length of from about 10 amino acids to about 50 amino acids, e.g., from about 10 amino acids to about 40 amino acids, from about 20 amino acids to about 40 amino acids, from about 15 amino acids to about 25 amino acids, e.g., from about 10 amino acids to about 15 amino acids, from about 15 amino acids to about 20 amino acids, from about 20 amino acids to about 25 amino acids, from about 25 amino acids to about 30 amino acids, from about 30 amino acids to about 35 amino acids, from about 35 amino acids to about 40 amino acids, from about 40 amino acids to about 45 amino acids, or from about 45 amino acids to about 50 amino acids.

A suitable calmodulin-binding polypeptide in some cases comprises an amino acid sequence having at least 90%, at least 95%, at least 98%, at least 99%, or 100%, amino acid sequence identity to the following amino acid sequence: KRRWKKNFIAVSAANRFKKISSSGAL (SEQ ID NO://); and has a length of from about 26 amino acids to about 30 amino acids.

In some cases, a suitable calmodulin-binding polypeptide comprises an amino acid sequence having at least 90%, at least 95%, at least 98%, at least 99%, or 100%, amino acid sequence identity to the following amino acid sequence: KRRWKKNFIAVSAANRFKKISSSGAL (SEQ ID NO://); and has a substitution of A14; and has a length of from about 26 amino acids to about 30 amino acids. In some cases, a suitable calmodulin-binding polypeptide comprises an amino acid sequence having at least 90%, at least 95%, at least 98%, at least 99%, or 100%, amino acid sequence identity to the following amino acid sequence: KRRWKKNFIAVSAANRFKKISSSGAL (SEQ ID NO://); and has an A14F substitution; and has a length of from about 26 amino acids to about 30 amino acids. In some cases, a suitable calmodulin-binding polypeptide comprises the following amino acid sequence: KRRWKKNFIAVSAFNRFKKISSSGAL (SEQ ID NO://); and has a length of 26 amino acids.

In some cases, a suitable calmodulin-binding polypeptide comprises an amino acid sequence having at least 90%, at least 95%, at least 98%, at least 99%, or 100%, amino acid sequence identity to the following amino acid sequence: FNARRKLKGAILTTMLFTRNFS (SEQ ID NO://); and has a length of from 22 amino acids to about 25 amino acids. In some cases, a suitable calmodulin-binding polypeptide comprises an amino acid sequence having at least 90%, at least 95%, at least 98%, at least 99%, or 100%, amino acid sequence identity to the following amino acid sequence: FNARRKLKGAILTTMLFTRNFS (SEQ ID NO://); and has a K8 amino acid substitution; and has a length of from 22 amino acids to about 25 amino acids. In some cases, a suitable calmodulin-binding polypeptide comprises an amino acid sequence having at least 90%, at least 95%, at least 98%, at least 99%, or 100%, amino acid sequence identity to the following amino acid sequence: FNARRKLKGAILTTMLFTRNFS (SEQ ID NO://); and has a K8A amino acid substitution; and has a length of from 22 amino acids to about 25 amino acids. In some cases, a suitable calmodulin-binding polypeptide comprises an amino acid sequence having at least 90%, at least 95%, at least 98%, at least 99%, or 100%, amino acid sequence identity to the following amino acid sequence: FNARRKLKGAILTTMLFTRNFS (SEQ ID NO://); and has a T13 substitution; and has a length of from 22 amino acids to about 25 amino acids. In some cases, a suitable calmodulin-binding polypeptide comprises an amino acid sequence having at least 90%, at least 95%, at least 98%, at least 99%, or 100%, amino acid sequence identity to the following amino acid sequence: FNARRKLKGAILTTMLFTRNFS (SEQ ID NO://); and has a T13F substitution; and has a length of from 22 amino acids to about 25 amino acids. In some cases, a suitable calmodulin-binding polypeptide comprises the following amino acid sequence: FNARRKLKGAILFTMLFTRNFS; and has a length of 22 amino acids. In some cases, a suitable calmodulin-binding polypeptide comprises the following amino acid sequence: FNARRKLAGAILFTMLFTRNFS; and has a length of 22 amino acids.

In some cases, two copies of a calmodulin-binding polypeptide are used. For example, a calmodulin-binding polypeptide can comprise the amino acid sequence FNARRKLAGAILFTMLATRNFSGSFNARRKLAGAILFTMLATRNFS (SEQ ID NO://) which contains two copies of FNARRKLAGAILFTMLATRNFS (SEQ ID NO://) and an intervening Gly-Ser (GS) linker.

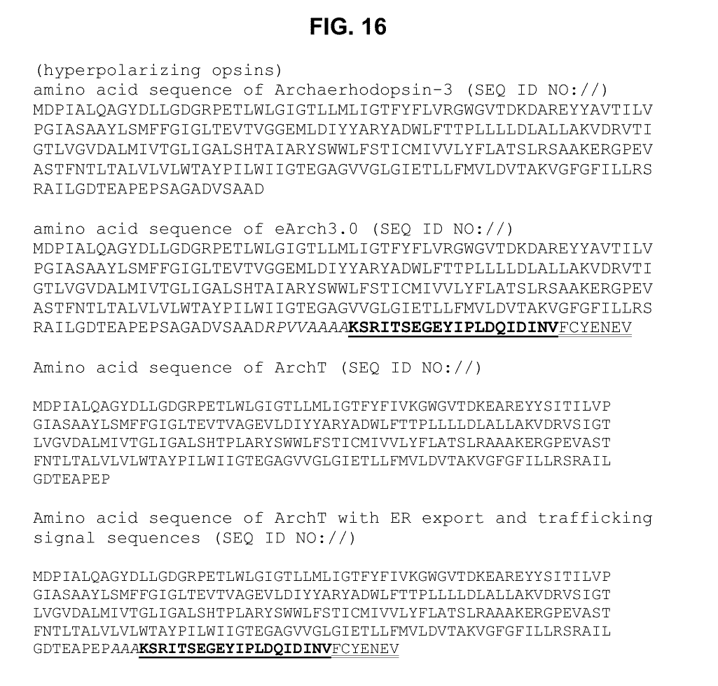

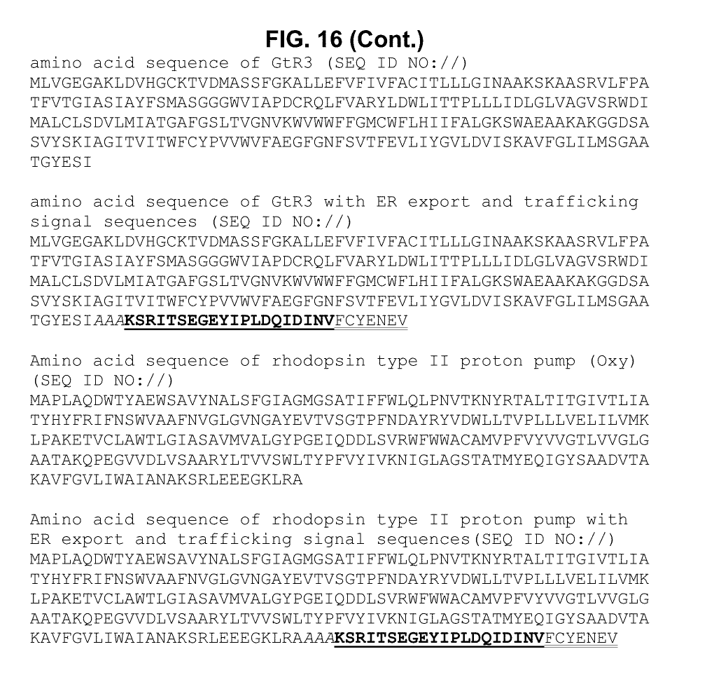

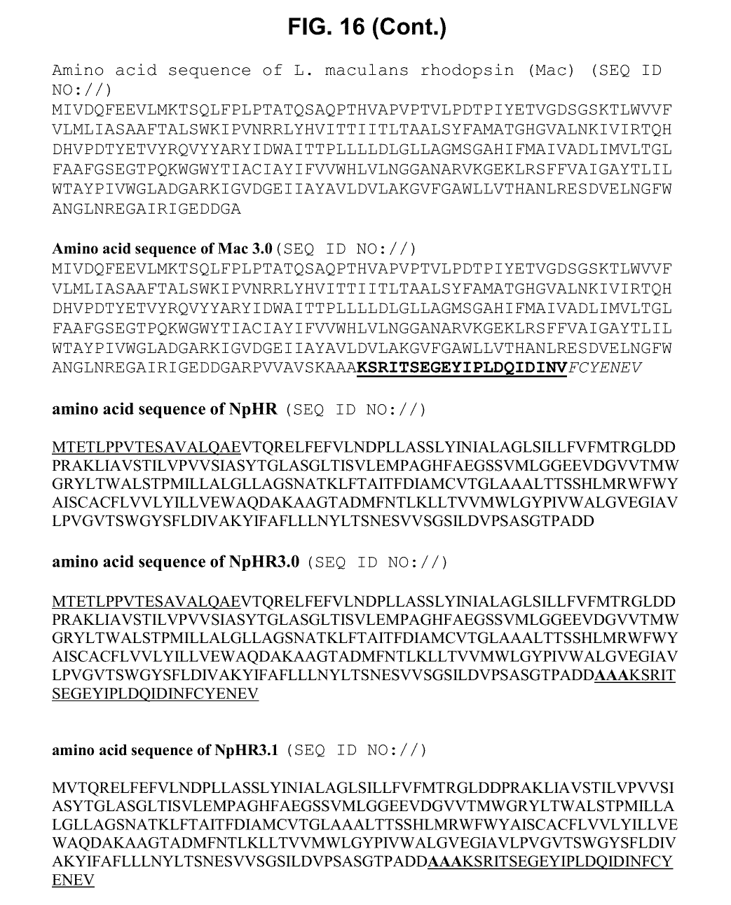

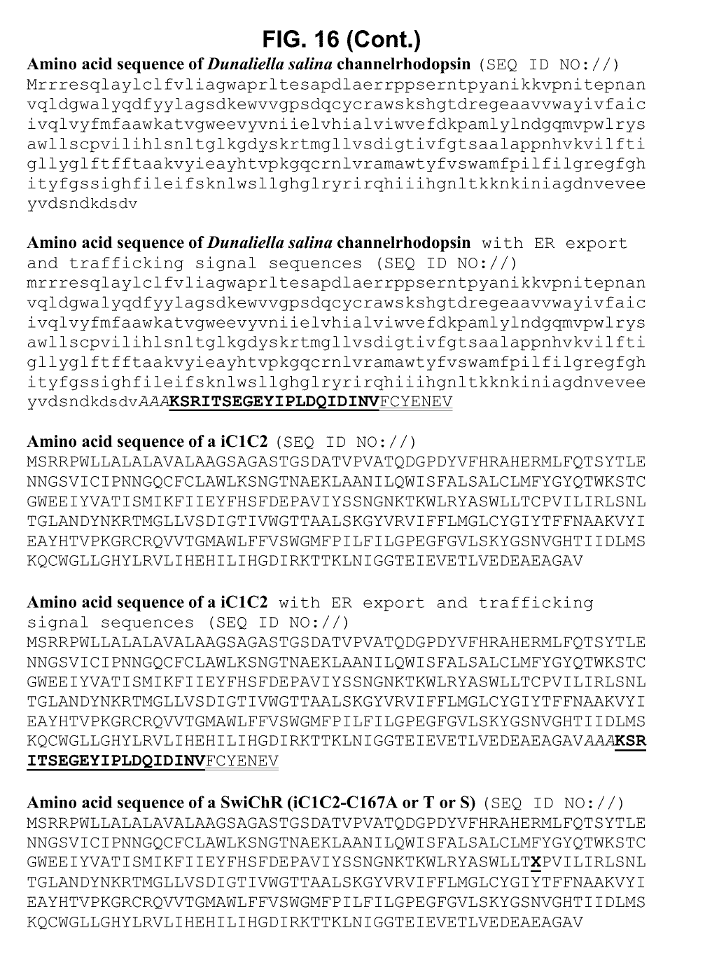

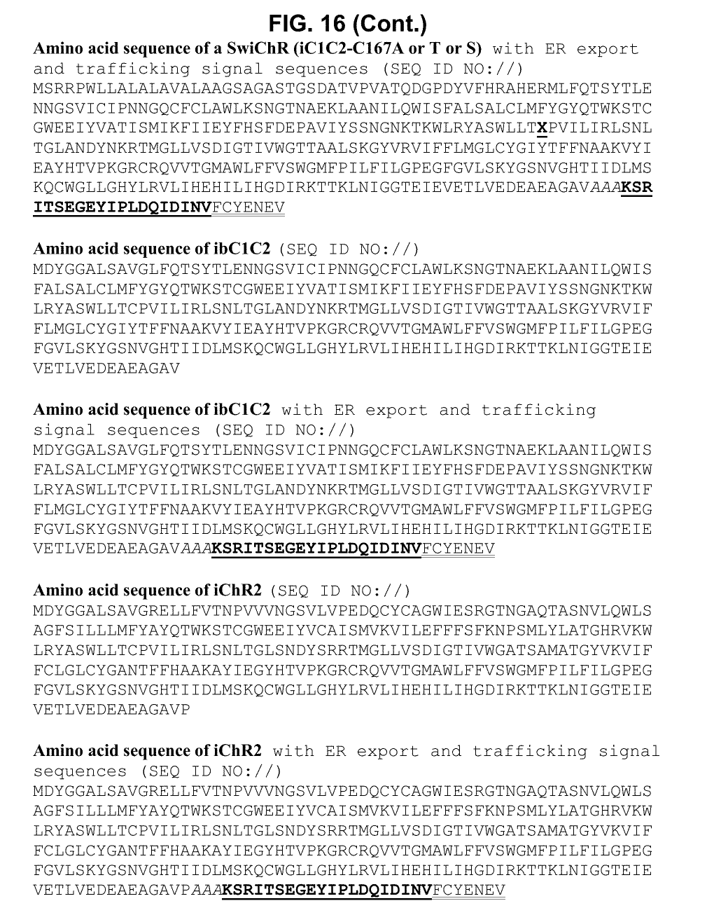

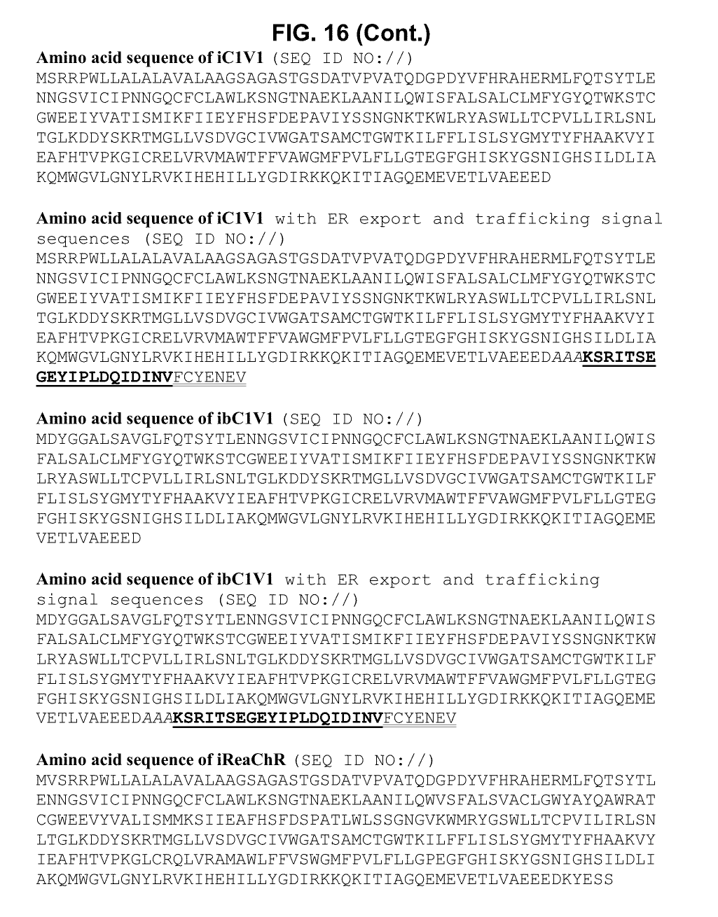

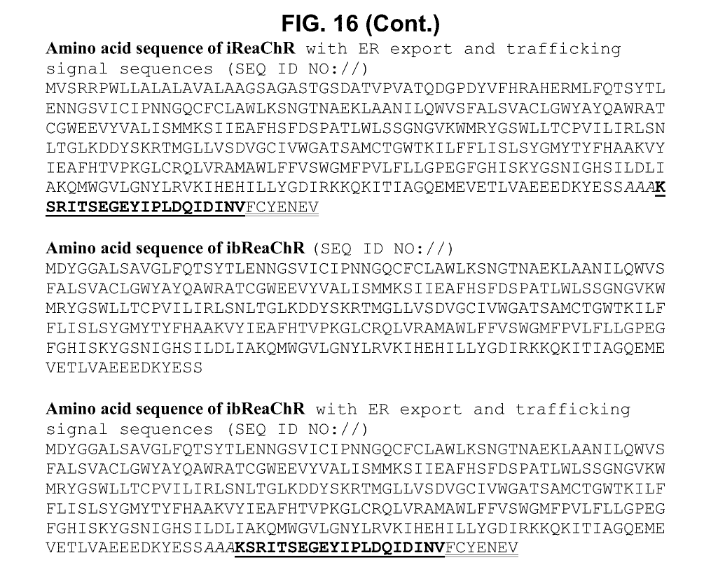

A suitable calmodulin polypeptide comprises an amino acid sequence having at least 80%, at least 85%, at least 90%, at least 95%, at least 98%, at least 99%, or 100%, amino acid sequence identity to the amino acid sequence depicted in FIG. 16A or FIG. 16B.

A suitable calmodulin polypeptide comprises an amino acid sequence having at least 80%, at least 85%, at least 90%, at least 95%, at least 98%, at least 99%, or 100%, amino acid sequence identity to the following calmodulin amino acid sequence: MDQLTEEQIAEFKEAFSLFDKDGDGTITTKELGTVMRSLGQNPTEAELQDMINEVDADGDGTID FPEFLTMMARKMKYTDSEEEIREAFRVFDKDGNGYISAAELRHVMTNLGEKLTDEEVDEMIRE ADIDGDGQVNYEEFVQMMTAK (SEQ ID NO://); and has a length of from about 148 amino acids to about 160 amino acids. In some cases, the calmodulin polypeptide has a length of 148 amino acids.

In some cases, a suitable calmodulin polypeptide comprises an amino acid sequence having at least 80%, at least 85%, at least 90%, at least 95%, at least 98%, at least 99%, or 100%, amino acid sequence identity to the following calmodulin amino acid sequence: MDQLTEEQIAEFKEAFSLFDKDGDGTITTKELGTVMRSLGQNPTEAELQDMINEVDADGDGTID FPEFLTMMARKMKYTDSEEEIREAFRVFDKDGNGYISAAELRHVMTNLGEKLTDEEVDEMIRE ADIDGDGQVNYEEFVQMMTAK (SEQ ID NO://); and has a substitution of F19; and has a length of from about 148 amino acids to about 160 amino acids. In some cases, the calmodulin polypeptide has a length of 148 amino acids. In some cases, the F19 substitution is an F19L substitution, an F19I substitution, an F19V substitution, or an F19A substitution.

In some cases, a suitable calmodulin polypeptide comprises an amino acid sequence having at least 80%, at least 85%, at least 90%, at least 95%, at least 98%, at least 99%, or 100%, amino acid sequence identity to the following calmodulin amino acid sequence: MDQLTEEQIAEFKEAFSLFDKDGDGTITTKELGTVMRSLGQNPTEAELQDMINEVDADGDGTID FPEFLTMMARKMKYTDSEEEIREAFRVFDKDGNGYISAAELRHVMTNLGEKLTDEEVDEMIRE ADIDGDGQVNYEEFVQMMTAK (SEQ ID NO://); and has a substitution of V35; and has a length of from about 148 amino acids to about 160 amino acids. In some cases, the calmodulin polypeptide has a length of 148 amino acids. In some cases, the V35 substitution is a V35G substitution, a V35A substitution, a V35L substitution, or a V35I substitution.

In some cases, a suitable calmodulin polypeptide comprises an amino acid sequence having at least 80%, at least 85%, at least 90%, at least 95%, at least 98%, at least 99%, or 100%, amino acid sequence identity to the following calmodulin amino acid sequence: MDQLTEEQIAEFKEAFSLFDKDGDGTITTKELGTVMRSLGQNPTEAELQDMINEVDADGDGTID FPEFLTMMARKMKYTDSEEEIREAFRVFDKDGNGYISAAELRHVMTNLGEKLTDEEVDEMIRE ADIDGDGQVNYEEFVQMMTAK (SEQ ID NO://); and has an F19 substitution (e.g., an F19L substitution, an F19I substitution, an F19V substitution, or an F19A substitution) and a V35 substitution (e.g., a V35G substitution, a V35A substitution, a V35L substitution, or a V35I substitution); and has a length of from about 148 amino acids to about 160 amino acids. In some cases, the calmodulin polypeptide has a length of 148 amino acids.

In some cases, a suitable calmodulin polypeptide comprises an amino acid sequence having at least 80%, at least 85%, at least 90%, at least 95%, at least 98%, at least 99%, or 100%, amino acid sequence identity to the following calmodulin amino acid sequence: MDQLTEEQIAEFKEAFSLLDKDGDGTITTKELGTGMRSLGQNPTEAELQDMINEVDADGDGTID FPEFLTMMARKMKYTDSEEEIREAFRVFDKDGNGYISAAELRHVMTNLGEKLTDEEVDEMIRE ADIDGDGQVNYEEFVQMMTAK (SEQ ID NO://); and comprises a Leu at amino acid 19 and a Gly at amino acid 35; and has a length of from about 148 amino acids to about 160 amino acids. In some cases, the calmodulin polypeptide has a length of 148 amino acids.

### Troponin C/Troponin I

In some cases, a calcium-binding protein interaction pair comprises a troponin I polypeptide and a troponin C polypeptide.

A suitable troponin I polypeptide binds a troponin C polypeptide under conditions of high Ca²⁺ concentration. For example, a suitable troponin I polypeptide binds a troponin C polypeptide when the concentration of Ca²⁺ is greater than 100 nM, greater than 150 nM, greater than 200 nM, greater than 250 nM, greater than 300 nM, greater than 350 nM, greater than 400 nM, greater than 500 nM, or greater than 750 nM.

A suitable troponin I polypeptide does not substantially bind a troponin C polypeptide under conditions of low Ca²⁺ concentration. For example, a suitable troponin I polypeptide does not substantially bind a troponin C polypeptide when the intracellular Ca²⁺ concentration is less than about 300 nM, less than about 250 nM, less than about 200 nM, less than about 110 nM, less than about 105 nM, or less than about 100 nM.

A troponin I polypeptide can have a length of from about 10 amino acids to about 200 amino acids, e.g., from about 10 amino acids to about 40 amino acids, from about 20 amino acids to about 40 amino acids, from about 15 amino acids to about 25 amino acids, e.g., from about 10 amino acids to about 15 amino acids, from about 15 amino acids to about 20 amino acids, from about 20 amino acids to about 25 amino acids, from about 25 amino acids to about 30 amino acids, from about 30 amino acids to about 35 amino acids, from about 35 amino acids to about 40 amino acids, from about 40 amino acids to about 45 amino acids, from about 45 amino acids to about 50 amino acids, from about amino acids to about 75 amino acids, from about 75 amino acids to about 100 amino acids, from about 100 amino acids to about 150 amino acids, or from about 150 amino acids to about 200 amino acids.

In some cases, a suitable troponin I polypeptide comprises an amino acid sequence having at least 80%, at least 85%, at least 90%, at least 95%, at least 98%, at least 99%, or 100%, amino acid sequence identity to the following troponin I amino acid sequence:

mpeverkpki tasrklllks lmlakakecw eqeheereae kvrylaerip tlqtrglsls alqdlcrelh akvevvdeer ydieakclhn treikdlklk vmdlrgkfkr pplrrvrvsa damlrallgs khkvsmdlra nlksvkkedt ekerpvevgd wrknveamsg megrkkmfda aksptsq (SEQ ID NO://).

A fragment of troponin I can be used. See, e.g., Tung et al. (2000) Protein Sci. 9:1312. For example, troponin I (95-114) can be used. Thus, for example, in some cases, the troponin I polypeptide can comprise an amino acid sequence having at least 80%, at least 85%, at least 90%, at least 95%, at least 98%, at least 99%, or 100%, amino acid sequence identity to the following troponin I amino acid sequence: KDLKLK VMDLRGKFKR PPLR (SEQ ID NO://); and has a length of about 20 amino acids to about 50 amino acids (e.g., from about 20 amino acids to about 25 amino acids, from about 25 amino acids to about 30 amino acids, from about 30 amino acids to about 35 amino acids, from about 35 amino acids to about 40 amino acids, from about 40 amino acids to about 45 amino acids, or from about 45 amino acids to about 50 amino acids). In some cases, the troponin I polypeptide has a length of 20 amino acids. In some cases, the troponin I polypeptide has the amino acid sequence: KDLKLK VMDLRGKFKR PPLR (SEQ ID NO://); and has a length of 20 amino acids.

In some cases, a suitable troponin I polypeptide comprises an amino acid sequence having at least 80%, at least 85%, at least 90%, at least 95%, at least 98%, at least 99%, or 100%, amino acid sequence identity to the following troponin I amino acid sequence: RMSADAMLKALLGSKHKVAMDLRAN (SEQ ID NO://); and has a length of from about 25 amino acids to about 50 amino acids (e.g., from about 25 amino acids to about 30 amino acids, from about 30 amino acids to about 35 amino acids, from about 35 amino acids to about 40 amino acids, from about 40 amino acids to about 45 amino acids, or from about 45 amino acids to about 50 amino acids). In some cases, the troponin I polypeptide has the amino acid sequence: RMSADAMLKALLGSKHKVAMDLRAN (SEQ ID NO://); and has a length of 25 amino acids.

In some cases, a suitable troponin I polypeptide comprises an amino acid sequence having at least 80%, at least 85%, at least 90%, at least 95%, at least 98%, at least 99%, or 100%, amino acid sequence identity to the following troponin I amino acid sequence: NQKLFDLRGKFKRPPLRRVRMSADAMLKALLGSKHKVAMDLRAN (SEQ ID NO://); and has a length of from about 44 amino acids to about 50 amino acids (e.g., 44, 45, 46, 47, 4, 49, or 50 amino acids). In some cases, the troponin I polypeptide has the amino acid sequence: NQKLFDLRGKFKRPPLRRVRMSADAMLKALLGSKHKVAMDLRAN (SEQ ID NO://); and has a length of 44 amino acids.

A suitable troponin C polypeptide comprises an amino acid sequence having at least 80%, at least 85%, at least 90%, at least 95%, at least 98%, at least 99%, or 100%, amino acid sequence identity to the following troponin C amino acid sequence: mtdqqaears ylseemiaef kaafdmfdad gggdisvkel gtvmrmlgqt ptkeeldaii eevdedgsgt idfeeflvmm vrqmkedakg kseeelaecf rifdrnadgy idpgelaeif rasgehvtde eieslmkdgd knndgridfd eflkmmegvq (SEQ ID NO://).

A suitable troponin C polypeptide can have a length of from about 100 amino acids to about 175 amino acids, e.g., from about 100 amino acids to about 125 amino acids, from about 125 amino acids to about 150 amino acids, or from about 150 amino acids to about 175 amino acids.

A suitable troponin C polypeptide comprises an amino acid sequence having at least 80%, at least 85%, at least 90%, at least 95%, at least 98%, at least 99%, or 100%, amino acid sequence identity to the following troponin C amino acid sequence: MTDQQAEARSYLSEEMIAEFKAAFDMFDADGGGDISVKELGTVMRMLGQTPTKEELDAIIEEV DEDGSGTIDFEEFLVMMVRQMKEDAKGKSEEELAECFRIFDRDANGYIDAEELAEIFRASGEHV TDEEIESLMKDGDKNNDGRIDFDEFLKMMEGVQ (SEQ ID NO://); and has a length of from about 160 amino acids to about 175 amino acids (e.g., from about 160 amino acids to about 165 amino acids, from about 165 amino acids to about 170 amino acids, or from about 170 amino acids to about 175 amino acids. In some cases, a suitable troponin C polypeptide comprises the amino acid sequence: MTDQQAEARSYLSEEMIAEFKAAFDMFDADGGGDISVKELGTVMRMLGQTPTKEELDAIIEEV DEDGSGTIDFEEFLVMMVRQMKEDAKGKSEEELAECFRIFDRDANGYIDAEELAEIFRASGEHV TDEEIESLMKDGDKNNDGRIDFDEFLKMMEGVQ (SEQ ID NO://); and has a length of 160 amino acids.

### Arrestin-GPCR Protein Interaction Pair

In some cases a first member of a protein interaction pair is a G-protein-coupled receptor (GPCR) and the second member of the protein interaction pair is an arrestin polypeptide. GPCRs and arrestins are known in the art; and any such GPCRs and arrestins can be used. See, e.g., Lohse and Hoffmann (2014) Handbook Exp. Pharmacol. 219:15

GPCRs that bind arrestin include, but are not limited to, rhodopsin; β₂-adrenergic receptor (β₂-AR); mm2 muscarinic cholinergic receptor (m2 mAchR); dopamine receptor D1 (DRD1); dopamine receptor D2 (DRD2); neuromedin B receptor (NMBR); β2-adrenergic receptor-2 (ADRB2); adrenoceptor alpha 1A (ADRA1A); vasopressin receptor 2 (AVPR2); vasopressin receptor 1B (AVPR1B); angiotensin receptor 2 (AGTR2); chemokine (C-C motif) receptor 5 (CCR5); kappa opioid receptor (OPRK); serotonin receptor (HTR); motilin receptor (MLNR); and the like.

Arrestins include arrestin1 arrestin4, β-arrestin1, β-arrestin2, arrestin3, and variants thereof that bind a GPCR.

Agents that induce or mediate binding of a GPCR to an arrestin polypeptide are known in the art. For example, arrestin-ADRB2 interaction can be induced or mediated by isoproterenol, epinephrine, cimaterol, clenbuterol, dobutamine, alprenolol, cyanopindolol, propanolol, sotalol, timolol, and the like; arrestin-ADRA1a interaction can be induced or mediated by norepinephrine; arrestin-MLNR interaction can be induced or mediated by motilin; arrestin-NMBR interaction can be induced or mediated by bombesin; arrestin-AGTR2 interaction can be induced or mediated by angiotensin-II; arrestin-DRD1 or arrestin-DRD2 interaction can be induced or mediated by dopamine; and arrestin-AVPR2 or arrestin-AVPR1B interaction can be induced or mediated by vasopressin.

Amino acid sequences of arrestin polypeptides are known in the art; any arrestin polypeptide that binds a GPCR is suitable for use.

In some cases, an arrestin polypeptide comprises an amino acid sequence having at least 80%, at least 85%, at least 90%, at least 95%, at least 98%, at least 99%, or 100%, amino acid sequence identity to the following amino acid sequence:

MGEKPGTRVFKKSSPNCKLTVYLGKRDFVDHLDKVDPVDGVVLVDPDYLKDRKVFVT LTCAFRYGREDLDVLGLSFRKDLFIATYQAFPPVPNPPRPPTRLQDRLLRKLGQHAHPFFFTIPQN LPCSVTLQPGPEDTGKACGVDFEIRAFCAKSLEEKSHKRNSVRLVIRKVQFAPEKPGPQPSAETT RHFLMSDRSLHLEASLDKELYYHGEPLNVNVHVTNNSTKTVKKIKVSVRQYADICLFSTAQYK CPVAQLEQDDQVSPSSTFCKVYTITPLLSDNREKRGLALDGKLKHEDTNLASSTIVKEGANKEV LGILVSYRVKVKLVVSRGGDVSVELPFVLMHPKPHDHIPLPRPQSAAPETDVPVDTNLIEFDTNY ATDDDIVFEDFARLRLKGMKDDDYDDQLC (SEQ ID NO://). An arrestin polypeptide can have a length of from about 300 amino acids to about 500 amino acids, e.g., from about 300 amino acids to about 350 amino acids, from about 350 amino acids to about 400 amino acids, from about 400 amino acids to about 425 amino acids, from about 425 amino acids to about 450 amino acids, or from about 450 amino acids to about 500 amino acids. An arrestin polypeptide can have a length of about 416 amino acids.

Binding-Inducing Agents

Binding-inducing agents that can provide for binding of a first polypeptide of a protein interaction pair to a second polypeptide of the protein interaction pair include, e.g. (where the binding-inducing agent is in parentheses following the protein interaction pair:

a) FKBP and FKBP (rapamycin);

b) FKBP and CnA (rapamycin);

c) FKBP and cyclophilin (rapamycin);

d) FKBP and FRG (rapamycin);

e) GyrB and GyrB (coumermycin);

f) DHFR and DHFR (methotrexate);

g) DmrB and DmrB (AP20187);

h) PYL and ABI (abscisic acid);

i) Cry2 and CIB1 (blue light); and

j) GAI and GID1 (gibberellin).

As noted above, rapamycin can serve as a binding-inducing agent. Alternatively, a rapamycin derivative or analog can be used. See, e.g., WO96/41865; WO 99/36553; WO 01/14387; and Ye et al (1999) Science 283:88-91. For example, analogs, homologs, derivatives and other compounds related structurally to rapamycin (“rapalogs”) include, among others, variants of rapamycin having one or more of the following modifications relative to rapamycin: demethylation, elimination or replacement of the methoxy at C7, C42 and/or C29; elimination, derivatization or replacement of the hydroxy at C13, C43 and/or C28; reduction, elimination or derivatization of the ketone at C14, C24 and/or C30; replacement of the 6-membered pipecolate ring with a 5-membered prolyl ring; and alternative substitution on the cyclohexyl ring or replacement of the cyclohexyl ring with a substituted cyclopentyl ring. Additional information is presented in, e.g., U.S. Pat. Nos. 5,525,610; 5,310,903 5,362,718; and 5,527,907. Selective epimerization of the C-28 hydroxyl group has been described; see, e.g., WO 01/14387. Additional synthetic binding-inducing agents suitable for use as an alternative to rapamycin include those described in U.S. Patent Publication No. 2012/0130076.

Rapamycin has the structure:

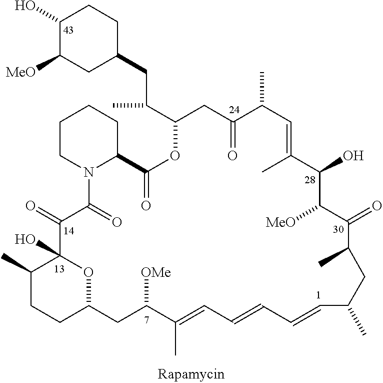

Suitable rapalogs include, e.g.,

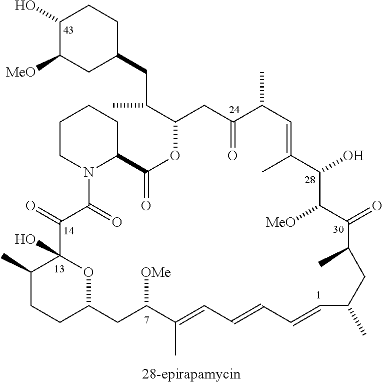

Also suitable as a rapalog is a compound of the formula:

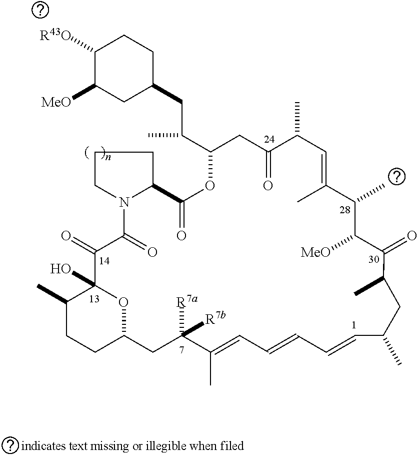

where n is 1 or 2; R²⁸ and R⁴³ are independently H, or a substituted or unsubstituted aliphatic or acyl moiety; one of R^{7a} and R^{7b} is H and the other is halo, R^{A}, OR^{A}, SR^{A}, —OC(O)R^{A}, —OC(O)NR^{A}R^{B}, —NR^{A}R^{B}, —NR^{B}C(OR)R^{A}, NR^{B}C(O)OR^{A}, —NR^{B}SO₂R^{A}, or NR^{B}SO₂NR^{A}R^{B′}; or R7a and R^{7b}, taken together, are H in the tetraene moiety:

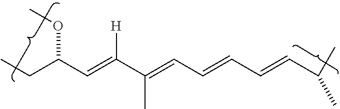

where R^{A} is H or a substituted or unsubstituted aliphatic, heteroaliphatic, aryl, or heteroaryl moiety and where R^{B} and R^{B′} are independently H, OH, or a substituted or unsubstituted aliphatic, heteroaliphatic, aryl, or heteroaryl moiety.

As noted above, coumermycin can serve as a binding-inducing agent. Alternatively, a coumermycin analog can be used. See, e.g., Farrar et al. (1996) Nature 383:178-181; and U.S. Pat. No. 6,916,846.

As noted above, in some cases, the binding-inducing agent is methotrexate, e.g., a non-cytotoxic, homo-bifunctional methotrexate dimer. See, e.g., U.S. Pat. No. 8,236,925.

In some cases, the binding-inducing agent is calcium, e.g., high intracellular calcium concentration. For example, where a protein-protein interaction pair comprises calmodulin or troponin C, members of the protein-protein interaction pair bind to one another when the concentration of Ca²⁺ is greater than 100 nM, greater than 150 nM, greater than 200 nM, greater than 250 nM, greater than 300 nM, greater than 350 nM, greater than 400 nM, greater than 500 nM, or greater than 750 nM. For example, where a protein-protein interaction pair comprises calmodulin or troponin C, members of the protein-protein interaction pair do not substantially bind to one another when the intracellular Ca²⁺ concentration is less than about 300 nM, less than about 250 nM, less than about 200 nM, less than about 110 nM, less than about 105 nM, or less than about 100 nM.

### LOV-Domain Light-Activated Polypeptide

A LOV domain light-activated polypeptide that can be encoded by a nucleotide sequence present in a nucleic acid of a system (System 1 or System 2) of the present disclosure is activatable by blue light, and can cage a proteolytically cleavable linker attached to the light-activated polypeptide. Thus, in the absence of blue light, the proteolytically cleavable linker is caged, i.e., inaccessible to a protease. In the presence of blue light, the light-activated polypeptide undergoes a conformational change, such that the proteolytically cleavable linker is uncaged and becomes accessible to a protease. A LOV domain light-activated polypeptide comprises a light, oxygen, or voltage (LOV) domain (a “LOV polypeptide”).

A suitable LOV domain light-activated polypeptide can have a length of from about 100 amino acids to about 150 amino acids. For example, a LOV polypeptide can comprise an amino acid sequence having at least 80%, at least 85%, at least 90%, at least 95%, at least 98%, at least 99%, or 100%, amino acid sequence identity to the LOV2 domain of Avena sativa phototropin 1 (AsLOV2).

In some cases, a suitable LOV domain light-activated polypeptide comprises an amino sequence having at least 80%, at least 85%, at least 90%, at least 95%, at least 98%, at least 99%, or 100%, amino acid sequence identity to the following LOV2 amino acid sequence: DLATTLERIEKNFVITDPRLPDNPIIFASDSFLQLTEYSREEILGRNCRFLQGPETDRATVRKI RDAIDNQTEVTVQLINYTKSGKKFWNLFHLQPMRDQKGDVQYFIGVQLDGTEHVRDAAEREGVM LIKKTAENIDEAAK (SEQ ID NO://); GenBank AF033096. In some cases, a suitable LOV polypeptide comprises an amino sequence having at least 80%, at least 85%, at least 90%, at least 95%, at least 98%, at least 99%, or 100%, amino acid sequence identity to the following LOV2 amino acid sequence: DLATTLERIEKNFVITDPRLPDNPIIFASDSFLQLTEYSREEILGRNCRFLQGPETDRATVRKI RDAIDNQTEVTVQLINYTKSGKKFWNLFHLQPMRDQKGDVQYFIGVQLDGTEHVRDAAEREGVM LIKKTAENIDEAAK (SEQ ID NO://); and has a length of from 142 amino acids to 150 amino acids. In some cases, a suitable LOV domain light-activated polypeptide comprises an amino sequence having at least 80%, at least 85%, at least 90%, at least 95%, at least 98%, at least 99%, or 100%, amino acid sequence identity to the following LOV2 amino acid sequence: DLATTLERIEKNFVITDPRLPDNPIIFASDSFLQLTEYSREEILGRNCRFLQGPETDRATVRKI RDAIDNQTEVTVQLINYTKSGKKFWNLFHLQPMRDQKGDVQYFIGVQLDGTEHVRDAAEREGVM LIKKTAENIDEAAK (SEQ ID NO://); and has a length of 142 amino acids.

In some cases, a suitable LOV domain light-activated polypeptide comprises an amino sequence having at least 80%, at least 85%, at least 90%, at least 95%, at least 98%, at least 99%, or 100%, amino acid sequence identity to the following amino acid sequence: SLATTLERIEKNFVITDPRLPDNPIIFASDSFLQLTEYSREEILGRNCRFLQGPETDRATVR KIRDAIDNQTEVTVQLINYTKSGKKFWNLFHLQPMRDQKGDVQYFIGVQLDGTEHVRD AAEREAVMLIKKTAEEIDEAAK (SEQ ID NO://). In some cases, a suitable LOV domain light-activated polypeptide comprises an amino sequence having at least 80%, at least 85%, at least 90%, at least 95%, at least 98%, at least 99%, or 100%, amino acid sequence identity to the following amino acid sequence: SLATTLERIEKNFVITDPRLPDNPIIFASDSFLQLTEYSREEILGRNCRFLQGPETDRATVR KIRDAIDNQTEVTVQLINYTKSGKKFWNLFHLQPMRDQKGDVQYFIGVQLDGTEHVRD AAEREAVMLIKKTAEEIDEAAK (SEQ ID NO://); and has a length of from about 142 amino acids to about 150 amino acids. In some cases, a suitable LOV domain light-activated polypeptide comprises an amino sequence having at least 80%, at least 85%, at least 90%, at least 95%, at least 98%, at least 99%, or 100%, amino acid sequence identity to the following amino acid sequence: SLATTLERIEKNFVITDPRLPDNPIIFASDSFLQLTEYSREEILGRNCRFLQGPETDRATVR KIRDAIDNQTEVTVQLINYTKSGKKFWNLFHLQPMRDQKGDVQYFIGVQLDGTEHVRD AAEREAVMLIKKTAEEIDEAAK (SEQ ID NO://); and has a length of 142 amino acids.

In some cases, a suitable LOV domain light-activated polypeptide comprises an amino sequence having at least 80%, at least 85%, at least 90%, at least 95%, at least 98%, at least 99%, or 100%, amino acid sequence identity to the following amino acid sequence: SLATTLERIEKNFVITDPRLPDNPIIFASDSFLQLTEYSREEILGRNCRFLQGPETDRATVR KIRDAIDNQTEVTVQLINYTKSGKKFWNLFHLQPMRDQKGDVQYFIGVQLDGTEHVRD AAEREAVMLIKKTAEEIDEAAK (SEQ ID NO://); and comprises a substitution at one or more of amino acids L2, N12, A28, H117, and I130, where the numbering is based on the amino acid sequence SLATTLERIEKNFVITDPRLPDNPIIFASDSFLQLTEYSREEILGRNCRFLQGPETDRATVR KIRDAIDNQTEVTVQLINYTKSGKKFWNLFHLQPMRDQKGDVQYFIGVQLDGTEHVRD AAEREAVMLIKKTAEEIDEAAK (SEQ ID NO://). In some cases, the LOV domain light-activated polypeptide comprises a substitution selected from an L2R substitution, an L2H substitution, an L2P substitution, and an L2K substitution. In some cases, the LOV polypeptide comprises a substitution selected from an N12S substitution, an N12T substitution, and an N12Q substitution. In some cases, the LOV polypeptide comprises a substitution selected from an A28V substitution, an A28I substitution, and an A28L substitution. In some cases, the LOV polypeptide comprises a substitution selected from an H117R substitution, and an H117K substitution. In some cases, the LOV polypeptide comprises a substitution selected from an I130V substitution, an I130A substitution, and an I130L substitution. In some cases, the LOV polypeptide comprises substitutions at amino acids L2, N12, and I130. In some cases, the LOV polypeptide comprises substitutions at amino acids L2, N12, H117, and I130. In some cases, the LOV polypeptide comprises substitutions at amino acids A28 and H117. In some cases, the LOV polypeptide comprises substitutions at amino acids N12 and I130. In some cases, the LOV polypeptide comprises an L2R substitution, an N12S substitution, and an I130V substitution. In some cases, the LOV polypeptide comprises an N12S substitution and an I130V substitution. In some cases, the LOV polypeptide comprises an A28V substitution and an H117R substitution. In some cases, the LOV polypeptide comprises an L2P substitution, an N12S substitution, an I130V substitution, and an H117R substitution. In some cases, the LOV polypeptide comprises an L2P substitution, an N12S substitution, an A28V substitution, an H117R substitution, and an I130V substitution. In some cases, the LOV polypeptide comprises an L2P substitution, an N12S substitution, an I130V substitution, and an H117R substitution. In some cases, the LOV polypeptide comprises an L2R substitution, an N12S substitution, an A28V substitution, an H117R substitution, and an I130V substitution. In some cases, the LOV polypeptide has a length of 142 amino acids, 143 amino acids, 144 amino acids, 145 amino acids, 146 amino acids, 147 amino acids, 148 amino acids, 149 amino acids, or 150 amino acids. In some cases, the LOV polypeptide has a length of 142 amino acids.

In some cases, a suitable LOV polypeptide comprises an amino sequence having at least 80%, at least 85%, at least 90%, at least 95%, at least 98%, at least 99%, or 100%, amino acid sequence identity to the following amino acid sequence: SRATTLERIEKSFVITDPRLPDNPIIFVSDSFLQLTEYSREEILGRNCRFLQGPETDRATVR KIRDAIDNQTEVTVQLINYTKSGKKFWNLFHLQPMRDQKGDVQYFIGVQLDGTERVRD AAEREAVMLVKKTAEEIDEAAK (SEQ ID NO://); and has an Arg at amino acid 2, a Ser at amino acid 12, a Val at amino acid 28, an Arg at amino acid 117, and a Val at amino acid 130, as indicated by bold and underlined letters; and has a length of 142 amino acids, 143 amino acids, 144 amino acids, 145 amino acids, 146 amino acids, 147 amino acids, 148 amino acids, 149 amino acids, or 150 amino acids. In some cases, a suitable LOV polypeptide comprises the following amino acid sequence: SRATTLERIEKSFVITDPRLPDNPIIFVSDSFLQLTEYSREEILGRNCRFLQGPETDRATVR KIRDAIDNQTEVTVQLINYTKSGKKFWNLFHLQPMRDQKGDVQYFIGVQLDGTERVRD AAEREAVMLVKKTAEEIDEAAK (SEQ ID NO://); and has a length of 142 amino acids.

In some cases, a suitable LOV polypeptide comprises an amino sequence having at least 80%, at least 85%, at least 90%, at least 95%, at least 98%, at least 99%, or 100%, amino acid sequence identity to the following amino acid sequence: SRATTLERIEKSFVITDPRLPDNPVIFVSDSFLQLTEYSREEILGRNCRFLQGPETDRATVR KIRDAIDNQTEVTVQLINYTKSGKKFWNLFHLQPMRDQKGDVQYFIGVQLDGTERVRD AAEREAVMLVKKTAEEIDEAAK (SEQ ID NO://); and has an Arg at amino acid 2, a Ser at amino acid 12, a Val at amino acid 25, a Val at amino acid 28, an Arg at amino acid 117, and a Val at amino acid 130, as indicated by bold and underlined letters; and has a length of 142 amino acids, 143 amino acids, 144 amino acids, 145 amino acids, 146 amino acids, 147 amino acids, 148 amino acids, 149 amino acids, or 150 amino acids. In some cases, a suitable LOV polypeptide comprises the following amino acid sequence: SRATTLERIEKSFVITDPRLPDNPVIFVSDSFLQLTEYSREEILGRNCRFLQGPETDRATVR KIRDAIDNQTEVTVQLINYTKSGKKFWNLFHLQPMRDQKGDVQYFIGVQLDGTERVRD AAEREAVMLVKKTAEEIDEAAK (SEQ ID NO://); and has a length of 142 amino acids.

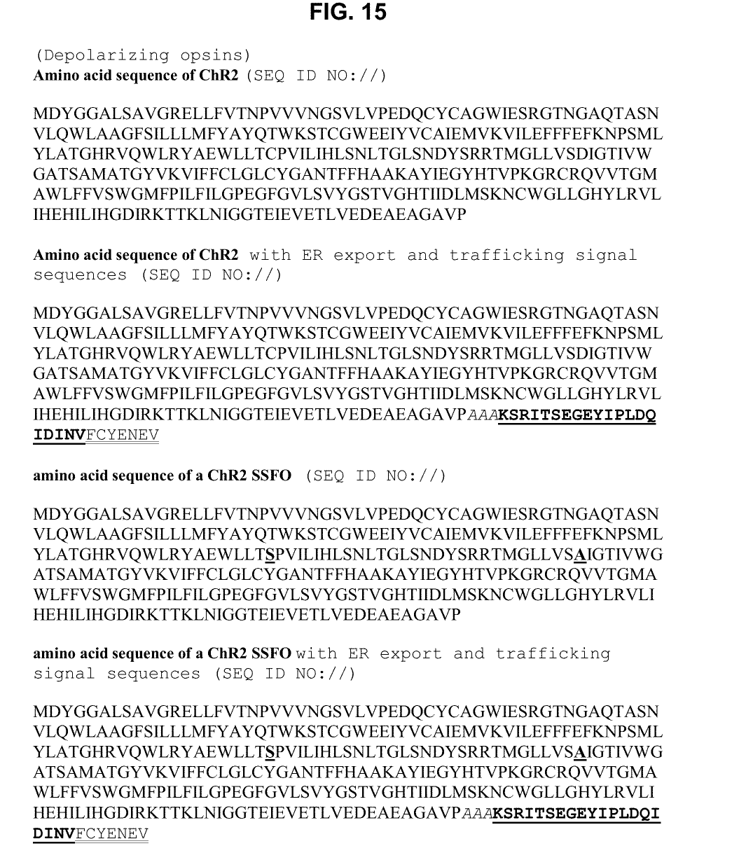

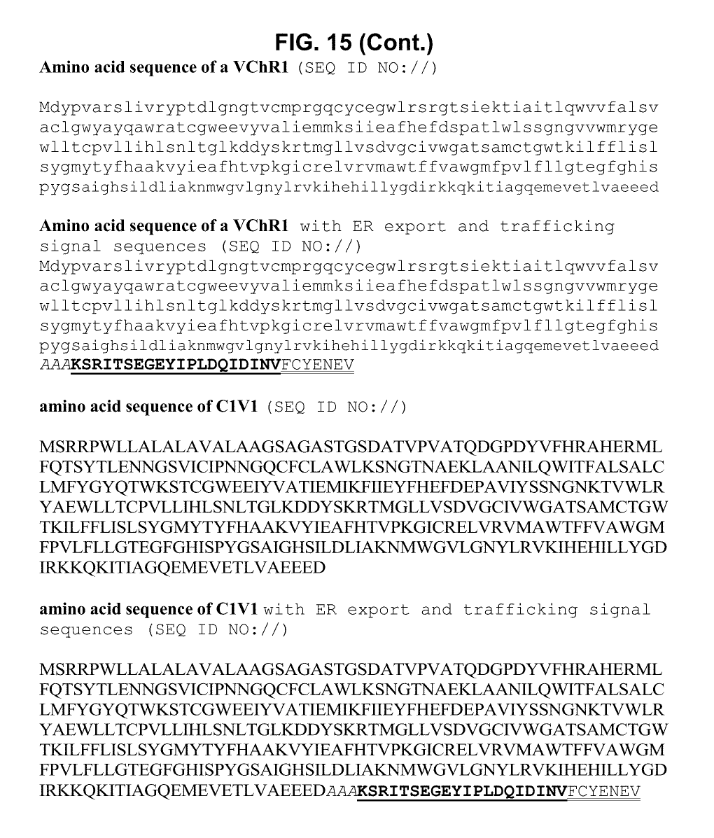

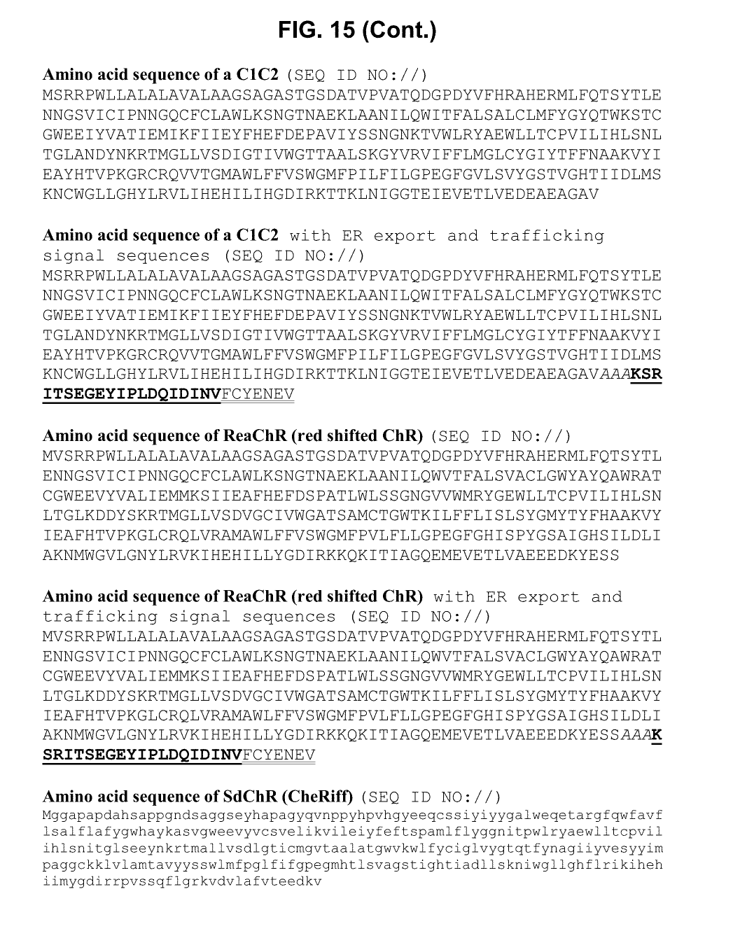

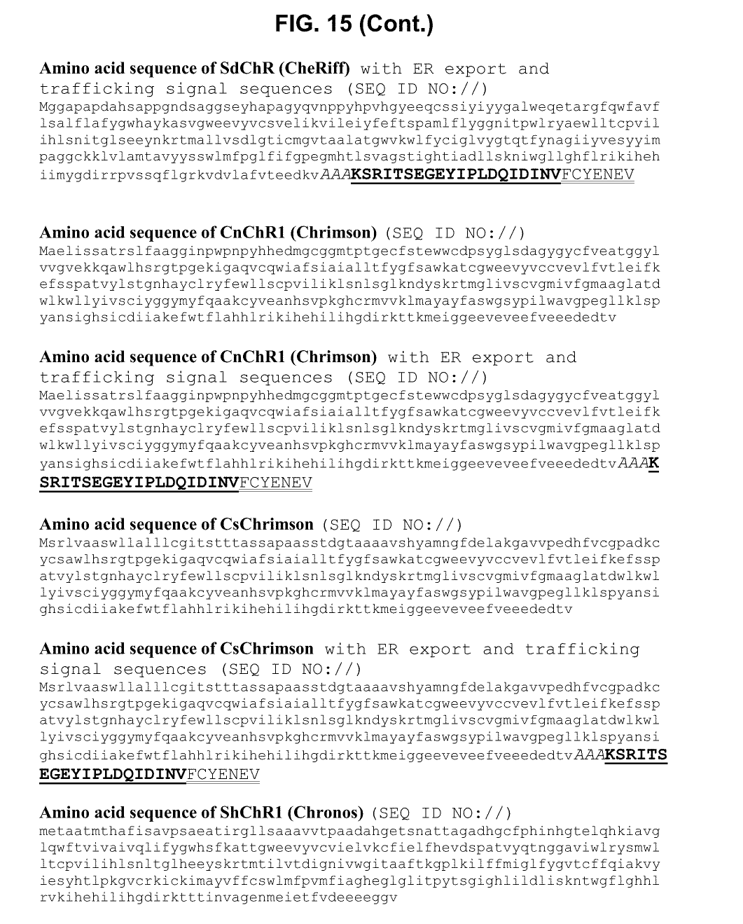

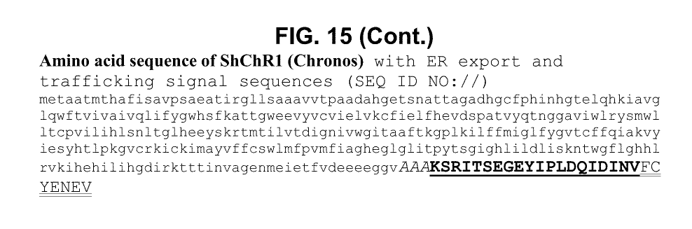

A suitable LOV domain light-activated polypeptide comprises one or more amino acid substitutions relative to the following LOV2 amino acid sequence: DLATTLERIEKNFVITDPRLPDNPIIFASDSFLQLTEYSREEILGRNCRFLQGPETDRATVRKI RDAIDNQTEVTVQLINYTKSGKKFWNLFHLQPMRDQKGDVQYFIGVQLDGTEHVRDAAEREGVM LIKKTAENIDEAAK (SEQ ID NO://). In some cases, a suitable LOV domain light-activated polypeptide comprises one or more amino acid substitutions at positions selected from 1, 2, 12, 25, 28, 91, 100, 117, 118, 119, 120, 126, 128, 135, 136, and 138, relative to the LOV2 amino acid sequence depicted in FIG. 15A. Suitable substitutions include, Asp→Ser at amino acid 1; Asp→Phe at amino acid 1; Leu→Arg at amino acid 2; Asn→Ser at amino acid 12; Ile→Val at amino acid 12; Ala→Val at amino acid 28; Leu→Val at amino acid 91; Gln→Tyr at amino acid 100; His→Arg at amino acid 117; Val→Leu at amino acid 118; Arg→His at amino acid 119; Asp→Gly at amino acid 120; Gly→Ala at amino acid 126; Met→Cys at amino acid 128; Glu→Phe at amino acid 135; Asn→Gln at amino acid 136; Asn→Glu at amino acid 136; and Asp→Ala at amino acid 138, where the amino acid numbering is based on the number of the following LOV2 amino acid sequence: DLATTLERIEKNFVITDPRLPDNPIIFASDSFLQLTEYSREEILGRNCRFLQGPETDRATVRKI RDAIDNQTEVTVQLINYTKSGKKFWNLFHLQPMRDQKGDVQYFIGVQLDGTEHVRDAAEREGVM LIKKTAENIDEAAK (SEQ ID NO://).

In some cases, a suitable LOV domain light-activated polypeptide comprises an amino acid sequence having at least 80%, at least 85%, at least 90%, at least 95%, at least 98%, at least 99%, or 100%, amino acid sequence identity to the following amino acid sequence: SLATTLERIEKNFVITDPRLPDNPIIFASDSFLQLTEYSREEILGRNCRFLQGPETDRATVR KIRDAIDNQTEVTVQLINYTKSGKKFWNLFHLQPMRDQKGDVQYFIGVQLDGTEHVRD AAEREAVMLIKKTAEEIDEAAK (SEQ ID NO://), where amino acid 1 is Ser, amino acid 28 is Ala, amino acid 126 is Ala, and amino acid 136 is Glu. In some case, the suitable LOV domain light-activated polypeptide has a length of 142 amino acids.

In some cases, a suitable LOV domain light-activated polypeptide comprises an amino acid sequence having at least 80%, at least 85%, at least 90%, at least 95%, at least 98%, at least 99%, or 100%, amino acid sequence identity to the following amino acid sequence: SRATTLERIEKSFVITDPRLPDNPIIFVSDSFLQLTEYSREEILGRNCRFLQGPETDRATVR KIRDAIDNQTEVTVQLINYTKSGKKFWNLFHLQPMRDQKGDVQYFIGVQLDGTERVRD AAEREAVMLVKKTAEEIDEAAK (SEQ ID NO://), where amino acid 1 is Ser; amino acid 2 is Arg; amino acid 12 is Ser; amino acid 28 is Ala; amino acid 117 is Arg; amino acid 126 is Ala; and amino acid 136 is Glu. In some case, the suitable LOV domain light-activated polypeptide has a length of 142 amino acids.

In some cases, a suitable LOV domain light-activated polypeptide comprises an amino acid sequence having at least 80%, at least 85%, at least 90%, at least 95%, at least 98%, at least 99%, or 100%, amino acid sequence identity to the following amino acid sequence: SRATTLERIEKSFVITDPRLPDNPVIFVSDSFLQLTEYSREEILGRNCRFLQGPETDRATVR KIRDAIDNQTEVTVQLINYTKSGKKFWNLFHLQPMRDQKGDVQYFIGVQLDGTERVRD AAEREAVMLVKKTAEEIDEAAK (SEQ ID NO://), where amino acid 1 is Ser; amino acid 2 is Arg; amino acid 12 is Ser; amino acid 25 is Val; amino acid 28 is Val; amino acid 117 is Arg; amino acid 126 is Ala; amino acid 130 is Val; and amino acid 136 is Glu. In some case, the LOV domain light-activated polypeptide has a length of 142 amino acids.

In some cases, a suitable LOV domain light-activated polypeptide comprises an amino acid sequence having at least 80%, at least 85%, at least 90%, at least 95%, at least 98%, at least 99%, or 100%, amino acid sequence identity to the following amino acid sequence: S\sqrt{R}ATTLERIEKSFVITDPRLPDNPIIFVSDSFLQLTEYSREEILGRNCRFLQGPETDRATVR KIRDAIDNQTEVTVQLINYTKSGKKFWNVFHLQPMRDYKGDVQYFIGVQLDGTERLHG AAEREAVCLVKKTAFQIAEAAK (SEQ ID NO://), where amino acid 1 is Ser; amino acid 2 is Arg; amino acid 12 is Ser; amino acid 28 is Ala; amino acid 91 is Val; amino acid 100 is Tyr; amino acid 117 is Arg; amino acid 118 is Leu; amino acid 119 is His; amino acid 120 is Gly; amino acid 126 is Ala; amino acid 128 is Cys; amino acid 130 is Val; amino acid 135 is Phe; amino acid 136 is Gln; and amino acid 138 is Ala. In some case, the LOV domain light-activated polypeptide has a length of 142 amino acids.

In some cases, a suitable LOV domain light-activated polypeptide comprises an amino acid sequence having at least 80%, at least 85%, at least 90%, at least 95%, at least 98%, at least 99%, or 100%, amino acid sequence identity to the following amino acid sequence: SRATTLERIEKSFVITDPRLPDNPIIFVSDSFLQLTEYSREEILGRNCRFLQGPETDRATVR KIRDAIDNQTEVTVQLINYTKSGKKFWNLFHLQPMRDQKGDVQYFIGVQLDGTERVRD AAEREAVMLVKKTAEEID (SEQ ID NO://), where amino acid 1 is Ser; amino acid 2 is Arg; amino acid 12 is Ser; amino acid 28 is Val; amino acid 117 is Arg; amino acid 126 is Ala; amino acid 130 is Val; and amino acid 136 is Glu. In some case, the LOV domain light-activated polypeptide has a length of 138 amino acids.

In some cases, a suitable LOV domain light-activated polypeptide comprises an amino acid sequence having at least 80%, at least 85%, at least 90%, at least 95%, at least 98%, at least 99%, or 100%, amino acid sequence identity to the following amino acid sequence: SRATTLERIEKSFVITDPRLPDNPIIFVSDSFLQLTEYSREEILGRNCRFLQGPETDRATVR KIRDAIDNQTEVTVQLINYTKSGKKFWNVFHLQPMRDYKGDVQYFIGVQLDGTERLHG AAEREAVCLVKKTAFQIA (SEQ ID NO://), where amino acid 1 is Ser; amino acid 2 is Arg; amino acid 12 is Ser; amino acid 28 is Val; amino acid 91 is Val; amino acid 100 is Tyr; amino acid 117 is Arg; amino acid 118 is Leu; amino acid 119 is His; amino acid 120 is Gly; amino acid 126 is Ala; amino acid 128 is Cys; amino acid 130 is Val; amino acid 135 is Phe; amino acid 136 is Gln; and amino acid 138 is Ala. In some case, the LOV domain light-activated polypeptide has a length of 138 amino acids.

In some cases, a LOV light-activated polypeptide comprises the following amino acid sequence:

(SEQ ID NO: //)
FRATTLERIEKSFVITDPRLPDNPIIFVSDSFLQLTEYSREEILGRNCRF
LQGPETDRATVRKIRDAIDNQTEVTVQLINYTKSGKKFWNVFHLQPMRDY
KGDVQYFIGVQLDGTERLHGAAEREAVCLVKKTAFQIA.

In some cases, a LOV light-activated polypeptide comprises the following amino acid sequence:

(SEQ ID NO: //)
SRATTLERIEKSFVITDPRLPDNPIIFVSDSFLQLTEYSREEILGRNCRF
LQGPETDRATVRKIRDAIDNQTEVTVQLINYTKSGKKFWNLFHLQPMRDQ
KGDVQYFIGVQLDGTERVRDAAEREAVMLVKKTAEEID.

In some cases, a LOV light-activated polypeptide comprises the following amino acid sequence:

(SEQ ID NO: //)
FRATTLERIEKSFVITDPRLPDNPIIFVSDSFLQLTEYSREEILGRNCRF
LQGPETDRATVRKIRDAIDNQTEVTVQLINYTKSGKKFWNVFHLQPMRDY
KGDVQYFIGVQLDGTERLHGAAEREAVCLVKKTAFQIA.

In some cases, a LOV light-activated polypeptide comprises the following amino acid

(SEQ ID NO: //)
SRATTLERIEKSFVITDPRLPDNPIIFVSDSFLQLTEYSREEILGRNCRF
LQGPETDRATVRKIRDAIDNQTEVTVQLINYTKSGKKFWNVFHLQPMRDY
KGDVQYFIGVQLDGTERLHGAAEREAVCLVKKTAFEIDEAAK.

In some cases, a LOV light-activated polypeptide comprises the following amino acid sequence:

(SEQ ID NO: //)
SRATTLERIEKSFVITDPRLPDNPIIFVSDSFLQLTEYSREEILGRNCRF
LQGPETDRATVRKIRDAIDNQTEVTVQLINYTKSGKKFWNLFHLQPMRDQ
KGDVQYFIGVQLDGTERVRDAAEREAVMLVKKTAEEIDEAAK.

LOV light-activated polypeptide cages the proteolytically cleavable linker in the absence of light of an activating wavelength, the proteolytically cleavable linker is substantially not accessible to the protease. Thus, e.g., in the absence of light of an activating wavelength (e.g., in the dark; or in the presence of light of a wavelength other than blue light), the proteolytically cleavable linker is cleaved, if at all, to a degree that is more than 50% less, more than 60% less, more than 70% less, more than 80% less, more than 90% less, more than 95% less, more than 98% less, or more than 99% less, than the degree of cleavage of the proteolytically cleavable linker in the presence of light of an activating wavelength (e.g., blue light, e.g., light of a wavelength in the range of from about 450 nm to about 495 nm, from about 460 nm to about 490 nm, from about 470 nm to about 480 nm, e.g., 473 nm).

Non-limiting examples of suitable polypeptides comprising: a) a LOV light-activated polypeptide; and b) a proteolytically cleavable linker include the following (where the proteolytically cleavable linker is underlined, and where the triangle indicates the cleavage site):

1)
(SEQ ID NO: //)
SRATTLERIEKSFVITDPRLPDNPIIFVSDSFLQLTEYSREEILGRNCRF
LQGPETDRATVRKIRDAIDNQTEVTVQLINYTKSGKKFWNLFHLQPMRDQ
KGDVQYFIGVQLDGTERVRDAAEREAVMLVKKTAEEIDEAAKENLYFQ_{▴}M;
2)
(SEQ ID NO: //)
SRATTLERIEKSFVITDPRLPDNPIIFVSDSFLQLTEYSREEILGRNCRF
LQGPETDRATVRKIRDAIDNQTEVTVQLINYTKSGKKFWNVFHLQPMRDY
KGDVQYFIGVQLDGTERLHGAAEREAVCLVKKTAFEIDEAAKENLYFQ_{▴}M;
3)
(SEQ ID NO: //)
FRATTLERIEKSFVITDPRLPDNPIIFVSDSFLQLTEYSREEILGRNCRF
LQGPETDRATVRKIRDAIDNQTEVTVQLINYTKSGKKFWNVFHLQPMRDY
KGDVQYFIGVQLDGTERLHGAAEREAVCLVKKTAFQIAENLYFQ_{▴}M;
4)
(SEQ ID NO: //)
SRATTLERIEKSFVITDPRLPDNPIIFVSDSFLQLTEYSREEILGRNCRF
LQGPETDRATVRKIRDAIDNQTEVTVQLINYTKSGKKFWNLFHLQPMRDQ
KGDVQYFIGVQLDGTERVRDAAEREAVMLVKKTAEEIDENLYFQ_{▴}G;
and
5)
(SEQ ID NO: //)
FRATTLERIEKSFVITDPRLPDNPIIFVSDSFLQLTEYSREEILGRNCRF
LQGPETDRATVRKIRDAIDNQTEVTVQLINYTKSGKKFWNVFHLQPMRDY
KGDVQYFIGVQLDGTERLHGAAEREAVCLVKKTAFQIAENLYFQ_{▴}G.

### Proteolytically Cleavable Linker

The proteolytically cleavable linker can include a protease recognition sequence recognized by a protease selected from the group consisting of alanine carboxypeptidase, Armillaria mellea astacin, bacterial leucyl aminopeptidase, cancer procoagulant, cathepsin B, clostripain, cytosol alanyl aminopeptidase, elastase, endoproteinase Arg-C, enterokinase, gastricsin, gelatinase, Gly-X carboxypeptidase, glycyl endopeptidase, human rhinovirus 3C protease, hypodermin C, IgA-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picornain 2A, picornain 3C, proendopeptidase, prolyl aminopeptidase, proprotein convertase I, proprotein convertase II, russellysin, saccharopepsin, semenogelase, T-plasminogen activator, thrombin, tissue kallikrein, tobacco etch virus (TEV), togavirin, tryptophanyl aminopeptidase, U-plasminogen activator, V8, venombin A, venombin AB, and Xaa-pro aminopeptidase.

For example, the proteolytically cleavable linker can comprise a matrix metalloproteinase (MMP) cleavage site, e.g., a cleavage site for a MMP selected from collagenase-1, -2, and -3 (MMP-1, -8, and -13), gelatinase A and B (MMP-2 and -9), stromelysin 1, 2, and 3 (MMP-3, -10, and -11), matrilysin (MMP-7), and membrane metalloproteinases (MT1-MMP and MT2-MMP). For example, the cleavage sequence of MMP-9 is Pro-X-X-Hy (wherein, X represents an arbitrary residue; Hy, a hydrophobic residue), e.g., Pro-X-X-Hy-(Ser/Thr), e.g., Pro-Leu/Gln-Gly-Met-Thr-Ser (SEQ ID NO://) or Pro-Leu/Gln-Gly-Met-Thr (SEQ ID NO://). Another example of a protease cleavage site is a plasminogen activator cleavage site, e.g., a uPA or a tissue plasminogen activator (tPA) cleavage site. Another example of a suitable protease cleavage site is a prolactin cleavage site. Specific examples of cleavage sequences of uPA and tPA include sequences comprising Val-Gly-Arg. Another example of a protease cleavage site that can be included in a proteolytically cleavable linker is a tobacco etch virus (TEV) protease cleavage site, e.g., ENLYFQS (SEQ ID NO://), where the protease cleaves between the glutamine and the serine; or ENLYFQY (SEQ ID NO://), where the protease cleaves between the glutamine and the tyrosine; or ENLYFQL (SEQ ID NO://), where the protease cleaves between the glutamine and the leucine. Another example of a protease cleavage site that can be included in a proteolytically cleavable linker is an enterokinase cleavage site, e.g., DDDDK (SEQ ID NO://), where cleavage occurs after the lysine residue. Another example of a protease cleavage site that can be included in a proteolytically cleavable linker is a thrombin cleavage site, e.g., LVPR (SEQ ID NO://) (e.g., where the proteolytically cleavable linker comprises the sequence LVPRGS (SEQ ID NO://)). Additional suitable linkers comprising protease cleavage sites include linkers comprising one or more of the following amino acid sequences: LEVLFQGP (SEQ ID NO://), cleaved by PreScission protease (a fusion protein comprising human rhinovirus 3C protease and glutathione-S-transferase; Walker et al. (1994) Biotechnol. 12:601); a thrombin cleavage site, e.g., CGLVPAGSGP (SEQ ID NO://); SLLKSRMVPNFN (SEQ ID NO://) or SLLIARRMPNFN (SEQ ID NO://), cleaved by cathepsin B; SKLVQASASGVN (SEQ ID NO://) or SSYLKASDAPDN (SEQ ID NO://), cleaved by an Epstein-Barr virus protease; RPKPQQFFGLMN (SEQ ID NO://) cleaved by MMP-3 (stromelysin); SLRPLALWRSFN (SEQ ID NO://) cleaved by MMP-7 (matrilysin); SPQGIAGQRNFN (SEQ ID NO://) cleaved by MMP-9; DVDERDVRGFASFL SEQ ID NO://) cleaved by a thermolysin-like MMP; SLPLGLWAPNFN (SEQ ID NO://) cleaved by matrix metalloproteinase 2(MMP-2); SLLIFRSWANFN (SEQ ID NO://) cleaved by cathespin L; SGVVIATVIVIT (SEQ ID NO://) cleaved by cathepsin D; SLGPQGIWGQFN (SEQ ID NO://) cleaved by matrix metalloproteinase 1(MMP-1); KKSPGRVVGGSV (SEQ ID NO://) cleaved by urokinase-type plasminogen activator; PQGLLGAPGILG (SEQ ID NO://) cleaved by membrane type 1 matrixmetalloproteinase (MT-MMP); HGPEGLRVGFYESDVMGRGHARLVHVEEPHT (SEQ ID NO://) cleaved by stromelysin 3 (or MMP-11), thermolysin, fibroblast collagenase and stromelysin-1; GPQGLAGQRGIV (SEQ ID NO://) cleaved by matrix metalloproteinase 13 (collagenase-3); GGSGQRGRKALE (SEQ ID NO://) cleaved by tissue-type plasminogen activator(tPA); SLSALLSSDIFN (SEQ ID NO://) cleaved by human prostate-specific antigen; SLPRFKIIGGFN (SEQ ID NO://) cleaved by kallikrein (hK3); SLLGIAVPGNFN (SEQ ID NO://) cleaved by neutrophil elastase; and FFKNIVTPRTPP (SEQ ID NO://) cleaved by calpain (calcium activated neutral protease).

Suitable proteolytically cleavable linkers also include ENLYFQX (SEQ ID NO://; where X is any amino acid), ENLYFQG (SEQ ID NO://), ENLYFQS (SEQ ID NO://), ENLYFQY (SEQ ID NO://), ENLYFQL (SEQ ID NO://), ENLYFQW (SEQ ID NO://), ENLYFQM (SEQ ID NO://), ENLYFQH (SEQ ID NO://), ENLYFQN (SEQ ID NO://), ENLYFQA (SEQ ID NO://), and ENLYFQQ (SEQ ID NO://).

Suitable proteolytically cleavable linkers also include NS3 protease cleavage sites such as: DEVVECS (SEQ ID NO://), DEAEDVVECS (SEQ ID NO://), EDAAEEVVECS (SEQ ID NO://).

Suitable proteolytically cleavable linkers also include calpain cleavage site, where suitable calpain cleavage sites include, e.g., PLFAAR (SEQ ID NO://) and QQEVYGMMPRD (SEQ ID NO://).

In some cases, the proteolytically cleavable linker comprises an amino acid sequence that is substantially not cleaved by any endogenous protease in a given cell (e.g., a eukaryotic cell; e.g., a mammalian cell; e.g., a particular type of mammalian cell). In some cases, the proteolytically cleavable linker comprises an amino acid sequence that is cleaved by a viral protease, and that is substantially not cleaved by any endogenous protease in a given cell (e.g., a eukaryotic cell; e.g., a mammalian cell; e.g., a particular type of mammalian cell). In some cases, the proteolytically cleavable linker comprises an amino acid sequence that is cleaved by a non-naturally occurring (e.g., engineered) protease, and that is substantially not cleaved by any endogenous protease in a given cell (e.g., a eukaryotic cell; e.g., a mammalian cell; e.g., a particular type of mammalian cell).

In some cases, the proteolytically cleavable linker comprises an amino acid sequence that is cleaved by a protease that is endogenous to a given cell (e.g., a eukaryotic cell; e.g., a mammalian cell; e.g., a particular type of mammalian cell).

### Proteases

In some cases, the protease is a protease that is not normally produced in a particular cell; e.g., the protease is heterologous to the cell. For example, in some cases, the protease is one that is not normally produced in a mammalian cell. Examples of such proteases include viral proteases, insect-specific proteases, venom proteases, and the like.

In some cases, the protease is a protease that is normally produced in a particular cell; e.g., the protease is an endogenous protease (e.g., a calpain protease; etc.).

Suitable proteases include, but are not limited to, alanine carboxypeptidase, Armillaria mellea astacin, bacterial leucyl aminopeptidase, cancer procoagulant, cathepsin B, clostripain, cytosol alanyl aminopeptidase, elastase, endoproteinase Arg-C, enterokinase, gastricsin, gelatinase, Gly-X carboxypeptidase, glycyl endopeptidase, human rhinovirus 3C protease, hypodermin C, IgA-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picornain 2A, picornain 3C, proendopeptidase, prolyl aminopeptidase, proprotein convertase I, proprotein convertase II, russellysin, saccharopepsin, semenogelase, T-plasminogen activator, thrombin, tissue kallikrein, tobacco etch virus (TEV), togavirin, tryptophanyl aminopeptidase, U-plasminogen activator, Factor Xa, V8, venombin A, venombin AB, a calpain protease, and an Xaa-pro aminopeptidase.

Suitable proteases include a matrix metalloproteinase (MMP) (e.g., an MMP selected from collagenase-1, -2, and -3 (MMP-1, -8, and -13), gelatinase A and B (MMP-2 and -9), stromelysin 1, 2, and 3 (MMP-3, -10, and -11), matrilysin (MMP-7), and membrane metalloproteinases (MT1-MMP and MT2-MMP); a plasminogen activator (e.g., a uPA or a tissue plasminogen activator (tPA)). Another example of a suitable protease is prolactin. Another example of a suitable protease is a tobacco etch virus (TEV) protease. Another example of suitable protease is enterokinase. Another example of suitable protease is thrombin. Additional examples of suitable protease are: a PreScission protease (a fusion protein comprising human rhinovirus 3C protease and glutathione-S-transferase; Walker et al. (1994) Biotechnol. 12:601); cathepsin B; an Epstein-Barr virus protease; cathespin L; cathepsin D; thermolysin; kallikrein (hK3); neutrophil elastase; calpain (calcium activated neutral protease); and NS3 protease.

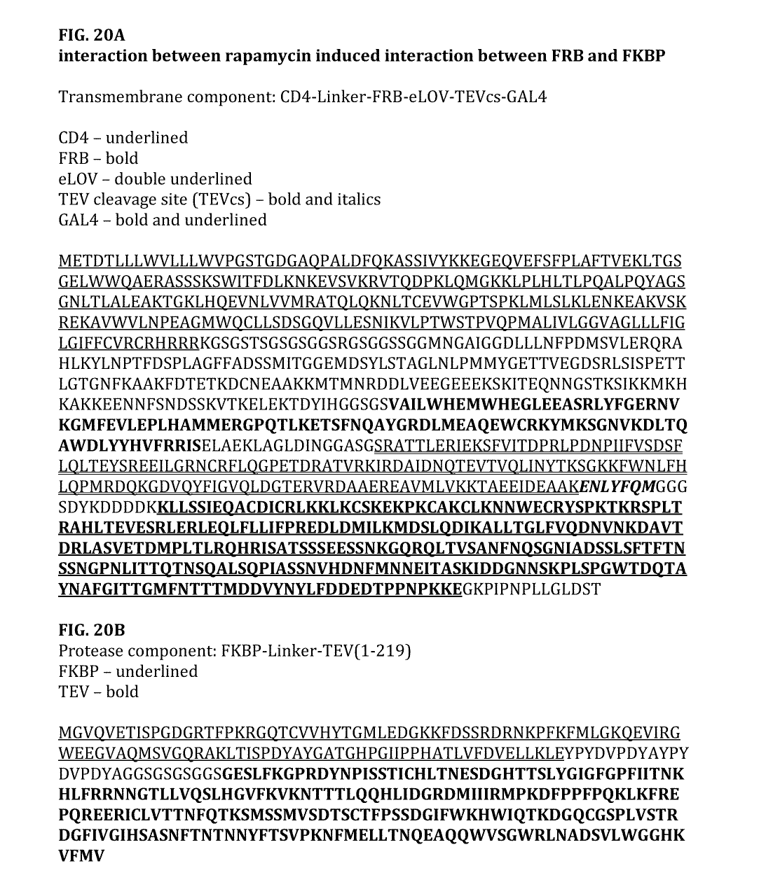

In some cases, a suitable protease is a TEV protease. In some cases, a suitable protease comprises an amino acid sequence having at least 80%, at least 85%, at least 90%, at least 95%, at least 98%, at least 99%, or 100%, amino acid sequence identity to the amino acid sequence depicted in FIG. 20A. In some cases, a suitable protease is a TEV protease. In some cases, a suitable protease comprises an amino acid sequence having at least 80%, at least 85%, at least 90%, at least 95%, at least 98%, at least 99%, or 100%, amino acid sequence identity to the amino acid sequence depicted in FIG. 20B. In some cases, a suitable protease is a TEV protease. In some cases, a suitable protease comprises an amino acid sequence having at least 80%, at least 85%, at least 90%, at least 95%, at least 98%, at least 99%, or 100%, amino acid sequence identity to the amino acid sequence depicted in FIG. 20C. In some cases, a suitable protease is a TEV protease. In some cases, a suitable protease comprises an amino acid sequence having at least 80%, at least 85%, at least 90%, at least 95%, at least 98%, at least 99%, or 100%, amino acid sequence identity to the amino acid sequence depicted in FIG. 20D.

In some cases, a suitable TEV protease comprises the amino acid sequence

(SEQ ID NO: //)
GESLFKGPRDYNPISSTICHLTNESDGHTTSLYGIGFGPFIITNKHLFRR
NNGTLLVQSLHGVFKVKNTTTLQQHLIDGRDMIIIRMPKDFPPFPQKLKF
REPQREERICLVTTNFQTKSMSSMVSDTSCTFPSSDGIFWKHWIQTKDGQ
CGSPLVSTRDGFIVGIHSASNFTNTNNYFTSVPKNFMELLTNQEAQQWVS
GWRLNADSVLWGGHKVFMV.

A suitable TEV protease can have a length of from about 200 amino acids to about 250 amino acids. For example, a suitable TEV protease can have a length of from about 200 amino acids to about 220 amino acids, from about 220 amino acids to about 240 amino acids, or from about 240 amino acids to about 250 amino acids. For example, a suitable TEV protease can have a length of 219 amino acids, 242 amino acids, or 238 amino acids.

### System Comprising a Nucleic Acid Comprising a Nucleotide Sequence Encoding a Polypeptide of Interest

As noted above, a system of present disclosure includes a nucleic acid system (“System 2”) comprising: a) a first nucleic acid comprising a nucleotide sequence encoding a first fusion polypeptide comprising, in order from amino terminus to carboxyl terminus: i) a tethering domain (e.g., a transmembrane domain); ii) a first polypeptide member of a protein-interaction pair; iii) a LOV light-activated polypeptide comprising an amino acid sequence having at least 80% amino acid sequence identity to the amino acid sequence depicted in any one of FIG. 11A-11G; iv) a proteolytically cleavable linker; and v) a polypeptide of interest; and b) a second nucleic acid comprising a nucleotide sequence encoding a second fusion polypeptide comprising: i) a second polypeptide member of the protein interaction pair; and ii) a protease that cleaves the proteolytically cleavable linker. Thus, in some cases, the present disclosure provides a nucleic acid system in which the first nucleic acid comprises a nucleotide sequence encoding a first fusion polypeptide that comprises a polypeptide of interest.

### Polypeptides of Interest

Suitable polypeptides of interest that can be encoded in a system of the present disclosure include, but are not limited to, a reporter gene product, an opsin, a DREADD, a toxin, an enzyme, a transcription factor, an antibiotic resistance factor, a genome editing endonuclease, an RNA-guided endonuclease, a protease, a kinase, a phosphatase, a phosphorylase, a lipase, a receptor, an antibody, a fluorescent protein, a biotin ligase, a peroxidase such as APEX or APEX2, a base editing enzyme, a recombinase, a synaptic marker, a signaling protein, an effector protein of a receptor, a protein that regulates synaptic vesicle fusion or protein trafficking or organelle trafficking, a portion (e.g., a split half) of any one of the aforementioned polypeptides. In some cases, the gene product is inactive until released from the first, light-activated, fusion polypeptide. In some cases, the gene product is a nuclear protein. In some cases, the gene product is a cytosolic protein. In some cases, the gene product is a mitochondrial protein. In some cases, the gene product is a transmembrane protein.

### Biotin Ligase

A suitable biotin ligase includes a BirA biotin-protein ligase polypeptide. A BirA biotin-protein ligase activates biotin to form biotinyl 5′ adenylate and transfers the biotin to a biotin-acceptor tag (BAT). A BAT can be present in a fusion protein, where the fusion protein comprises: a) a BAT; and b) a heterologous polypeptide. Suitable BATs include, e.g., GLNDIFEAQKIEWHE (SEQ ID NO://; see, e.g., Fairhead and Howarth (2015) Methods Mol. Biol. 1266:171).

A suitable BirA biotin-protein ligase polypeptide can comprise an amino acid sequence having at least at least 70%, at least 80%, at least 85%, at least 90%, at least 95%, at least 98%, at least 99%, or 100%, amino acid sequence identity to the following amino acid sequence:

(SEQ ID NO: //)
MKDNTVPLKL IALLANGEFH SGEQLGETLG MSRAAINKHI
QTLRDWGVDV FTVPGKGYSL PEPIQLLNAE EILSQLDGGS
VAVLPVIDST NQYLLDRIGE LKSGDACVAE YQQAGRGRRG
RKWFSPFGAN LYLSMFWRLE QGPAAAIGLS LVIGIVMAEV
LRKLGADKVR VKWPNDLYLQ DRKLAGILVE LTGKTGDAAQ
IVIGAGINMA MRRVEESVVN QGWITLQEAG INLDRNTLAA
MLIRELRAAL ELFEQEGLAP YLSRWEKLDN FINRPVKLII
GDKEIFGISR GIDKQGALLL EQDGIIKPWM GGEISLRSAE
K.

### Synaptic Markers

In some cases, a polypeptide of interest is a synaptic marker. Synaptic markers include, but are not limited to, PSD-95, SV2, homer, bassoon, synapsin I, synaptotagmin, synaptophysin, synaptobrevin, SAP102, α-adaptin, GluA1, NMDA receptor, LRRTM1, LRRTM2, SLITRK, neuroligin-1, neuroligin-2, gephyrin, GABA receptor, and the like.

### Nucleic Acid Editing Enzymes

In some cases, a polypeptide of interest is a nucleic acid-editing enzyme. Suitable nucleic acid-editing enzymes include, e.g., a DNA-editing enzyme, a cytidine deaminase, an adenosine deaminase, an apolipoprotein B mRNA-editing complex (APOBEC) family deaminase, an activation-induced cytidine deaminase (AID), an ACF1/ASE deaminase, and an ADAT family deaminase.

### Peroxidases

A suitable polypeptide of interest is in some cases a peroxidase, where suitable peroxidases include, e.g., horse radish peroxidase, yeast cytochrome c peroxidase (CCP), ascorbate peroxidase (APX), bacterial catalase-peroxidase (BCP), APEX, and APEX2. See, e.g., U.S. Patent Publication No. 2014/0206013.

An example of a suitable peroxidase is an APX, which has the following amino acid sequence: MGKSYPTVSA DYQKAVEKAK KKLRGFIAEK RCAPLMLRLA WHSAGTFDKG TKTGGPFGTI KHPAELAHSA NNGLDIAVRL LEPLKAEFPI LSYADFYQLA GVVAVEVTGG PEVPFHPGRE DKPEPPPEGR LPDATKGSDH LRDVFGKAMG LTDQDIVALS GGHTIGAAHK ERSGFEGPWT SNPLIFDNSY FTELLSGEKE GLLQLPSDKA LLSDPVFRPL VDKYAADEDA FFADYAEAHQ KLSELGFADA (SEQ ID NO://). In some cases, the peroxidase comprises a K14D substitution. In some cases, the peroxidase can contain a combination of (a) K14D, E112K, E228K, D229K, K14D/E112K, K14D/E228K, K14D/D229K, E17N/K20A/R21L, or K14D/W41F/E112K, and (b) S69F, G174F, W41F/S69F, D133A/T135F/K136F, W41F/D133A/T135F/K136F, S69F/D133A/T135F/K136F, or W41F/S69F/D133A/T135F/K136F. In some cases, the peroxidase can contain a combination of (a) single mutant K14D, single mutant E112K, single mutant E228K, single mutant D229K, double mutant K14D/E112K, double mutant K14D/E228K, double mutant K14D/D229K, triple mutant E17N/K20A/R21L, or triple mutant K14D/W41F/E112K, and (b) single mutant W41F, single mutant S69F, single mutant G174F, double mutant W41F/S69F, triple mutant D133A/T135F/K136F, quadruple mutant W41F/D133A/T135F/K136F, quadruple mutant S69F/D133A/T135F/K136F, or quintuple mutant W41F/S69F/D133A/T135F/K136F. Examples of such combined mutants include, but are not limited to, K14D/E112K/W41F (APEX), and K 14D/E112K/W41F/D133A/T135F/K136F. The amino acid numbering is based on the above-provided APX amino acid sequence.

### Antibodies

A suitable polypeptide of interest is in some cases an antibody. The terms “antibodies” and “immunoglobulin” include antibodies or immunoglobulins of any isotype, fragments of antibodies that retain specific binding to antigen, including, but not limited to, Fab, Fv, scFv, and Fd fragments, chimeric antibodies, humanized antibodies, single-chain antibodies (scAb), single domain antibodies (dAb), single domain heavy chain antibodies, a single domain light chain antibodies, nanobodies, bi-specific antibodies, multi-specific antibodies, and fusion proteins comprising an antigen-binding (also referred to herein as antigen binding) portion of an antibody and a non-antibody protein. Also encompassed by the term are Fab′, Fv, F(ab′)₂, and or other antibody fragments that retain specific binding to antigen, and monoclonal antibodies.

The term “nanobody” (Nb), as used herein, refers to the smallest antigen binding fragment or single variable domain (V_{HH}) derived from naturally occurring heavy chain antibody and is known to the person skilled in the art. They are derived from heavy chain only antibodies, seen in camelids (Hamers-Casterman et al., 1993; Desmyter et al., 1996). In the family of “camelids” immunoglobulins devoid of light polypeptide chains are found. “Camelids” comprise old world camelids (Camelus bactrianus and Camelus dromedarius) and new world camelids (for example, Llama paccos, Llama glama, Llama guanicoe and Llama vicugna). A single variable domain heavy chain antibody is referred to herein as a nanobody or a V_{HH} antibody.

“Antibody fragments” comprise a portion of an intact antibody, for example, the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062 (1995)); domain antibodies (dAb; Holt et al. (2003) Trends Biotechnol. 21:484); single-chain antibody molecules; and multi-specific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen. Antibody fragments include, e.g., scFv, sdAb, dAb, Fab, Fab′, Fab′₂, F(ab′)₂, Fd, Fv, Feb, and SMIP. An example of an sdAb is a camelid VHH.

“Fv” is the minimum antibody fragment that contains a complete antigen-recognition and -binding site. This region consists of a dimer of one heavy- and one light-chain variable domain in tight, non-covalent association. It is in this configuration that the three complementarity determining regions (CDRs) of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

“Single-chain Fv” or “sFv” or “scFv” antibody fragments comprise the V_{H} and V_{L} domains of antibody, wherein these domains are present in a single polypeptide chain. In some embodiments, the Fv polypeptide further comprises a polypeptide linker between the V_{H} and V_{L} domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al. (1993) Proc. Natl. Acad. Sci. USA 90:6444-6448.

### DREADDs

A suitable polypeptide of interest is in some cases a Designer Receptors Exclusively Activated by Designer Drugs (DREADD; also known as a “RASSL”). See e.g., Roth (2016) Neuron 89:683; Bang et al. (2016) Exp. Neurobiol. 25:205; Whissell et al. (2016) Front. Genet. 7:70; and U.S. Pat. No. 6,518,480. For example, a modified G protein-coupled receptor (GPCR) is genetically engineered so that it: 1) retains binding affinity for a synthetic small molecule; and 2) has decreased binding affinity for a selected naturally occurring peptide or nonpeptide ligand relative to binding by its corresponding wild-type GPCR (e.g., the GPCR from which the modified GPCR was derived). Synthetic small molecule binding to the modified receptor induces the target cell to respond with a specific physiological response (e.g., cellular proliferation, cellular secretion, cell migration, cell contraction, or pigment production).

Any G protein-coupled receptor having separable domains for: 1) natural ligand (e.g., a natural peptide ligand) binding; 2) synthetic small molecule binding; and 3) G protein interaction can be modified to produce a DREADD.

GPCRs that bind peptide as their natural ligand are in some cases used to generate a DREADD. Such GPCRs, include, but are not limited to: Type-1 Angiotensin II Receptor, Type-1a Angiotensin II Receptor, Type-1B Angiotensin II Receptor, Type-1C Angiotensin II Receptor, Type-2 Angiotensin II Receptor, Neuromedin-B Receptor, Gastrin-releasing Peptide Receptor, Bombesin Subtype-3 Receptor, B1 Bradykinin Receptor, B2 Bradykinin Receptor, Interleukin-8 A Receptor, Interleukin-8 B Receptor, FMet-Leu-Phe Receptor, Monocyte Chemoattractant Protein 1 Receptor, C-C Chemokine Receptor Type 1 Receptor, C5a Anaphylatoxin Receptor, Cholecystokinin Type A Receptor, Gastrin/cholecystokinin Type B Receptor, Endothelin-1 Receptor, Endothelin B Receptor, Follicle Stimulating Hormone (FSH-R) Receptor, Lutropin-choriogonadotropic Hormone (LH/CG-R) Receptor, Adrenocorticotropic Hormone Receptor (ACTH-R), Melanocyte Stimulating Hormone Receptor (MSH-R), Melanocortin-3 Receptor, Melanocortin-4 Receptor, Melanocortin-5 Receptor, Melatonin Type 1A Receptor, Melatonin Type 1B Receptor, Melatonin Type 1C Receptor, Neuropeptide Y Type 1 Receptor, Neuropeptide Y Type 2 Receptor, Neurotensin Receptor, Delta-type Opioid Receptor, Kappa-type Opioid Receptor, Mu-type Opioid, Nociceptin Receptor, Gonadotropin-releasing Hormone Receptor, Somatostatin Type 1 Receptor, Somatostatin Type 2 Receptor, Somatostatin Type 3 Receptor, Somatostatin Type 4 Receptor, Somatostatin Type 5 Receptor, Substance-P Receptor, Substance-K Receptor, Neuromedin K Receptor, Vasopressin V1a Receptor, Vasopressin V1B Receptor, Vasopressin V2 Receptor, Oxytocin Receptor, Galanin Receptor, Calcitonin Receptor, Calcitonin A Receptor, Calcitonin B Receptor, Growth Hormone-releasing Hormone Receptor, Parathyroid Hormone/parathyroid Hormone-related Peptide Receptor, Pituitary Adenylate Cyclase Activating Polypeptide Type I Receptor, Secretin Receptor, Vasoactive Intestinal Polypeptide 1 Receptor, and Vasoactive Intestinal Polypeptide 2 Receptor.

A DREADD can interact with a G protein selected from Gi, Gq, and Gs. Thus, a DREADD can be a Gi-coupled DREADD, a Gq-coupled DREADD, or a Gs-coupled DREADD.

DREADDs include, but are not limited to, hM3Dq, a DREADD generated from the human M3 muscarinic receptor; hM4Di, a DREADD generated from the Gi-coupled human M4 muscarinic; a DREADD generated from a kappa opioid receptor (see U.S. Pat. No. 6,518,480); KORD; and the like.

### Transcription Factors

Suitable transcription factors include naturally-occurring transcription factors and recombinant (e.g., non-naturally occurring, engineered, artificial, synthetic) transcription factors. In some cases, the transcription is a transcriptional activator. In some cases, the transcriptional activator is an engineered protein, such as a zinc finger or TALE based DNA binding domain fused to an effector domain such as VP64 (transcriptional activation).

A transcription factor can comprise: i) a DNA binding domain (DBD); and ii) an activation domain (AD). The DBD can be any DBD with a known response element, including synthetic and chimeric DNA binding domains, or analogs, combinations, or modifications thereof. Suitable DNA binding domains include, but are not limited to, a GAL4 DBD, a LexA DBD, a transcription factor DBD, a Group H nuclear receptor member DBD, a steroid/thyroid hormone nuclear receptor superfamily member DBD, a bacterial LacZ DBD, an EcR DBD, a GALA DBD, and a LexA DBD. Suitable ADs include, but are not limited to, a Group H nuclear receptor member AD, a steroid/thyroid hormone nuclear receptor AD, a CJ7 AD, a p65-TA1 AD, a synthetic or chimeric AD, a polyglutamine AD, a basic or acidic amino acid AD, a VP16 AD, a GAL4 AD, an NF-κB AD, a BP64 AD, a B42 acidic activation domain (B42AD), a p65 transactivation domain (p65AD), SAD, NF-1, AP-2, SP1-A, SP1-B, Oct-1, Oct-2, MTF-1, BTEB-2, and LKLF, or an analog, combination, or modification thereof.

Suitable transcription factors include transcriptional activators, where suitable transcriptional activators include, but are not limited to, GAL4-VP16, GAL5-VP64, Tbx21, tTA-VP16, VP16, VP64, GAL4, p65, LexA-VP16, GAL4-NFκB, and the like.

Suitable transcription factors include transcriptional repressors, where suitable transcriptional repressors (e.g., a transcription repressor domain) include, but are not limited to, Kruppel-associated box (KRAB); the Mad mSIN3 interaction domain (SID); the ERF repressor domain (ERD); MDB-2B; v-ErbA; MBD3; and the like.

### Reporter Gene Products

Suitable reporter gene products include polypeptides that generate a detectable signal. Suitable detectable signal-producing proteins include, e.g., fluorescent proteins; enzymes that catalyze a reaction that generates a detectable signal as a product; and the like.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilised ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Other examples of fluorescent proteins include mHoneydew, mBanana, mOrange, dTomato, tdTomato, mTangerine, mStrawberry, mCherry, mGrape1, mRaspberry, mGrape2, mPlum (Shaner et al. (2005) Nat. Methods 2:905-909), and the like. Any of a variety of fluorescent and colored proteins from Anthozoan species, as described in, e.g., Matz et al. (1999) Nature Biotechnol. 17:969-973, is suitable for use.

Suitable enzymes include, but are not limited to, horse radish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, glucose oxidase (GO), and the like.

### Genome-Editing Endonuclease

A “genome editing endonuclease” is an endonuclease, e.g., sequence-specific endonuclease, which can be used for the editing of a cell's genome (e.g., by cleaving at a targeted location within the cell's genomic DNA). Examples of genome editing endonucleases include but are not limited to: (i) Zinc finger nucleases, (ii) TAL endonucleases, and (iii) CRISPR/Cas endonucleases. Examples of CRISPR/Cas endonucleases include class 2 CRISPR/Cas endonucleases such as: (a) type II CRISPR/Cas proteins, e.g., a Cas9 protein; (b) type V CRISPR/Cas proteins, e.g., a Cpf1 polypeptide, a C2c1 polypeptide, a C2c3 polypeptide, and the like; and (c) type VI CRISPR/Cas proteins, e.g., a C2c2 polypeptide.

Examples of suitable sequence-specific, e.g., genome editing, endonucleases include, but are not limited to, zinc finger nucleases, meganucleases, TAL-effector DNA binding domain-nuclease fusion proteins (transcription activator-like effector nucleases (TALEN®s)), and CRISPR/Cas endonucleases (e.g., class 2 CRISPR/Cas endonucleases such as a type II, type V, or type VI CRISPR/Cas endonucleases). Thus, in some cases, a gene product is a sequence-specific genome editing endonuclease, e.g., genome editing, endonucleases selected from: a zinc finger nuclease, a TAL-effector DNA binding domain-nuclease fusion protein (TALEN), and a CRISPR/Cas endonuclease (e.g., a class 2 CRISPR/Cas endonuclease such as a type II, type V, or type VI CRISPR/Cas endonuclease). In some cases, a sequence-specific genome editing endonuclease includes a zinc finger nuclease or a TALEN. In some cases, a sequence-specific genome editing endonuclease includes a class 2 CRISPR/Cas endonuclease. In some cases, a sequence-specific genome editing endonuclease includes a class 2 type II CRISPR/Cas endonuclease (e.g., a Cas9 protein). In some cases, a sequence-specific genome editing endonuclease includes a class 2 type V CRISPR/Cas endonuclease (e.g., a Cpf1 protein, a C2c1 protein, or a C2c3 protein). In some cases, a sequence-specific genome editing endonuclease includes a class 2 type VI CRISPR/Cas endonuclease (e.g., a C2c2 protein).

RNA-mediated adaptive immune systems in bacteria and archaea rely on Clustered Regularly Interspaced Short Palindromic Repeat (CRISPR) genomic loci and CRISPR-associated (Cas) proteins that function together to provide protection from invading viruses and plasmids. In some cases, an RNA-guided endonuclease is a class 2 CRISPR/Cas endonuclease. In class 2 CRISPR systems, the functions of the effector complex (e.g., the cleavage of target DNA) are carried out by a single endonuclease (e.g., see Zetsche et al, Cell. 2015 Oct. 22; 163(3):759-71; Makarova et al, Nat Rev Microbiol. 2015 November; 13(11):722-36; and Shmakov et al., Mol Cell. 2015 Nov. 5; 60(3):385-97). As such, the term “class 2 CRISPR/Cas protein” is used herein to encompass the endonuclease (the target nucleic acid cleaving protein) from class 2 CRISPR systems. Thus, the term “class 2 CRISPR/Cas endonuclease” as used herein encompasses type II CRISPR/Cas proteins (e.g., Cas9), type V CRISPR/Cas proteins (e.g., Cpf1, C2c1, C2C3), and type VI CRISPR/Cas proteins (e.g., C2c2). To date, class 2 CRISPR/Cas proteins encompass type II, type V, and type VI CRISPR/Cas proteins, but the term is also meant to encompass any class 2 CRISPR/Cas protein suitable for binding to a corresponding guide RNA and forming an RNP complex.

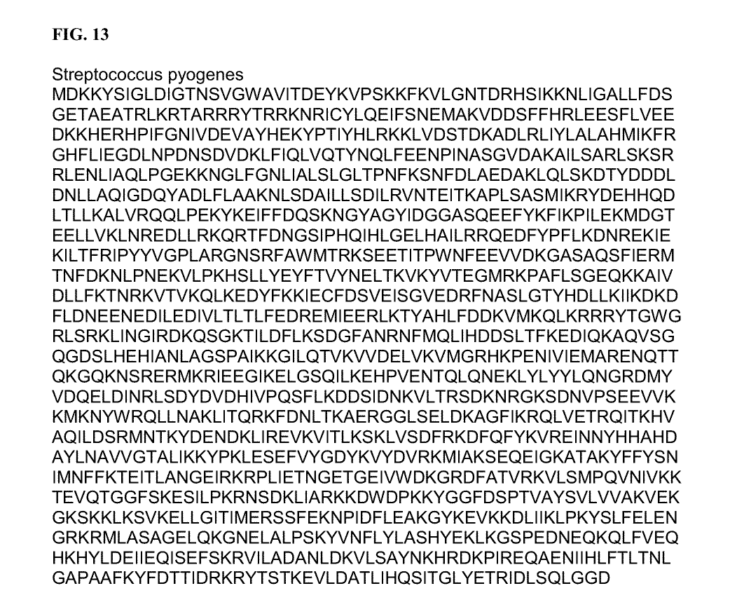

In some cases, a suitable RNA-guided endonuclease comprises an amino acid sequence having at least 30%, at least 40%, at least 50%, at least 60%, at least 70%, at least 80%, at least 85%, at least 90%, at least 95%, at least 98%, at least 99%, or 100%, amino acid sequence identity to the Streptococcus pyogenes Cas9 amino acid sequence depicted in FIG. 13.

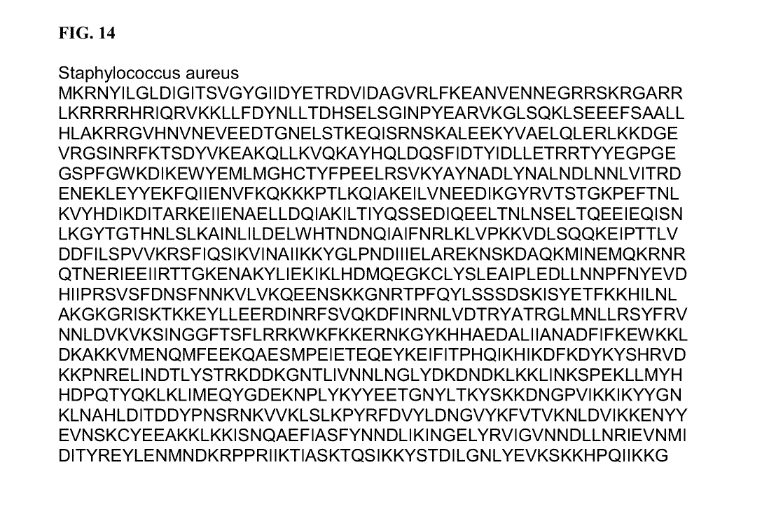

In some cases, a suitable RNA-guided endonuclease comprises an amino acid sequence having at least 30%, at least 40%, at least 50%, at least 60%, at least 70%, at least 80%, at least 85%, at least 90%, at least 95%, at least 98%, at least 99%, or 100%, amino acid sequence identity to the Staphylococcus aureus Cas9 amino acid sequence depicted in FIG. 14.

In some cases, the RNA-guided endonuclease is a nickase. Jinek et al., Science. 2012 Aug. 17; 337(6096):816-21).

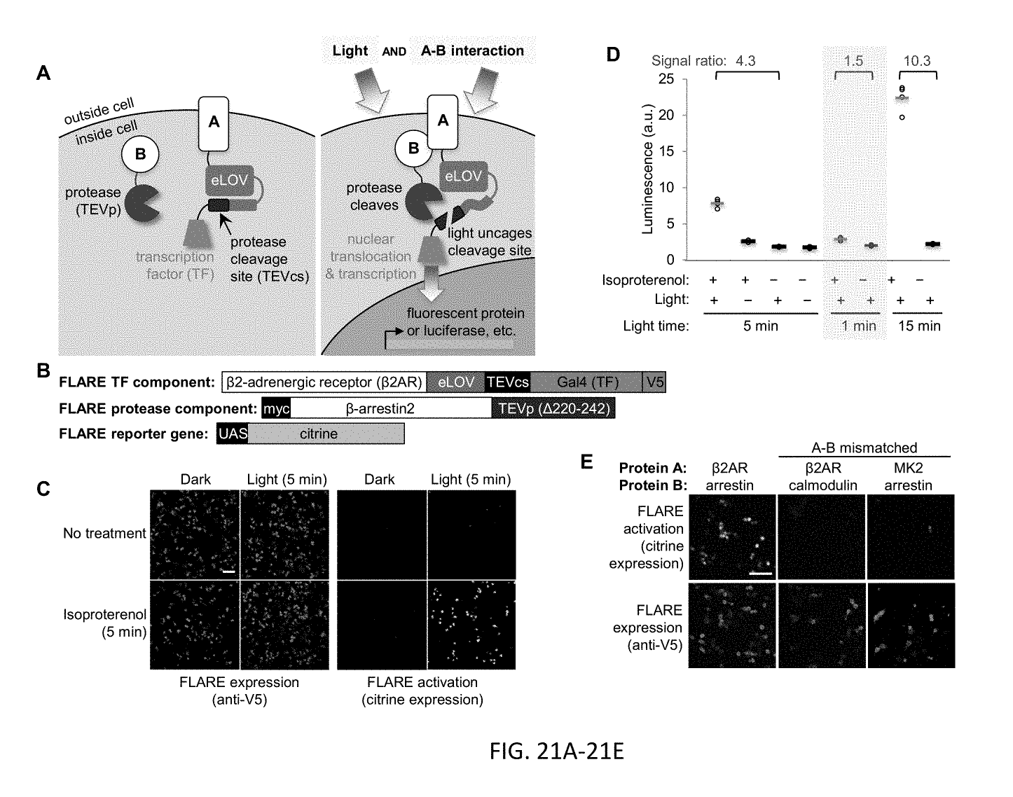

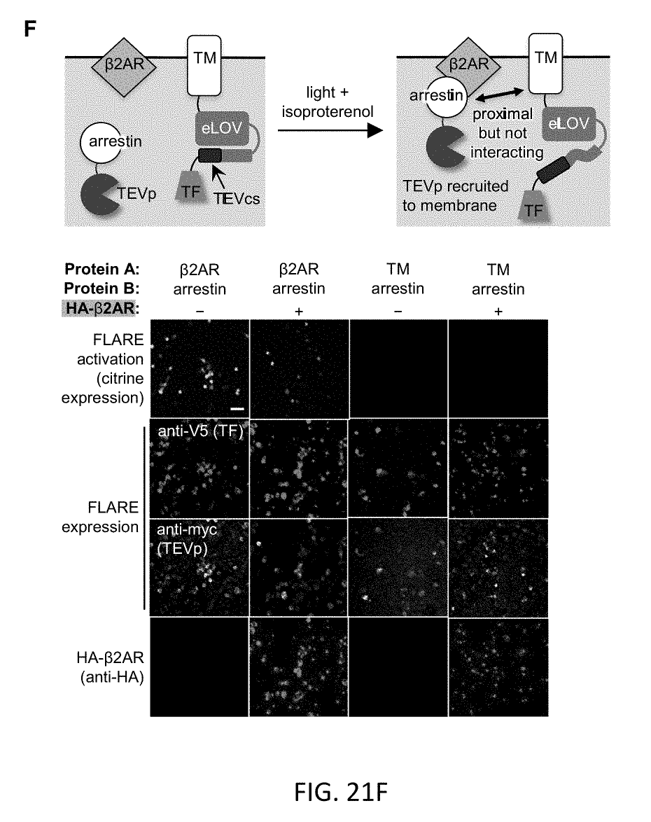

In some cases, the RNA-guided endonuclease is a variant Cas9 protein that has reduced catalytic activity (e.g., when a Cas9 protein has a D10, G12, G17, E762, H840, N854, N863, H982, H983, A984, D986, and/or a A987 mutation of the amino acid sequence depicted in FIG. 21, e.g., D10A, G12A, G17A, E762A, H840A, N854A, N863A, H982A, H983A, A984A, and/or D986A); and the variant Cas9 protein retains the ability to bind to target nucleic acid in a site-specific manner (e.g., when complexed with a guide RNA.

In some cases, the RNA-guided endonuclease is a type V CRISPR/Cas protein. In some cases, the RNA-guided endonuclease is a type VI CRISPR/Cas protein. Examples and guidance related to type V and type VI CRISPR/Cas proteins (e.g., Cpf1, C2c1, C2c2, and C2c3 guide RNAs) can be found in the art, for example, see Zetsche et al, Cell. 2015 Oct. 22; 163(3):759-71; Makarova et al, Nat Rev Microbiol. 2015 November; 13(11):722-36; and Shmakov et al., Mol Cell. 2015 Nov. 5; 60(3):385-97.

In some cases, the RNA-guided endonuclease is a chimeric polypeptide (e.g., a fusion polypeptide) comprising: a) an RNA-guided endonuclease; and b) a fusion partner, where the fusion partner provides a functionality or activity other than an endonuclease activity. For example, the fusion partner can be a polypeptide having an enzymatic activity that modifies a polypeptide (e.g., a histone) associated with, or proximal to, a target nucleic acid (e.g., methyltransferase activity, deaminase activity (e.g., cytidine deaminase activity), demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity).

In some cases, the RNA-guided endonuclease is a base editor; for example, in some cases, the RNA-guided endonuclease is a fusion polypeptide comprising: a) an RNA-guided endonuclease; and b) a cytidine deaminase. See, e.g., Komor et al. (2016) Nature 533:420.

### Opsins

In some cases, a gene product encoded in a system of the present disclosure is a hyperpolarizing or a depolarizing light-activated polypeptide (an “opsin”). The light-activated polypeptide may be a light-activated ion channel or a light-activated ion pump. The light-activated ion channel polypeptides are adapted to allow one or more ions to pass through the plasma membrane of a neuron when the polypeptide is illuminated with light of an activating wavelength. Light-activated proteins may be characterized as ion pump proteins, which facilitate the passage of a small number of ions through the plasma membrane per photon of light, or as ion channel proteins, which allow a stream of ions to freely flow through the plasma membrane when the channel is open. In some embodiments, the light-activated polypeptide depolarizes the neuron when activated by light of an activating wavelength. Suitable depolarizing light-activated polypeptides, without limitation, are shown in FIG. 15. In some embodiments, the light-activated polypeptide hyperpolarizes the neuron when activated by light of an activating wavelength. Suitable hyperpolarizing light-activated polypeptides, without limitation, are shown in FIG. 16.

In some cases, a light-activated polypeptide comprises an amino acid sequence having at least 80%, at least 85%, at least 90%, at least 95%, at least 98%, at least 99%, or 100%, amino acid sequence identity to an opsin amino acid sequence depicted in FIG. 15. In some cases, a light-activated polypeptide comprises an amino acid sequence having at least 80%, at least 85%, at least 90%, at least 95%, at least 98%, at least 99%, or 100%, amino acid sequence identity to an opsin amino acid sequence depicted in FIG. 16.

In some embodiments, the light-activated polypeptides are activated by blue light. In some embodiments, the light-activated polypeptides are activated by green light. In some embodiments, the light-activated polypeptides are activated by yellow light. In some embodiments, the light-activated polypeptides are activated by orange light. In some embodiments, the light-activated polypeptides are activated by red light.

In some embodiments, the light-activated polypeptide expressed in a cell can be fused to one or more amino acid sequence motifs selected from the group consisting of a signal peptide, an endoplasmic reticulum (ER) export signal, a membrane trafficking signal, and/or an N-terminal golgi export signal. The one or more amino acid sequence motifs which enhance light-activated protein transport to the plasma membranes of mammalian cells can be fused to the N-terminus, the C-terminus, or to both the N- and C-terminal ends of the light-activated polypeptide. In some cases, the one or more amino acid sequence motifs which enhance light-activated polypeptide transport to the plasma membranes of mammalian cells is fused internally within a light-activated polypeptide. Optionally, the light-activated polypeptide and the one or more amino acid sequence motifs may be separated by a linker.

In some embodiments, the light-activated polypeptide can be modified by the addition of a trafficking signal (ts) which enhances transport of the protein to the cell plasma membrane. In some embodiments, the trafficking signal can be derived from the amino acid sequence of the human inward rectifier potassium channel Kir2.1. In other embodiments, the trafficking signal can comprise the amino acid sequence KSRITSEGEYIPLDQIDINV (SEQ ID NO:56). Trafficking sequences that are suitable for use can comprise an amino acid sequence having at least 85%, 90%, 91%, 92%, 93%, 94%, 95%, 96%, 97%, 98%, 99%, or 100%, amino acid sequence identity to an amino acid sequence such a trafficking sequence of human inward rectifier potassium channel Kir2.1 (e.g., KSRITSEGEYIPLDQIDINV (SEQ ID NO:56)).

A trafficking sequence can have a length of from about 10 amino acids to about 50 amino acids, e.g., from about 10 amino acids to about 20 amino acids, from about 20 amino acids to about 30 amino acids, from about 30 amino acids to about 40 amino acids, or from about 40 amino acids to about 50 amino acids.

ER export sequences that are suitable for use with a light-activated polypeptide include, e.g., VXXSL (where X is any amino acid; SEQ ID NO:52) (e.g., VKESL (SEQ ID NO:53); VLGSL (SEQ ID NO:54); etc.); NANSFCYENEVALTSK (SEQ ID NO:55); FXYENE (SEQ ID NO:57) (where X is any amino acid), e.g., FCYENEV (SEQ ID NO:58); and the like. An ER export sequence can have a length of from about 5 amino acids to about 25 amino acids, e.g., from about 5 amino acids to about 10 amino acids, from about 10 amino acids to about 15 amino acids, from about 15 amino acids to about 20 amino acids, or from about 20 amino acids to about 25 amino acids.

In some cases, a light-activated polypeptide is a fusion polypeptide that comprises an endoplasmic reticulum (ER) export signal (e.g., FCYENEV). In some cases, a light-activated polypeptide is a fusion polypeptide that comprises a membrane trafficking signal (e.g., KSRITSEGEYIPLDQIDINV). In some cases, a light-activated polypeptide is a fusion polypeptide comprising, in order from N-terminus to C-terminus: a) a light-activated polypeptide comprising an amino acid sequence having at least 80%, at least 85%, at least 90%, at least 95%, at least 98%, at least 99%, or 100%, amino acid sequence identity to an opsin amino acid sequence depicted in FIG. 15 or FIG. 16; b) an ER export signal; and c) a membrane trafficking signal.

### Toxins

Suitable toxins include polypeptide toxins present in a natural source (e.g., naturally-occurring), recombinantly produced toxins, and synthetically produced toxins. Suitable toxins include ribosome inactivating proteins (RIPs); a bacterial toxin; and the like.

Suitable toxins include, e.g., anthopleurin B (GVPCLCDSDG-PRPRGNTLSG-ILWFYPSGCP-SGWHNCKAHG-PNIGWCCKK; SEQ ID NO://), anthopleurin C, anthopleurin Q, calitoxin (MKTQVLALFV LCVLFCLAES RTTLNKRNDI EKRIECKCEG DAPDLSHMTG TVYFSCKGGD GSWSKCNTYT AVADCCHQA; SEQ ID NO://), a conotoxin, ectatomin, HsTx1, omega-atracotoxin, a raventoxin, a scorpion toxin, and the like.

Suitable bacterial toxins include, e.g., cholera toxin, botulinum toxin, diphtheria toxin (produced by Corynebacterium diphtheriae), tetanospasmin, an enterotoxin, hemolysin, shiga toxin, erythrogenic toxin, adenylate cyclase toxin, pertussis toxin, ST toxin, LT toxin, ricin, abrin, tetanus toxin, and the like.

Exemplary Type I RIPS include, but are not limited to, gelonin, dodecandrin, tricosanthin, tricokirin, bryodin, Mirabilis antiviral protein (MAP), barley ribosome-inactivating protein (BRIP), pokeweed antiviral proteins (PAPS), saporins, luffins, and momordins. Exemplary Type II RIPS include, but are not limited to, ricin and abrin.

### Antibiotic Resistance Factors

As noted above, in some cases, the gene product of interest is an antibiotic resistance factor, e.g., a polypeptide that confers antibiotic resistance to a cell that produces the polypeptide.

Suitable antibiotic resistance factors include, but are not limited to, polypeptides that confer resistance to kanamycin, gentamicin, rifampin, trimethoprim, chloramphenicol, tetracycline, penicillin, methicillin, blasticidin, puromycin, hygromycin, or other antimicrobial agent. Suitable antibiotic resistance factors include, but are not limited to, aminoglycoside acetyltransferases, rifampin ADP-ribosyltransferases, dihydrofolate reductases, transporters, β-lactamases, chloramphenicol acetyltransferases, and efflux pumps. See, e.g., McGarvey et al. (2012) Applied Environ. Microbiol. 78:1708. Suitable antibiotic resistance factors include, but are not limited to, aminoglycoside 6′-N-acetyltransferase; gentamycin 3′-N-acetyltransferase; rifampin ADP-ribosyltransferase; dihydrofolate reductase; MFS transporter; ABC transporter; blasticidin-S deaminase; blasticidin acetyltransferase; puromycin N-acetyl-transferease; hygromycin kinase; and the like.

### Recombinases

In some cases, the gene product of interest is a recombinase. The term “recombinase” refers to an enzyme that catalyzes DNA exchange at a specific target site, for example, a palindromic sequence, by excision/insertion, inversion, translocation, and exchange.

Suitable recombinases include, but are not limited to, Cre recombinase; a FLP recombinase; a Tel recombinase; and the like. A suitable recombinase is one that targets (and cleaves) a target site selected from a telRL site, a loxP site, a phi pK02 telRL site, an FRT site, phiC31 attP site, and a λattP site.

A suitable recombinase can be selected from the group consisting of: TelN; Tel; Tel (gp26 K02 phage); Cre; Flp; phiC31; Int; and a lambdoid phage integrase (e.g. a phi 80 recombinase, a HK022 recombinase; an HP1 recombinase).

Examples of target sites for such recombinases include, e.g.: a telRL site (targeted by a TelN recombinase): TATCAGCACACAATTGCCCATTATACGCGCGTATAATGGACTAT TGTGTGCTGA (SEQ ID NO://); a pal site: ACCTATTTCAGCATACTACGCGCGTAGTATGCTGAAATAGGT (SEQ ID NO://); a phi K02 telRL site: CCATTATACGCGCGTATAATGG (SEQ ID NO://); a loxP site (targeted by a Cre recombinase): TAACTTCGTATAGCATACATTATACGAAGTTAT (SEQ ID NO://); a FRT site (targeted by a Flp recombinase): GAAGTTCCTATTCTCTAGAAAGTATAGGAACTTC (SEQ ID NO://); a phiC31 attP site (targeted by a phiC31 recombinase):

(SEQ ID NO: //)
CCCAGGTCAGAAGCGGTTTTCGGGAGTAGTGCCCCAACTGGGGT
AACCTTTGAGTTCTCTCAGTTGGGGGCGTAGGGTCGCCGACAYGA
CACAAGGGGTT;
a λ attP site:
(SEQ ID NO: //)
TGATAGTGACCTGTTCGTTTGCAACACATTGATGAGCAATGCTT
TTTTATAATGCCAACTTTGTACAAAAAAGCTGAACGAGAAACGT
AAAATGATATAAA.

### Additional Amino Acid Sequences

In some cases, the gene product is a fusion polypeptide comprising a fusion partner, where the fusion partner can be, e.g., a soma localization signal, a nuclear localization signal, a protein transduction domain, a mitochondrial localization signal, a chloroplast localization signal, an endoplasmic reticulum retention signal, an epitope tag, etc. For example, a suitable mitochondrial localization sequence is LGRVIPRKIASRASLM (SEQ ID NO://); or MSVLTPLLLRGLTGSARRLPVPRAKIHSLL (SEQ ID NO:/).

### Soma Localization Signal

In some cases, the transcription factor includes a soma localization signal. For example, a 66 amino acid C-terminal sequence of Kv2.1 or a 27 amino acid sequence of Nav1.6 induces localization to the soma of a neuron. For example, the Nav1.6 soma localization signal comprises the amino acid sequence: TVRVPIAVGESDFENLNTEDVSSESDP (SEQ ID NO://).

### Nuclear Localization Signals

Non-limiting examples of NLSs include an NLS sequence derived from: the NLS of the SV40 virus large T-antigen, having the amino acid sequence PKKKRKV (SEQ ID NO://); the NLS from nucleoplasmin (e.g. the nucleoplasmin bipartite NLS with the sequence KRPAATKKAGQAKKKK (SEQ ID NO://)); the c-myc NLS having the amino acid sequence PAAKRVKLD (SEQ ID NO://) or RQRRNELKRSP (SEQ ID NO://); the hRNPA1 M9 NLS having the sequence NQSSNFGPMKGGNFGGRSSGPYGGGGQYFAKPRNQGGY (SEQ ID NO://); the sequence RMRIZFKNKGKDTAELRRRRVEVSVELRKAKKDEQILKRRNV (SEQ ID NO://) of the IBB domain from importin-alpha; the sequences VSRKRPRP (SEQ ID NO://) and PPKKARED (SEQ ID NO:/) of the myoma T protein; the sequence PQPKKKPL (SEQ ID NO://) of human p53; the sequence SALIKKKKKMAP (SEQ ID NO://) of mouse c-abl IV; the sequences DRLRR (SEQ ID NO://) and PKQKKRK (SEQ ID NO://) of the influenza virus NS1; the sequence RKLKKKIKKL (SEQ ID NO://) of the Hepatitis virus delta antigen; the sequence REKKKFLKRR (SEQ ID NO://) of the mouse Mx1 protein; the sequence KRKGDEVDGVDEVAKKKSKK (SEQ ID NO://) of the human poly(ADP-ribose) polymerase; and the sequence RKCLQAGMNLEARKTKK (SEQ ID NO://) of the steroid hormone receptors (human) glucocorticoid.

A gene product can include a “Protein Transduction Domain” or PTD (also known as a CPP-cell penetrating peptide), which refers to a polypeptide that facilitates traversing a lipid bilayer, micelle, cell membrane, organelle membrane, or vesicle membrane. A PTD attached to another polypeptide (a polypeptide gene product of interest) facilitates the polypeptide traversing a membrane, for example going from extracellular space to intracellular space, or cytosol to within an organelle. In some cases, a PTD attached to a polypeptide gene product of interest facilitates entry of the polypeptide into the nucleus (e.g., in some cases, a PTD includes a nuclear localization signal). In some cases, a PTD is covalently linked to the amino terminus of a polypeptide gene product of interest. In some cases, a PTD is covalently linked to the carboxyl terminus of a polypeptide gene product of interest. In some cases, a PTD is covalently linked to the amino terminus and to the carboxyl terminus of a polypeptide gene product of interest. Exemplary PTDs include but are not limited to a minimal undecapeptide protein transduction domain (corresponding to residues 47-57 of HIV-1 TAT comprising YGRKKRRQRRR; SEQ ID NO://); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); an Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008); RRQRRTSKLMKR (SEQ ID NO://); Transportan GWTLNSAGYLLGKINLKALAALAKKIL (SEQ ID NO://); KALAWEAKLAKALAKALAKHLAKALAKALKCEA (SEQ ID NO://); and RQIKIWFQNRRMKWKK (SEQ ID NO://). Exemplary PTDs include but are not limited to, YGRKKRRQRRR (SEQ ID NO://), RKKRRQRRR (SEQ ID NO://); an arginine homopolymer of from 3 arginine residues to 50 arginine residues; Exemplary PTD domain amino acid sequences include, but are not limited to, any of the following: YGRKKRRQRRR (SEQ ID NO://); RKKRRQRR (SEQ ID NO://); YARAAARQARA (SEQ ID NO://); THRLPRRRRRR (SEQ ID NO://); and GGRRARRRRRR (SEQ ID NO://).

### Target Genes

As noted above, in some cases, a polypeptide of interest is a transcription factor. In such cases, the transcription factor can control expression of any of a variety of gene products. “Gene products” as used herein, include polypeptide gene products and nucleic acid gene products.

Suitable nucleic acid gene products include, but are not limited to, an inhibitory nucleic acid, a ribozyme, a guide RNA that binds a target nucleic acid and an RNA-guided endonuclease, a microRNA, and the like.

### Polypeptide Gene Products

In some cases, a transcription factor, when released from the first (light-activated) polypeptide by cleavage of the proteolytically cleavable linker, controls transcription of a nucleotide sequence encoding a polypeptide.

Suitable polypeptide gene products include, but are not limited to, a reporter gene product, an opsin, a DREADD, a toxin, an enzyme, a transcription factor, an antibiotic resistance factor, a genome editing endonuclease, an RNA-guided endonuclease, a protease, a kinase, a phosphatase, a phosphorylase, a lipase, a receptor, an antibody, a fluorescent protein, a peroxidase such as APEX or APEX2, a base editing enzyme, a biotin ligase, a recombinase, a synaptic marker, a signaling protein, an effector protein of a receptor, a protein that regulates synaptic vesicle fusion or protein trafficking or organelle trafficking, a portion (e.g., a split half) of any one of the aforementioned polypeptides. Such polypeptides are described above.

### Nucleic Acid Gene Products

In some cases, a transcription factor present in a first fusion polypeptide of the present disclosure, when released from the first fusion polypeptide by cleavage of the proteolytically cleavable linker, controls transcription of a nucleotide sequence encoding a nucleic acid gene product.

Suitable nucleic acid gene products include, but are not limited to, an inhibitory nucleic acid, a ribozyme, a guide RNA that binds a target nucleic acid and an RNA-guided endonuclease, a microRNA (miRNA), an antisense RNA, a ribozyme, a decoy RNA, an anti-mir RNA, a long non-coding RNA, and the like. Typically, the nucleic acid gene product is not translated.

### Guide RNAs

Guide RNAs include RNAs (where a guide RNA can be a single RNA molecule or two RNA molecules) that comprise a first segment that comprises a nucleotide sequence that is complementary to (and hybridizes with) a target nucleotide sequence (e.g., a target nucleotide sequence present in genomic DNA), and a second segment that comprises a nucleotide sequence that binds to an RNA-guided endonuclease (e.g., a Cas9 polypeptide, a Cpf1 polypeptide, a C2c2 polypeptide, as described above).

In some cases, the guide RNA(s) bind to a Cas9 polypeptide. The first segment (targeting segment) of a Cas9 guide RNA includes a nucleotide sequence (a guide sequence) that is complementary to (and therefore hybridizes with) a specific sequence (a target site) within a target nucleic acid (e.g., a target ssRNA, a target ssDNA, the complementary strand of a double stranded target DNA, etc.). The protein-binding segment (or “protein-binding sequence”) interacts with (binds to) a Cas9 polypeptide. The protein-binding segment of a Cas9 guide RNA includes two complementary stretches of nucleotides that hybridize to one another to form a double stranded RNA duplex (dsRNA duplex). Site-specific binding and/or cleavage of a target nucleic acid (e.g., genomic DNA) can occur at locations (e.g., target sequence of a target locus) determined by base-pairing complementarity between the Cas9 guide RNA (the guide sequence of the Cas9 guide RNA) and the target nucleic acid.

In some cases, a guide RNA includes two separate nucleic acid molecules: an “activator” and a “targeter” and is referred to herein as a “dual guide RNA”, a “double-molecule guide RNA”, a “two-molecule guide RNA”, or a “dgRNA.” In some cases, the guide RNA is one molecule (e.g., for some class 2 CRISPR/Cas proteins, the corresponding guide RNA is a single molecule; and in some cases, an activator and targeter are covalently linked to one another, e.g., via intervening nucleotides), and the guide RNA is referred to as a “single guide RNA”, a “single-molecule guide RNA,” a “one-molecule guide RNA”, or simply “sgRNA.”

A “target nucleic acid” as used herein is a polynucleotide (e.g. a chromosomal DNA sequence; or an extrachromosomal sequence, e.g., an episomal sequence, a minicircle sequence, a mitochondrial sequence, a chloroplast sequence, etc.) that includes a site (“target site” “target sequence” or “endonuclease-recognized sequence”) targeted by a sequence-specific endonuclease, e.g., genome-editing endonuclease. When the sequence-specific endonuclease, e.g., genome editing endonuclease, is a CRISPR/Cas endonuclease, the target sequence is the sequence to which the guide sequence of a CRISPR/Cas guide RNA (e.g., a Cas9 guide RNA) will hybridize. For example, the target site (or target sequence) 5′-GAGCAUAUC-3′ within a target nucleic acid is targeted by (or is bound by, or hybridizes with, or is complementary to) the sequence 5′-GAUAUGCUC-3′. Suitable hybridization conditions include physiological conditions normally present in a cell. For a double stranded target nucleic acid, the strand of the target nucleic acid that is complementary to and hybridizes with the guide RNA is referred to as the “complementary strand” or “target strand”; while the strand of the target nucleic acid that is complementary to the “target strand” (and is therefore not complementary to the guide RNA) is referred to as the “non-target strand” or “non-complementary strand”.

Guide RNAs are well known in the art. Nucleotide sequences of the portion of the guide RNA that binds to a particular RNA-guided endonuclease (e.g., Cas9, Cpf1, C2c2, etc.) are known in the art. The portion of the guide RNA that hybridizes to a target nucleic acid can be designed based on the sequence of the target nucleic acid.

### Inhibitory RNAs

Inhibitory RNAs are well known in the art. RNAi is the sequence-specific, post-transcriptional silencing of a gene's expression by double-stranded RNA. RNAi is mediated by 21- to 25-nucleotide, double-stranded RNA molecules referred to as small interfering RNAs (siRNAs). siRNAs can be derived by enzymatic cleavage of double-stranded precursor short interfering RNAs (shRNA) expressed from genetic constructs or micro RNA precursors in cells.

### Examples of PPI Detection Systems of the Present Disclosure

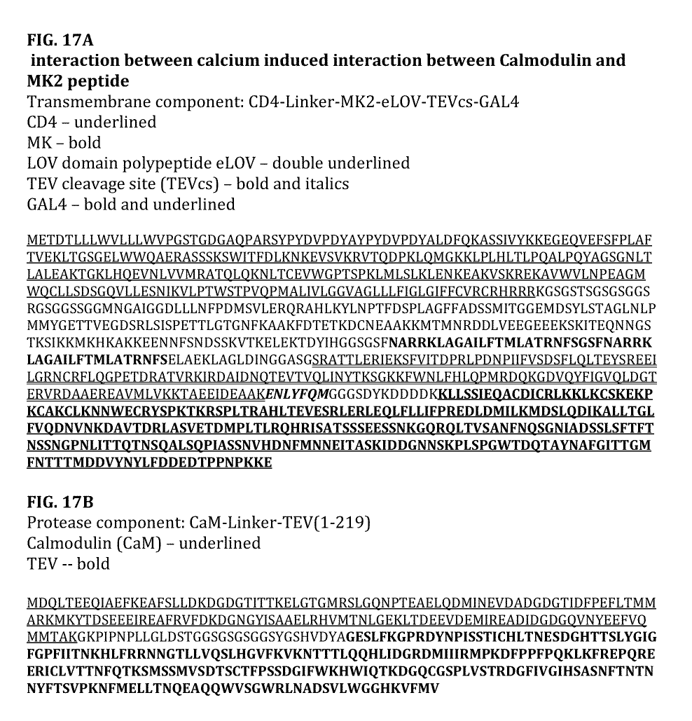

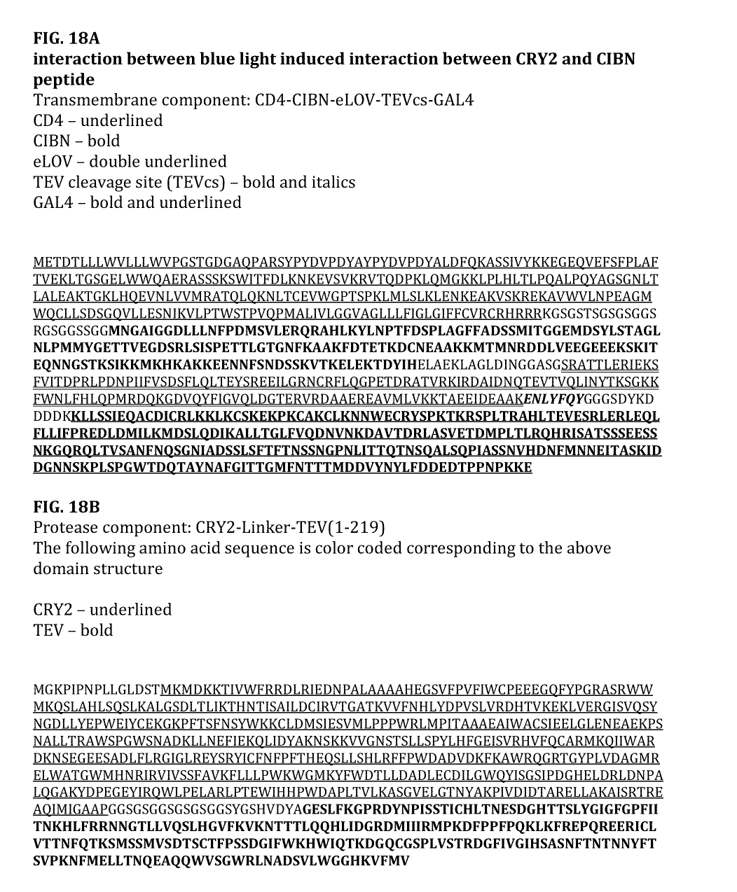

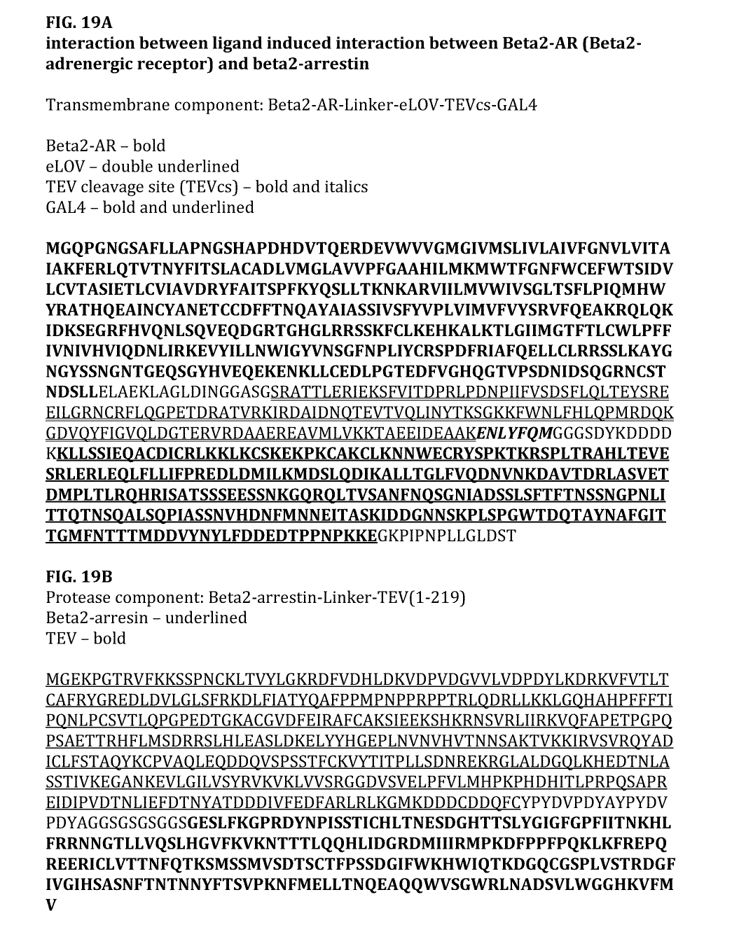

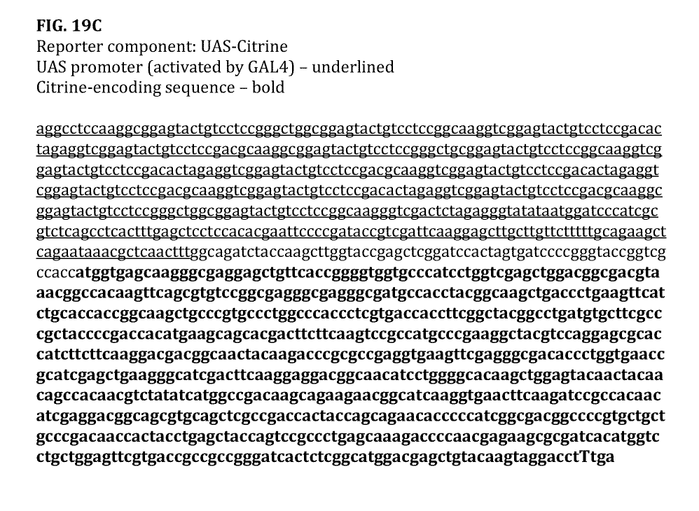

Non-limiting examples of PPI detection systems of the present disclosure are depicted in FIG. 17-20.

### Nucleic Acids

As noted above, a nucleic acid system of the present disclosure (e.g., System 1; System 2; as described above) comprises two nucleic acids.

In some cases, the nucleotide sequence encoding the first (light-activated) fusion polypeptide and/or the nucleotide sequence encoding the second fusion polypeptide (the second fusion polypeptide comprising a second polypeptide member of the protein-interaction pair fused to a protease) is operably linked to a transcriptional control element (e.g., a promoter; an enhancer; etc.). In some cases, the transcriptional control element is inducible. In some cases, the transcriptional control element is constitutive. In some cases, the promoters are functional in eukaryotic cells. In some cases, the promoters are cell type-specific promoters. In some cases, the promoters are tissue-specific promoters. In some cases, the promoter to which the nucleotide sequence encoding the first fusion polypeptide is operably linked, and the promoter to which the nucleotide sequence encoding the second fusion polypeptide is operably linked, are substantially the same. In other cases, the promoter to which the nucleotide sequence encoding the first fusion polypeptide is operably linked is different from the promoter to which the nucleotide sequence encoding the second fusion polypeptide is operably linked.

Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein.), it may be a spatially restricted promoter (i.e., transcriptional control element, enhancer, etc.)(e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hair follicle cycle in mice).

Suitable promoter and enhancer elements are known in the art. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue-specific promoters. Suitable promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6) (Miyagishi et al., Nature Biotechnology 20, 497-500 (2002)), an enhanced U6 promoter (e.g., Xia et al., Nucleic Acids Res. 2003 Sep. 1; 31(17)), a human H1 promoter (H1), and the like.

Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

Inducible promoters suitable for use include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

In some cases, the promoter is a neuron-specific promoter. Suitable neuron-specific control sequences include, but are not limited to, a neuron-specific enolase (NSE) promoter (see, e.g., EMBL HSENO2, X51956; see also, e.g., U.S. Pat. No. 6,649,811, U.S. Pat. No. 5,387,742); an aromatic amino acid decarboxylase (AADC) promoter; a neurofilament promoter (see, e.g., GenBank HUMNFL, L04147); a synapsin promoter (see, e.g., GenBank HUMSYNIB, M55301); a thy-1 promoter (see, e.g., Chen et al. (1987) Cell 51:7-19; and Llewellyn et al. (2010) Nat. Med. 16:1161); a serotonin receptor promoter (see, e.g., GenBank S62283); a tyrosine hydroxylase promoter (TH) (see, e.g., Nucl. Acids. Res. 15:2363-2384 (1987) and Neuron 6:583-594 (1991)); a GnRH promoter (see, e.g., Radovick et al., Proc. Natl. Acad. Sci. USA 88:3402-3406 (1991)); an L7 promoter (see, e.g., Oberdick et al., Science 248:223-226 (1990)); a DNMT promoter (see, e.g., Bartge et al., Proc. Natl. Acad. Sci. USA 85:3648-3652 (1988)); an enkephalin promoter (see, e.g., Comb et al., EMBO J. 17:3793-3805 (1988)); a myelin basic protein (MBP) promoter; a CMV enhancer/platelet-derived growth factor-β promoter (see, e.g., Liu et al. (2004) Gene Therapy 11:52-60); a motor neuron-specific gene Hb9 promoter (see, e.g., U.S. Pat. No. 7,632,679; and Lee et al. (2004) Development 131:3295-3306); and an alpha subunit of Ca(²⁺)-calmodulin-dependent protein kinase II (CaMKIIα) promoter (see, e.g., Mayford et al. (1996) Proc. Natl. Acad. Sci. USA 93:13250). Other suitable promoters include elongation factor (EF) 1α and dopamine transporter (DAT) promoters.

In some cases, a nucleic acid of a system of the present disclosure is a recombinant expression vector. In some cases, the recombinant expression vector is a viral construct, e.g., a recombinant adeno-associated virus (AAV) construct, a recombinant adenoviral construct, a recombinant lentiviral construct, a recombinant retroviral construct, etc. In some cases, a nucleic acid of a system of the present disclosure is a recombinant lentivirus vector. In some cases, a nucleic acid of a system of the present disclosure is a recombinant AAV vector.

Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., Hum Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. In some cases, the vector is a lentivirus vector. Also suitable are transposon-mediated vectors, such as piggyback and sleeping beauty vectors.

In some cases, a nucleic acid system of the present disclosure is packaged in a viral particle. For example, in some cases, the nucleic acids of a nucleic acid system of the present disclosure are recombinant AAV vectors, and are packaged in recombinant AAV particles. Thus, the present disclosure provides a recombinant viral particle comprising a nucleic acid system of the present disclosure.

### Genetically Modified Host Cells

The present disclosure provides a genetically modified host cell (e.g., an in vitro genetically modified host cell; or an in vivo genetically modified host cell) comprising a nucleic acid system of the present disclosure. In some cases, one or both of the first and the second nucleic acid of a nucleic acid system of the present disclosure is stably integrated into the genome of the host cell. In some instances, one or both of the first and the second nucleic acid of a nucleic acid system of the present disclosure is present episomally in the genetically modified host cell.

In some cases, the genetically modified host cell is a primary (non-immortalized) cell. In some cases, the genetically modified host cell is an immortalized cell line.

Suitable host cells include mammalian cells, insect cells, reptile cells, amphibian cells, arachnid cells, plant cells, bacterial cells, archaeal cells, yeast cells, algal cells, fungal cells, and the like.

In some cases, the genetically modified host cell is a mammalian cell, e.g., a human cell, a non-human primate cell, a rodent cell, a feline (e.g., a cat) cell, a canine (e.g., a dog) cell, an ungulate cell, an equine (e.g., a horse) cell, an ovine cell, a caprine cell, a bovine cell, etc. In some cases, the genetically modified host cell is a rodent cell (e.g., a rat cell; a mouse cell). In some cases, the genetically modified host cell is a human cell. In some cases, the genetically modified host cell is a non-human primate cell.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

Suitable host cells include cells of, e.g., Bacteria (e.g., Eubacteria); Archaebacteria; Protista; Fungi; Plantae; and Animalia. Suitable host cells include cells of plant-like members of the kingdom Protista, including, but not limited to, algae (e.g., green algae, red algae, glaucophytes, cyanobacteria); fungus-like members of Protista, e.g., slime molds, water molds, etc.; animal-like members of Protista, e.g., flagellates (e.g., Euglena), amoeboids (e.g., amoeba), sporozoans (e.g, Apicomplexa, Myxozoa, Microsporidia), and ciliates (e.g., Paramecium). Suitable host cells include cells of members of the kingdom Fungi, including, but not limited to, members of any of the phyla: Basidiomycota (club fungi; e.g., members of Agaricus, Amanita, Boletus, Cantherellus, etc.); Ascomycota (sac fungi, including, e.g., Saccharomyces); Mycophycophyta (lichens); Zygomycota (conjugation fungi); and Deuteromycota. Suitable host cells include cells of members of the kingdom Plantae, including, but not limited to, members of any of the following divisions: Bryophyta (e.g., mosses), Anthocerotophyta (e.g., hornworts), Hepaticophyta (e.g., liverworts), Lycophyta (e.g., club mosses), Sphenophyta (e.g., horsetails), Psilophyta (e.g., whisk ferns), Ophioglossophyta, Pterophyta (e.g., ferns), Cycadophyta, Gingkophyta, Pinophyta, Gnetophyta, and Magnoliophyta (e.g., flowering plants). Suitable host cells include cells of members of the kingdom Animalia, including, but not limited to, members of any of the following phyla: Porifera (sponges); Placozoa; Orthonectida (parasites of marine invertebrates); Rhombozoa; Cnidaria (corals, anemones, jellyfish, sea pens, sea pansies, sea wasps); Ctenophora (comb jellies); Platyhelminthes (flatworms); Nemertina (ribbon worms); Ngathostomulida (jawed worms)p Gastrotricha; Rotifera; Priapulida; Kinorhyncha; Loricifera; Acanthocephala; Entoprocta; Nemotoda; Nematomorpha; Cycliophora; Mollusca (mollusks); Sipuncula (peanut worms); Annelida (segmented worms); Tardigrada (water bears); Onychophora (velvet worms); Arthropoda (including the subphyla: Chelicerata, Myriapoda, Hexapoda, and Crustacea, where the Chelicerata include, e.g., arachnids, Merostomata, and Pycnogonida, where the Myriapoda include, e.g., Chilopoda (centipedes), Diplopoda (millipedes), Paropoda, and Symphyla, where the Hexapoda include insects, and where the Crustacea include shrimp, krill, barnacles, etc.; Phoronida; Ectoprocta (moss animals); Brachiopoda; Echinodermata (e.g. starfish, sea daisies, feather stars, sea urchins, sea cucumbers, brittle stars, brittle baskets, etc.); Chaetognatha (arrow worms); Hemichordata (acorn worms); and Chordata. Suitable members of Chordata include any member of the following subphyla: Urochordata (sea squirts; including Ascidiacea, Thaliacea, and Larvacea); Cephalochordata (lancelets); Myxini (hagfish); and Vertebrata, where members of Vertebrata include, e.g., members of Petromyzontida (lampreys), Chondrichthyces (cartilaginous fish), Actinopterygii (ray-finned fish), Actinista (coelocanths), Dipnoi (lungfish), Reptilia (reptiles, e.g., snakes, alligators, crocodiles, lizards, etc.), Aves (birds); and Mammalian (mammals). Suitable plant cells include cells of any monocotyledon and cells of any dicotyledon. Plant cells include, e.g., a cell of a leaf, a root, a tuber, a flower, and the like. In some cases, the genetically modified host cell is a plant cell. In some cases, the genetically modified host cell is a bacterial cell. In some cases, the genetically modified host cell is an archaeal cell.

Suitable eukaryotic host cells include, but are not limited to, Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Neurospora crassa, Chlamydomonas reinhardtii, and the like. In some cases, subject genetically modified host cell is a yeast cell. In some instances, the yeast cell is Saccharomyces cerevisiae.

Suitable prokaryotic cells include any of a variety of bacteria, including laboratory bacterial strains, pathogenic bacteria, etc. Suitable prokaryotic hosts include, but are not limited, to any of a variety of gram-positive, gram-negative, or gram-variable bacteria. Examples include, but are not limited to, cells belonging to the genera: Agrobacterium, Alicyclobacillus, Anabaena, Anacystis, Arthrobacter, Azobacter, Bacillus, Brevibacterium, Chromatium, Clostridium, Corynebacterium, Enterobacter, Erwinia, Escherichia, Lactobacillus, Lactococcus, Mesorhizobium, Methylobacterium, Microbacterium, Phormidium, Pseudomonas, Rhodobacter, Rhodopseudomonas, Rhodospirillum, Rhodococcus, Salmonella, Scenedesmun, Serratia, Shigella, Staphylococcus, Strepromyces, Synnecoccus, and Zymomonas. Examples of prokaryotic strains include, but are not limited to: Bacillus subtilis, Bacillus amyloliquefacines, Brevibacterium ammoniagenes, Brevibacterium immariophilum, Clostridium beigerinckii, Enterobacter sakazakii, Escherichia coli, Lactococcus lactis, Mesorhizobium loti, Pseudomonas aeruginosa, Pseudomonas mevalonii, Pseudomonas pudica, Rhodobacter capsulatus, Rhodobacter sphaeroides, Rhodospirillum rubrum, Salmonella enterica, Salmonella typhi, Salmonella typhimurium, Shigella dysenteriae, Shigella flexneri, Shigella sonnei, and Staphylococcus aureus. One example of a suitable bacterial host cell is Escherichia coli cell.

Suitable plant cells include cells of a monocotyledon; cells of a dicotyledon; cells of an angiosperm; cells of a gymnosperm; etc.

### Nucleic Acids, Expression Vectors, and Host Cells

The present disclosure provides nucleic acid(s) comprising nucleotide sequences encoding one or more components of a PPI detection system of the present disclosure. The present disclosure provides host cells genetically modified with the one or more nucleic acid(s).

The present disclosure provides a nucleic acid comprising: a) a nucleotide sequence encoding a transmembrane domain or other tethering domain; b) an insertion site for a nucleic acid comprising a nucleotide sequence encoding a first member of a protein interaction pair; c) a light-activated polypeptide comprising a LOV domain comprising an amino acid sequence having at least 80% amino acid sequence identity to any one of the amino acid sequences set forth in FIG. 11A-11G; d) a proteolytically cleavable linker; and e) an insertion site for a nucleic acid comprising a nucleotide sequence encoding a polypeptide of interest. The present disclosure provides a nucleic acid comprising: a) a nucleotide sequence encoding a transmembrane domain or other tethering domain; b) an insertion site for a nucleic acid comprising a nucleotide sequence encoding a first member of a protein interaction pair; c) a light-activated polypeptide comprising a LOV domain comprising an amino acid sequence having at least 80% amino acid sequence identity to any one of the amino acid sequences set forth in FIG. 11A-11G; d) a proteolytically cleavable linker; and e) a transcription factor.

The present disclosure provides a nucleic acid system comprising: a) a first nucleic acid comprising a nucleotide sequence encoding a first fusion polypeptide comprising: i) a transmembrane domain (or other tethering domain); ii) a first polypeptide member of a protein-interaction pair; ii) a light-activated polypeptide comprising a LOV domain; iii) a proteolytically cleavable linker that is caged by the light-activated polypeptide in the absence of blue light; and iv) a transcription factor; and b) a second nucleic acid comprising a nucleotide sequence encoding a second fusion polypeptide comprising: a) a second member of the protein interaction pair; and b) a protease that cleaves the proteolytically cleavable linker under certain conditions.

The present disclosure provides a nucleic acid comprising: a nucleic acid comprising: a) a nucleotide sequence encoding a fusion polypeptide comprising: i) a transmembrane domain; ii) a first polypeptide member of a protein-interaction pair; ii) a light-activated polypeptide comprising a LOV domain; and iii) a proteolytically cleavable linker that is caged by the light-activated polypeptide in the absence of blue light; and b) an insertion site for inserting a nucleic acid comprising a nucleotide sequence encoding a polypeptide of interest. The insertion site is within 10 nucleotides (nt), within 9 nt, within 8 nt, within 7 nt, within 6 nt, within 5 nt, within 4 nt, within 3 nt, within 2 nt, or 1 nt, of the 3′ end of the nucleotide sequence encoding the light-activated, calcium-gated fusion polypeptide. The insertion site is positioned relative to the nucleotide sequence encoding the first polypeptide such that, after insertion of a nucleic acid comprising a nucleotide sequence encoding a polypeptide of interest, and after transcription and translation, a fusion polypeptide comprising: i) a transmembrane domain; ii) a first polypeptide member of a protein-interaction pair; iii) a LOV-domain light-activated polypeptide comprising an amino acid sequence having at least 80% amino acid sequence identity to the amino acid sequence depicted in any one of FIG. 11A-11G; iv) a proteolytically cleavable linker; and v) the polypeptide of interest, is produced. In some cases, the insertion site is a multiple cloning site.

In any of the above embodiments, the nucleic acid(s) can be present in a recombinant expression vector. In some cases, the recombinant expression vector is a viral construct, e.g., a recombinant adeno-associated virus (AAV) construct, a recombinant adenoviral construct, a recombinant lentiviral construct, a recombinant retroviral construct, etc. In some cases, a nucleic acid of a system of the present disclosure is a recombinant lentivirus vector. In some cases, a nucleic acid of a system of the present disclosure is a recombinant AAV vector.

Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., Hum Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. In some cases, the vector is a lentivirus vector. Also suitable are transposon-mediated vectors, such as piggyback and sleeping beauty vectors.

In some cases, a nucleic acid or a nucleic acid system of the present disclosure is packaged in a viral particle. For example, in some cases, one or more of the nucleic acids of a nucleic acid system of the present disclosure are recombinant AAV vectors, and are packaged in recombinant AAV particles. Thus, the present disclosure provides a recombinant viral particle comprising a nucleic acid or a nucleic acid system of the present disclosure.

The present disclosure provides genetically modified host cells, where a host cell is genetically modified with a nucleic acid(s) comprising nucleotide sequences encoding one or more PPI detection system components, as described above. In some cases, a nucleic acid(s) comprising nucleotide sequences encoding one or more PPI detection system components, as described above, is stably integrated into the genome of the host cell. In some cases, a nucleic acid(s) comprising nucleotide sequences encoding one or more PPI detection system components, as described above, is present in the host cell episomally. The genetically modified cell can be in vitro or in vivo.

In some cases, the genetically modified host cell is a primary (non-immortalized) cell. In some cases, the genetically modified host cell is an immortalized cell line.

A genetically modified host cell of the present disclosure is a eukaryotic cell. Suitable host cells include mammalian cells, insect cells, reptile cells, amphibian cells, arachnid cells, and the like.

In some cases, the genetically modified host cell is a mammalian cell, e.g., a human cell, a non-human primate cell, a rodent cell, a feline (e.g., a cat) cell, a canine (e.g., a dog) cell, an ungulate cell, an equine (e.g., a horse) cell, an ovine cell, a caprine cell, a bovine cell, etc. In some cases, the genetically modified host cell is a rodent cell (e.g., a rat cell; a mouse cell). In some cases, the genetically modified host cell is a human cell. In some cases, the genetically modified host cell is a non-human primate cell.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

Suitable host cells include cells of, e.g., Bacteria (e.g., Eubacteria); Archaebacteria; Protista; Fungi; Plantae; and Animalia. Suitable host cells include cells of plant-like members of the kingdom Protista, including, but not limited to, algae (e.g., green algae, red algae, glaucophytes, cyanobacteria); fungus-like members of Protista, e.g., slime molds, water molds, etc.; animal-like members of Protista, e.g., flagellates (e.g., Euglena), amoeboids (e.g., amoeba), sporozoans (e.g, Apicomplexa, Myxozoa, Microsporidia), and ciliates (e.g., Paramecium). Suitable host cells include cells of members of the kingdom Fungi, including, but not limited to, members of any of the phyla: Basidiomycota (club fungi; e.g., members of Agaricus, Amanita, Boletus, Cantherellus, etc.); Ascomycota (sac fungi, including, e.g., Saccharomyces); Mycophycophyta (lichens); Zygomycota (conjugation fungi); and Deuteromycota. Suitable host cells include cells of members of the kingdom Plantae, including, but not limited to, members of any of the following divisions: Bryophyta (e.g., mosses), Anthocerotophyta (e.g., hornworts), Hepaticophyta (e.g., liverworts), Lycophyta (e.g., club mosses), Sphenophyta (e.g., horsetails), Psilophyta (e.g., whisk ferns), Ophioglossophyta, Pterophyta (e.g., ferns), Cycadophyta, Gingkophyta, Pinophyta, Gnetophyta, and Magnoliophyta (e.g., flowering plants). Suitable host cells include cells of members of the kingdom Animalia, including, but not limited to, members of any of the following phyla: Porifera (sponges); Placozoa; Orthonectida (parasites of marine invertebrates); Rhombozoa; Cnidaria (corals, anemones, jellyfish, sea pens, sea pansies, sea wasps); Ctenophora (comb jellies); Platyhelminthes (flatworms); Nemertina (ribbon worms); Ngathostomulida (jawed worms)p Gastrotricha; Rotifera; Priapulida; Kinorhyncha; Loricifera; Acanthocephala; Entoprocta; Nemotoda; Nematomorpha; Cycliophora; Mollusca (mollusks); Sipuncula (peanut worms); Annelida (segmented worms); Tardigrada (water bears); Onychophora (velvet worms); Arthropoda (including the subphyla: Chelicerata, Myriapoda, Hexapoda, and Crustacea, where the Chelicerata include, e.g., arachnids, Merostomata, and Pycnogonida, where the Myriapoda include, e.g., Chilopoda (centipedes), Diplopoda (millipedes), Paropoda, and Symphyla, where the Hexapoda include insects, and where the Crustacea include shrimp, krill, barnacles, etc.; Phoronida; Ectoprocta (moss animals); Brachiopoda; Echinodermata (e.g. starfish, sea daisies, feather stars, sea urchins, sea cucumbers, brittle stars, brittle baskets, etc.); Chaetognatha (arrow worms); Hemichordata (acorn worms); and Chordata. Suitable members of Chordata include any member of the following subphyla: Urochordata (sea squirts; including Ascidiacea, Thaliacea, and Larvacea); Cephalochordata (lancelets); Myxini (hagfish); and Vertebrata, where members of Vertebrata include, e.g., members of Petromyzontida (lampreys), Chondrichthyces (cartilaginous fish), Actinopterygii (ray-finned fish), Actinista (coelocanths), Dipnoi (lungfish), Reptilia (reptiles, e.g., snakes, alligators, crocodiles, lizards, etc.), Aves (birds); and Mammalian (mammals). Suitable plant cells include cells of any monocotyledon and cells of any dicotyledon. Plant cells include, e.g., a cell of a leaf, a root, a tuber, a flower, and the like. In some cases, the genetically modified host cell is a plant cell. In some cases, the genetically modified host cell is a bacterial cell. In some cases, the genetically modified host cell is an archaeal cell.

Suitable eukaryotic host cells include, but are not limited to, Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Neurospora crassa, Chlamydomonas reinhardtii, and the like. In some cases, subject genetically modified host cell is a yeast cell. In some instances, the yeast cell is Saccharomyces cerevisiae.

Suitable prokaryotic cells include any of a variety of bacteria, including laboratory bacterial strains, pathogenic bacteria, etc. Suitable prokaryotic hosts include, but are not limited, to any of a variety of gram-positive, gram-negative, or gram-variable bacteria. Examples include, but are not limited to, cells belonging to the genera: Agrobacterium, Alicyclobacillus, Anabaena, Anacystis, Arthrobacter, Azobacter, Bacillus, Brevibacterium, Chromatium, Clostridium, Corynebacterium, Enterobacter, Erwinia, Escherichia, Lactobacillus, Lactococcus, Mesorhizobium, Methylobacterium, Microbacterium, Phormidium, Pseudomonas, Rhodobacter, Rhodopseudomonas, Rhodospirillum, Rhodococcus, Salmonella, Scenedesmun, Serratia, Shigella, Staphylococcus, Strepromyces, Synnecoccus, and Zymomonas. Examples of prokaryotic strains include, but are not limited to: Bacillus subtilis, Bacillus amyloliquefacines, Brevibacterium ammoniagenes, Brevibacterium immariophilum, Clostridium beigerinckii, Enterobacter sakazakii, Escherichia coli, Lactococcus lactis, Mesorhizobium loti, Pseudomonas aeruginosa, Pseudomonas mevalonii, Pseudomonas pudica, Rhodobacter capsulatus, Rhodobacter sphaeroides, Rhodospirillum rubrum, Salmonella enterica, Salmonella typhi, Salmonella typhimurium, Shigella dysenteriae, Shigella flexneri, Shigella sonnei, and Staphylococcus aureus. One example of a suitable bacterial host cell is Escherichia coli cell.

Suitable plant cells include cells of a monocotyledon; cells of a dicotyledon; cells of an angiosperm; cells of a gymnosperm; etc.

### Genetically Modified Non-Human Organisms

The present disclosure provides genetically modified non-human organism, where the non-human organism is genetically modified with one or more nucleic acids of the present disclosure. The genetically modified non-human organism can be a vertebrate or an invertebrate animal. The genetically modified non-human organism can be a plant.

The genetically modified non-human organism can be an animal, e.g., a vertebrate animal. In some cases, the genetically modified non-human organism is a mammal. In some cases, the genetically modified non-human organism is an amphibian. In some cases, the genetically modified non-human organism is a reptile. In some cases, the genetically modified non-human organism is an insect. In some cases, the genetically modified non-human organism is an arachnid.

A nucleic acid of the present disclosure can be integrated into the genome of the genetically modified non-human organism. In some cases, the genetically modified non-human organism is heterozygous for the integration of the nucleic acid. In some cases, the genetically modified non-human organism is homozygous for the integration of the nucleic acid.

In some embodiments, a subject genetically modified non-human host cell can generate a subject genetically modified non-human organism (e.g., a mouse, a fish, a frog, a fly, a worm, etc.). For example, if the genetically modified host cell is a pluripotent stem cell (i.e., PSC) or a germ cell (e.g., sperm, oocyte, etc.), an entire genetically modified organism can be derived from the genetically modified host cell. In some embodiments, the genetically modified host cell is a pluripotent stem cell (e.g., embryonic stem cell (ESC), induced PSC (iPSC), pluripotent plant stem cell, etc.) or a germ cell (e.g., sperm cell, oocyte, etc.), either in vivo or in vitro, that can give rise to a genetically modified organism. In some embodiments the genetically modified host cell is a vertebrate PSC (e.g., ESC, iPSC, etc.) and is used to generate a genetically modified organism (e.g. by injecting a PSC into a blastocyst to produce a chimeric/mosaic animal, which could then be mated to generate non-chimeric/non-mosaic genetically modified organisms; grafting in the case of plants; etc.). Any convenient method/protocol for producing a genetically modified organism is suitable for producing a genetically modified host cell comprising a nucleic acid(s) of the present disclosure.

Methods of producing genetically modified organisms are known in the art. For example, see Cho et al., Curr Protoc Cell Biol. 2009 March; Chapter 19:Unit 19.11: Generation of transgenic mice; Gama et al., Brain Struct Funct. 2010 March; 214(2-3):91-109. Epub 2009 Nov. 25: Animal transgenesis: an overview; Husaini et al., GM Crops. 2011 June-December; 2(3): 150-62. Epub 2011 Jun. 1: Approaches for gene targeting and targeted gene expression in plants. A CRISPR/Cas9 system can be used to generate a transgenic organism. See, e.g., U.S. Patent Publication Nos. 2014/0068797 and 2015/0232882.

In some cases, a genetically modified organism comprises a target cell, and thus can be considered a source for target cells. For example, if a genetically modified cell comprising one or more nucleic acids of the present disclosure is used to generate a genetically modified organism, then the cells of the genetically modified organism comprise the one or more exogenous nucleic acids comprising nucleotide sequences encoding a polypeptide of the present disclosure. In some such embodiments, the DNA of a cell or cells of the genetically modified organism can be targeted for modification by introducing into the cell or cells a nucleic acid(s) of the present disclosure.

A subject genetically modified non-human organism can be any organism other than a human, including for example, a plant; algae; an invertebrate (e.g., a cnidarian, an echinoderm, a worm, a fly, etc.); a vertebrate (e.g., a fish (e.g., zebrafish, puffer fish, gold fish, etc.), an amphibian (e.g., salamander, frog, etc.), a reptile, a bird, a mammal, etc.); an ungulate (e.g., a goat, a pig, a sheep, a cow, etc.); a rodent (e.g., a mouse, a rat, a hamster, a guinea pig); a lagomorpha (e.g., a rabbit); etc.

### Methods

The present disclosure provides methods of detecting protein-protein interaction. The present disclosure provides methods of identifying a polypeptide that interacts with a known polypeptide (e.g., a “bait” polypeptide). The present disclosure provides methods of identifying a polypeptide variant that that interacts with a known polypeptide (e.g., a “bait” polypeptide). The present disclosure provides methods of identifying an agent or condition that modulates (increases, decreases, induces, or inhibits) a protein-protein interaction. The present disclosure provides methods of controlling an activity of a cell.

A method of the present disclosure involves use of a cell comprising a nucleic acid or a nucleic acid system of the present disclosure. In some cases, the cell (also referred to as a “target cell”) comprising a PPI detection system of the present disclosure is in vitro. In some cases, the cell (also referred to as a “target cell”) comprising a PPI detection system of the present disclosure is in vivo. The target cell is generally a eukaryotic cell. The target cell can be a mammalian cell, e.g., a human cell, a non-human primate cell, a rodent cell (e.g., a mouse cell; a rat cell), a lagomorph (e.g., rabbit) cell, etc.; a reptile cell; an amphibian cell; an insect cell; an arachnid cell; etc.

Where the cell is in vitro, binding of the second polypeptide member to the first polypeptide member of a protein-interaction pair can be detected by detecting a signal produced by a reporter gene product, e.g., using standard instrumentation (e.g., a colorimeter; a fluorimeter; a luminometer) for detecting such signals.

Where the cell is in vivo, binding of the second polypeptide member to the first polypeptide member of a protein-interaction pair can be detected by detecting a signal produced by a reporter gene product (e.g., such as any fluorescent protein (BFP, GFP, RFP, Venus, Neptune, Citrine, mCherry, dsRed, Tomato), an polypeptide with an epitope tag, luciferase, APEX, beta-galactosidase, beta-lactamase, HRP, peroxidase, chloramphenicol transferase, etc., and other reporter gene products listed elsewhere herein). Suitable reporter genes include those that complement a defect in an auxotroph (e.g., uracil, histidine, or leucine biosynthetic enzymes). Suitable reporter genes include drug resistance, antibiotic resistance, and the like.

Suitable target cells include, but are not limited to, neurons, endothelial cells, epithelial cells, astrocytes, glial cells, muscle cells, cardiomyocytes, keratinocytes, hepatocytes, retinal cells, adipocytes, chondrocytes, mesenchymal cells, osteoclasts, osteoblasts, stem cells, adult stem cells, and the like.

Suitable target cells include primary cells and immortalized cells (e.g., cells of an immortalized cell line).

In some cases, the target cell is a mammalian cell, e.g., a human cell, a non-human primate cell, a rodent cell, a feline (e.g., a cat) cell, a canine (e.g., a dog) cell, an ungulate cell, an equine (e.g., a horse) cell, an ovine cell, a caprine cell, a bovine cell, etc. In some cases, the target cell is a rodent cell (e.g., a rat cell; a mouse cell). In some cases, the target cell is a human cell. In some cases, the target host cell is a non-human primate cell.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

In some case, the target cell is in a particular tissue, e.g., brain tissue, kidney, liver, skin, blood, bone, skeletal muscle, cardiac muscle, breast tissue, lung, eye, or other tissue.

In some cases, the tissue is a brain tissue selected from the thalamus (including the central thalamus), sensory cortex (including the somatosensory cortex), zona incerta (ZI), ventral tegmental area (VTA), prefontal cortex (PFC), nucleus accumbens (NAc), amygdala (BLA), substantia nigra, ventral pallidum, globus pallidus, dorsal striatum, ventral striatum, subthalamic nucleus, hippocampus, dentate gyrus, cingulate gyrus, entorhinal cortex, olfactory cortex, primary motor cortex, and cerebellum.

Suitable target cells include stem cells, including iPS cells, ES cells, adult stem cells (e.g., cardiac stem cells; mesenchymal stem cells; etc.), etc.

Suitable target cells include cells of, e.g., Bacteria (e.g., Eubacteria); Archaebacteria; Protista; Fungi; Plantae; and Animalia. Suitable host cells include cells of plant-like members of the kingdom Protista, including, but not limited to, algae (e.g., green algae, red algae, glaucophytes, cyanobacteria); fungus-like members of Protista, e.g., slime molds, water molds, etc.; animal-like members of Protista, e.g., flagellates (e.g., Euglena), amoeboids (e.g., amoeba), sporozoans (e.g, Apicomplexa, Myxozoa, Microsporidia), and ciliates (e.g., Paramecium). Suitable host cells include cells of members of the kingdom Fungi, including, but not limited to, members of any of the phyla: Basidiomycota (club fungi; e.g., members of Agaricus, Amanita, Boletus, Cantherellus, etc.); Ascomycota (sac fungi, including, e.g., Saccharomyces); Mycophycophyta (lichens); Zygomycota (conjugation fungi); and Deuteromycota. Suitable host cells include cells of members of the kingdom Plantae, including, but not limited to, members of any of the following divisions: Bryophyta (e.g., mosses), Anthocerotophyta (e.g., hornworts), Hepaticophyta (e.g., liverworts), Lycophyta (e.g., club mosses), Sphenophyta (e.g., horsetails), Psilophyta (e.g., whisk ferns), Ophioglossophyta, Pterophyta (e.g., ferns), Cycadophyta, Gingkophyta, Pinophyta, Gnetophyta, and Magnoliophyta (e.g., flowering plants). Suitable host cells include cells of members of the kingdom Animalia, including, but not limited to, members of any of the following phyla: Porifera (sponges); Placozoa; Orthonectida (parasites of marine invertebrates); Rhombozoa; Cnidaria (corals, anemones, jellyfish, sea pens, sea pansies, sea wasps); Ctenophora (comb jellies); Platyhelminthes (flatworms); Nemertina (ribbon worms); Ngathostomulida (jawed worms)p Gastrotricha; Rotifera; Priapulida; Kinorhyncha; Loricifera; Acanthocephala; Entoprocta; Nemotoda; Nematomorpha; Cycliophora; Mollusca (mollusks); Sipuncula (peanut worms); Annelida (segmented worms); Tardigrada (water bears); Onychophora (velvet worms); Arthropoda (including the subphyla: Chelicerata, Myriapoda, Hexapoda, and Crustacea, where the Chelicerata include, e.g., arachnids, Merostomata, and Pycnogonida, where the Myriapoda include, e.g., Chilopoda (centipedes), Diplopoda (millipedes), Paropoda, and Symphyla, where the Hexapoda include insects, and where the Crustacea include shrimp, krill, barnacles, etc.; Phoronida; Ectoprocta (moss animals); Brachiopoda; Echinodermata (e.g. starfish, sea daisies, feather stars, sea urchins, sea cucumbers, brittle stars, brittle baskets, etc.); Chaetognatha (arrow worms); Hemichordata (acorn worms); and Chordata. Suitable members of Chordata include any member of the following subphyla: Urochordata (sea squirts; including Ascidiacea, Thaliacea, and Larvacea); Cephalochordata (lancelets); Myxini (hagfish); and Vertebrata, where members of Vertebrata include, e.g., members of Petromyzontida (lampreys), Chondrichthyces (cartilaginous fish), Actinopterygii (ray-finned fish), Actinista (coelocanths), Dipnoi (lungfish), Reptilia (reptiles, e.g., snakes, alligators, crocodiles, lizards, etc.), Aves (birds); and Mammalian (mammals). Suitable plant cells include cells of any monocotyledon and cells of any dicotyledon. Plant cells include, e.g., a cell of a leaf, a root, a tuber, a flower, and the like. In some cases, the genetically modified host cell is a plant cell. In some cases, the genetically modified host cell is a bacterial cell. In some cases, the genetically modified host cell is an archaeal cell.

Suitable eukaryotic host cells include, but are not limited to, Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Neurospora crassa, Chlamydomonas reinhardtii, and the like. In some cases, subject genetically modified host cell is a yeast cell. In some instances, the yeast cell is Saccharomyces cerevisiae.

Suitable prokaryotic cells include any of a variety of bacteria, including laboratory bacterial strains, pathogenic bacteria, etc. Suitable prokaryotic hosts include, but are not limited, to any of a variety of gram-positive, gram-negative, or gram-variable bacteria. Examples include, but are not limited to, cells belonging to the genera: Agrobacterium, Alicyclobacillus, Anabaena, Anacystis, Arthrobacter, Azobacter, Bacillus, Brevibacterium, Chromatium, Clostridium, Corynebacterium, Enterobacter, Erwinia, Escherichia, Lactobacillus, Lactococcus, Mesorhizobium, Methylobacterium, Microbacterium, Phormidium, Pseudomonas, Rhodobacter, Rhodopseudomonas, Rhodospirillum, Rhodococcus, Salmonella, Scenedesmun, Serratia, Shigella, Staphylococcus, Strepromyces, Synnecoccus, and Zymomonas. Examples of prokaryotic strains include, but are not limited to: Bacillus subtilis, Bacillus amyloliquefacines, Brevibacterium ammoniagenes, Brevibacterium immariophilum, Clostridium beigerinckii, Enterobacter sakazakii, Escherichia coli, Lactococcus lactis, Mesorhizobium loti, Pseudomonas aeruginosa, Pseudomonas mevalonii, Pseudomonas pudica, Rhodobacter capsulatus, Rhodobacter sphaeroides, Rhodospirillum rubrum, Salmonella enterica, Salmonella typhi, Salmonella typhimurium, Shigella dysenteriae, Shigella flexneri, Shigella sonnei, and Staphylococcus aureus. One example of a suitable bacterial host cell is Escherichia coli cell.

Suitable plant cells include cells of a monocotyledon; cells of a dicotyledon; cells of an angiosperm; cells of a gymnosperm; etc.

In some cases, a PPI detection system of the present disclosure provides a high signal-to-noise (S/N) ratio. For example, as described above, in some cases, a cell comprising a PPI detection system of the present disclosure comprises: a) a first fusion polypeptide comprising: i) a TM domain; ii) a first polypeptide member of a protein interaction pair; iii) a LOV domain light-activated polypeptide; iv) a proteolytically cleavable linker; and v) a transcription factor; and b) a second fusion polypeptide comprising: i) a second polypeptide member of the protein interaction pair; and ii) a protease; and where the cell is genetically modified with a heterologous nucleic acid comprising nucleotide sequence encoding a reporter, where the nucleotide sequence is operably linked to a promoter, and where the promoter is activated by the transcription factor when the transcription factor is released from the first fusion polypeptide. For example, following exposure (substantially simultaneously) of such a cell comprising a PPI detection system of the present disclosure to blue light and a second stimulus (such that the first and second members of the protein interaction pair bind to one another), the transcription factor is released from the first fusion polypeptide (by cleavage of the proteolytically cleavable linker by the protease), and induces transcription of the heterologous nucleic acid, such that the reporter polypeptide is produced in the cell. The signal produced by the reporter polypeptide in a cell exposed substantially simultaneously to blue light and the second stimulus is at least 3-fold, at least 4-fold, at least 5-fold, at least 6-fold, at least 7-fold, at least 8-fold, at least 9-fold, at least 10-fold, or more than 10-fold, higher than the signal produced by the reporter polypeptide in a control cell not exposed substantially simultaneously to blue light and the second stimulus (e.g., in a control cell exposed to blue light and not to the second stimulus; in a control cell exposed to the second stimulus but not the blue light; or in a control cell exposed to both blue light and the second stimulus, but where the exposure is not substantially simultaneous).

A PPI detection system of the present disclosure, when present in a cell, can provide for temporal information regarding a PPI. Thus, a method of the present disclosure can be carried out over time. For example, a signal generated by a PPI system of the present disclosure can be detected for a continuous period of time following exposure to a first and second stimulus; e.g., for a continuous period of time of from 1 minute to several hours or days (e.g., from 1 minute to 15 minutes, from 15 minutes to 30 minutes, from 30 minutes to 1 hour, from 1 hour to 4 hours, from 4 hours to 8 hours, etc.) following exposure to a first and second stimulus. A signal generated by a PPI system of the present disclosure can be detected periodically over a period of time following exposure to a first and second stimulus; e.g., periodically (e.g., once every 0.5 seconds, once every second, once every 15 seconds, once every 30 seconds, once every 60 seconds, once every 15 minutes, once every 30 minutes, once every hour, etc.) over a period of time of from 1 minute to several hours or days (e.g., from 1 minute to 15 minutes, from 15 minutes to 30 minutes, from 30 minutes to 1 hour, from 1 hour to 4 hours, from 4 hours to 8 hours, etc.) following exposure to a first and second stimulus.

Methods of Detecting Protein-Protein Interaction

The present disclosure provides methods of detecting protein-protein interaction in a cell. The methods generally involve exposing a cell, which cell comprises a PPI system of the present disclosure, to two stimuli substantially simultaneously: the first stimulus is blue light; and the second stimulus is any condition, agent, or other stimulus that effects binding of a second polypeptide member of a protein interaction pair to the first polypeptide member of the protein-protein interaction pair. Following the substantially simultaneous exposure of the cell to the first and the second stimuli, the polypeptide of interest is released from the first fusion polypeptide, and generates (directly or indirectly) a signal that serves as a readout for the binding of the first fusion polypeptide to the second polypeptide, and hence as a readout for interaction of the first polypeptide member of the protein-protein interaction pair with the second polypeptide member of the protein-protein interaction pair.

The second stimulus (the stimulus that induces binding of a second polypeptide member of a protein interaction pair to the first polypeptide member of the protein-protein interaction pair) can be any of a variety of stimuli. For example, the second stimulus can be: 1) binding of a ligand to a cell surface receptor present on the surface of the cell; 2) binding of a neurotransmitter to the cell (e.g., to a cell surface receptor for the neurotransmitter); 3) a change in temperature; 4) interaction of the target cell with a second cell (e.g., an effector cell); 5) binding of a hormone to the cell; 6) binding of a cytokine to the cell; 7) binding of a chemokine to the cell; 8) binding of a drug (e.g., a pharmaceutical agent) to the cell; 9) binding of an antibody to the cell (e.g., an antibody specific for an epitope present on the surface of the cell); 10) a change in oxygen concentration in the external environment of the cell (e.g., hypoxic conditions); 11) a change in the ion concentration in the liquid environment of the cell; 12) an electrical charge (e.g., producing a voltage change in the membrane of the cell); 13) a nutrient (e.g., a nutrient present in the external environment of the cell); 14) an adhesion polypeptide; 15) an extracellular matrix; 16) a pathogen (e.g., a virus, a protozoan, a bacterium); 17) a toxin; 18) a mitogen; 19) a drug, such as histamine, that triggers release of calcium from intracellular stores; 20) an ionophore (e.g., ionomycin, etc.); 21) external electrode stimulation; etc.

Reporter Polypeptides

Suitable reporter polypeptides include polypeptides that generate a detectable signal. Suitable detectable signal-producing proteins include, e.g., fluorescent proteins; enzymes that catalyze a reaction that generates a detectable signal as a product; and the like.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilised ECFP (dECFP), destabilized EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Other examples of fluorescent proteins include mHoneydew, mBanana, mOrange, dTomato, tdTomato, mTangerine, mStrawberry, mCherry, mGrape1, mRaspberry, mGrape2, mPlum (Shaner et al. (2005) Nat. Methods 2:905-909), Neptune, and the like. Any of a variety of fluorescent and colored proteins from Anthozoan species, as described in, e.g., Matz et al. (1999) Nature Biotechnol. 17:969-973, or Rodriguez et al. (2016) Trends Biochem. Sci. is suitable for use.

Suitable enzymes include, but are not limited to, horse radish peroxidase (HRP), alkaline phosphatase (AP), beta-galactosidase (GAL), β-lactamase, glucose-6-phosphate dehydrogenase, beta-N-acetylglucosaminidase, β-glucuronidase, invertase, Xanthine Oxidase, luciferase, glucose oxidase (GO), engineered ascorbate peroxidase (e.g., APEX; APEX2); and the like. In some cases, the enzyme acts on a substrate to produce a colored product (e.g., a product that can be detected colorimetrically). In some cases, the enzyme acts on a substrate to produce a fluorescent product. In some cases, the enzyme acts on a substrate to produce a luminescent product.

Methods of Identifying a Polypeptide that Interacts with a Known Polypeptide

The present disclosure provides methods of identifying a polypeptide that interacts with a known polypeptide (e.g., a “bait” polypeptide). The methods generally involve exposing a cell, which cell comprises a PPI system of the present disclosure, to two stimuli substantially simultaneously: the first stimulus is blue light; and the second stimulus is any condition, agent, or other stimulus that effects binding of a second polypeptide member of a protein interaction pair to the first polypeptide member of a protein-protein interaction pair. Following the substantially simultaneous exposure of the cell to the first and the second stimuli, the polypeptide of interest is released from the first fusion polypeptide, and generates (directly or indirectly) a signal that serves as a readout for the binding of the first fusion polypeptide to the second polypeptide, and hence as a readout for interaction of the first polypeptide member of the protein-protein interaction pair with the second polypeptide member of the protein-protein interaction pair.

The cell is exposed to the first and the second stimulus substantially simultaneously, e.g., the cell is exposed to the first stimulus within about 1 second to about 60 seconds of the second stimulus, e.g., within about 1 second to about 5 seconds, within about 5 seconds to about 10 seconds, within about 10 seconds to about 15 seconds, within about 15 seconds to about 20 seconds, within about 20 seconds to about 30 seconds, within about 30 seconds to about 45 seconds, or within about 45 seconds to about 60 seconds, of the exposure to the cell of the second stimulus. In some cases, the cell is exposed to the first stimulus within less than 1 second of the exposure of the cell to the second stimulus, e.g., within 900 milliseconds, within 800 milliseconds, within 700 milliseconds, within 600 milliseconds, within 500 milliseconds, within 250 milliseconds, within 100 milliseconds, within 50 milliseconds, within 25 milliseconds, or within 10 milliseconds.

In some cases, the cell comprises a) a first nucleic acid comprising a nucleotide sequence encoding a first fusion polypeptide comprising, in order from amino terminus to carboxyl terminus: i) a transmembrane domain; ii) a first member of a protein interaction pair; iii) a LOV light-activated polypeptide comprising an amino acid sequence having at least 80% amino acid sequence identity to the amino acid sequence depicted in any one of FIG. 11A-11G; iv) a proteolytically cleavable linker; and v) a polypeptide of interest; and b) a second nucleic acid comprising a nucleotide sequence encoding a second fusion polypeptide comprising: i) a second member of the protein interaction pair; and ii) a protease that cleaves the proteolytically cleavable linker, wherein the first member of the protein interaction pair and the second member of the protein interaction pair bind to one another in the presence of a binding-inducing agent or condition. The cell expresses the first fusion polypeptide and the second fusion polypeptide. In some cases, the polypeptide of interest is a transcription factor. In some cases, the cell also comprises a nucleic acid comprising: a) a promoter that is activated by the transcription factor; and b) a nucleotide sequence that is operably linked to the promoter, and that encodes a gene product that is directly or indirectly detectable. For example, in some cases, the nucleotide sequence encodes a fluorescent polypeptide. In such cases, the fluorescent polypeptide is produced only when the first and second polypeptide members of the protein interaction pair bind to one another.

In some of these embodiments, as described above, the second fusion polypeptide is encoded by a member of a library of nucleic acids comprising a plurality of members. In some cases, each member comprises a nucleotide sequence that encodes a different second fusion polypeptide, where the second fusion polypeptides differ in the second member of the protein interaction pair. In some cases, each member of the library is bar-coded. Thus, the present disclosure provides a method of identifying a polypeptide that interacts with a “bait” protein.

In some of these embodiments, as described above, the second fusion polypeptide comprises: a) an unknown protein, to be tested for binding to a first polypeptide member of a protein interaction pair. The unknown (“prey”) protein can be a member of a protein library, where the protein library can have from 10 to 10⁹ protein members, e.g., from 10 proteins to 10² proteins, from 10² proteins to 10³ proteins, from 10³ proteins to 10⁴ proteins, from 10⁴ proteins to 10⁵ proteins, from 10⁵ proteins to 10⁶ proteins, from 10⁶ proteins to 10⁷ proteins, from 10⁷ proteins to 10⁸ proteins, or from 10⁸ proteins to 10⁹ proteins. In some cases, the library has more than 10⁹ proteins.

The library can be a library of proteins from a particular organism. For example, a library can be a library of proteins of, e.g., Bacteria (e.g., Eubacteria); Archaebacteria; Protista; Fungi; Plantae; and Animalia. A library can be a library of proteins of plant-like members of the kingdom Protista, including, but not limited to, algae (e.g., green algae, red algae, glaucophytes, cyanobacteria); fungus-like members of Protista, e.g., slime molds, water molds, etc.; animal-like members of Protista, e.g., flagellates (e.g., Euglena), amoeboids (e.g., amoeba), sporozoans (e.g, Apicomplexa, Myxozoa, Microsporidia), and ciliates (e.g., Paramecium). A library can be a library of proteins of the kingdom Fungi, including, but not limited to, members of any of the phyla: Basidiomycota (club fungi; e.g., members of Agaricus, Amanita, Boletus, Cantherellus, etc.); Ascomycota (sac fungi, including, e.g., Saccharomyces); Mycophycophyta (lichens); Zygomycota (conjugation fungi); and Deuteromycota. A library can be a library of proteins of a member of the kingdom Plantae, including, but not limited to, members of any of the following divisions: Bryophyta (e.g., mosses), Anthocerotophyta (e.g., hornworts), Hepaticophyta (e.g., liverworts), Lycophyta (e.g., club mosses), Sphenophyta (e.g., horsetails), Psilophyta (e.g., whisk ferns), Ophioglossophyta, Pterophyta (e.g., ferns), Cycadophyta, Gingkophyta, Pinophyta, Gnetophyta, and Magnoliophyta (e.g., flowering plants). A library can be a library of proteins of a member of the kingdom Animalia, including, but not limited to, members of any of the following phyla: Porifera (sponges); Placozoa; Orthonectida (parasites of marine invertebrates); Rhombozoa; Cnidaria (corals, anemones, jellyfish, sea pens, sea pansies, sea wasps); Ctenophora (comb jellies); Platyhelminthes (flatworms); Nemertina (ribbon worms); Ngathostomulida (jawed worms)p Gastrotricha; Rotifera; Priapulida; Kinorhyncha; Loricifera; Acanthocephala; Entoprocta; Nemotoda; Nematomorpha; Cycliophora; Mollusca (mollusks); Sipuncula (peanut worms); Annelida (segmented worms); Tardigrada (water bears); Onychophora (velvet worms); Arthropoda (including the subphyla: Chelicerata, Myriapoda, Hexapoda, and Crustacea, where the Chelicerata include, e.g., arachnids, Merostomata, and Pycnogonida, where the Myriapoda include, e.g., Chilopoda (centipedes), Diplopoda (millipedes), Paropoda, and Symphyla, where the Hexapoda include insects, and where the Crustacea include shrimp, krill, barnacles, etc.; Phoronida; Ectoprocta (moss animals); Brachiopoda; Echinodermata (e.g. starfish, sea daisies, feather stars, sea urchins, sea cucumbers, brittle stars, brittle baskets, etc.); Chaetognatha (arrow worms); Hemichordata (acorn worms); and Chordata. Suitable members of Chordata include any member of the following subphyla: Urochordata (sea squirts; including Ascidiacea, Thaliacea, and Larvacea); Cephalochordata (lancelets); Myxini (hagfish); and Vertebrata, where members of Vertebrata include, e.g., members of Petromyzontida (lampreys), Chondrichthyces (cartilaginous fish), Actinopterygii (ray-finned fish), Actinista (coelocanths), Dipnoi (lungfish), Reptilia (reptiles, e.g., snakes, alligators, crocodiles, lizards, etc.), Aves (birds); and Mammalian (mammals). A library can be a library of proteins of any monocotyledon and cells of any dicotyledon.

A library can be a library of proteins of a diseased cell or organism. For example, a protein library can be a library of proteins from a cancer cell, from a muscle cell comprising a defect in a muscle protein, and the like. A library can be a library of proteins of a healthy cell or organism.

A library can be a library of proteins of a cell or organism that has been exposed to any of a variety of stimuli, stresses, etc.

Methods of Identifying a Polypeptide Variant that that Interacts with a Known Polypeptide

The present disclosure provides methods of identifying a polypeptide variant that that interacts with a known polypeptide.

The methods generally involve exposing a cell, which cell comprises a PPI system of the present disclosure, to two stimuli substantially simultaneously: the first stimulus is blue light; and the second stimulus is any condition, agent, or other stimulus that effects binding of a second polypeptide member of a protein interaction pair to the first polypeptide member of a protein-protein interaction pair. Following the substantially simultaneous exposure of the cell to the first and the second stimuli, the polypeptide of interest is released from the first fusion polypeptide, and generates (directly or indirectly) a signal that serves as a readout for the binding of the first fusion polypeptide to the second polypeptide, and hence as a readout for interaction of the first polypeptide member of the protein-protein interaction pair with the second polypeptide member of the protein-protein interaction pair.

The cell is exposed to the first and the second stimulus substantially simultaneously, e.g., the cell is exposed to the first stimulus within about 1 second to about 60 seconds of the second stimulus, e.g., within about 1 second to about 5 seconds, within about 5 seconds to about 10 seconds, within about 10 seconds to about 15 seconds, within about 15 seconds to about 20 seconds, within about 20 seconds to about 30 seconds, within about 30 seconds to about 45 seconds, or within about 45 seconds to about 60 seconds, of the exposure to the cell of the second stimulus. In some cases, the cell is exposed to the first stimulus within less than 1 second of the exposure of the cell to the second stimulus, e.g., within 900 milliseconds, within 800 milliseconds, within 700 milliseconds, within 600 milliseconds, within 500 milliseconds, within 250 milliseconds, within 100 milliseconds, within 50 milliseconds, within 25 milliseconds, or within 10 milliseconds.

In some cases, the cell comprises a) a first nucleic acid comprising a nucleotide sequence encoding a first fusion polypeptide comprising, in order from amino terminus to carboxyl terminus: i) a transmembrane domain; ii) a first member of a protein interaction pair; iii) a LOV light-activated polypeptide comprising an amino acid sequence having at least 80% amino acid sequence identity to the amino acid sequence depicted in any one of FIG. 11A-11G; iv) a proteolytically cleavable linker; and v) a polypeptide of interest; and b) a second nucleic acid comprising a nucleotide sequence encoding a second fusion polypeptide comprising: i) a second member of the protein interaction pair; and ii) a protease that cleaves the proteolytically cleavable linker, wherein the first member of the protein interaction pair and the second member of the protein interaction pair bind to one another in the presence of a binding-inducing agent or condition. The cell expresses the first fusion polypeptide and the second fusion polypeptide. In some cases, the polypeptide of interest is a transcription factor. In some cases, the cell also comprises a nucleic acid comprising: a) a promoter that is activated by the transcription factor; and b) a nucleotide sequence that is operably linked to the promoter, and that encodes a gene product that is directly or indirectly detectable. For example, in some cases, the nucleotide sequence encodes a fluorescent polypeptide. In such cases, the fluorescent polypeptide is produced only when the first and second polypeptide members of the protein interaction pair bind to one another.

In some of these embodiments, as described above, the second fusion polypeptide comprises: a) a variant of a polypeptide that interacts with a first polypeptide member of a protein interaction pair. In some of these embodiments, as described above, the second fusion polypeptide is encoded by a member of a library of nucleic acids comprising a plurality of members. In some cases, each member comprises a nucleotide sequence that encodes a different second fusion polypeptide, where the second fusion polypeptides differ in the second member of the protein interaction pair. In some cases, each member of the library is bar-coded. Thus, the present disclosure provides a method of identifying a polypeptide that interacts with a “bait” protein.

In some cases, the second member of the protein interaction pair is a member of a library of proteins (“variant proteins”), each of which contains a single amino acid substitution relative to a reference protein, where the reference protein that is known to interact with the first member of the protein interaction pair. The variant (“prey”) protein can be a member of a protein library, where the protein library can have from 10 to 10⁹ protein members, e.g., from 10 proteins to 10² proteins, from 10² proteins to 10³ proteins, from 10³ proteins to 10⁴ proteins, from 10⁴ proteins to 10⁵ proteins, from 10⁵ proteins to 10⁶ proteins, from 10⁶ proteins to 10⁷ proteins, from 10⁷ proteins to 10⁸ proteins, or from 10⁸ proteins to 10⁹ proteins. In some cases, the library has more than 10⁹ proteins. In some cases, each member of the library is bar-coded.

In some cases, a single amino acid in a variant protein is mutated relative to the reference protein.

In some cases, the single amino acid is mutated to a different coded amino acid; for example, a library can comprise variant proteins, each of which contains substitution of a single amino acid to a different coded amino acid. For example, a protein variant library can comprise: a first member comprising a first substitution of amino acid X of the reference protein; a second member comprising a second substitution of amino acid X of the reference protein; a third member comprising a third substitution of amino acid X of the reference protein; etc., such that the library comprises all possible substitutions of amino acid X of the reference protein.

In other cases, a library of variant proteins comprises members each of which comprises a single amino acid substitution in a different amino acid of the reference protein. For example, where a reference protein comprises 200 amino acids, a library of variant proteins can comprise a first member comprising a substitution of amino acid 1 of the reference protein; a second member comprising a substitution of amino acid 2 of the reference protein; a third member comprising a substitution of amino acid 3 of the reference protein; etc., such that variants of each of the 200 amino acids is represented in the library.

The variant protein library can comprise members each of which comprises a different amino acid substitution in a different amino acid of the reference protein. For example, where a reference protein comprises 200 amino acids, a library of variant proteins can comprise: A) a first member comprising a first substitution of amino acid 1 of the reference protein; a second member comprising a second substitution of amino acid 1 of the reference protein; etc., up to a 19^{th} member comprising a 19^{th} substitution of amino acid 1 of the reference protein, such that the library comprises all possible substitutions of amino acid 1 of the reference protein; B) a 20th member comprising a first substitution of amino acid 2 of the reference protein; a 21st member comprising a second substitution of amino acid 2 of the reference protein; etc., such that the library comprises all possible substitutions of amino acid 2 of the reference protein; etc., such that the variant protein library contains individual members, where, for each amino acid of the reference protein, the library comprises a plurality of members each of which comprises a single amino acid substitution covering all possible substitutions (e.g., all coded amino acids) of each amino acid in the reference protein. Such a library could include, e.g., 3800 members (200 amino acid positions×19 amino acids).

As another example, in some cases, the second member of the protein interaction pair is a member of a library of proteins, each of which contains from 2 to 5 amino acid substitutions substitution relative to a reference protein that is known to interact with the first member of the protein interaction pair. In some cases, the from 2 to 5 amino acid substitutions are random. In some cases, the from 2 to 5 amino acid substitutions are in defined locations of a reference protein.

As another example, in some cases, the second member of the protein interaction pair is a member of a library of proteins, each of which contains an insertion (e.g., an insertion of 1, 2, 3, 4, 5, 6, 7, 8, 9, or 10 amino acids) at a different site relative to a reference protein that is known to interact with the first member of the protein interaction pair.

Whether a given variant binds to the “bait” protein can be determined by detecting the readout, e.g., a fluorescent protein, etc.

Method of Identifying an Agent or Condition that Modulates a Protein-Protein Interaction

The present disclosure provides methods of identifying an agent or condition that modulates (increases, decreases, induces, or inhibits) a protein-protein interaction.

The methods generally involve exposing a cell, which cell comprises a PPI system of the present disclosure, to two stimuli substantially simultaneously: the first stimulus is blue light; and the second stimulus is any condition, agent, or other stimulus that affects binding of a second polypeptide member of a protein interaction pair to the first polypeptide member of a protein-protein interaction pair. Following the substantially simultaneous exposure of the cell to the first and the second stimuli, the polypeptide of interest is released from the first fusion polypeptide, and generates (directly or indirectly) a signal that serves as a readout for the binding of the first fusion polypeptide to the second polypeptide, and hence as a readout for interaction of the first polypeptide member of the protein-protein interaction pair with the second polypeptide member of the protein-protein interaction pair.

In some cases, the method comprises exposing the cell to: a) a first stimulus, wherein the first stimulus is blue light; and b) a second stimulus, where the second stimulus is a test agent that is being tested for its effect on binding of the first and second polypeptide members of the protein interaction pair to one another. In some cases, exposure of the cell to the first stimulus and the test agent results in binding of the first and second polypeptide members of the protein interaction pair to one another. In some cases, exposure of the cell to the first stimulus and the test agent results in inhibition of binding of the first and second polypeptide members of the protein interaction pair to one another.

The cell is exposed to the first and the second stimulus substantially simultaneously, e.g., the cell is exposed to the first stimulus within about 1 second to about 60 seconds of the second stimulus, e.g., within about 1 second to about 5 seconds, within about 5 seconds to about 10 seconds, within about 10 seconds to about 15 seconds, within about 15 seconds to about 20 seconds, within about 20 seconds to about 30 seconds, within about 30 seconds to about 45 seconds, or within about 45 seconds to about 60 seconds, of the exposure to the cell of the second stimulus. In some cases, the cell is exposed to the first stimulus within less than 1 second of the exposure of the cell to the second stimulus, e.g., within 900 milliseconds, within 800 milliseconds, within 700 milliseconds, within 600 milliseconds, within 500 milliseconds, within 250 milliseconds, within 100 milliseconds, within 50 milliseconds, within 25 milliseconds, or within 10 milliseconds.

In some cases, the method comprises exposing the cell to: a) a first stimulus, wherein the first stimulus is blue light; b) a second stimulus, where the second stimulus is an agent that is known to induce binding of the first and second polypeptide members of the protein interaction pair to one another; and c) a test agent. In some cases, exposure of the cell to the first stimulus and the second stimulus results in binding of the first and second polypeptide members of the protein interaction pair to one another; and the test agent inhibits binding of the first and second polypeptide members of the protein interaction pair to one another.

Where the cell is exposed to a first and a second stimulus and a test agent, the cell is exposed to the first and the second stimulus, and the test agent, substantially simultaneously, e.g., the cell is exposed to the first stimulus within about 1 second to about 60 seconds of the second stimulus, e.g., within about 1 second to about 5 seconds, within about 5 seconds to about 10 seconds, within about 10 seconds to about 15 seconds, within about 15 seconds to about 20 seconds, within about 20 seconds to about 30 seconds, within about 30 seconds to about 45 seconds, or within about 45 seconds to about 60 seconds, of the exposure to the cell of the second stimulus. In some cases, the cell is exposed to the first stimulus within less than 1 second of the exposure of the cell to the second stimulus, e.g., within 900 milliseconds, within 800 milliseconds, within 700 milliseconds, within 600 milliseconds, within 500 milliseconds, within 250 milliseconds, within 100 milliseconds, within 50 milliseconds, within 25 milliseconds, or within 10 milliseconds.

A “test agent” can be a small molecule (e.g., a molecule having a molecular weight of less than about 5000 Daltons (Da), less than 2500 Da, less than 1000 Da, or less than 500 Da); an ion; light (e.g., light of a wavelength other than blue light); a hormone; a peptide; a nucleic acid; a lipid; and the like. A “test agent” Generally, a plurality of assay mixtures is run in parallel with different agents or agent concentrations to obtain a differential response to the various agents or agent concentrations. In some cases, one of these samples serves as a negative control, e.g., at zero concentration or below the level of detection.

Compounds of interest for screening include biologically active agents of numerous chemical classes, primarily organic molecules, which may include organometallic molecules, inorganic molecules, etc. Test agents can encompass numerous chemical classes, such as organic molecules, e.g., small organic compounds having a molecular weight of more than 50 and less than about 2,500 daltons, or less than about 5000 daltons. Test agents can comprise functional groups necessary for structural interaction with proteins, particularly hydrogen bonding, and may include at least an amine, carbonyl, hydroxyl or carboxyl group, or at least two of the functional chemical groups. The candidate agents can comprise cyclical carbon or heterocyclic structures and/or aromatic or polyaromatic structures substituted with one or more of the above functional groups. Test agents are also found among biomolecules including peptides, saccharides, fatty acids, steroids, purines, pyrimidines, derivatives, structural analogs or combinations thereof.

Test agents are obtained from a wide variety of sources including libraries of synthetic or natural compounds. For example, numerous means are available for random and directed synthesis of a wide variety of organic compounds and biomolecules. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Of interest in certain embodiments are compounds that pass cellular membranes.

### Methods of Controlling an Activity of a Cell

The present disclosure provides methods of controlling an activity of a cell. The methods generally involve: a) detecting a protein-protein interaction, as described above; and b) modulating an activity of the cell, e.g., where the “protein of interest” is a protein that modulates an activity of the cell, or where the “protein of interest” is a protein that induces expression of a gene product that modulates an activity of the cell. A protein that modulates an activity of a cell is also referred to herein as an “effector polypeptide.” A gene product that modulates an activity of the cell is also referred to herein as an “effector gene product.” An effector gene product can be an effector polypeptide or an effector nucleic acid.

For example, in some cases, the target cell is further genetically modified with a heterologous nucleic acid comprising a nucleotide sequence encoding an “effector polypeptide” where the nucleotide sequence is operably linked to the same promoter to which the nucleotide sequence encoding the reporter gene product is operably linked, e.g., is operably linked to a promoter that is activated by the transcription factor that is released from the first fusion polypeptide.

In other instances, the target cell is further genetically modified with a heterologous nucleic acid comprising a nucleotide sequence encoding an “effector gene product” where the nucleotide sequence encoding the effector gene product is operably linked to a different promoter than the promoter to which the nucleotide sequence encoding the reporter gene product is operably linked, e.g., is operably linked to a promoter that is not activated by the transcription factor that is released from the first fusion polypeptide. An effector gene product can be an effector polypeptide or an effector nucleic acid.

Suitable effector polypeptides include, but are not limited to: 1) an opsin, e.g., a hyperpolarizing opsin or a depolarizing opsin, where suitable opsins are known in the art and are described above; in some cases, the opsin is one that is activated by light of a wavelength that is different from the wavelength of light that activates a LOV-domain light-activated polypeptide; 2) a toxin; 3) an apoptosis-inducing polypeptide; 4) a receptor; 5) a cytokine; 6) a chemokine; 7) an RNA-guided endonuclease (e.g., a Cas9 polypeptide, a Cpf1 polypeptide, a C2c2 polypeptide, etc.); 8) a recombinase (e.g., a Cre recombinase that acts on Lox sites); 9) a kinase; 10) a phosphatase; 11) a DREADD; 12) an antibody; etc.

Suitable effector nucleic acids include, but are not limited to: 1) a guide RNA (e.g., a guide RNA that binds an RNA-guided endonuclease (e.g., a Cas9 polypeptide, a Cpf1 polypeptide, a C2c2 polypeptide, etc.); 2) a ribozyme; 3) an inhibitory RNA; and 4) a microRNA.

Activities of a target cell that can be modulated using a method of the present disclosure include, but are not limited to: 1) proliferation; 2) secretion of a cytokine; 3) secretion of a chemokine; 4) secretion of a neurotransmitter; 4) cell behavior; 5) cell death; 6) cellular differentiation; 7) cell killing of another cell; 8) interaction with another cell; 9) transcription; 10) translation; 11) biosynthesis; 12) metabolism; etc.

### Kits

The present disclosure provides a kit for using a PPI detection system of the present disclosure, e.g., for carrying out a method of the present disclosure. A kit of the present disclosure provides one or more components of a PPI detection system of the present disclosure and/or one or more nucleic acids comprising a nucleotide sequence(s) encoding one or more components of a PPI detection system of the present disclosure.

In some cases, a kit of the present disclose comprises nucleic acid system comprising: A) a first nucleic acid comprising, in order from 5′ to 3′: a) a nucleotide sequence encoding a first (light-activated) fusion polypeptide of the present disclosure, e.g., a first fusion polypeptide comprising, in order from amino terminus to carboxyl terminus: i) a transmembrane domain (or other tethering polypeptide); ii) a first polypeptide member of a protein interaction pair; iii) a LOV-domain light-activated polypeptide comprising an amino acid sequence having at least 80%, at least 85%, at least 90%, at least 95%, at least 98%, at least 99%, or 100% amino acid sequence identity to the amino acid sequence depicted in any one of FIG. 11A-11G; and iv) a proteolytically cleavable linker; and b) an insertion site for a nucleic acid comprising a nucleotide sequence encoding a polypeptide of interest; and B) a second nucleic acid comprising a nucleotide sequence encoding a second fusion polypeptide comprising: i) a second polypeptide member of the protein interaction pair; and ii) a protease that cleaves the proteolytically cleavable linker. In some cases, one or both of the first and the second nucleic acids are stably integrated into the genome of a cell; and the kit provides the cell (e.g., an in vitro cell; e.g., an in vitro mammalian cell) with one or both of the first and the second nucleic acids stably integrated into its genome. In some cases, one or both of the first and the second nucleic acids are present in a recombinant expression vector, e.g., a recombinant viral vector such as a recombinant AAV vector, a recombinant lentiviral vector, etc. In some cases, the polypeptide of interest is a transcription factor, and the kit further comprises a cell that is genetically modified with a nucleic acid comprising: a) a nucleotide sequence encoding a polypeptide; and b) a promoter that is responsive to the transcription factor, where the nucleotide sequence encoding the polypeptide is operably linked to the promoter; in some of these embodiments, the polypeptide is a fluorescent protein or other polypeptide that can be detected. Components of the kit can be provided in one or more containers, e.g., tubes, vials, etc.

In some cases, a kit of the present disclosure comprises a nucleic acid system comprising: a) a first nucleic acid comprising a nucleotide sequence encoding a light-activated, calcium-gated transcription control polypeptide comprising, in order from amino terminus to carboxyl terminus: i) a transmembrane domain; ii) a first polypeptide member of a protein interaction pair; iii) a LOV light-activated polypeptide comprising an amino acid sequence having at least 80%, at least 85%, at least 90%, at least 95%, at least 98%, at least 99%, or 100% amino acid sequence identity to the amino acid sequence depicted in one of FIG. 11A-11G; iv) a proteolytically cleavable linker; and v) a transcription factor; and b) a second nucleic acid comprising a nucleotide sequence encoding a fusion polypeptide comprising: i) a second polypeptide member of the protein interaction pair; and ii) a protease that cleaves the proteolytically cleavable linker. In some cases, one or both of the first and the second nucleic acids are stably integrated into the genome of a cell; and the kit provides the cell (e.g., an in vitro cell; e.g., an in vitro mammalian cell)) with one or both of the first and the second nucleic acids stably integrated into its genome. In some cases, one or both of the first and the second nucleic acids are present in a recombinant expression vector, e.g., a recombinant viral vector such as a recombinant AAV vector, a recombinant lentiviral vector, etc. In some cases, the kit further comprises a cell that is genetically modified with a nucleic acid comprising: a) a nucleotide sequence encoding a polypeptide; and b) a promoter that is responsive to the transcription factor, where the nucleotide sequence encoding the polypeptide is operably linked to the promoter; in some of these embodiments, the polypeptide is a fluorescent protein or other polypeptide that can be detected. Components of the kit can be provided in one or more containers, e.g., tubes, vials, etc. In some cases, instead of the second nucleic acid described above, the kit comprises a nucleic acid library comprising a plurality of nucleic acid members, each of which comprises a nucleotide sequence encoding a fusion polypeptide comprising: i) a test polypeptide, to be tested for binding to the first member of the protein interaction pair; and ii) a protease that cleaves the proteolytically cleavable linker, where each of the members comprises a nucleotide sequence encoding a different test polypeptide.

The present disclosure provides a kit comprising a nucleic acid comprising: a) a nucleotide sequence encoding a first fusion polypeptide comprising, in order from amino terminus to carboxyl terminus: i) a transmembrane domain; ii) a first polypeptide member of a protein interaction pair; iii) a LOV-domain light-activated polypeptide comprising an amino acid sequence having at least 80%, at least 85%, at least 90%, at least 95%, at least 98%, at least 99%, or 100% amino acid sequence identity to the amino acid sequence depicted in FIG. 11A-11G; and iv) a proteolytically cleavable linker; and b) an insertion site for a nucleic acid comprising a nucleotide sequence encoding a polypeptide of interest. In some cases, the kit further comprises a second nucleic acid comprising a nucleotide sequence encoding a fusion polypeptide comprising: i) a second polypeptide member of the protein interaction pair; and ii) a protease that cleaves the proteolytically cleavable linker. One or both of the nucleic acids can be present in a recombinant expression vector, e.g., a recombinant viral vector such as a recombinant AAV vector, a recombinant lentiviral vector, etc. In some cases, one or both of the nucleic acids is stably integrated into the genome of a cell; and the kit provides the cell (e.g., an in vitro cell; e.g., an in vitro mammalian cell)) with one or both of the nucleic acids stably integrated into its genome. In some cases, instead of the second nucleic acid described above, the kit comprises a nucleic acid library comprising a plurality of nucleic acid members, each of which comprises a nucleotide sequence encoding a fusion polypeptide comprising: i) a test polypeptide, to be tested for binding to the first member of the protein interaction pair; and ii) a protease that cleaves the proteolytically cleavable linker, where each of the members comprises a nucleotide sequence encoding a different test polypeptide.

In some cases, a kit of the present disclosure comprises: a nucleic acid comprising: a) a nucleotide sequence encoding a transmembrane domain or other tethering domain; b) an insertion site for a nucleic acid comprising a nucleotide sequence encoding a first member of a protein interaction pair; c) a light-activated polypeptide comprising a LOV domain comprising an amino acid sequence having at least 80% amino acid sequence identity to any one of the amino acid sequences set forth in FIG. 11A-11G; d) a proteolytically cleavable linker; and e) an insertion site for a nucleic acid comprising a nucleotide sequence encoding a polypeptide of interest. In some cases, the nucleic acid is present in a recombinant expression vector. In some cases, the kit comprises a second nucleic acid comprising: a)) an insertion site for: i) a nucleic acid comprising a nucleotide sequence encoding a second member of the protein interaction pair; or ii) a nucleic acid comprising a nucleotide sequence encoding a polypeptide to be tested for binding to the first member of the protein interaction pair. In some cases, the second nucleic acid is present in a recombinant expression vector. In some cases, the second nucleic acid is present in a cell.

In some cases, a kit of the present disclosure comprises: a nucleic acid comprising: a) a nucleotide sequence encoding a transmembrane domain or other tethering domain; b) an insertion site for a nucleic acid comprising a nucleotide sequence encoding a first member of a protein interaction pair; c) a light-activated polypeptide comprising a LOV domain comprising an amino acid sequence having at least 80% amino acid sequence identity to any one of the amino acid sequences set forth in FIG. 11A-11G; d) a proteolytically cleavable linker; and e) a transcription factor. In some cases, the nucleic acid is present in a recombinant expression vector. In some cases, the kit comprises a second nucleic acid comprising: a)) an insertion site for: i) a nucleic acid comprising a nucleotide sequence encoding a second member of the protein interaction pair; or ii) a nucleic acid comprising a nucleotide sequence encoding a polypeptide to be tested for binding to the first member of the protein interaction pair. In some cases, the second nucleic acid is present in a recombinant expression vector. In some cases, the second nucleic acid is present in a cell. In some cases, the kit further comprises a third nucleic acid. In some cases, the third nucleic acid comprises: a) a promoter that is activated by the transcription factor; and b) a nucleotide sequence encoding a fluorescent protein. In some cases, the kit further comprises a third nucleic acid. In some cases, the third nucleic acid comprises: a) a promoter that is activated by the transcription factor; and b) a nucleotide sequence encoding a polypeptide of interest.

A kit of the present disclosure can further include one or more additional reagents, where such additional reagents can be selected from: a buffer; a wash buffer; a control reagent; a positive control; a negative control; a reagent(s) for detecting production of a cleavage product of enzymatic cleavage of a substrate; and the like.

A suitable positive control can comprise: a) one or more nucleic acids comprising nucleotide sequences encoding: i) a first polypeptide comprising, in order from N-terminus to C-terminus: a TM domain, a first polypeptide member of a protein interaction pair, a LOV domain polypeptide (a LOV-domain light-activated polypeptide comprising an amino acid sequence having at least 80%, at least 85%, at least 90%, at least 95%, at least 98%, at least 99%, or 100% amino acid sequence identity to the amino acid sequence depicted in FIG. 11A-11G), a proteolytically cleavable linker, and a transcription factor; and ii) a second polypeptide comprising, in order from N-terminus to C-terminus: a second polypeptide member of the protein interaction pair, and a protease that cleaves the proteolytically cleavable linker; and B) a nucleic acid comprising: a) a nucleotide sequence encoding a fluorescent polypeptide; and b) a promoter that is responsive to the transcription factor, where the nucleotide sequence encoding the polypeptide is operably linked to the promoter. Those skilled in the art would be aware of other suitable positive controls.

Components of a subject kit can be in separate containers; or can be combined in a single container.

In addition to above-mentioned components, a subject kit can further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, flash drive, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-72 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A nucleic acid system comprising: A) a first nucleic acid comprising, in order from 5′ to 3′: a) a nucleotide sequence encoding a first, light-activated, fusion polypeptide comprising, in order from amino terminus to carboxyl terminus: i) a transmembrane domain; ii) a first member of a protein interaction pair; iii) a LOV-domain light-activated polypeptide comprising an amino acid sequence having at least 80% amino acid sequence identity to the amino acid sequence depicted in FIG. 11A-11G; iv) a proteolytically cleavable linker; and b) an insertion site for a nucleic acid comprising a nucleotide sequence encoding a polypeptide of interest; and B) a second nucleic acid comprising a nucleotide sequence encoding a second fusion polypeptide comprising: i) a second member of the protein interaction pair; and ii) a protease that cleaves the proteolytically cleavable linker, wherein the first member of the protein interaction pair and the second member of the protein interaction pair bind to one another in the presence of an agent.

Aspect 2. A nucleic acid system comprising: a) a first nucleic acid comprising a nucleotide sequence encoding a first fusion polypeptide comprising, in order from amino terminus to carboxyl terminus: i) a transmembrane domain; ii) a first member of a protein interaction pair; iii) a LOV light-activated polypeptide comprising an amino acid sequence having at least 80% amino acid sequence identity to the amino acid sequence depicted in any one of FIG. 11A-11G; iv) a proteolytically cleavable linker; and v) a polypeptide of interest; and b) a second nucleic acid comprising a nucleotide sequence encoding a second fusion polypeptide comprising: i) a second member of the protein interaction pair; and ii) a protease that cleaves the proteolytically cleavable linker, wherein the first member of the protein interaction pair and the second member of the protein interaction pair bind to one another in the presence of a binding-inducing agent.

Aspect 3. The nucleic acid system of aspect 1, wherein the insertion site is a multiple cloning site.

Aspect 4. The nucleic acid system of any one of aspects 1-3, wherein the first member of the protein interaction pair is an N-terminal portion of a polypeptide; and wherein the second member of the protein interaction pair is a C-terminal portion of the polypeptide.

Aspect 5. The nucleic acid system of any one of aspects 1-3, wherein the first and second polypeptides of the protein interaction pair bind to one another in the presence of a small molecule agent.

Aspect 6. The nucleic acid system of any one of aspects 1-3, wherein the first and second polypeptides of the protein interaction pair bind to one another in the presence of light of an activating wavelength.

Aspect 7. The nucleic acid system of any one of aspects 1-3, wherein the first and second polypeptides of the protein interaction pair bind to one another in the presence of a hormone.

Aspect 8. The nucleic acid system of any one of aspects 1-3, wherein the first and second polypeptides of the protein interaction pair bind to one another in the presence of an ion.

Aspect 9. The nucleic acid system of any one of aspects 1-3, wherein the protein interaction pair is selected from: a) FK506 binding protein (FKBP) and FKBP; b) FKBP and calcineurin catalytic subunit A (CnA); c) FKBP and cyclophilin; d) FKBP and FKBP-rapamycin associated protein (FRB); e) gyrase B (GyrB) and GyrB; f) dihydrofolate reductase (DHFR) and DHFR; g) DmrB and DmrB; h) PYL and ABI; i) Cry2 and CIB1; j) GAI and GID1; k) mineralcorticoid receptor (MR) ligand-binding domain (LBD) and an SRC1-2 peptide; 1) a PPAR-γ LBD and an SRC1 peptide; m) an androgen receptor LBF and an SRC3-1 peptide; n) a PPAR-γ LBD and an SRC3 peptide; o) an MR LBD and a PGC1a peptide; p) an MR LBD and a TRAP220-1 peptide; q) a progesterone receptor LBD and an NCoR peptide; r) an estrogen receptor-β LBD and an NR0B1 peptide; s) a PPAR-γ LBD and a TIF2 peptide; t) an ERα LBD and a CoRNR box peptide; u) an ERα LBD and an abV peptide; v) a G protein-coupled receptor (GPCR) and a G protein; w) a GPCR and a beta-arrestin polypeptide; x) an epidermal growth factor receptor (EGFR) and Src/Shc/Grb2; y) calmodulin and calmodulin binding polypeptide; and z) troponin C and troponin I.

Aspect 10. The nucleic acid system of any one of aspects 1-9, wherein the LOV-domain light-activated polypeptide comprises one or more amino acid substitutions selected from L2R, N12S, A28V, H117R, and I130V substitutions relative to the amino acid sequence depicted in FIG. 11B.

Aspect 11. The nucleic acid system of any one of aspects 1-9, wherein the LOV domain light-activated polypeptide comprises L2R, N12S, I130V, A28V, and H117R substitutions relative to the amino acid sequence depicted in FIG. 11B.

Aspect 12. The nucleic acid system of any one of aspects 1-11, wherein the proteolytically cleavable linker comprises an amino acid sequence cleaved by a viral protease, a mammalian protease, or a recombinant protease.

Aspect 13. The nucleic acid system of any one of aspects 1-12, wherein the protease is a viral protease, a mammalian protease, or a recombinant protease.

Aspect 14. The nucleic acid system of any one of aspects 1-13, wherein the first nucleic acid is present in a first expression vector, and the second nucleic acid is present in a second expression vector.

Aspect 15. The nucleic acid system of aspect 14, wherein the first expression vector and the second expression vector are recombinant viral vectors.

Aspect 16. The nucleic acid system of aspect 15, wherein the recombinant viral vector is a lentiviral vector, a retroviral vector, an adeno-associated viral vector, an adenoviral vector, or a herpes simplex virus vector.

Aspect 17. The nucleic acid system of any one of aspects 2-16, wherein the polypeptide of interest is a reporter polypeptide, a light-activated polypeptide, a transcription factor, a toxin, a calcium sensor, a recombinase, an antibiotic resistance factor, a DREADD, an RNA-guided endonuclease, a drug resistance factor, a biotin ligase, a kinase, a phosphorylase, or a peroxidase.

Aspect 18. The nucleic acid system of aspect 17, wherein the polypeptide of interest is a reporter polypeptide selected from a fluorescent polypeptide, an enzyme that produces a colored product, an enzyme that produces a luminescent product, and an enzyme that produces a fluorescent product.

Aspect 19. The nucleic acid system of aspect 17, wherein the polypeptide of interest is a transcriptional activator or a transcriptional repressor.

Aspect 20. The nucleic acid system of aspect 17, wherein the polypeptide of interest is an antibiotic resistance factor.

Aspect 21. The nucleic acid system of aspect 17, wherein the polypeptide of interest is an RNA-guided endonuclease selected from a Cas9 polypeptide, a C2C2 polypeptide, or a Cpf1 polypeptide.

Aspect 22. A genetically modified host cell, wherein the host cell is genetically modified with the nucleic acid system of any one of aspects 1-21.

Aspect 23. The genetically modified host cell of aspect 22, wherein the cell is in vitro.

Aspect 24. The genetically modified host cell of aspect 22, wherein the cell is in vivo.

Aspect 25. The genetically modified host cell of any one of aspects 22-24, wherein the cell is an animal cell

Aspect 26. The genetically modified host cell of aspect 25, wherein the cell is a mammalian cell.

Aspect 27. The genetically modified host cell of aspect 25, wherein the cell is an insect cell, a reptile cell, an amphibian cell, or an avian cell.

Aspect 28. The genetically modified host cell of aspect 25, wherein the cell is a cell of an invertebrate animal.

Aspect 29. The genetically modified host cell of any one of aspects 22-24, wherein the cell is a single celled organism.

Aspect 30. The genetically modified host cell of any one of aspects 22-24, wherein the cell is a plant cell.

Aspect 31. The genetically modified host cell of any one of aspects 28-30, wherein the first and/or the second nucleic acid is stably integrated into the genome of the host cell.

Aspect 32. A nucleic acid comprising: a) a nucleotide sequence encoding a fusion polypeptide comprising, in order from amino terminus to carboxyl terminus: i) a transmembrane domain; ii) a first member of a protein interaction pair; iii) a LOV-domain light-activated polypeptide comprising an amino acid sequence having at least 80% amino acid sequence identity to the amino acid sequence depicted FIG. 11A-11G; and iv) a proteolytically cleavable linker; and b) an insertion site for a nucleic acid comprising a nucleotide sequence encoding a polypeptide of interest

Aspect 33. A recombinant expression vector comprising the nucleic acid of aspect 32.

Aspect 34. A genetically modified host cell, wherein the host cell is genetically modified with the nucleic acid of aspect 32 or the recombinant expression vector of aspect 33.

Aspect 35. A nucleic acid comprising a nucleotide sequence encoding a fusion polypeptide comprising, in order from amino terminus to carboxyl terminus: i) a transmembrane domain; ii) a first member of a protein interaction pair; iii) a LOV light-activated polypeptide comprising an amino acid sequence having at least 80% amino acid sequence identity to the amino acid sequence depicted in any one of FIG. 11A-11G; iv) a proteolytically cleavable linker; and v) a gene product of interest.

Aspect 36. A recombinant expression vector comprising the nucleic acid of aspect 35.

Aspect 37. A genetically modified host cell, wherein the host cell is genetically modified with the nucleic acid of aspect 35 or the recombinant expression vector of aspect 36.

Aspect 38. A nucleic acid system comprising: A) a first nucleic acid comprising a nucleotide sequence encoding a first fusion polypeptide comprising, in order from amino terminus to carboxyl terminus: i) a transmembrane domain; ii) a first member of a protein interaction pair; iii) a LOV light-activated polypeptide comprising an amino acid sequence having at least 80% amino acid sequence identity to the amino acid sequence depicted in any one of FIG. 11A-11G; iv) a proteolytically cleavable linker; and v) a signal polypeptide; and B) a second nucleic acid comprising, in order from 5′ to 3′: a) an insertion site for a nucleic acid comprising a nucleotide sequence encoding a second member of the protein interaction pair; and b) a nucleotide sequence encoding a protease that cleaves the proteolytically cleavable linker, wherein the first member of the protein interaction pair and the second member of the protein interaction pair bind to one another in the presence of a binding-inducing agent, and wherein the signal polypeptide provides a signal when cleaved from the fusion polypeptide.

Aspect 39. The nucleic acid system of aspect 38, wherein the insertion site is a multiple cloning site.

Aspect 40. The nucleic acid system of aspect 38 or aspect 39, wherein the second member of the protein interaction pair is encoded by a member of a library comprising a plurality of nucleic acids.

Aspect 41. The nucleic acid system of any one of aspects 38-40, wherein the signal polypeptide is a fluorescent protein, a transcription factor, or an enzyme.

Aspect 42. The nucleic acid system of any one of aspects 38-41, wherein one or both of the first and the second nucleic acids are in expression vectors.

Aspect 43. The nucleic acid system of aspect 42, wherein one or both of the expression vectors are recombinant viral vectors.

Aspect 44. The nucleic acid system of aspect 43, wherein one or both of the recombinant viral vectors is a recombinant lentiviral vector, a recombinant retroviral vector, or a recombinant adenoassociated viral vector.

Aspect 45. A genetically modified host cell, wherein the host cell is genetically modified with the nucleic acid system of any one of aspects 38-44.

Aspect 46. A polypeptide system comprising: a) a first fusion polypeptide comprising: i) a transmembrane domain; ii) a first member of a protein interaction pair; iii) a LOV light-activated polypeptide comprising an amino acid sequence having at least 80% amino acid sequence identity to the amino acid sequence depicted in any one of FIG. 11A-11G; iv) a proteolytically cleavable linker; and v) a polypeptide of interest; and b) a second fusion polypeptide comprising: i) a second member of the protein interaction pair; and ii) a protease that cleaves the proteolytically cleavable linker.

Aspect 47. The system of aspect 46, wherein the LOV-domain light-activated polypeptide comprises one or more amino acid substitutions selected from L2R, N12S, A28V, H117R, and I130V substitutions relative to the amino acid sequence depicted in FIG. 11B.

Aspect 48. The system of aspect 46 or aspect 47, wherein the LOV domain light-activated polypeptide comprises L2R, N12S, I130V, A28V, and H117R substitutions relative to the amino acid sequence depicted in FIG. 11B.

Aspect 49. The system of any one of aspects 46-48, wherein the protease is not naturally produced by a mammalian cell.

Aspect 50. The system of aspect 59, wherein the protease is a viral protease.

Aspect 51. The system of aspect 50, wherein the viral protease is a tobacco etch virus (TEV) protease.

Aspect 52. The system of any one of aspects 46-48, wherein the protease is naturally produced by a mammalian cell.

Aspect 53. The system of any one of aspects 46-52, wherein the first member of the protein interaction pair is an N-terminal portion of a polypeptide; and wherein the second member of the protein interaction pair is a C-terminal portion of the polypeptide.

Aspect 54. The system of any one of aspects 46-52, wherein the first and second polypeptides of the protein interaction pair bind to one another in the presence of a small molecule agent.

Aspect 55. The system of any one of aspects 46-52, wherein the first and second polypeptides of the protein interaction pair bind to one another in the presence of light of an activating wavelength.

Aspect 56. The system of any one of aspects 46-52, wherein the first and second polypeptides of the protein interaction pair bind to one another in the presence of a hormone.

Aspect 57. The system of any one of aspects 46-52, wherein the first and second polypeptides of the protein interaction pair bind to one another in the presence of an ion.

Aspect 58. The system of any one of aspects 46-52, wherein the protein interaction pair is selected from: a) FK506 binding protein (FKBP) and FKBP; b) FKBP and calcineurin catalytic subunit A (CnA); c) FKBP and cyclophilin; d) FKBP and FKBP-rapamycin associated protein (FRB); e) gyrase B (GyrB) and GyrB; f) dihydrofolate reductase (DHFR) and DHFR; g) DmrB and DmrB; h) PYL and ABI; i) Cry2 and CIB1; j) GAI and GID1; k) mineralcorticoid receptor (MR) ligand-binding domain (LBD) and an SRC1-2 peptide; 1) a PPAR-γ LBD and an SRC1 peptide; m) an androgen receptor LBF and an SRC3-1 peptide; n) a PPAR-γ LBD and an SRC3 peptide; o) an MR LBD and a PGC1a peptide; p) an MR LBD and a TRAP220-1 peptide; q) a progesterone receptor LBD and an NCoR peptide; r) an estrogen receptor-β LBD and an NR0B1 peptide; s) a PPAR-γ LBD and a TIF2 peptide; t) an ERα LBD and a CoRNR box peptide; u) an ERα LBD and an abV peptide; v) a G protein-coupled receptor (GPCR) and a G protein; w) a GPCR and a beta-arrestin polypeptide; x) an epidermal growth factor receptor (EGFR) and Src/Shc/Grb2; y) calmodulin and calmodulin binding polypeptide; and z) troponin C and troponin I.

Aspect 59. A mammalian cell comprising the system of any one of aspects 46-58.

Aspect 60. The mammalian cell aspect 59, wherein the cell is in vitro.

Aspect 61. A genetically modified non-human organism that comprises, integrated into the genome of one or more cells of the organism, the nucleic acid system of any one of aspects 1-21 and 38-44, or the nucleic acid of aspect 32 or aspect 35.

Aspect 62. The genetically modified non-human organism of aspect 61, wherein the organism is a mammal.

Aspect 63. The genetically modified non-human organism of aspect 62, wherein the mammal is a rodent.

Aspect 64. A method for detecting protein-protein interaction in a cell in response to a stimulus, the method comprising: A) exposing the cell to the stimulus, wherein the cell comprises: a) a first fusion polypeptide comprising: i) a transmembrane domain; ii) a first member of a protein interaction pair; iii) a LOV light-activated polypeptide comprising an amino acid sequence having at least 80% amino acid sequence identity to the amino acid sequence depicted in any one of FIG. 11A-11G; iv) a proteolytically cleavable linker; and v) a signal polypeptide that produces a signal only following release from the first fusion polypeptide; and b) a second fusion polypeptide comprising: i) a second member of the protein interaction pair; and ii) a protease that cleaves the proteolytically cleavable linker; B) substantially simultaneously exposing the cell to light of a wavelength that activates the LOV domain polypeptide; and C) detecting a signal produced by the signal polypeptide, wherein an increase in a signal produced by the signal polypeptide, compared to a control level of the signal, indicates that exposure of the cell to the stimulus results in binding of the first member to the second member of the protein interaction pair.

Aspect 65. The method of aspect 64, wherein the stimulus is a ligand, a drug, a toxin, a neurotransmitter, contact with a second cell, heat, or hypoxia.

Aspect 66. The method of aspect 64 or aspect 65, wherein the signal polypeptide is a transcription factor that induces transcription of a detectable polypeptide.

Aspect 67. The method of aspect 66, wherein the detectable polypeptide is a fluorescent protein.

Aspect 68. The method of any one of aspects 64-67, wherein the cell is in vitro.

Aspect 69. The method of any one of aspects 64-67, wherein the cell is in vivo.

Aspect 70. The method of any one of aspects 64-69, wherein the cell is a human cell.

Aspect 71. The method of any one of aspects 64-69, wherein the cell is a non-human animal cell.

Aspect 72. The method of any one of aspects 64-69, wherein the second member of the protein interaction pair is encoded by a member of a library comprising a plurality of nucleic acids.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: PPI Detection Systems

FIGS. 17-20 provide sequence information regarding exemplary PPI detection systems.

FIG. 1 is a schematic depiction of the requirement for two input signals for functioning of a system of the present disclosure.

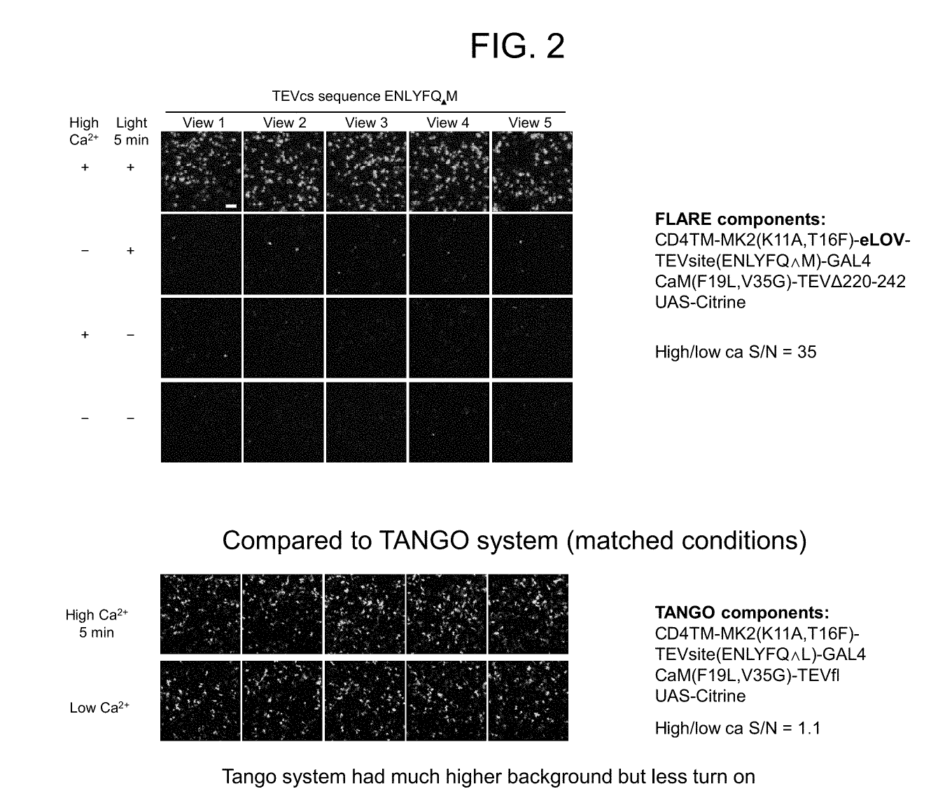

FIG. 2 presents a comparison of a calcium-induced protein-protein interaction (PPI) detection system of the present disclosure to the TANGO system.

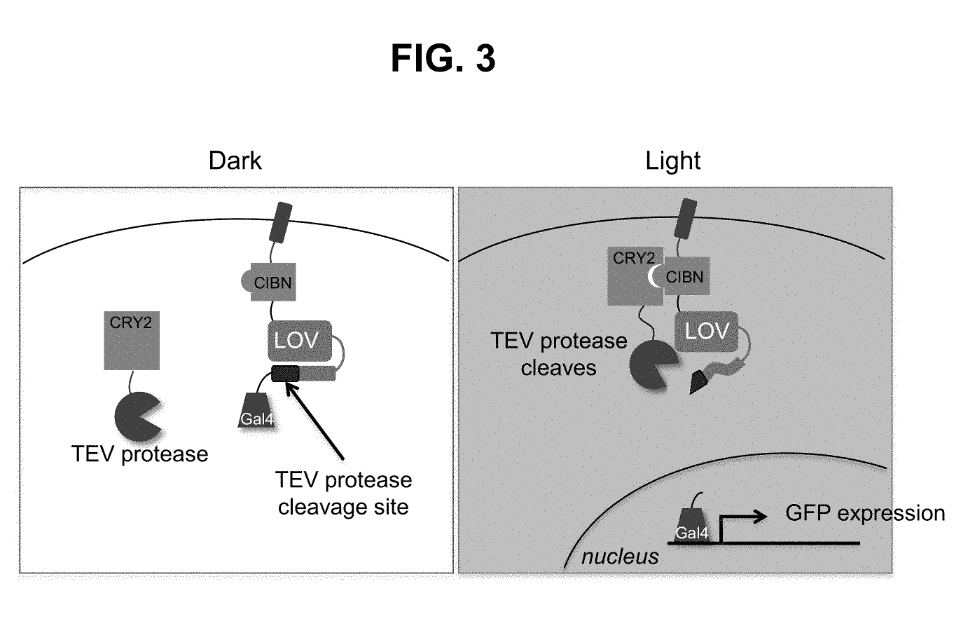

FIG. 3 is a schematic depiction of an example of a blue light induced CRY2-CIBN PPI detection system.

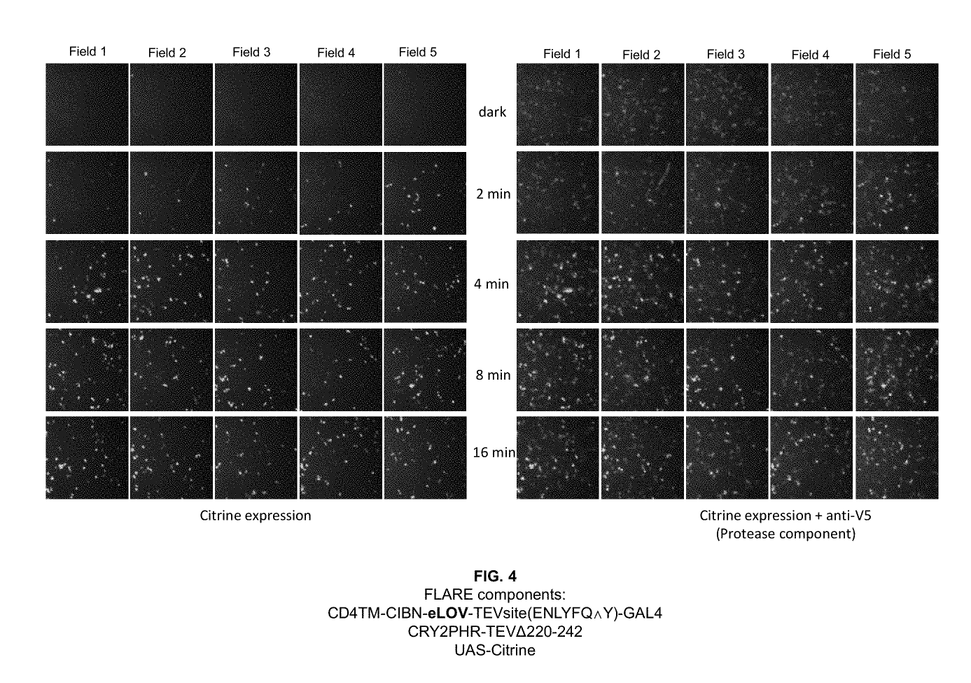

FIG. 4 depicts PPI detection using a PPI detection system as schematically depicted in FIG. 3.

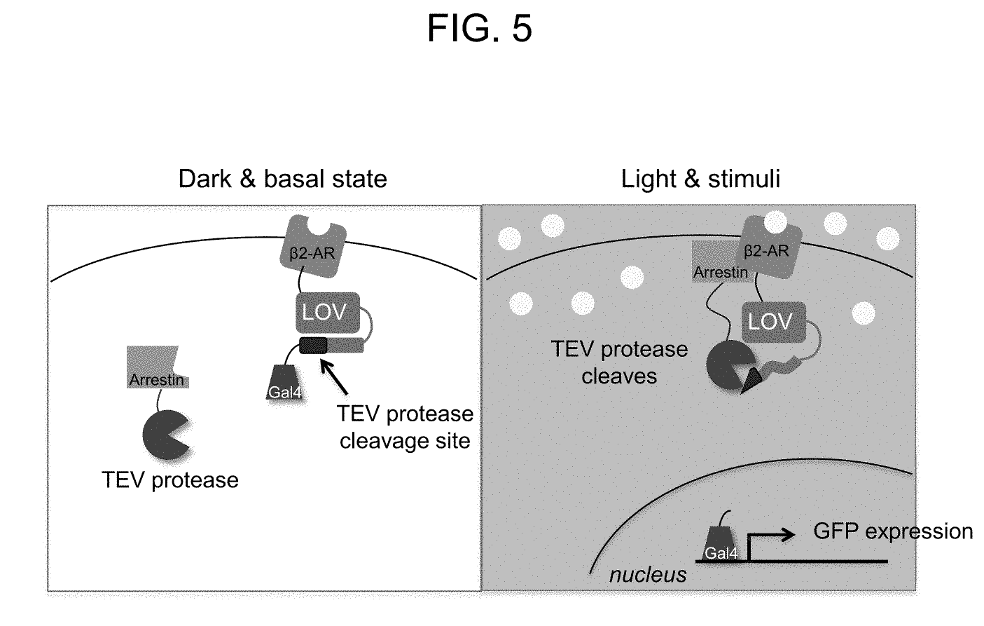

FIG. 5 is a schematic depiction of an isoproterenol induced beta2-AR and beta2-arrestin PPI detection system of the present disclosure.

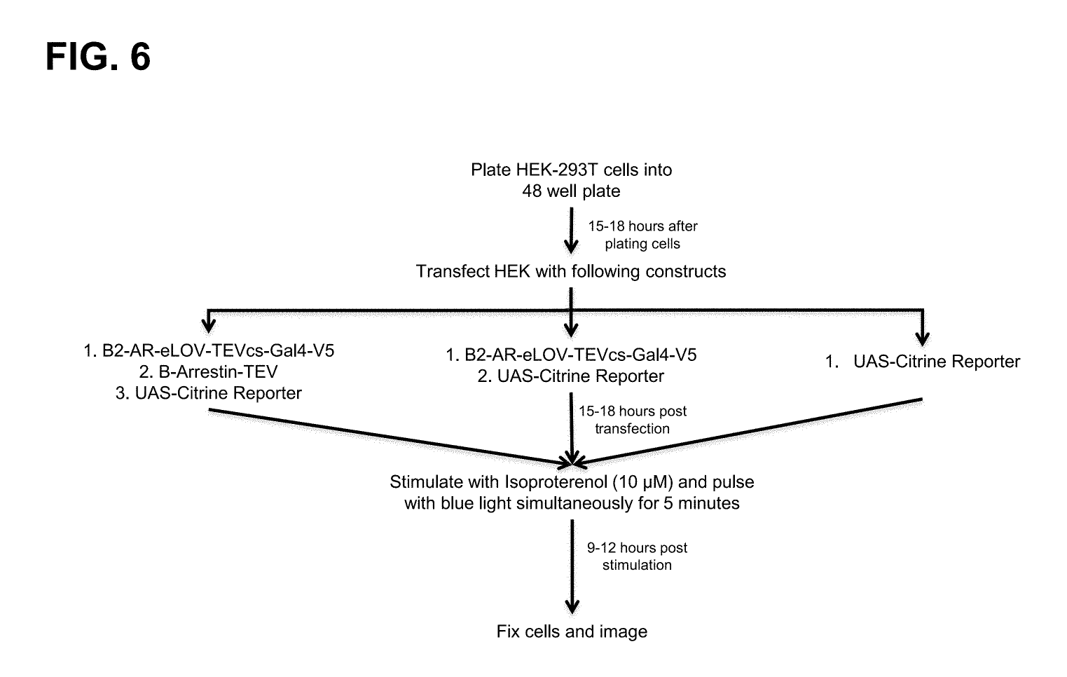

FIG. 6 is a workflow diagram for use of a PPI detection system as schematically depicted in FIG. 5.

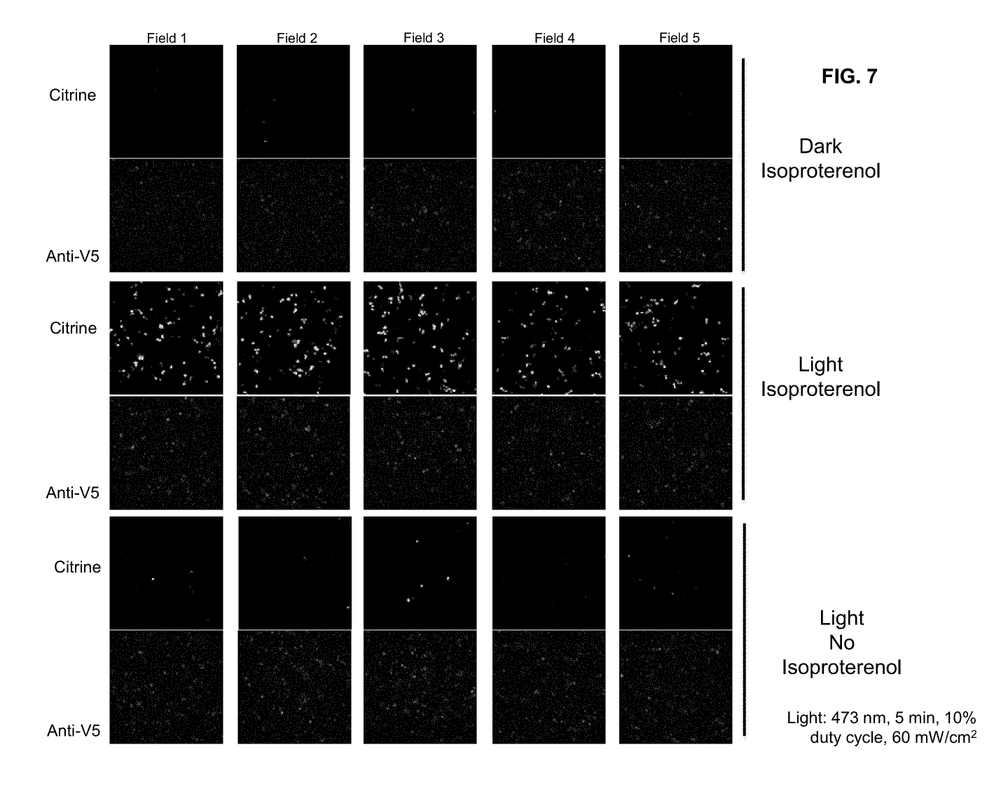

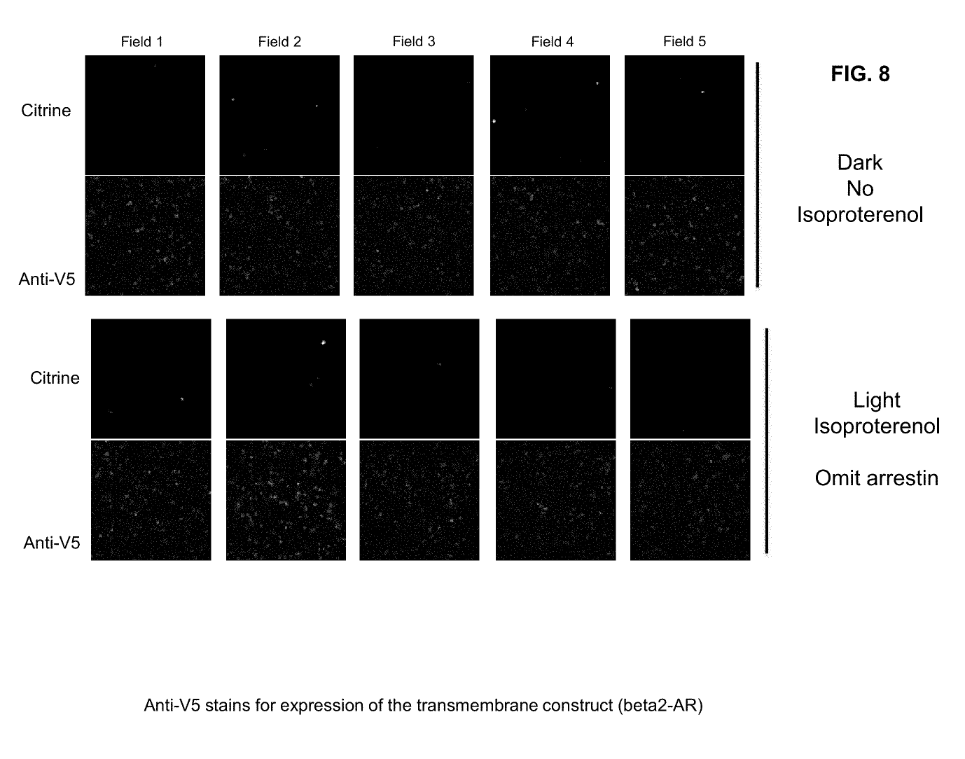

FIG. 7 and FIG. 8 depict PPI detection using a PPI detection system as schematically depicted in FIG. 5.

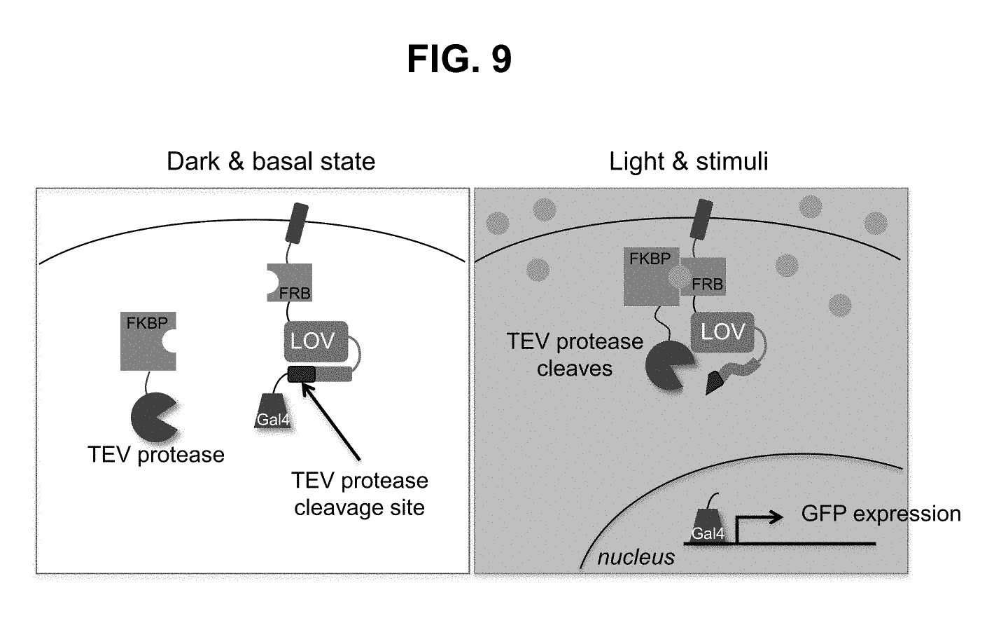

FIG. 9 is a schematic depiction of a rapamycin induced FRB-FKBP PPI detection system of the present disclosure.

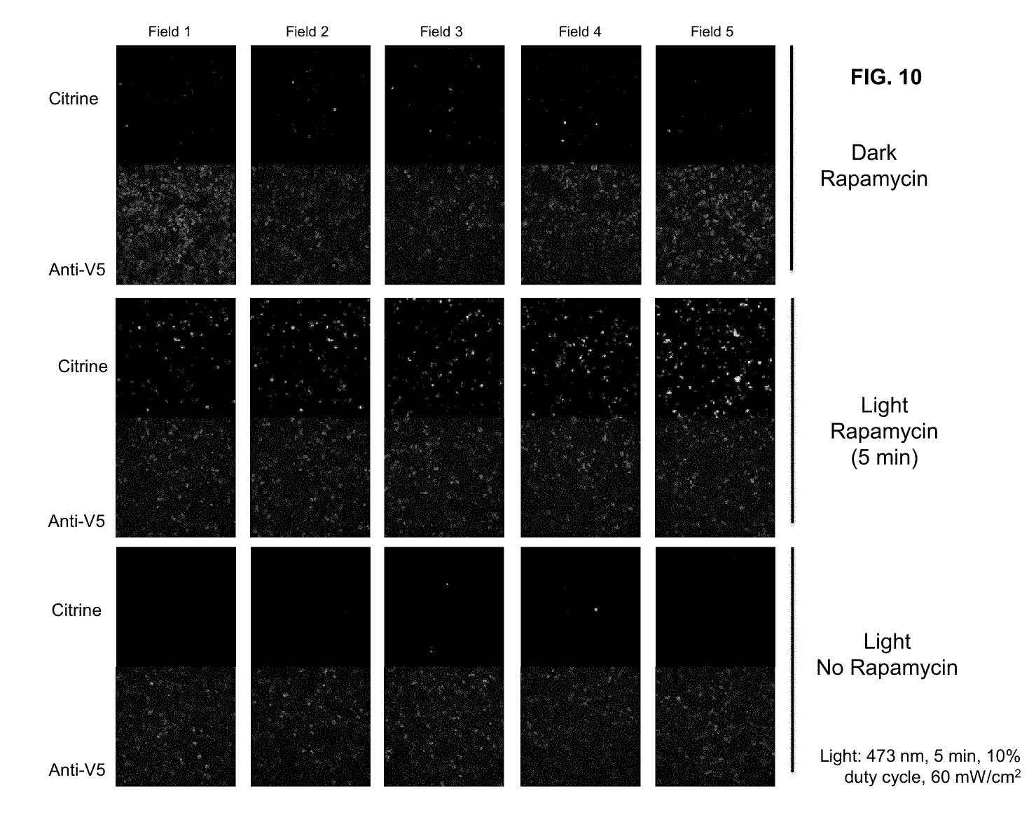

FIG. 10 depicts PPI detection using a PPI detection system as schematically depicted in FIG. 9.

### Example 2

FIG. 21A-21F: Design of FLARE-PPI and Application to Light- and Agonist-Dependent Detection of β2-Adrenergic Receptor (β2AR)-Arrestin2 Interaction.

(A) Scheme. A and B are proteins that interact under certain conditions. Protein A is membrane-associated and is fused to a light-sensitive eLOV domain, a protease cleavage site (TEVcs), and a transcription factor (TF). These comprise the “FLARE TF component.” Protein B is fused to a truncated variant of TEV protease (TEVp) (“FLARE protease component”). When A and B interact (right), TEVp is recruited to the vicinity of TEVcs. When blue light is applied to the cells, eLOV reversibly unblocks TEVcs. Hence, the coincidence of light and A-B interaction permits cleavage of TEVcs by TEVp, resulting in the release of the TF, which translocates to the nucleus and drives transcription of a reporter gene of interest. (B) FLARE-PPI constructs for studying the β2AR-arrestin interaction. V5 and myc are epitope tags. UAS is a promoter recognized by the TF Gal4. (C) Imaging of FLARE activation by β2AR-arrestin interaction under four conditions. HEK 293T cells were transiently transfected with the three FLARE components shown in (B). β2AR-arrestin interaction was induced with addition of 10 μM isoproterenol for 5 minutes. Light stimulation was via 473 nm light-emitting diode (LED) at 60 mW/cm² and 10% duty cycle (0.5 second of light every 5 seconds) for 5 minutes. Nine hours after stimulation, cells were fixed and imaged. (D) Same as (C), but HEK 293T cells were stably expressing the FLARE protease component and transiently expressing FLARE TF component and UAS-luciferase. Results of shorter and longer irradiation times are also shown. ±isoproterenol signal ratio was quantified for each time point. Each datapoint reflects one well of a 96-well plate containing >6,000 transfected cells. Four replicates per condition. (E) FLARE is specific for PPIs over non-interacting protein pairs. Same experiment as in (C), except arrestin was replaced by calmodulin protein (which does not interact with β2AR) in the second column, and β2AR was replaced by the calmodulin effector peptide MK2 (which does not interact with arrestin) in the third column. Anti-V5 antibodies stain for the FLARE TF component. (F) FLARE is activated by direct interactions and not merely proximity. Top: experimental scheme. To drive proximity but not interaction, FLARE constructs were created in which A and B domains were a transmembrane (TM) segment of the CD4 protein, and arrestin, respectively. TM and arrestin do not interact. HEK 293T cells expressing these FLARE constructs were also transfected with an expression plasmid for HA-tagged β2AR. Upon isoproterenol addition, arrestin-TEVp is recruited to the plasma membrane via interaction with β2AR, but it does not interact directly with the FLARE TF component. Bottom: Images of HEK 293T cells 9 hours after stimulation with isoproterenol and light (for 5 minutes). The last column shows the experiment depicted in the scheme. The first two columns are positive controls with FLARE constructs containing β2AR and arrestin (which do interact). The third column is a negative control with omission of the HA-β2AR construct. Anti-V5, anti-myc, and anti-HA antibodies stain for FLARE TF component, FLARE protease component, and HA-β2AR proteins, respectively. All scale bars, 100 μm.

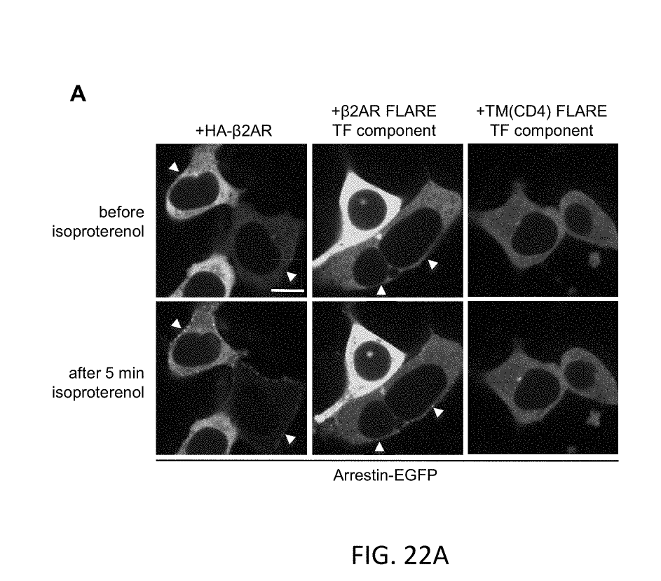

FIG. 22A-22B: (A) HA-β2AR construct recruits arrestin-EGFP to the plasma membrane. GFP images of HEK 293T cells transiently expressing rat arrestin2-EGFP along with one of the following: HA-β2AR, β2AR FLARE TF component (from FIG. 21B), or TM FLARE TF component (TM from CD4, used in FIG. 21F). Live cell GFP images were acquired before and after incubation with 10 μM isoproterenol to activate β2AR. Arrowheads point to regions showing re-localization of arrestin-GFP. Scale bar, 10 μm. (B) Additional fields of view for the experiment shown in FIG. 21F. Scale bar, 100 μm.

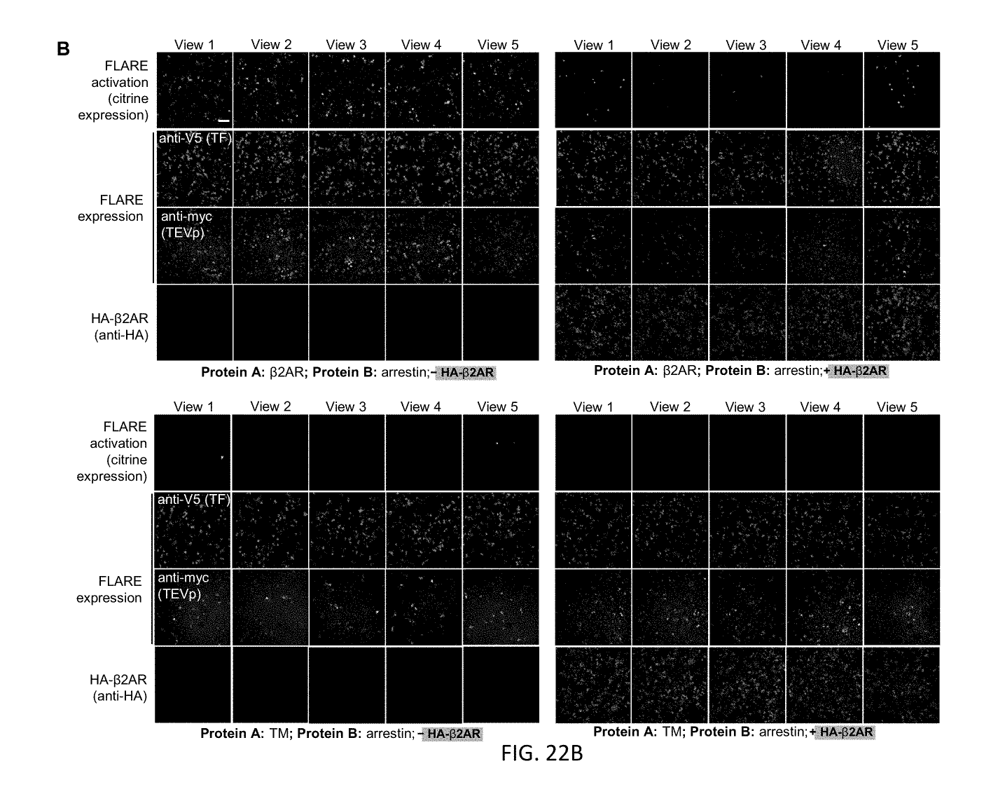

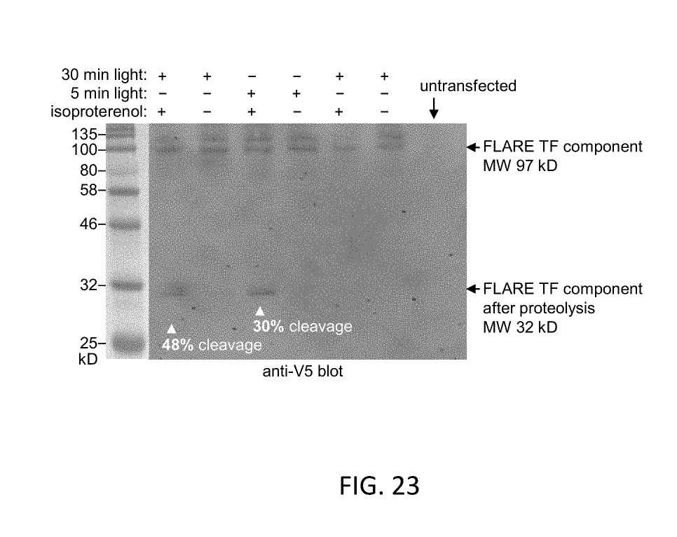

FIG. 23: Western Blot Quantification of Cleavage Extent.

HEK 293T cells were transiently transfected (using PEI max) with the FLARE-PPI constructs shown in FIG. 21B. 18 hrs post-transfection, cells were stimulated with 10 μM isoproterenol and blue light (473 nm, 60 mW/cm², 10% duty cycle) for 5 or 30 minutes total. Cells were then immediately lysed in the presence of 20 mM iodoacetamide TEVp inhibitor and run on 8% sodium dodecyl sulfate-polyacrylamide gel electrophoresis (SDS-PAGE). Anti-V5 blot visualizes the FLARE TF component, which is 97 kD before cleavage and 32 kD after cleavage at the TEVcs. Negative controls omit isoproterenol or light.

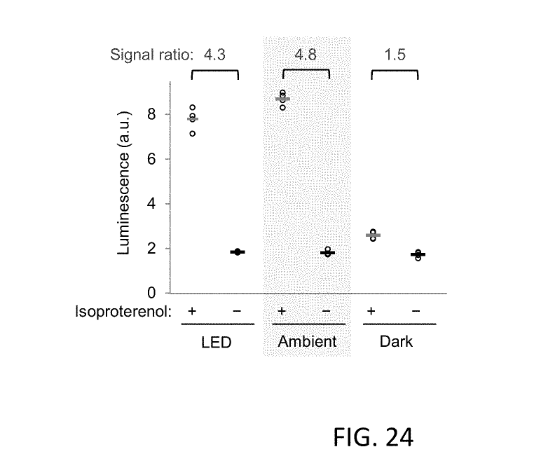

FIG. 24: Ambient Light Activates FLARE.

HEK 293T cells were prepared as in FIG. 21D. 15 hours post-transfection, cells were stimulated with 5 minutes of either ambient room light or blue LED light (473 nm, 60 mW/cm², 10% duty cycle) concurrently with 10 μM isoproterenol. Nine hours later, cells were analyzed for luciferase activity. Each condition was replicated four times.

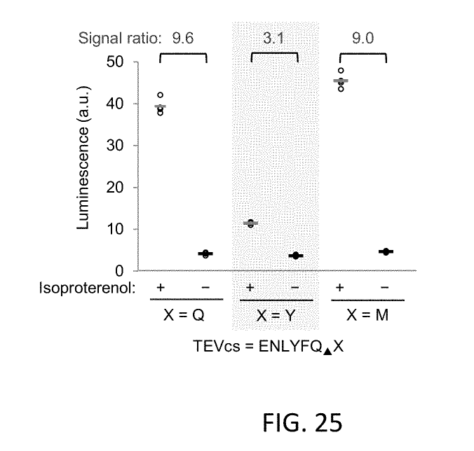

FIG. 25: Testing Alternative TEVcs Sequences.

Three alternative TEVcs sequences that differ at the P1′ site were tested in the context of β2AR-arrestin FLARE. HEK cells were prepared as in FIG. 21D and stimulated with 10 μM isoproterenol and blue LED light for 5 minutes. Nine hours later, cells were analyzed for luciferase activity. Each condition was replicated four times. The TEVcs sequence was used with X=M for all experiments in this Example, except where indicated.

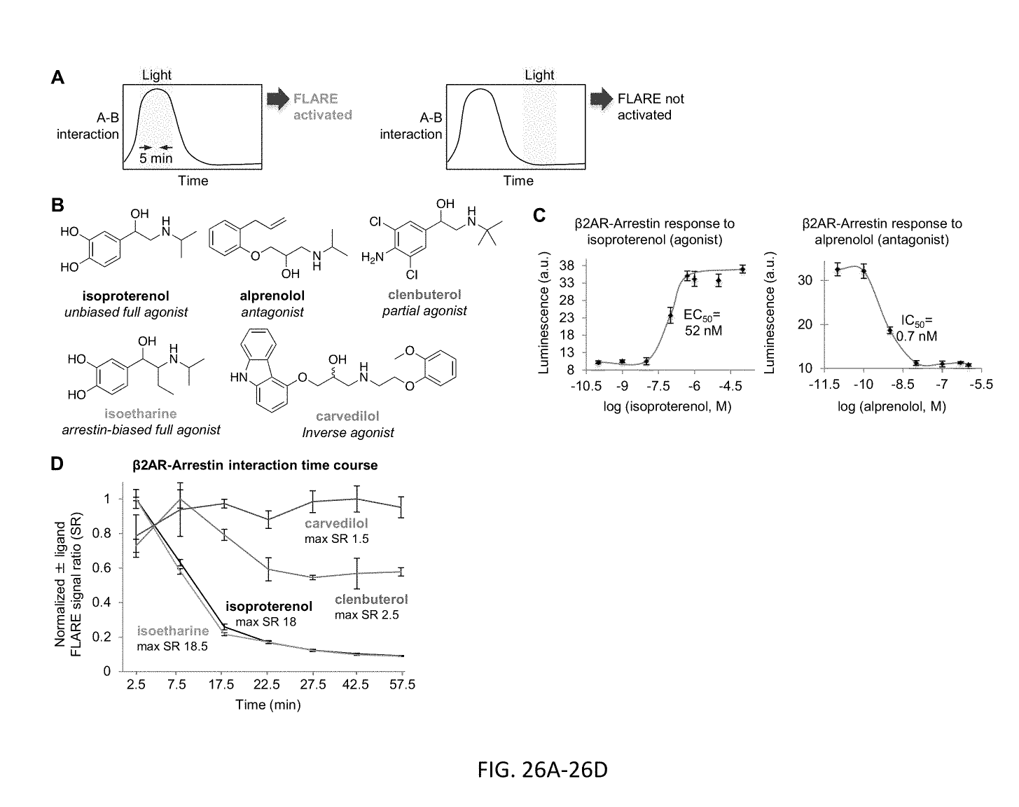

FIG. 26A-26D: Light Gating of FLARE-PPI Permits Analysis of the Dynamic GPCR-Arrestin2 Interaction.

(A) Scheme. By shifting the light window, it is possible to read out different time regimes of protein A-protein B interaction. On the left, light coincides with a period of high A-B interaction, resulting in FLARE activation and transcription of a reporter gene. On the right, light coincides with a period of low A-B interaction, so FLARE is not activated. (B) Panel of β2AR agonists, partial agonists, and antagonist. Biased agonists preferentially recruit one downstream effector (such as arrestin2) over another. (C) Isoproterenol and alprenolol dose-response curves with β2AR-arrestin2 FLARE readout. HEK 293T cells were prepared and stimulated as in FIG. 21D, with 5 minute light window. Four replicates per concentration. Errors, STE. EC₅₀² and IC₅₀³ are close to published values. (D) β2AR-arrestin2 interaction timecourse with various ligands. HEK 293T cells expressing FLARE constructs were prepared as in FIG. 21D. 15 hours after transfection, 10 μM ligand was added at time=0 minutes and remained on the cells for the duration of the experiment. The light window was 5 minutes, centered around the timepoint given on the x axis. 9 hours after initial addition of ligand, cells were mixed with luciferin substrate and analyzed for luciferase activity. Each datapoint represents the mean of 4 replicates. Errors, STE. Time courses are normalized so that max signal ratio (SR) of each is set to 1. Actual (non-normalized) max SRs are given next to each curve.

### Example 3: Application of FLARE-PPI to a Variety of PPIs

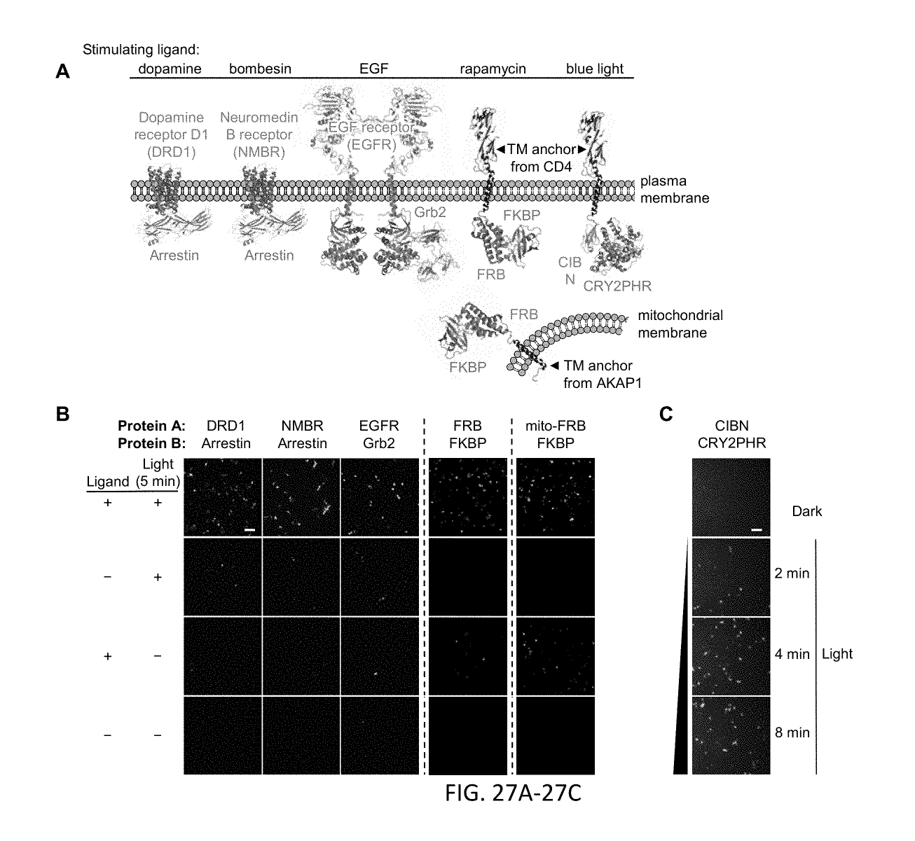

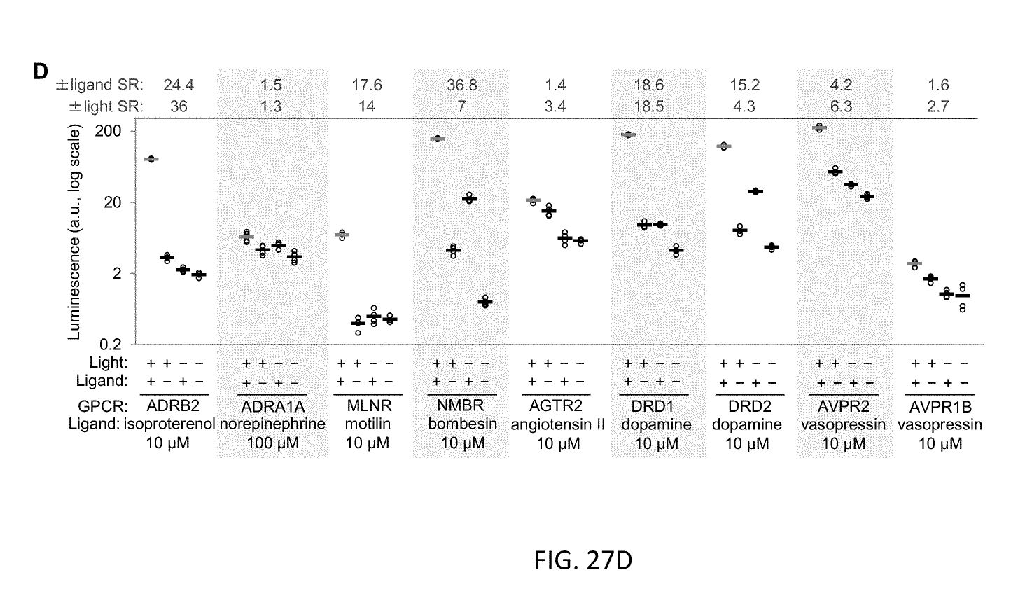

FIG. 27A-27D: FLARE-PPI can be Applied to a Variety of PPIs.

(A) PPI pairs studied with FLARE. DRD1 and NMBR are GPCRs that interact with arrestin2. EGFR is a receptor tyrosine kinase that recruits Grb2 upon stimulation with EGF ligand. FKBP and FRB are soluble proteins that heterodimerize upon addition of the drug rapamycin; to keep FRB FLARE out of the nucleus in the basal state, the FRB-FLARE was fused to either a plasma membrane anchor (TM from CD4) or a mitochondrial membrane anchor (TM from AKAP1). CIBN-CRY2 PHR is a light-inducible PPI. Kennedy et al. (2011) Nat. Methods 7:973-975. (B) FLARE data corresponding to PPIs depicted in (A). FLARE constructs were the same as those shown in FIG. 21B, except β2AR and arrestin2 were replaced by the A and B proteins indicated, respectively. HEK 293T cells transiently expressing FLARE constructs were stimulated with light and the ligand indicated in (A) for 5 minutes, then fixed and imaged 9 hours later. Citrine fluorescence images are shown. Dashed lines separate experiments that were performed separately and shown with different Citrine intensity scales. Scale bar, 100 μm. (C) FLARE detection of CIBN-CRY2 PHR interaction. Blue light (473 nm, 60 mW/cm², 33% duty cycle (2 seconds light every 6 seconds)) simultaneously uncages the eLOV domain and induces the CIBN-CRY2 PHR interaction. Scale bar, 100 μm. (D) FLARE applied to 9 different GPCRs. HEK 293T cells were prepared as in FIG. 21D. The FLARE protease component is arrestin2-TEVp. The FLARE TF component contains the indicated GPCR (no vasopressin V2 domain). Light (ambient) and ligand were applied for 15 minutes total, then cells were analyzed for luciferase activity 9 hours later. Four replicates per condition. ±ligand signal ratios (SR) and ±light signal ratios for each GPCR quantified across top.

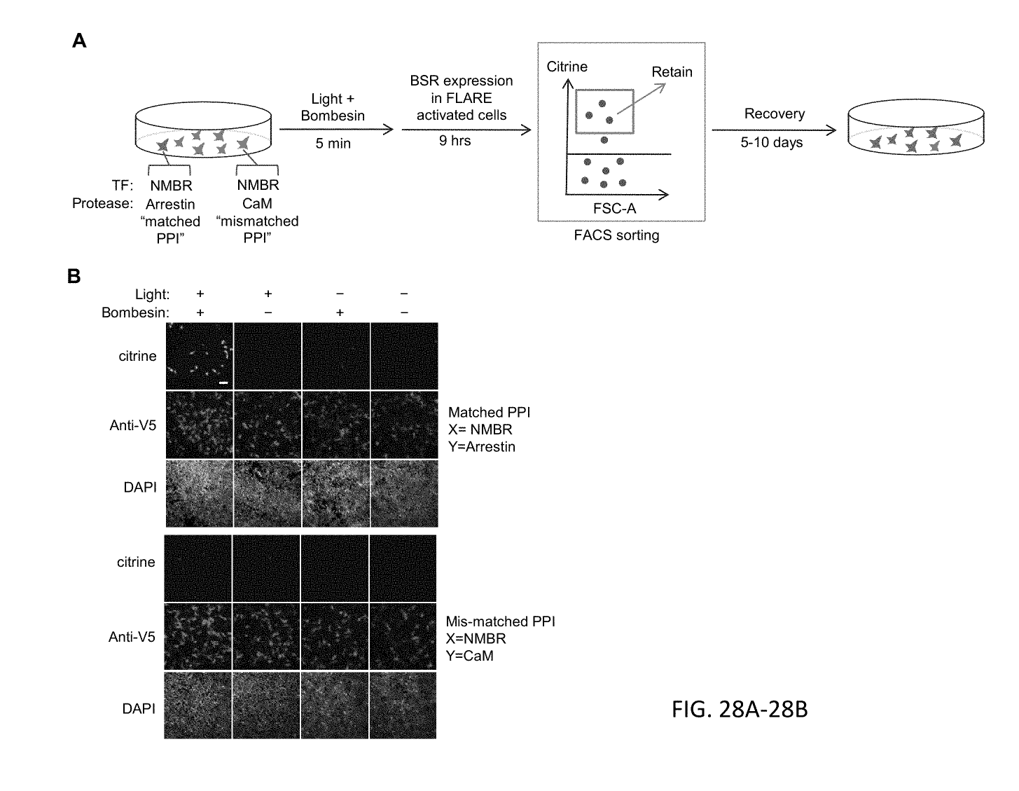

FIG. 28A-28B: FLARE can be Coupled to Genetic Selections.

A: Scheme. B: GFP images of cells expressed matched vs. mismatched PPI constructs before fluorescence activated cell sorting (FACS).

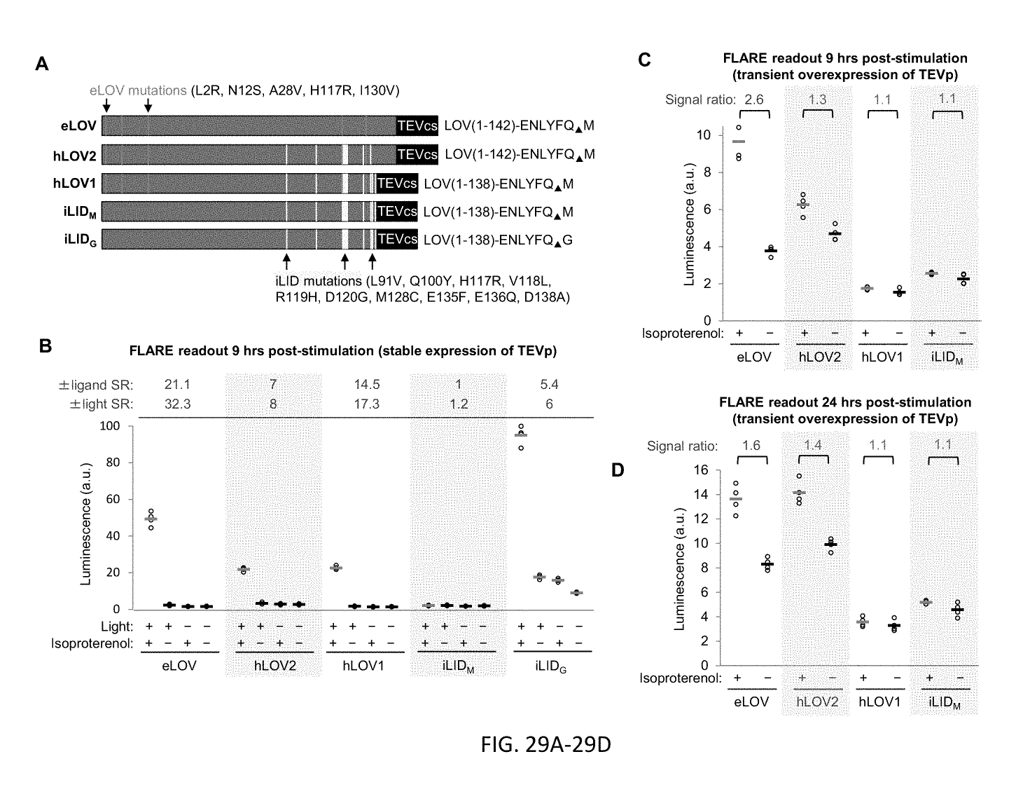

FIG. 29A-29D: Testing Alternative LOV Domains.

(A) Five LOV-TEVcs fusions compared. eLOV (top) was engineered by directed evolution, and was used in all FLARE experiments in this Example, except where indicated. The red lines indicate where the eLOV sequence differs from that of AsLOV2(G126A/N136E)⁵, the template used for directed evolution. iTANGO uses the LOV domain from iLID⁷ (bottom two constructs) and its TEVcs “bites back” 6 amino acids into LOV's Jα helix. Yellow lines indicate where iLID's LOV sequence differs from that of AsLOV2. hLOV1 and hLOV2 are two hybrid LOV domains that merge the features of eLOV and iLID. TEVcs is the same in the top four constructs but has Gly instead of Met in the P1′ position in the bottom construct. (B) Comparison of five LOV-TEVcs fusions, with luciferase readout, and stable/low expression of arrestin-TEVp. HEK 293T cells were prepared as in FIG. 21D, with arrestin-TEVp stably expressed and FLARE β2AR-TF (containing one of five LOV-TEVcs sequences from (A)) and UAS-luciferase transiently expressed. 18 hours post-transfection, cells were stimulated with 5 minutes of isoproterenol and ambient light. Nine hours later, cells were analyzed for luciferase activity. Each condition was replicated four times. ±ligand signal ratios (SR) and ±light signal ratios for each construct quantified across top. (C) Same as (B), but with transient overexpression of arrestin-TEVp component, instead of stable/low expression. (D) Same as (C) but luciferase activity was measured 24 hours post-stimulation instead of 9 hours post-stimulation.

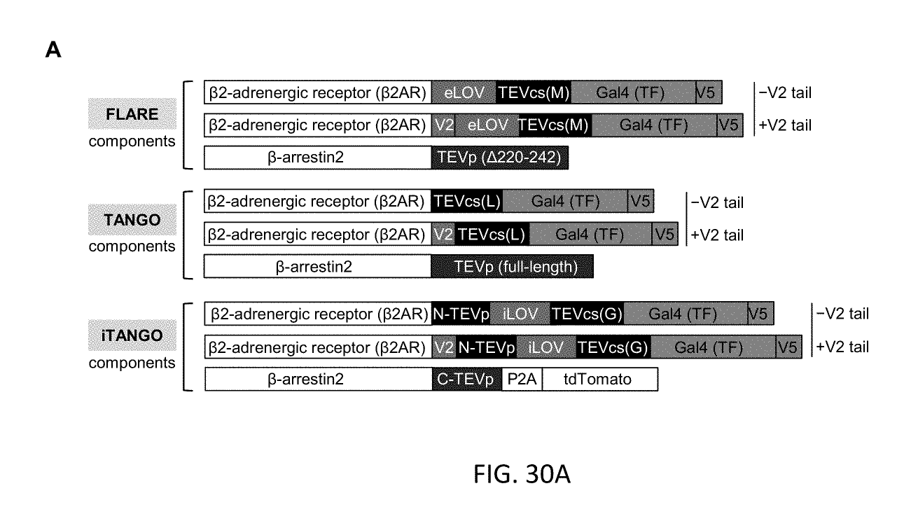

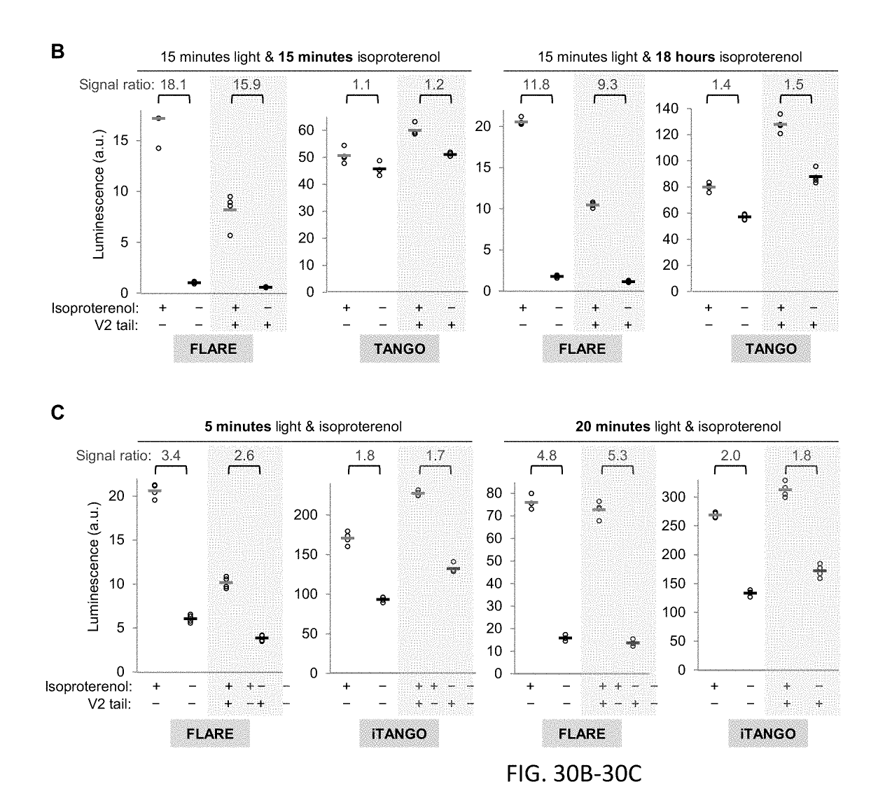

FIG. 30A-30C. FLARE-PPI Comparison to TANGO and iTango.

(A) FLARE, TANGO, and iTANGO constructs used to detect β2AR-arrestin2 interaction. The β2AR fusions were each prepared with and without the vasopressin receptor tail (V2, purple) that enhances arrestin recruitment (Kroeze et al. (2015) Nat. Struct. Mol. Biol. 22:362. FLARE, TANGO, and iTANGO constructs differ only in their TEVcs, TEVp, and LOV sequences; arrestin, β2AR, and TF domains are constant. In comparison to FLARE, TANGO uses full-length TEVp and a lower-affinity TEVcs with Leu instead of Met at the P1′ site. TANGO has no light gating. In comparison to FLARE, iTango uses a split TEVp, a higher-affinity TEVcs with Gly at the P1′ site, and the LOV sequence from iLID (iLOV) (Guntas et al. (2015) Proc. Natl. Acad. Sci. USA 112:112). (B) FLARE versus TANGO comparison. HEK 293T cells stably expressing the protease component of FLARE or TANGO were transiently transfected with the corresponding TF component and UAS-luciferase. 18 hours post-transfection, cells were stimulated with 15 minutes of light (473 nm, 60 mW/cm², 10% duty cycle) and isoproterenol, then analyzed for luciferase activity 9 hours later (left). Alternatively (right), cells were stimulated with 15 minutes of light in the presence of isoproterenol, and isoproterenol remained on the cells for another 18 hours, before luciferase detection (to match published conditions for TANGO (Barnea et al. (2008) Proc. Natl. Acad. Sci. USA 105:64; Inagaki et al. (2012) Cell 148:583). Each condition was replicated four times. ±isoproterenol signal ratios are quantified at top. (C) FLARE versus iTANGO comparison. Constructs shown in (A) were introduced by lipofectamine transfection into HEK 293T cells along with UAS-luciferase. 18 hrs post-transfection, cells were stimulated with either 5 minutes (left) or 20 minutes (right) of isoproterenol and light (473 nm, 60 mW/cm², 10% duty cycle). Nine hours later, cells were analyzed for luciferase activity. Each condition was replicated four times. ±isoproterenol signal ratios are quantified at top.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A nucleic acid system comprising:
A) a first nucleic acid comprising, in order from 5′ to 3′:
a) a nucleotide sequence encoding a first, light-activated, fusion polypeptide comprising, in order from amino terminus to carboxyl terminus:
i) a transmembrane domain;
ii) a first member of a protein interaction pair;
iii) a LOV-domain light-activated polypeptide comprising an amino acid sequence having at least 80% amino acid sequence identity to an amino acid sequence selected from the group consisting of SEQ ID NOS:142-148; and
iv) a proteolytically cleavable linker; and
b) an insertion site for a nucleic acid comprising a nucleotide sequence encoding a polypeptide of interest; and
B) a second nucleic acid comprising a nucleotide sequence encoding a second fusion polypeptide comprising:
i) a second member of the protein interaction pair; and
ii) a protease that cleaves the proteolytically cleavable linker,
wherein the first member of the protein interaction pair and the second member of the protein interaction pair bind to one another in the presence of an agent.

2. A nucleic acid system comprising:
a) a first nucleic acid comprising a nucleotide sequence encoding a first fusion polypeptide comprising, in order from amino terminus to carboxyl terminus:
i) a transmembrane domain;
ii) a first member of a protein interaction pair;
iii) a LOV light-activated polypeptide comprising an amino acid sequence having at least 80% amino acid sequence identity to an amino acid sequence selected from the group consisting of SEQ ID NOS:142-148;
iv) a proteolytically cleavable linker; and
v) a polypeptide of interest; and
b) a second nucleic acid comprising a nucleotide sequence encoding a second fusion polypeptide comprising:
i) a second member of the protein interaction pair; and
ii) a protease that cleaves the proteolytically cleavable linker,
wherein the first member of the protein interaction pair and the second member of the protein interaction pair bind to one another in the presence of a binding-inducing agent.

3. The nucleic acid system of claim 1, wherein the insertion site is a multiple cloning site.

4. The nucleic acid system of claim 2, wherein the first member of the protein interaction pair is an N-terminal portion of a polypeptide; and wherein the second member of the protein interaction pair is a C-terminal portion of the polypeptide.

5. The nucleic acid system of claim 2, wherein the first and second polypeptides of the protein interaction pair bind to one another in the presence of a small molecule agent, a hormone, or an ion.

6. The nucleic acid system of claim 2, wherein the first and second polypeptides of the protein interaction pair bind to one another in the presence of light of an activating wavelength.

7-8. (canceled)

9. The nucleic acid system of claim 2, wherein the protein interaction pair is selected from:
a) FK506 binding protein (FKBP) and FKBP;
b) FKBP and calcineurin catalytic subunit A (CnA);
c) FKBP and cyclophilin;
d) FKBP and FKBP-rapamycin associated protein (FRB);
e) gyrase B (GyrB) and GyrB;
f) dihydrofolate reductase (DHFR) and DHFR;
g) DmrB and DmrB;
h) PYL and ABI;
i) Cry2 and CIB1;
j) GAI and GID1;
k) mineralcorticoid receptor (MR) ligand-binding domain (LBD) and an SRC1-2 peptide;
l) a PPAR-γ LBD and an SRC1 peptide;
m) an androgen receptor LBF and an SRC3-1 peptide;
n) a PPAR-γ LBD and an SRC3 peptide;
o) an MR LBD and a PGC1a peptide;
p) an MR LBD and a TRAP220-1 peptide;
q) a progesterone receptor LBD and an NCoR peptide;
r) an estrogen receptor-β LBD and an NR0B1 peptide;
s) a PPAR-γ LBD and a TIF2 peptide;
t) an ERα LBD and a CoRNR box peptide;
u) an ERα LBD and an abV peptide;
v) a G protein-coupled receptor (GPCR) and a G protein;
w) a GPCR and a beta-arrestin polypeptide; and
x) an epidermal growth factor receptor (EGFR) and Src/Shc/Grb2.

10. The nucleic acid system of claim 2, wherein the LOV-domain light-activated polypeptide comprises one or more amino acid substitutions selected from L2R, N12S, A28V, H117R, and I130V substitutions relative to the amino acid sequence of SEQ ID NO:143.

11. The nucleic acid system of claim 2, wherein the LOV domain light-activated polypeptide comprises L2R, N12S, I130V, A28V, and H117R substitutions relative to the amino acid sequence of SEQ ID NO: 143.

12. The nucleic acid system of claim 2, wherein the proteolytically cleavable linker comprises an amino acid sequence cleaved by a viral protease, a mammalian protease, or a recombinant protease.

13. The nucleic acid system of claim 2, wherein the protease is a viral protease, a mammalian protease, or a recombinant protease.

14. The nucleic acid system of claim 2, wherein the first nucleic acid is present in a first expression vector, and the second nucleic acid is present in a second expression vector.

15-16. (canceled)

17. The nucleic acid system of claim 2, wherein the polypeptide of interest is a reporter polypeptide, a light-activated polypeptide, a transcription factor, a toxin, a calcium sensor, a recombinase, an antibiotic resistance factor, a DREADD, an RNA-guided endonuclease, a drug resistance factor, a biotin ligase, a kinase, a phosphorylase, or a peroxidase.

18. The nucleic acid system of claim 17, wherein the polypeptide of interest is a reporter polypeptide selected from a fluorescent polypeptide, an enzyme that produces a colored product, an enzyme that produces a luminescent product, and an enzyme that produces a fluorescent product.

19. The nucleic acid system of claim 17, wherein the polypeptide of interest is a transcriptional activator or a transcriptional repressor.

20. The nucleic acid system of claim 17, wherein the polypeptide of interest is an antibiotic resistance factor.

21. The nucleic acid system of claim 17, wherein the polypeptide of interest is an RNA-guided endonuclease selected from a Cas9 polypeptide, a C2C2 polypeptide, or a Cpf1 polypeptide.

22. A genetically modified host cell, wherein the host cell is genetically modified with the nucleic acid system of claim 2.

23. The genetically modified host cell of claim 22, wherein the cell is in vitro or in vivo.

24. (canceled)

25. The genetically modified host cell claim 22, wherein the cell is an animal cell or a plant cell.

26. The genetically modified host cell of claim 25, wherein the cell is a mammalian cell, an insect cell, a reptile cell, an amphibian cell, or an avian cell.

27. (canceled)

28. The genetically modified host cell of claim 25, wherein the cell is a cell of an invertebrate animal.

29. The genetically modified host cell of claim 22, wherein the cell is a single celled organism.

30. (canceled)

31. The genetically modified host cell of claim 22, wherein the first and/or the second nucleic acid is stably integrated into the genome of the host cell.

32-37. (canceled)

38. A nucleic acid system comprising:
A) a first nucleic acid comprising a nucleotide sequence encoding a first fusion polypeptide comprising, in order from amino terminus to carboxyl terminus:
i) a transmembrane domain;
ii) a first member of a protein interaction pair;
iii) a LOV light-activated polypeptide comprising an amino acid sequence having at least 80% amino acid sequence identity to an amino acid sequence selected from the group consisting of SEQ ID NOS:142-148;
iv) a proteolytically cleavable linker; and
v) a signal polypeptide; and
B) a second nucleic acid comprising, in order from 5′ to 3′:
a) an insertion site for a nucleic acid comprising a nucleotide sequence encoding a second member of the protein interaction pair; and
b) a nucleotide sequence encoding a protease that cleaves the proteolytically cleavable linker,
wherein the first member of the protein interaction pair and the second member of the protein interaction pair bind to one another in the presence of a binding-inducing agent, and wherein the signal polypeptide provides a signal when cleaved from the fusion polypeptide.

39. The nucleic acid system of claim 38, wherein the insertion site is a multiple cloning site.

40. The nucleic acid system of claim 38, wherein the second member of the protein interaction pair is encoded by a member of a library comprising a plurality of nucleic acids.

41. The nucleic acid system of claim 38, wherein the signal polypeptide is a fluorescent protein, a transcription factor, or an enzyme.

42. The nucleic acid system of claim 38, wherein one or both of the first and the second nucleic acids are in expression vectors.

43-44. (canceled)

45. A genetically modified host cell, wherein the host cell is genetically modified with the nucleic acid system of claim 38.

46. A polypeptide system comprising
a) a first fusion polypeptide comprising:
i) a transmembrane domain;
ii) a first member of a protein interaction pair;
iii) a LOV light-activated polypeptide comprising an amino acid sequence having at least 80% amino acid sequence identity to an amino acid sequence selected from the group consisting of SEQ ID NOS:142-148;
iv) a proteolytically cleavable linker; and
v) a polypeptide of interest; and
b) a second fusion polypeptide comprising:
i) a second member of the protein interaction pair; and
ii) a protease that cleaves the proteolytically cleavable linker.

47. The system of claim 46, wherein the LOV-domain light-activated polypeptide comprises one or more amino acid substitutions selected from L2R, N12S, A28V, H117R, and I130V substitutions relative to the amino acid sequence depicted in FIG. 11B.

48. The system of claim 47, wherein the LOV domain light-activated polypeptide comprises L2R, N12S, I130V, A28V, and H117R substitutions relative to the amino acid sequence of SEQ ID NO: 143.

49. (canceled)

50. The system of claim 46, wherein the protease is a viral protease.

51-52. (canceled)

53. The system of claim 46, wherein the first member of the protein interaction pair is an N-terminal portion of a polypeptide; and wherein the second member of the protein interaction pair is a C-terminal portion of the polypeptide.

54. The system of claim 46, wherein the first and second polypeptides of the protein interaction pair bind to one another in the presence of a small molecule agent, a hormone, or an ion.

55. The system of claim 46, wherein the first and second polypeptides of the protein interaction pair bind to one another in the presence of light of an activating wavelength.

56-57. (canceled)

58. The system of claim 46, wherein the protein interaction pair is selected from:
a) FK506 binding protein (FKBP) and FKBP;
b) FKBP and calcineurin catalytic subunit A (CnA);
c) FKBP and cyclophilin;
d) FKBP and FKBP-rapamycin associated protein (FRB);
e) gyrase B (GyrB) and GyrB;
f) dihydrofolate reductase (DHFR) and DHFR;
g) DmrB and DmrB;
h) PYL and ABI;
i) Cry2 and CIB1;
j) GAI and GID1;
k) mineralcorticoid receptor (MR) ligand-binding domain (LBD) and an SRC1-2 peptide;
l) a PPAR-γ LBD and an SRC1 peptide;
m) an androgen receptor LBF and an SRC3-1 peptide;
n) a PPAR-γ LBD and an SRC3 peptide;
o) an MR LBD and a PGC1a peptide;
p) an MR LBD and a TRAP220-1 peptide;
q) a progesterone receptor LBD and an NCoR peptide;
r) an estrogen receptor-β LBD and an NR0B1 peptide;
s) a PPAR-γ LBD and a TIF2 peptide;
t) an ERα LBD and a CoRNR box peptide;
u) an ERα LBD and an abV peptide;
v) a G protein-coupled receptor (GPCR) and a G protein;
w) a GPCR and a beta-arrestin polypeptide; and
x) an epidermal growth factor receptor (EGFR) and Src/Shc/Grb2.

59. A mammalian cell comprising the system of claim 46.

60. The mammalian cell of claim 59, wherein the cell is in vitro.

61. A genetically modified non-human organism that comprises, integrated into the genome of one or more cells of the organism, the nucleic acid system of claim 2.

62. The genetically modified non-human organism of claim 61, wherein the organism is a mammal.

63. The genetically modified non-human organism of claim 62, wherein the mammal is a rodent.

64. A method for detecting protein-protein interaction in a cell in response to a stimulus, the method comprising:
A) exposing the cell to the stimulus, wherein the cell comprises:
a) a first fusion polypeptide comprising:
i) a transmembrane domain;
ii) a first member of a protein interaction pair;
iii) a LOV light-activated polypeptide comprising an amino acid sequence having at least 80% amino acid sequence identity to an amino acid sequence selected from the group consisting of SEQ ID NOS:142-148;
iv) a proteolytically cleavable linker; and
v) a signal polypeptide that produces a signal only following release from the first fusion polypeptide; and
b) a second fusion polypeptide comprising:
i) a second member of the protein interaction pair; and
ii) a protease that cleaves the proteolytically cleavable linker;
B) substantially simultaneously exposing the cell to light of a wavelength that activates the LOV domain polypeptide; and
C) detecting a signal produced by the signal polypeptide,
wherein an increase in a signal produced by the signal polypeptide, compared to a control level of the signal, indicates that exposure of the cell to the stimulus results in binding of the first member to the second member of the protein interaction pair.

65. The method of claim 64, wherein the stimulus is a ligand, a drug, a toxin, a neurotransmitter, contact with a second cell, heat, or hypoxia.

66. The method of claim 64, wherein the signal polypeptide is a transcription factor that induces transcription of a detectable polypeptide.

67. The method of claim 66, wherein the detectable polypeptide is a fluorescent protein.

68. The method of claim 64, wherein the cell is in vitro or in vivo.

69. (canceled)

70. The method of claim 64, wherein the cell is a human cell or a non-human animal cell.

71. (canceled)

72. The method of claim 64, wherein the second member of the protein interaction pair is encoded by a member of a library comprising a plurality of nucleic acids.


SEQUENCE LISTING (302 sequences)

SEQ ID NO: 1 - protein, 22 aa, Artificial sequence
  CD4 TM domain
MALIVLGGVAGLLLFIGLGIFF

SEQ ID NO: 2 - protein, 21 aa, Artificial sequence
  CD4 TM domain
IYIWAPLAGTCGVLLLSLVIT

SEQ ID NO: 3 - protein, 21 aa, Artificial sequence
  CD4 TM domain
GMVVGIVAAAALCILILLYAM

SEQ ID NO: 4 - protein, 21 aa, Artificial sequence
  CD4 TM domain
FMYVAAAAFVLLFFVGCGVLL

SEQ ID NO: 5 - protein, 10 aa, Artificial sequence
  nuclear exclusion signal
MVKELQEIRL

SEQ ID NO: 6 - protein, 11 aa, Artificial sequence
  nuclear exclusion signal
MTASALARMEV

SEQ ID NO: 7 - protein, 10 aa, Artificial sequence
  nuclear exclusion signal
LALKLAGLDI

SEQ ID NO: 8 - protein, 10 aa, Artificial sequence
  nuclear exclusion signal
LQKKLEELEL

SEQ ID NO: 9 - protein, 10 aa, Artificial sequence
  nuclear exclusion signal
LESNLRELQI

SEQ ID NO: 10 - protein, 10 aa, Artificial sequence
  nuclear exclusion signal
LCQAFSDVLI

SEQ ID NO: 11 - protein, 12 aa, Artificial sequence
  nuclear exclusion signal
MVKELQEIRLEP

SEQ ID NO: 12 - protein, 11 aa, Artificial sequence
  nuclear exclusion signal
LQKKLEELELA

SEQ ID NO: 13 - protein, 11 aa, Artificial sequence
  nuclear exclusion signal
LALKLAGLDIN

SEQ ID NO: 14 - protein, 12 aa, Artificial sequence
  nuclear exclusion signal
LQLPPLERLTLD

SEQ ID NO: 15 - protein, 11 aa, Artificial sequence
  nuclear exclusion signal
LQKKLEELELE

SEQ ID NO: 16 - protein, 10 aa, Artificial sequence
  nuclear exclusion signal
MTKKFGTLTI

SEQ ID NO: 17 - protein, 10 aa, Artificial sequence
  nuclear exclusion signal
LAEMLEDLHI

SEQ ID NO: 18 - protein, 10 aa, Artificial sequence
  nuclear exclusion signal
LDQQFAGLDL

SEQ ID NO: 19 - protein, 10 aa, Artificial sequence
  nuclear exclusion signal
LCQAFSDVIL

SEQ ID NO: 20 - protein, 9 aa, Artificial sequence
  nuclear exclusion signal
LPVLENLTL

SEQ ID NO: 21 - protein, 16 aa, Artificial sequence
  nuclear exclusion signal
IQQQLGQLTLENLQML

SEQ ID NO: 22 - protein, 108 aa, Artificial sequence
  FKBP
MGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 23 - protein, 292 aa, Artificial Sequence
  PP2Ac domain
LEESVALRIITEGASILRQEKNLLDIDAPVTVCGDIHGQFFDLMKLFEVGGSPANTRYLFLGDYVDRGYFSIECVLYLWALKILYPKTLFLLRGNHECRHLTEYFTFKQECKIKYSERVYDACMDAFDCLPLAALMNQQFLCVHGGLSPEINTLDDIRKLDRFKEPPAYGPMCDILWSDPLEDFGNEKTQEHFTHNTVRGCSYFYSYPAVCEFLQHNNLLSILRAHEAQDAGYRMYRKSQTTGFPSLITIFSAPNYLDVYNNKAAVLKYENNVMNIRQFNCSPHPYWLPNFM

SEQ ID NO: 24 - protein, 165 aa, Piliocolobus tephrosceles
MVNPTVFFDIAVDGEPLGRVSFELFADKVPKTAENFRALSTGEKGFGYKGSCFHRIIPGFMCQGGDFTRHNGTGGKSIYGEKFEDENFILKHTGPGILSMANAGPNTNGSQFFICTAKTEWLDGKHVVFGKVKEGMNIVEAMERFGSRNGKTSKKITIADCGQLE

SEQ ID NO: 25 - protein, 94 aa, Artificial sequence
  Fkbp-Rapamycin Binding Domain
MILWHEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 26 - protein, 804 aa, Escherichia coli
MSNSYDSSSIKVLKGLDAVRKRPGMYIGDTDDGTGLHHMVFEVVDNAIDEALAGHCKEIIVTIHADNSVSVQDDGRGIPTGIHPEEGVSAAEVIMTVLHAGGKFDDNSYKVSGGLHGVGVSVVNALSQKLELVIQREGKIHRQIYEHGVPQAPLAVTGETEKTGTMVRFWPSLETFTNVTEFEYEILAKRLRELSFLNSGVSIRLRDKRDGKEDHFHYEGGIKAFVEYLNKNKTPIHPNIFYFSTEKDGIGVEVALQWNDGFQENIYCFTNNIPQRDGGTHLAGFRAAMTRTLNAYMDKEGYSKKAKVSATGDDAREGLIAVVSVKVPDPKFSSQTKDKLVSSEVKSAVEQQMNELLAEYLLENPTDAKIVVGKIIDAARAREAARRAREMTRRKGALDLAGLPGKLADCQERDPALSELYLVEGDSAGGSAKQGRNRKNQAILPLKGKILNVEKARFDKMLSSQEVATLITALGCGIGRDEYNPDKLRYHSIIIMTDADVDGSHIRTLLLTFFYRQMPEIVERGHVYIAQPPLYKVKKGKQEQYIKDDEAMDQYQISIALDGATLHTNASAPALAGEALEKLVSEYNATQKMINRMERRYPKAMLKELIYQPTLTEADLSDEQTVTRWVNALVSELNDKEQHGSQWKFDVHTNAEQNLFEPIVRVRTHGVDTDYPLDHEFITGGEYRRICTLGEKLRGLLEEDAFIERGERRQPVASFEQALDWLVKESRRGLSIQRYKGLGEMNPEQLWETTMDPESRRMLRVTVKDAIAADQLFTTLMGDAVEPRRAFIEENALKAANIDI

SEQ ID NO: 27 - protein, 187 aa, Homo sapiens
MVGSLNCIVAVSQNMGIGKNGDLPWPPLRNEFRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKND

SEQ ID NO: 28 - protein, 111 aa, Artificial sequence
  DmrB polypeptide
MASRGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 29 - protein, 183 aa, Arabidopsis thaliana
MNGDETKKVESEYIKKHHRHELVESQCSSTLVKHIKAPLHLVWSIVRRFDEPQKYKPFISRCVVQGKKLEVGSVREVDLKSGLPATKSTEVLEILDDNEHILGIRIVGGDHRLKNYSSTISLHSETIDGKTGTLAIESFVVDVPEGNTKEETCFFVEALIQCNLNSLADVTERLQAESMEKKI

SEQ ID NO: 30 - protein, 161 aa, Arabidopsis thaliana
METSQKYHTCGSTLVQTIDAPLSLVWSILRRFDNPQAYKQFVKTCNLSSGDGGEGSVREVTVVSGLPAEFSRERLDELDDESHVMMISIIGGDHRLVNYRSKTMAFVAADTEEKTVVVESYVVDVPEGNSEEETTSFADTIVGFNLKSLAKLSERVAHLKL

SEQ ID NO: 31 - protein, 159 aa, Arabidopsis thaliana
MKTSQEQHVCGSTVVQTINAPLPLVWSILRRFDNPKTFKHFVKTCKLRSGDGGEGSVREVTVVSDLPASFSLERLDELDDESHVMVISIIGGDHRLVNYQSKTTVFVAAEEEKTVVVESYVVDVPEGNTEEETTLFADTIVGCNLRSLAKLSEKMMELT

SEQ ID NO: 32 - protein, 164 aa, Arabidopsis thaliana
MESSKQKRCRSSVVETIEAPLPLVWSILRSFDKPQAYQRFVKSCTMRSGGGGGKGGEGKGSVRDVTLVSGFPADFSTERLEELDDESHVMVVSIIGGNHRLVNYKSKTKVVASPEDMAKKTVVVESYVVDVPEGTSEEDTIFFVDNIIRYNLTSLAKLTKKMMK

SEQ ID NO: 33 - protein, 221 aa, Arabidopsis thaliana
MANSESSSSPVNEEENSQRISTLHHQTMPSDLTQDEFTQLSQSIAEFHTYQLGNGRCSSLLAQRIHAPPETVWSVVRRFDRPQIYKHFIKSCNVSEDFEMRVGCTRDVNVISGLPANTSRERLDLLDDDRRVTGFSITGGEHRLRNYKSVTTVHRFEKEEEEERIWTVVLESYVVDVPEGNSEEDTRLFADTVIRLNLQKLASITEAMNRNNNNNNSSQVR

SEQ ID NO: 34 - protein, 190 aa, Arabidopsis thaliana
MSSSPAVKGLTDEEQKTLEPVIKTYHQFEPDPTTCTSLITQRIHAPASVVWPLIRRFDNPERYKHFVKRCRLISGDGDVGSVREVTVISGLPASTSTERLEFVDDDHRVLSFRVVGGEHRLKNYKSVTSVNEFLNQDSGKVYTVVLESYTVDIPEGNTEEDTKMFVDTVVKLNLQKLGVAATSAPMHDDE

SEQ ID NO: 35 - protein, 209 aa, Arabidopsis thaliana
MNLAPIHDPSSSSTTTTSSSTPYGLTKDEFSTLDSIIRTHHTFPRSPNTCTSLIAHRVDAPAHAIWRFVRDFANPNKYKHFIKSCTIRVNGNGIKEIKVGTIREVSVVSGLPASTSVEILEVLDEEKRILSFRVLGGEHRLNNYRSVTSVNEFVVLEKDKKKRVYSVVLESYIVDIPQGNTEEDTRMFVDTVVKSNLQNLAVISTASPT

SEQ ID NO: 36 - protein, 207 aa, Arabidopsis thaliana
MLAVHRPSSAVSDGDSVQIPMMIASFQKRFPSLSRDSTAARFHTHEVGPNQCCSAVIQEISAPISTVWSVVRRFDNPQAYKHFLKSCSVIGGDGDNVGSLRQVHVVSGLPAASSTERLDILDDERHVISFSVVGGDHRLSNYRSVTTLHPSPISGTVVVESYVVDVPPGNTKEETCDFVDVIVRCNLQSLAKIAENTAAESKKKMSL

SEQ ID NO: 37 - protein, 203 aa, Arabidopsis thaliana
MRSPVQLQHGSDATNGFHTLQPHDQTDGPIKRVCLTRGMHVPEHVAMHHTHDVGPDQCCSSVVQMIHAPPESVWALVRRFDNPKVYKNFIRQCRIVQGDGLHVGDLREVMVVSGLPAVSSTERLEILDEERHVISFSVVGGDHRLKNYRSVTTLHASDDEGTVVVESYIVDVPPGNTEEETLSFVDTIVRCNLQSLARSTNRQ

SEQ ID NO: 38 - protein, 215 aa, Arabidopsis thaliana
MPTSIQFQRSSTAAEAANATVRNYPHHHQKQVQKVSLTRGMADVPEHVELSHTHVVGPSQCFSVVVQDVEAPVSTVWSILSRFEHPQAYKHFVKSCHVVIGDGREVGSVREVRVVSGLPAAFSLERLEIMDDDRHVISFSVVGGDHRLMNYKSVTTVHESEEDSDGKKRTRVVESYVVDVPAGNDKEETCSFADTIVRCNLQSLAKLAENTSKFS

SEQ ID NO: 39 - protein, 211 aa, Arabidopsis thaliana
MEMIGGDDTDTEMYGALVTAQSLRLRHLHHCRENQCTSVLVKYIQAPVHLVWSLVRRFDQPQKYKPFISRCTVNGDPEIGCLREVNVKSGLPATTSTERLEQLDDEEHILGINIIGGDHRLKNYSSILTVHPEMIDGRSGTMVMESFVVDVPQGNTKDDTCYFVESLIKCNLKSLACVSERLAAQDITNSIATFCNASNGYREKNHTETNL

SEQ ID NO: 40 - protein, 188 aa, Arabidopsis thaliana
MEANGIENLTNPNQEREFIRRHHKHELVDNQCSSTLVKHINAPVHIVWSLVRRFDQPQKYKPFISRCVVKGNMEIGTVREVDVKSGLPATRSTERLELLDDNEHILSIRIVGGDHRLKNYSSIISLHPETIEGRIGTLVIESFVVDVPEGNTKDETCYFVEALIKCNLKSLADISERLAVQDTTESRV

SEQ ID NO: 41 - protein, 187 aa, Arabidopsis thaliana
MMDGVEGGTAMYGGLETVQYVRTHHQHLCRENQCTSALVKHIKAPLHLVWSLVRRFDQPQKYKPFVSRCTVIGDPEIGSLREVNVKSGLPATTSTERLELLDDEEHILGIKIIGGDHRLKNYSSILTVHPEIIEGRAGTMVIESFVVDVPQGNTKDETCYFVEALIRCNLKSLADVSERLASQDITQ

SEQ ID NO: 42 - protein, 191 aa, Arabidopsis thaliana
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSIIGGEHRLTNYKSVTTVHRFEKENRIWTVVLESYVVDMPEGNSEDDTRMFADTVVKLNLQKLATVAEAMARNSGDGSGSQVT

SEQ ID NO: 43 - protein, 434 aa, Arabidopsis thaliana
MEEVSPAIAGPFRPFSETQMDFTGIRLGKGYCNNQYSNQDSENGDLMVSLPETSSCSVSGSHGSESRKVLISRINSPNLNMKESAAADIVVVDISAGDEINGSDITSEKKMISRTESRSLFEFKSVPLYGFTSICGRRPEMEDAVSTIPRFLQSSSGSMLDGRFDPQSAAHFFGVYDGHGGSQVANYCRERMHLALAEEIAKEKPMLCDGDTWLEKWKKALFNSFLRVDSEIESVAPETVGSTSVVAVVFPSHIFVANCGDSRAVLCRGKTALPLSVDHKPDREDEAARIEAAGGKVIQWNGARVFGVLAMSRSIGDRYLKPSIIPDPEVTAVKRVKEDDCLILASDGVWDVMTDEEACEMARKRILLWHKKNAVAGDASLLADERRKEGKDPAAMSAAEYLSKLAIQRGSKDNISVVVVDLKPRRKLKSKPLN

SEQ ID NO: 44 - protein, 423 aa, Arabidopsis thaliana
MDEVSPAVAVPFRPFTDPHAGLRGYCNGESRVTLPESSCSGDGAMKDSSFEINTRQDSLTSSSSAMAGVDISAGDEINGSDEFDPRSMNQSEKKVLSRTESRSLFEFKCVPLYGVTSICGRRPEMEDSVSTIPRFLQVSSSSLLDGRVTNGFNPHLSAHFFGVYDGHGGSQVANYCRERMHLALTEEIVKEKPEFCDGDTWQEKWKKALFNSFMRVDSEIETVAHAPETVGSTSVVAVVFPTHIFVANCGDSRAVLCRGKTPLALSVDHKPDRDDEAARIEAAGGKVIRWNGARVFGVLAMSRSIGDRYLKPSVIPDPEVTSVRRVKEDDCLILASDGLWDVMTNEEVCDLARKRILLWHKKNAMAGEALLPAEKRGEGKDPAAMSAAEYLSKMALQKGSKDNISVVVVDLKGIRKFKSKSLN

SEQ ID NO: 45 - protein, 533 aa, Arabidopsis thaliana
MKRDHHHHHHQDKKTMMMNEEDDGNGMDELLAVLGYKVRSSEMADVAQKLEQLEVMMSNVQEDDLSQLATETVHYNPAELYTWLDSMLTDLNPPSSNAEYDLKAIPGDAILNQFAIDSASSSNQGGGGDTYTTNKRLKCSNGVVETTTATAESTRHVVLVDSQENGVRLVHALLACAEAVQKENLTVAEALVKQIGFLAVSQIGAMRKVATYFAEALARRIYRLSPSQSPIDHSLSDTLQMHFYETCPYLKFAHFTANQAILEAFQGKKRVHVIDFSMSQGLQWPALMQALALRPGGPPVFRLTGIGPPAPDNFDYLHEVGCKLAHLAEAIHVEFEYRGFVANTLADLDASMLELRPSEIESVAVNSVFELHKLLGRPGAIDKVLGVVNQIKPEIFTVVEQESNHNSPIFLDRFTESLHYYSTLFDSLEGVPSGQDKVMSEVYLGKQICNVVACDGPDRVERHETLSQWRNRFGSAGFAAAHIGSNAFKQASMLLALFNGGEGYRVEESDGCLMLGWHTRPLIATSAWKLSTN

SEQ ID NO: 46 - protein, 345 aa, Arabidopsis thaliana
MAASDEVNLIESRTVVPLNTWVLISNFKVAYNILRRPDGTFNRHLAEYLDRKVTANANPVDGVFSFDVLIDRRINLLSRVYRPAYADQEQPPSILDLEKPVDGDIVPVILFFHGGSFAHSSANSAIYDTLCRRLVGLCKCVVVSVNYRRAPENPYPCAYDDGWIALNWVNSRSWLKSKKDSKVHIFLAGDSSGGNIAHNVALRAGESGIDVLGNILLNPMFGGNERTESEKSLDGKYFVTVRDRDWYWKAFLPEGEDREHPACNPFSPRGKSLEGVSFPKSLVVVAGLDLIRDWQLAYAEGLKKAGQEVKLMHLEKATVGFYLLPNNNHFHNVMDEISAFVNAEC

SEQ ID NO: 47 - protein, 358 aa, Arabidopsis thaliana
MAGGNEVNLNECKRIVPLNTWVLISNFKLAYKVLRRPDGSFNRDLAEFLDRKVPANSFPLDGVFSFDHVDSTTNLLTRIYQPASLLHQTRHGTLELTKPLSTTEIVPVLIFFHGGSFTHSSANSAIYDTFCRRLVTICGVVVVSVDYRRSPEHRYPCAYDDGWNALNWVKSRVWLQSGKDSNVYVYLAGDSSGGNIAHNVAVRATNEGVKVLGNILLHPMFGGQERTQSEKTLDGKYFVTIQDRDWYWRAYLPEGEDRDHPACNPFGPRGQSLKGVNFPKSLVVVAGLDLVQDWQLAYVDGLKKTGLEVNLLYLKQATIGFYFLPNNDHFHCLMEELNKFVHSIEDSQSKSSPVLLTP

SEQ ID NO: 48 - protein, 344 aa, Arabidopsis thaliana
MAGSEEVNLIESKTVVPLNTWVLISNFKLAYNLLRRPDGTFNRHLAEFLDRKVPANANPVNGVFSFDVIIDRQTNLLSRVYRPADAGTSPSITDLQNPVDGEIVPVIVFFHGGSFAHSSANSAIYDTLCRRLVGLCGAVVVSVNYRRAPENRYPCAYDDGWAVLKWVNSSSWLRSKKDSKVRIFLAGDSSGGNIVHNVAVRAVESRIDVLGNILLNPMFGGTERTESEKRLDGKYFVTVRDRDWYWRAFLPEGEDREHPACSPFGPRSKSLEGLSFPKSLVVVAGLDLIQDWQLKYAEGLKKAGQEVKLLYLEQATIGFYLLPNNNHFHTVMDEIAAFVNAECQ

SEQ ID NO: 49 - protein, 612 aa, Arabidopsis thaliana
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTREAQIMIGAAPDEIVADSFEALGANTIKEPGLCPSVSSNDQQVPSAVRYNGSKRVKPEEEEERDMKKSRGFDERELFSTAESSSSSSVFFVSQSCSLASEGKNLEGIQDSSDQITTSLGKNGCK

SEQ ID NO: 50 - protein, 335 aa, Arabidopsis thaliana
MNGAIGGDLLLNFPDMSVLERQRAHLKYLNPTFDSPLAGFFADSSMITGGEMDSYLSTAGLNLPMMYGETTVEGDSRLSISPETTLGTGNFKKRKFDTETKDCNEKKKKMTMNRDDLVEEGEEEKSKITEQNNGSTKSIKKMKHKAKKEENNFSNDSSKVTKELEKTDYIHVRARRGQATDSHSIAERVRREKISERMKFLQDLVPGCDKITGKAGMLDEIINYVQSLQRQIEFLSMKLAIVNPRPDFDMDDIFAKEVASTPMTVVPSPEMVLSGYSHEMVHSGYSSEMVNSGYLHVNPMQQVNTSSDPLSCFNNGEAPSMWDSHVQNLYGNLGV

SEQ ID NO: 51 - protein, 299 aa, Artificial Sequence
  ligand-binding domain (LBD) of a mineralocorticoid receptor
EEQPQQQQPPPPPPPPQSPEEGTTYIAPAKEPSVNTALVPQLSTISRALTPSPVMVLENIEPEIVYAGYDSSKPDTAENLLSTLNRLAGKQMIQVVKWAKVLPGFKNLPLEDQITLIQYSWMCLSSFALSWRSYKHTNSQFLYFAPDLVFNEEKMHQSAMYELCQGMHQISLQFVRLQLTFEEYTIMKVLLLLSTIPKDGLKSQAAFEEMRTNYIKELRKMVTKCPNNSGQSWQRFYQLTKLLDSMHDLVSDLLEFCFYTFRESHALKVEFPAMLVEIISDQLPKVESGNAKPLYFHRK

SEQ ID NO: 52 - protein, 5 aa, Artificial sequence
  endoplasmic reticulum (ER) export signal
  misc_feature (2)..(3): Xaa can be any naturally occurring amino acid
VXXSL

SEQ ID NO: 53 - protein, 5 aa, Artificial sequence
  endoplasmic reticulum (ER) export signal
VKESL

SEQ ID NO: 54 - protein, 5 aa, Artificial sequence
  endoplasmic reticulum (ER) export signal
VLGSL

SEQ ID NO: 55 - protein, 16 aa, Artificial sequence
  endoplasmic reticulum (ER) export signal
NANSFCYENEVALTSK

SEQ ID NO: 56 - protein, 20 aa, Artificial sequence
  endoplasmic reticulum (ER) export signal
KSRITSEGEYIPLDQIDINV

SEQ ID NO: 57 - protein, 6 aa, Artificial sequence
  endoplasmic reticulum (ER) export signal
  misc_feature (2)..(2): Xaa can be any naturally occurring amino acid
FXYENE

SEQ ID NO: 58 - protein, 7 aa, Artificial sequence
  endoplasmic reticulum (ER) export signal
FCYENEV

SEQ ID NO: 59 - protein, 21 aa, Artificial sequence
  SRC1-2
SLTARHKILHRLLQEGSPSDI

SEQ ID NO: 60 - protein, 21 aa, Artificial sequence
  PGC1a
QEAEEPSLLKKLLLAPANTQL

SEQ ID NO: 61 - protein, 21 aa, Artificial sequence
  TRAP220-1
SKVSQNPILTSLLQITGNGGS

SEQ ID NO: 62 - protein, 19 aa, Artificial sequence
  SRC3-1
ESKGHKKLLQLLTCSSDDR

SEQ ID NO: 63 - protein, 196 aa, Artificial Sequence
  co-regulator peptide
GQDIQLIPPLINLLMSIEPDVIYAGHDNTKPDTSSSLLTSLNQDLILNEQRMKESSFYSLCLTMWQIPQEFVKLQVSQEEFLCMKVLLLLNTIPLEGLRSQTQFEEMRSSYIRELIKAIGLRQKGVVSSSQRFYQLTKLLDNLHDLVKQLHLYCLNTFIQSRALSVEFPEMMSEVIAAQLPKILAGMVKPLLFHKK

SEQ ID NO: 64 - protein, 25 aa, Artificial sequence
  NCoR (2051-2075)
GHSFADPASNLGLEDIIRKALMGSF

SEQ ID NO: 65 - protein, 25 aa, Artificial sequence
  SRC1
CPSSHSSLTERHKILHRLLQEGSPS

SEQ ID NO: 66 - protein, 21 aa, Artificial sequence
  SRC3
PKKENNALLRYLLDRDDPSDV

SEQ ID NO: 67 - protein, 17 aa, Artificial sequence
  PGC-1
AEEPSLLKKLLLAPANT

SEQ ID NO: 68 - protein, 17 aa, Artificial sequence
  NR0B1
PRQGSILYSMLTSAKQT

SEQ ID NO: 69 - protein, 17 aa, Artificial sequence
  NRIP1
AANNSLLLHLLKSQTIP

SEQ ID NO: 70 - protein, 21 aa, Artificial sequence
  GRIP1-3
PKKKENALLRYLLDKDDTKDI

SEQ ID NO: 71 - protein, 16 aa, Artificial sequence
  CoRNR Box
DAFQLRQLILRGLQDD

SEQ ID NO: 72 - protein, 13 aa, Artificial sequence
  abV
SPGSREWFKDMLS

SEQ ID NO: 73 - protein, 21 aa, Artificial sequence
  TRAP220-2
GNTKNHPMLMNLLKDNPAQDF

SEQ ID NO: 74 - protein, 16 aa, Artificial sequence
  EA2
SSKGVLWRMLAEPVSR

SEQ ID NO: 75 - protein, 15 aa, Artificial sequence
  TA1
SRTLQLDWGTLYWSR

SEQ ID NO: 76 - protein, 15 aa, Artificial sequence
  EAB1
SSNHQSSRLIELLSR

SEQ ID NO: 77 - protein, 19 aa, Artificial sequence
  SRC2
LKEKHKILHRLLQDSSSPV

SEQ ID NO: 78 - protein, 14 aa, Artificial sequence
  SRC1-3
QAQQKSLLQQLLTE

SEQ ID NO: 79 - protein, 21 aa, Artificial sequence
  SRC1-1
KYSQTSHKLVQLLTTTAEQQL

SEQ ID NO: 80 - protein, 21 aa, Artificial sequence
  SRC1-3
KESKDHQLLRYLLDKDEKDLR

SEQ ID NO: 81 - protein, 15 aa, Artificial sequence
  SRC1-4a
PQAQQKSLLQQLLTE

SEQ ID NO: 82 - protein, 15 aa, Artificial sequence
  SRC1-4b
PQAQQKSLRQQLLTE

SEQ ID NO: 83 - protein, 21 aa, Artificial sequence
  GRIP1-1
HDSKGQTKLLQLLTTKSDQME

SEQ ID NO: 84 - protein, 21 aa, Artificial sequence
  GRIP1-2
SLKEKHKILHRLLQDSSSPVD

SEQ ID NO: 85 - protein, 21 aa, Artificial sequence
  AIB1-1
LESKGHKKLLQLLTCSSDDRG

SEQ ID NO: 86 - protein, 21 aa, Artificial sequence
  AIB1-2
LLQEKHRILHKLLQNGNSPAE

SEQ ID NO: 87 - protein, 21 aa, Artificial sequence
  AIB1-3
KKKENNALLRYLLDRDDPSDA

SEQ ID NO: 88 - protein, 21 aa, Artificial sequence
  PGC1b
PEVDELSLLQKLLLATSYPTS

SEQ ID NO: 89 - protein, 21 aa, Artificial sequence
  PRC
VSPREGSSLHKLLTLSRTPPE

SEQ ID NO: 90 - protein, 21 aa, Artificial sequence
  ASC2-1
DVTLTSPLLVNLLQSDISAGH

SEQ ID NO: 91 - protein, 21 aa, Artificial sequence
  ASC2-2
AMREAPTSLSQLLDNSGAPNV

SEQ ID NO: 92 - protein, 21 aa, Artificial sequence
  CBP-1
DAASKHKQLSELLRGGSGSSI

SEQ ID NO: 93 - protein, 21 aa, Artificial sequence
  CBP-2
KRKLIQQQLVLLLHAHKCQRR

SEQ ID NO: 94 - protein, 21 aa, Artificial sequence
  P300
DAASKHKQLSELLRSGSSPNL

SEQ ID NO: 95 - protein, 21 aa, Artificial sequence
  CIA
GHPPAIQSLINLLADNRYLTA

SEQ ID NO: 96 - protein, 21 aa, Artificial sequence
  ARA70-1
TLQQQAQQLYSLLGQFNCLTH

SEQ ID NO: 97 - protein, 21 aa, Artificial sequence
  ARA70-2
GSRETSEKFKLLFQSYNVNDW

SEQ ID NO: 98 - protein, 21 aa, Artificial sequence
  TIF1
NANYPRSILTSLLLNSSQSST

SEQ ID NO: 99 - protein, 21 aa, Artificial sequence
  NSD1
IPIEPDYKFSTLLMMLKDMHD

SEQ ID NO: 100 - protein, 21 aa, Artificial sequence
  SMAP
ATPPPSPLLSELLKKGSLLPT

SEQ ID NO: 101 - protein, 21 aa, Artificial sequence
  Tip60
VDGHERAMLKRLLRIDSKCLH

SEQ ID NO: 102 - protein, 21 aa, Artificial sequence
  ERAP140
HEDLDKVKLIEYYLTKNKEGP

SEQ ID NO: 103 - protein, 21 aa, Artificial sequence
  Nix1
ESPEFCLGLQTLLSLKCCIDL

SEQ ID NO: 104 - protein, 21 aa, Artificial sequence
  LCoR
AATTQNPVLSKLLMADQDSPL

SEQ ID NO: 105 - protein, 30 aa, Artificial sequence
  CoRNR1 (N-CoR)
MGQVPRTHRLITLADHICQIITQDFARNQV

SEQ ID NO: 106 - protein, 14 aa, Artificial sequence
  CoRNR2 (N-CoR)
NLGLEDIIRKALMG

SEQ ID NO: 107 - protein, 40 aa, Artificial sequence
  CoRNR1 (SMRT)
APGVKGHQRVVTLAQHISEVITQDTYRHHPQQLSAPLPAP

SEQ ID NO: 108 - protein, 14 aa, Artificial sequence
  CoRNR2 (SMRT)
NMGLEAIIRKALMG

SEQ ID NO: 109 - protein, 21 aa, Artificial sequence
  RIP140-C
RLTKTNPILYYMLQKGGNSVA

SEQ ID NO: 110 - protein, 21 aa, Artificial sequence
  RIP140-1
QDSIVLTYLEGLLMHQAAGGS

SEQ ID NO: 111 - protein, 21 aa, Artificial sequence
  RIP140-2
KGKQDSTLLASLLQSFSSRLQ

SEQ ID NO: 112 - protein, 21 aa, Artificial sequence
  RIP140-3
CYGVASSHLKTLLKKSKVKDQ

SEQ ID NO: 113 - protein, 21 aa, Artificial sequence
  RIP140-4
KPSVACSQLALLLSSEAHLQQ

SEQ ID NO: 114 - protein, 21 aa, Artificial sequence
  RIP140-5
KQAANNSLLLHLLKSQTIPKP

SEQ ID NO: 115 - protein, 21 aa, Artificial sequence
  RIP140-6
NSHQKVTLLQLLLGHKNEENV

SEQ ID NO: 116 - protein, 21 aa, Artificial sequence
  RIP140-7
NLLERRTVLQLLLGNPTKGRV

SEQ ID NO: 117 - protein, 21 aa, Artificial sequence
  RIP140-8
FSFSKNGLLSRLLRQNQDSYL

SEQ ID NO: 118 - protein, 21 aa, Artificial sequence
  RIP140-9
RESKSFNVLKQLLLSENCVRD

SEQ ID NO: 119 - protein, 21 aa, Artificial sequence
  PRIC285-1
ELNADDAILRELLDESQKVMV

SEQ ID NO: 120 - protein, 21 aa, Artificial sequence
  PRIC285-2
YENLPPAALRKLLRAEPERYR

SEQ ID NO: 121 - protein, 21 aa, Artificial sequence
  PRIC285-3
MAFAGDEVLVQLLSGDKAPEG

SEQ ID NO: 122 - protein, 21 aa, Artificial sequence
  PRIC285-4
SCCYLCIRLEGLLAPTASPRP

SEQ ID NO: 123 - protein, 21 aa, Artificial sequence
  PRIC285-5
PSNKSVDVLAGLLLRRMELKP

SEQ ID NO: 124 - protein, 219 aa, Artificial sequence
  calmodulin polypeptide
GESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMV

SEQ ID NO: 125 - protein, 21 aa, Artificial sequence
  calmodulin-binding polypeptide
NARRKLAGAILFTMLATRNFS

SEQ ID NO: 126 - protein, 26 aa, Artificial sequence
  calmodulin-binding polypeptide
KRRWKKNFIAVSAANRFKKISSSGAL

SEQ ID NO: 127 - protein, 26 aa, Artificial sequence
  calmodulin-binding polypeptide with A14F substitution
KRRWKKNFIAVSAFNRFKKISSSGAL

SEQ ID NO: 128 - protein, 22 aa, Artificial sequence
  calmodulin-binding polypeptide
FNARRKLKGAILTTMLFTRNFS

SEQ ID NO: 129 - protein, 46 aa, Artificial sequence
  calmodulin-binding polypeptide  with K8A substitution
FNARRKLAGAILFTMLATRNFSGSFNARRKLAGAILFTMLATRNFS

SEQ ID NO: 130 - protein, 22 aa, Artificial sequence
  calmodulin-binding polypeptide with T13F substitution
FNARRKLAGAILFTMLATRNFS

SEQ ID NO: 131 - protein, 148 aa, Artificial sequence
  calmodulin
MDQLTEEQIAEFKEAFSLFDKDGDGTITTKELGTVMRSLGQNPTEAELQDMINEVDADGDGTIDFPEFLTMMARKMKYTDSEEEIREAFRVFDKDGNGYISAAELRHVMTNLGEKLTDEEVDEMIREADIDGDGQVNYEEFVQMMTAK

SEQ ID NO: 132 - protein, 148 aa, Artificial sequence
  calmodulin polypeptide with F19L substitution
MDQLTEEQIAEFKEAFSLLDKDGDGTITTKELGTVMRSLGQNPTEAELQDMINEVDADGDGTIDFPEFLTMMARKMKYTDSEEEIREAFRVFDKDGNGYISAAELRHVMTNLGEKLTDEEVDEMIREADIDGDGQVNYEEFVQMMTAK

SEQ ID NO: 133 - protein, 148 aa, Artificial sequence
  calmodulin polypeptide with F19L and  V35G substitutions
MDQLTEEQIAEFKEAFSLLDKDGDGTITTKELGTGMRSLGQNPTEAELQDMINEVDADGDGTIDFPEFLTMMARKMKYTDSEEEIREAFRVFDKDGNGYISAAELRHVMTNLGEKLTDEEVDEMIREADIDGDGQVNYEEFVQMMTAK

SEQ ID NO: 134 - protein, 187 aa, Homo sapiens
MPEVERKPKITASRKLLLKSLMLAKAKECWEQEHEEREAEKVRYLAERIPTLQTRGLSLSALQDLCRELHAKVEVVDEERYDIEAKCLHNTREIKDLKLKVMDLRGKFKRPPLRRVRVSADAMLRALLGSKHKVSMDLRANLKSVKKEDTEKERPVEVGDWRKNVEAMSGMEGRKKMFDAAKSPTSQ

SEQ ID NO: 135 - protein, 20 aa, Artificial sequence
  troponin I polypeptide
KDLKLKVMDLRGKFKRPPLR

SEQ ID NO: 136 - protein, 230 aa, Artificial Sequence
  ligand binding domain of an androgen receptor
DNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 137 - protein, 25 aa, Artificial sequence
  troponin I polypeptide
RMSADAMLKALLGSKHKVAMDLRAN

SEQ ID NO: 138 - protein, 44 aa, Artificial sequence
  troponin I polypeptide
NQKLFDLRGKFKRPPLRRVRMSADAMLKALLGSKHKVAMDLRAN

SEQ ID NO: 139 - protein, 160 aa, Homo sapiens
MTDQQAEARSYLSEEMIAEFKAAFDMFDADGGGDISVKELGTVMRMLGQTPTKEELDAIIEEVDEDGSGTIDFEEFLVMMVRQMKEDAKGKSEEELAECFRIFDRNADGYIDPGELAEIFRASGEHVTDEEIESLMKDGDKNNDGRIDFDEFLKMMEGVQ

SEQ ID NO: 140 - protein, 160 aa, Rattus norvegicus
MTDQQAEARSYLSEEMIAEFKAAFDMFDADGGGDISVKELGTVMRMLGQTPTKEELDAIIEEVDEDGSGTIDFEEFLVMMVRQMKEDAKGKSEEELAECFRIFDRDANGYIDAEELAEIFRASGEHVTDEEIESLMKDGDKNNDGRIDFDEFLKMMEGVQ

SEQ ID NO: 141 - protein, 409 aa, Homo sapiens
MGEKPGTRVFKKSSPNCKLTVYLGKRDFVDHLDKVDPVDGVVLVDPDYLKDRKVFVTLTCAFRYGREDLDVLGLSFRKDLFIATYQAFPPVPNPPRPPTRLQDRLLRKLGQHAHPFFFTIPQNLPCSVTLQPGPEDTGKACGVDFEIRAFCAKSLEEKSHKRNSVRLVIRKVQFAPEKPGPQPSAETTRHFLMSDRSLHLEASLDKELYYHGEPLNVNVHVTNNSTKTVKKIKVSVRQYADICLFSTAQYKCPVAQLEQDDQVSPSSTFCKVYTITPLLSDNREKRGLALDGKLKHEDTNLASSTIVKEGANKEVLGILVSYRVKVKLVVSRGGDVSVELPFVLMHPKPHDHIPLPRPQSAAPETDVPVDTNLIEFDTNYATDDDIVFEDFARLRLKGMKDDDYDDQLC

SEQ ID NO: 142 - protein, 142 aa, Artificial sequence
  LOV2 polypeptide
DLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDEAAK

SEQ ID NO: 143 - protein, 142 aa, Artificial sequence
  LOV domain light-activated polypeptide
SLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREAVMLIKKTAEEIDEAAK

SEQ ID NO: 144 - protein, 142 aa, Artificial sequence
  LOV polypeptide
SRATTLERIEKSFVITDPRLPDNPIIFVSDSFLQLTEYSREEILGRNCRFLQGPETDRATVRKIRDAIDNQTEVTVQLINYTKSGKKFWNLFHLQPMRDQKGDVQYFIGVQLDGTERVRDAAEREAVMLVKKTAEEIDEAAK

SEQ ID NO: 145 - protein, 142 aa, Artificial sequence
  LOV polypeptide
SRATTLERIEKSFVITDPRLPDNPVIFVSDSFLQLTEYSREEILGRNCRFLQGPETDRATVRKIRDAIDNQTEVTVQLINYTKSGKKFWNLFHLQPMRDQKGDVQYFIGVQLDGTERVRDAAEREAVMLVKKTAEEIDEAAK

SEQ ID NO: 146 - protein, 142 aa, Artificial sequence
  LOV polypeptide
SRATTLERIEKSFVITDPRLPDNPIIFVSDSFLQLTEYSREEILGRNCRFLQGPETDRATVRKIRDAIDNQTEVTVQLINYTKSGKKFWNVFHLQPMRDYKGDVQYFIGVQLDGTERLHGAAEREAVCLVKKTAFQIAEAAK

SEQ ID NO: 147 - protein, 138 aa, Artificial sequence
  LOV polypeptide
SRATTLERIEKSFVITDPRLPDNPIIFVSDSFLQLTEYSREEILGRNCRFLQGPETDRATVRKIRDAIDNQTEVTVQLINYTKSGKKFWNLFHLQPMRDQKGDVQYFIGVQLDGTERVRDAAEREAVMLVKKTAEEID

SEQ ID NO: 148 - protein, 138 aa, Artificial sequence
  LOV polypeptide
SRATTLERIEKSFVITDPRLPDNPIIFVSDSFLQLTEYSREEILGRNCRFLQGPETDRATVRKIRDAIDNQTEVTVQLINYTKSGKKFWNVFHLQPMRDYKGDVQYFIGVQLDGTERLHGAAEREAVCLVKKTAFQIA

SEQ ID NO: 149 - protein, 138 aa, Artificial sequence
  LOV polypeptide
FRATTLERIEKSFVITDPRLPDNPIIFVSDSFLQLTEYSREEILGRNCRFLQGPETDRATVRKIRDAIDNQTEVTVQLINYTKSGKKFWNVFHLQPMRDYKGDVQYFIGVQLDGTERLHGAAEREAVCLVKKTAFQIA

SEQ ID NO: 150 - protein, 142 aa, Artificial sequence
  LOV polypeptide
SRATTLERIEKSFVITDPRLPDNPIIFVSDSFLQLTEYSREEILGRNCRFLQGPETDRATVRKIRDAIDNQTEVTVQLINYTKSGKKFWNVFHLQPMRDYKGDVQYFIGVQLDGTERLHGAAEREAVCLVKKTAFEIDEAAK

SEQ ID NO: 151 - protein, 149 aa, Artificial sequence
  LOV polypeptide
SRATTLERIEKSFVITDPRLPDNPIIFVSDSFLQLTEYSREEILGRNCRFLQGPETDRATVRKIRDAIDNQTEVTVQLINYTKSGKKFWNLFHLQPMRDQKGDVQYFIGVQLDGTERVRDAAEREAVMLVKKTAEEIDEAAKENLYFQM

SEQ ID NO: 152 - protein, 149 aa, Artificial sequence
  LOV polypeptide
SRATTLERIEKSFVITDPRLPDNPIIFVSDSFLQLTEYSREEILGRNCRFLQGPETDRATVRKIRDAIDNQTEVTVQLINYTKSGKKFWNVFHLQPMRDYKGDVQYFIGVQLDGTERLHGAAEREAVCLVKKTAFEIDEAAKENLYFQM

SEQ ID NO: 153 - protein, 145 aa, Artificial sequence
  LOV polypeptide
FRATTLERIEKSFVITDPRLPDNPIIFVSDSFLQLTEYSREEILGRNCRFLQGPETDRATVRKIRDAIDNQTEVTVQLINYTKSGKKFWNVFHLQPMRDYKGDVQYFIGVQLDGTERLHGAAEREAVCLVKKTAFQIAENLYFQM

SEQ ID NO: 154 - protein, 145 aa, Artificial sequence
  LOV polypeptide
SRATTLERIEKSFVITDPRLPDNPIIFVSDSFLQLTEYSREEILGRNCRFLQGPETDRATVRKIRDAIDNQTEVTVQLINYTKSGKKFWNLFHLQPMRDQKGDVQYFIGVQLDGTERVRDAAEREAVMLVKKTAEEIDENLYFQG

SEQ ID NO: 155 - protein, 145 aa, Artificial sequence
  LOV polypeptide
FRATTLERIEKSFVITDPRLPDNPIIFVSDSFLQLTEYSREEILGRNCRFLQGPETDRATVRKIRDAIDNQTEVTVQLINYTKSGKKFWNVFHLQPMRDYKGDVQYFIGVQLDGTERLHGAAEREAVCLVKKTAFQIAENLYFQG

SEQ ID NO: 156 - protein, 7 aa, Artificial sequence
  cleavage sequence of MMP-9
PLQGMTS

SEQ ID NO: 157 - protein, 6 aa, Artificial sequence
  cleavage sequence of MMP-9
PLQGMT

SEQ ID NO: 158 - protein, 7 aa, Artificial sequence
  tobacco etch virus (TEV) protease cleavage site
ENLYFQS

SEQ ID NO: 159 - protein, 7 aa, Artificial sequence
  tobacco etch virus (TEV) protease cleavage site
ENLYFQY

SEQ ID NO: 160 - protein, 7 aa, Artificial sequence
  tobacco etch virus (TEV) protease cleavage site
ENLYFQL

SEQ ID NO: 161 - protein, 5 aa, Artificial sequence
  enterokinase cleavage site
DDDDK

SEQ ID NO: 162 - protein, 4 aa, Artificial sequence
  thrombin cleavage site
LVPR

SEQ ID NO: 163 - protein, 6 aa, Artificial sequence
  proteolytically cleavable linker
LVPRGS

SEQ ID NO: 164 - protein, 8 aa, Artificial sequence
  proteolytically cleavable linker
LEVLFQGP

SEQ ID NO: 165 - protein, 10 aa, Artificial sequence
  proteolytically cleavable linker
CGLVPAGSGP

SEQ ID NO: 166 - protein, 12 aa, Artificial sequence
  proteolytically cleavable linker
SLLKSRMVPNFN

SEQ ID NO: 167 - protein, 12 aa, Artificial sequence
  proteolytically cleavable linker
SLLIARRMPNFN

SEQ ID NO: 168 - protein, 12 aa, Artificial sequence
  proteolytically cleavable linker
SKLVQASASGVN

SEQ ID NO: 169 - protein, 12 aa, Artificial sequence
  proteolytically cleavable linker
SSYLKASDAPDN

SEQ ID NO: 170 - protein, 12 aa, Artificial sequence
  proteolytically cleavable linker
RPKPQQFFGLMN

SEQ ID NO: 171 - protein, 12 aa, Artificial sequence
  proteolytically cleavable linker
SLRPLALWRSFN

SEQ ID NO: 172 - protein, 12 aa, Artificial sequence
  proteolytically cleavable linker
SPQGIAGQRNFN

SEQ ID NO: 173 - protein, 14 aa, Artificial sequence
  proteolytically cleavable linker
DVDERDVRGFASFL

SEQ ID NO: 174 - protein, 12 aa, Artificial sequence
  proteolytically cleavable linker
SLPLGLWAPNFN

SEQ ID NO: 175 - protein, 12 aa, Artificial sequence
  proteolytically cleavable linker
SLLIFRSWANFN

SEQ ID NO: 176 - protein, 12 aa, Artificial sequence
  proteolytically cleavable linker
SGVVIATVIVIT

SEQ ID NO: 177 - protein, 12 aa, Artificial sequence
  proteolytically cleavable linker
SLGPQGIWGQFN

SEQ ID NO: 178 - protein, 12 aa, Artificial sequence
  proteolytically cleavable linker
KKSPGRVVGGSV

SEQ ID NO: 179 - protein, 12 aa, Artificial sequence
  proteolytically cleavable linker
PQGLLGAPGILG

SEQ ID NO: 180 - protein, 31 aa, Artificial sequence
  proteolytically cleavable linker
HGPEGLRVGFYESDVMGRGHARLVHVEEPHT

SEQ ID NO: 181 - protein, 12 aa, Artificial sequence
  proteolytically cleavable linker
GPQGLAGQRGIV

SEQ ID NO: 182 - protein, 12 aa, Artificial sequence
  proteolytically cleavable linker
GGSGQRGRKALE

SEQ ID NO: 183 - protein, 12 aa, Artificial sequence
  proteolytically cleavable linker
SLSALLSSDIFN

SEQ ID NO: 184 - protein, 12 aa, Artificial sequence
  proteolytically cleavable linker
SLPRFKIIGGFN

SEQ ID NO: 185 - protein, 12 aa, Artificial sequence
  proteolytically cleavable linker
SLLGIAVPGNFN

SEQ ID NO: 186 - protein, 12 aa, Artificial sequence
  proteolytically cleavable linker
FFKNIVTPRTPP

SEQ ID NO: 187 - protein, 7 aa, Artificial sequence
  proteolytically cleavable linker
  misc_feature (7)..(7): Xaa can be any naturally occurring amino acid
ENLYFQX

SEQ ID NO: 188 - protein, 7 aa, Artificial sequence
  proteolytically cleavable linker
ENLYFQG

SEQ ID NO: 189 - protein, 7 aa, Artificial sequence
  proteolytically cleavable linker
ENLYFQW

SEQ ID NO: 190 - protein, 7 aa, Artificial sequence
  proteolytically cleavable linker
ENLYFQM

SEQ ID NO: 191 - protein, 7 aa, Artificial sequence
  proteolytically cleavable linker
ENLYFQH

SEQ ID NO: 192 - protein, 7 aa, Artificial sequence
  proteolytically cleavable linker
ENLYFQN

SEQ ID NO: 193 - protein, 7 aa, Artificial sequence
  proteolytically cleavable linker
ENLYFQA

SEQ ID NO: 194 - protein, 7 aa, Artificial sequence
  proteolytically cleavable linker
ENLYFQQ

SEQ ID NO: 195 - protein, 7 aa, Artificial sequence
  proteolytically cleavable linker
DEVVECS

SEQ ID NO: 196 - protein, 10 aa, Artificial sequence
  proteolytically cleavable linker
DEAEDVVECS

SEQ ID NO: 197 - protein, 11 aa, Artificial sequence
  proteolytically cleavable linker
EDAAEEVVECS

SEQ ID NO: 198 - protein, 6 aa, Artificial sequence
  proteolytically cleavable linker
PLFAAR

SEQ ID NO: 199 - protein, 11 aa, Artificial sequence
  proteolytically cleavable linker
QQEVYGMMPRD

SEQ ID NO: 200 - protein, 321 aa, Escherichia coli
MKDNTVPLKLIALLANGEFHSGEQLGETLGMSRAAINKHIQTLRDWGVDVFTVPGKGYSLPEPIQLLNAEEILSQLDGGSVAVLPVIDSTNQYLLDRIGELKSGDACVAEYQQAGRGRRGRKWFSPFGANLYLSMFWRLEQGPAAAIGLSLVIGIVMAEVLRKLGADKVRVKWPNDLYLQDRKLAGILVELTGKTGDAAQIVIGAGINMAMRRVEESVVNQGWITLQEAGINLDRNTLAAMLIRELRAALELFEQEGLAPYLSRWEKLDNFINRPVKLIIGDKEIFGISRGIDKQGALLLEQDGIIKPWMGGEISLRSAEK

SEQ ID NO: 201 - protein, 250 aa, Glycine max
MGKSYPTVSADYQKAVEKAKKKLRGFIAEKRCAPLMLRLAWHSAGTFDKGTKTGGPFGTIKHPAELAHSANNGLDIAVRLLEPLKAEFPILSYADFYQLAGVVAVEVTGGPEVPFHPGREDKPEPPPEGRLPDATKGSDHLRDVFGKAMGLTDQDIVALSGGHTIGAAHKERSGFEGPWTSNPLIFDNSYFTELLSGEKEGLLQLPSDKALLSDPVFRPLVDKYAADEDAFFADYAEAHQKLSELGFADA

SEQ ID NO: 202 - protein, 49 aa, Anthopleura xanthogrammica
GVPCLCDSDGPRPRGNTLSGILWFYPSGCPSGWHNCKAHGPNIGWCCKK

SEQ ID NO: 203 - protein, 79 aa, Anthopleura xanthogrammica
MKTQVLALFVLCVLFCLAESRTTLNKRNDIEKRIECKCEGDAPDLSHMTGTVYFSCKGGDGSWSKCNTYTAVADCCHQA

SEQ ID NO: 204 - DNA, 54 bp, Artificial Sequence
  telRL site
tatcagcacacaattgcccattatacgcgcgtataatggactattgtgtgctga

SEQ ID NO: 205 - DNA, 42 bp, Artificial sequence
  pal site
acctatttcagcatactacgcgcgtagtatgctgaaataggt

SEQ ID NO: 206 - DNA, 22 bp, Artificial sequence
  phi K02 telRL site
ccattatacgcgcgtataatgg

SEQ ID NO: 207 - DNA, 33 bp, Artificial sequence
  loxP site
taacttcgtatagcatacattatacgaagttat

SEQ ID NO: 208 - DNA, 34 bp, Artificial sequence
  FRT site
gaagttcctattctctagaaagtataggaacttc

SEQ ID NO: 209 - DNA, 100 bp, Artificial Sequence
  phiC31 attP site
cccaggtcagaagcggttttcgggagtagtgccccaactggggtaacctttgagttctctcagttgggggcgtagggtcgccgacaygacacaaggggtt

SEQ ID NO: 210 - DNA, 101 bp, Artificial Sequence
  ? attP site
tgatagtgacctgttcgtttgcaacacattgatgagcaatgcttttttataatgccaactttgtacaaaaaagctgaacgagaaacgtaaaatgatataaa

SEQ ID NO: 211 - protein, 16 aa, Artificial sequence
  mitochondrial localization sequence
LGRVIPRKIASRASLM

SEQ ID NO: 212 - protein, 30 aa, Artificial sequence
  mitochondrial localization sequence
MSVLTPLLLRGLTGSARRLPVPRAKIHSLL

SEQ ID NO: 213 - protein, 27 aa, Artificial sequence
  Nav1.6 soma localization signal
TVRVPIAVGESDFENLNTEDVSSESDP

SEQ ID NO: 214 - protein, 7 aa, Artificial sequence
  NLS of the SV40 virus large T-antigen
PKKKRKV

SEQ ID NO: 215 - protein, 16 aa, Artificial sequence
  NLS from nucleoplasmin
KRPAATKKAGQAKKKK

SEQ ID NO: 216 - protein, 9 aa, Artificial sequence
  NLS from nucleoplasmin
PAAKRVKLD

SEQ ID NO: 217 - protein, 11 aa, Artificial sequence
  NLS from nucleoplasmin
RQRRNELKRSP

SEQ ID NO: 218 - protein, 38 aa, Artificial sequence
  hRNPA1 M9 NLS
NQSSNFGPMKGGNFGGRSSGPYGGGGQYFAKPRNQGGY

SEQ ID NO: 219 - protein, 42 aa, Artificial sequence
  IBB domain from importin-alpha
RMRIZFKNKGKDTAELRRRRVEVSVELRKAKKDEQILKRRNV

SEQ ID NO: 220 - protein, 8 aa, Artificial sequence
  myoma T protein sequence
VSRKRPRP

SEQ ID NO: 221 - protein, 8 aa, Artificial sequence
  myoma T protein sequence
PPKKARED

SEQ ID NO: 222 - protein, 8 aa, Homo sapiens
PQPKKKPL

SEQ ID NO: 223 - protein, 12 aa, Mus musculus
SALIKKKKKMAP

SEQ ID NO: 224 - protein, 5 aa, Influenza A virus
DRLRR

SEQ ID NO: 225 - protein, 7 aa, Influenza A virus
PKQKKRK

SEQ ID NO: 226 - protein, 10 aa, Hepatitis D virus
RKLKKKIKKL

SEQ ID NO: 227 - protein, 10 aa, Mus musculus
REKKKFLKRR

SEQ ID NO: 228 - protein, 20 aa, Homo sapiens
KRKGDEVDGVDEVAKKKSKK

SEQ ID NO: 229 - protein, 17 aa, Homo sapiens
RKCLQAGMNLEARKTKK

SEQ ID NO: 230 - protein, 11 aa, Human immunodeficiency virus
YGRKKRRQRRR

SEQ ID NO: 231 - protein, 12 aa, Artificial sequence
  protein transduction domain
RRQRRTSKLMKR

SEQ ID NO: 232 - protein, 27 aa, Artificial sequence
  Transportan
GWTLNSAGYLLGKINLKALAALAKKIL

SEQ ID NO: 233 - protein, 33 aa, Artificial sequence
  protein transduction domain
KALAWEAKLAKALAKALAKHLAKALAKALKCEA

SEQ ID NO: 234 - protein, 16 aa, Artificial sequence
  protein transduction domain
RQIKIWFQNRRMKWKK

SEQ ID NO: 235 - protein, 11 aa, Artificial sequence
  protein transduction domain
YGRKKRRQRRR

SEQ ID NO: 236 - protein, 9 aa, Artificial sequence
  protein transduction domain
RKKRRQRRR

SEQ ID NO: 237 - protein, 8 aa, Artificial sequence
  protein transduction domain
RKKRRQRR

SEQ ID NO: 238 - protein, 11 aa, Artificial sequence
  protein transduction domain
YARAAARQARA

SEQ ID NO: 239 - protein, 11 aa, Artificial sequence
  protein transduction domain
THRLPRRRRRR

SEQ ID NO: 240 - protein, 11 aa, Artificial sequence
  protein transduction domain
GGRRARRRRRR

SEQ ID NO: 241 - protein, 310 aa, Artificial sequence
  ChR2 depolarizing opsin
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVILIHLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVP

SEQ ID NO: 242 - protein, 340 aa, Artificial sequence
  ChR2 depolarizing opsin with ER export and trafficking signal sequence
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVILIHLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVPAAAKSRITSEGEYIPLDQIDINVFCYENEV

SEQ ID NO: 243 - protein, 310 aa, Artificial sequence
  ChR2SSFO
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTSPVILIHLSNLTGLSNDYSRRTMGLLVSAIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVP

SEQ ID NO: 244 - protein, 340 aa, Artificial sequence
  ChR2SSFO with ER export and trafficking signal sequences
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTSPVILIHLSNLTGLSNDYSRRTMGLLVSAIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVPAAAKSRITSEGEYIPLDQIDINVFCYENEV

SEQ ID NO: 245 - protein, 300 aa, Volvox carteri
MDYPVARSLIVRYPTDLGNGTVCMPRGQCYCEGWLRSRGTSIEKTIAITLQWVVFALSVACLGWYAYQAWRATCGWEEVYVALIEMMKSIIEAFHEFDSPATLWLSSGNGVVWMRYGEWLLTCPVLLIHLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEED

SEQ ID NO: 246 - protein, 330 aa, Artificial sequence
  VChR1 with ER export and trafficking signal sequences
MDYPVARSLIVRYPTDLGNGTVCMPRGQCYCEGWLRSRGTSIEKTIAITLQWVVFALSVACLGWYAYQAWRATCGWEEVYVALIEMMKSIIEAFHEFDSPATLWLSSGNGVVWMRYGEWLLTCPVLLIHLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEEDAAAKSRITSEGEYIPLDQIDINVFCYENEV

SEQ ID NO: 247 - protein, 344 aa, Artificial sequence
  C1V1
MSRRPWLLALALAVALAAGSAGASTGSDATVPVATQDGPDYVFHRAHERMLFQTSYTLENNGSVICIPNNGQCFCLAWLKSNGTNAEKLAANILQWITFALSALCLMFYGYQTWKSTCGWEEIYVATIEMIKFIIEYFHEFDEPAVIYSSNGNKTVWLRYAEWLLTCPVLLIHLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEED

SEQ ID NO: 248 - protein, 374 aa, Artificial sequence
  C1V1 with ER export and trafficking signal sequences
MSRRPWLLALALAVALAAGSAGASTGSDATVPVATQDGPDYVFHRAHERMLFQTSYTLENNGSVICIPNNGQCFCLAWLKSNGTNAEKLAANILQWITFALSALCLMFYGYQTWKSTCGWEEIYVATIEMIKFIIEYFHEFDEPAVIYSSNGNKTVWLRYAEWLLTCPVLLIHLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEEDAAAKSRITSEGEYIPLDQIDINVFCYENEV

SEQ ID NO: 249 - protein, 348 aa, Artificial sequence
  C1C2
MSRRPWLLALALAVALAAGSAGASTGSDATVPVATQDGPDYVFHRAHERMLFQTSYTLENNGSVICIPNNGQCFCLAWLKSNGTNAEKLAANILQWITFALSALCLMFYGYQTWKSTCGWEEIYVATIEMIKFIIEYFHEFDEPAVIYSSNGNKTVWLRYAEWLLTCPVILIHLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAV

SEQ ID NO: 250 - protein, 378 aa, Artificial sequence
  C1C2 with ER export and trafficking signal sequences
MSRRPWLLALALAVALAAGSAGASTGSDATVPVATQDGPDYVFHRAHERMLFQTSYTLENNGSVICIPNNGQCFCLAWLKSNGTNAEKLAANILQWITFALSALCLMFYGYQTWKSTCGWEEIYVATIEMIKFIIEYFHEFDEPAVIYSSNGNKTVWLRYAEWLLTCPVILIHLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVAAAKSRITSEGEYIPLDQIDINVFCYENEV

SEQ ID NO: 251 - protein, 350 aa, Artificial sequence
  ReaChR (red-shifted ChR)
MVSRRPWLLALALAVALAAGSAGASTGSDATVPVATQDGPDYVFHRAHERMLFQTSYTLENNGSVICIPNNGQCFCLAWLKSNGTNAEKLAANILQWVTFALSVACLGWYAYQAWRATCGWEEVYVALIEMMKSIIEAFHEFDSPATLWLSSGNGVVWMRYGEWLLTCPVILIHLSNLTGLKDDYSKRTMGLLVSDVGCIVWGATSAMCTGWTKILFFLISLSYGMYTYFHAAKVYIEAFHTVPKGLCRQLVRAMAWLFFVSWGMFPVLFLLGPEGFGHISPYGSAIGHSILDLIAKNMWGVLGNYLRVKIHEHILLYGDIRKKQKITIAGQEMEVETLVAEEEDKYESS

SEQ ID NO: 252 - protein, 380 aa, Artificial sequence
  ReaChR (red-shifted ChR) with ER export and trafficking signal sequences
MVSRRPWLLALALAVALAAGSAGASTGSDATVPVATQDGPDYVFHRAHERMLFQTSYTLENNGSVICIPNNGQCFCLAWLKSNGTNAEKLAANILQWVTFALSVACLGWYAYQAWRATCGWEEVYVALIEMMKSIIEAFHEFDSPATLWLSSGNGVVWMRYGEWLLTCPVILIHLSNLTGLKDDYSKRTMGLLVSDVGCIVWGATSAMCTGWTKILFFLISLSYGMYTYFHAAKVYIEAFHTVPKGLCRQLVRAMAWLFFVSWGMFPVLFLLGPEGFGHISPYGSAIGHSILDLIAKNMWGVLGNYLRVKIHEHILLYGDIRKKQKITIAGQEMEVETLVAEEEDKYESSAAAKSRITSEGEYIPLDQIDINVFCYENEV

SEQ ID NO: 253 - protein, 316 aa, Artificial sequence
  SdChR (CheRiff)
MGGAPAPDAHSAPPGNDSAGGSEYHAPAGYQVNPPYHPVHGYEEQCSSIYIYYGALWEQETARGFQWFAVFLSALFLAFYGWHAYKASVGWEEVYVCSVELIKVILEIYFEFTSPAMLFLYGGNITPWLRYAEWLLTCPVILIHLSNITGLSEEYNKRTMALLVSDLGTICMGVTAALATGWVKWLFYCIGLVYGTQTFYNAGIIYVESYYIMPAGGCKKLVLAMTAVYYSSWLMFPGLFIFGPEGMHTLSVAGSTIGHTIADLLSKNIWGLLGHFLRIKIHEHIIMYGDIRRPVSSQFLGRKVDVLAFVTEEDKV

SEQ ID NO: 254 - protein, 346 aa, Artificial sequence
  SdChR (CheRiff) with ER export and trafficking signal sequences
MGGAPAPDAHSAPPGNDSAGGSEYHAPAGYQVNPPYHPVHGYEEQCSSIYIYYGALWEQETARGFQWFAVFLSALFLAFYGWHAYKASVGWEEVYVCSVELIKVILEIYFEFTSPAMLFLYGGNITPWLRYAEWLLTCPVILIHLSNITGLSEEYNKRTMALLVSDLGTICMGVTAALATGWVKWLFYCIGLVYGTQTFYNAGIIYVESYYIMPAGGCKKLVLAMTAVYYSSWLMFPGLFIFGPEGMHTLSVAGSTIGHTIADLLSKNIWGLLGHFLRIKIHEHIIMYGDIRRPVSSQFLGRKVDVLAFVTEEDKVAAAKSRITSEGEYIPLDQIDINVFCYENEV

SEQ ID NO: 255 - protein, 350 aa, Artificial sequence
  CnChR1 (Chrimson)
MAELISSATRSLFAAGGINPWPNPYHHEDMGCGGMTPTGECFSTEWWCDPSYGLSDAGYGYCFVEATGGYLVVGVEKKQAWLHSRGTPGEKIGAQVCQWIAFSIAIALLTFYGFSAWKATCGWEEVYVCCVEVLFVTLEIFKEFSSPATVYLSTGNHAYCLRYFEWLLSCPVILIKLSNLSGLKNDYSKRTMGLIVSCVGMIVFGMAAGLATDWLKWLLYIVSCIYGGYMYFQAAKCYVEANHSVPKGHCRMVVKLMAYAYFASWGSYPILWAVGPEGLLKLSPYANSIGHSICDIIAKEFWTFLAHHLRIKIHEHILIHGDIRKTTKMEIGGEEVEVEEFVEEEDEDTV

SEQ ID NO: 256 - protein, 380 aa, Artificial sequence
  CnChR1 (Chrimson) with ER export and trafficking signal sequences
MAELISSATRSLFAAGGINPWPNPYHHEDMGCGGMTPTGECFSTEWWCDPSYGLSDAGYGYCFVEATGGYLVVGVEKKQAWLHSRGTPGEKIGAQVCQWIAFSIAIALLTFYGFSAWKATCGWEEVYVCCVEVLFVTLEIFKEFSSPATVYLSTGNHAYCLRYFEWLLSCPVILIKLSNLSGLKNDYSKRTMGLIVSCVGMIVFGMAAGLATDWLKWLLYIVSCIYGGYMYFQAAKCYVEANHSVPKGHCRMVVKLMAYAYFASWGSYPILWAVGPEGLLKLSPYANSIGHSICDIIAKEFWTFLAHHLRIKIHEHILIHGDIRKTTKMEIGGEEVEVEEFVEEEDEDTVAAAKSRITSEGEYIPLDQIDINVFCYENEV

SEQ ID NO: 257 - protein, 345 aa, Artificial sequence
  Cs Chrimson
MSRLVAASWLLALLLCGITSTTTASSAPAASSTDGTAAAAVSHYAMNGFDELAKGAVVPEDHFVCGPADKCYCSAWLHSRGTPGEKIGAQVCQWIAFSIAIALLTFYGFSAWKATCGWEEVYVCCVEVLFVTLEIFKEFSSPATVYLSTGNHAYCLRYFEWLLSCPVILIKLSNLSGLKNDYSKRTMGLIVSCVGMIVFGMAAGLATDWLKWLLYIVSCIYGGYMYFQAAKCYVEANHSVPKGHCRMVVKLMAYAYFASWGSYPILWAVGPEGLLKLSPYANSIGHSICDIIAKEFWTFLAHHLRIKIHEHILIHGDIRKTTKMEIGGEEVEVEEFVEEEDEDTV

SEQ ID NO: 258 - protein, 375 aa, Artificial sequence
  Cs Chrimson with ER export and trafficking signal sequences
MSRLVAASWLLALLLCGITSTTTASSAPAASSTDGTAAAAVSHYAMNGFDELAKGAVVPEDHFVCGPADKCYCSAWLHSRGTPGEKIGAQVCQWIAFSIAIALLTFYGFSAWKATCGWEEVYVCCVEVLFVTLEIFKEFSSPATVYLSTGNHAYCLRYFEWLLSCPVILIKLSNLSGLKNDYSKRTMGLIVSCVGMIVFGMAAGLATDWLKWLLYIVSCIYGGYMYFQAAKCYVEANHSVPKGHCRMVVKLMAYAYFASWGSYPILWAVGPEGLLKLSPYANSIGHSICDIIAKEFWTFLAHHLRIKIHEHILIHGDIRKTTKMEIGGEEVEVEEFVEEEDEDTVAAAKSRITSEGEYIPLDQIDINVFCYENEV

SEQ ID NO: 259 - protein, 325 aa, Artificial sequence
  ShChR1 (Chronos)
METAATMTHAFISAVPSAEATIRGLLSAAAVVTPAADAHGETSNATTAGADHGCFPHINHGTELQHKIAVGLQWFTVIVAIVQLIFYGWHSFKATTGWEEVYVCVIELVKCFIELFHEVDSPATVYQTNGGAVIWLRYSMWLLTCPVILIHLSNLTGLHEEYSKRTMTILVTDIGNIVWGITAAFTKGPLKILFFMIGLFYGVTCFFQIAKVYIESYHTLPKGVCRKICKIMAYVFFCSWLMFPVMFIAGHEGLGLITPYTSGIGHLILDLISKNTWGFLGHHLRVKIHEHILIHGDIRKTTTINVAGENMEIETFVDEEEEGGV

SEQ ID NO: 260 - protein, 355 aa, Artificial sequence
  ShChR1 (Chronos) with ER export and trafficking signalsequences
METAATMTHAFISAVPSAEATIRGLLSAAAVVTPAADAHGETSNATTAGADHGCFPHINHGTELQHKIAVGLQWFTVIVAIVQLIFYGWHSFKATTGWEEVYVCVIELVKCFIELFHEVDSPATVYQTNGGAVIWLRYSMWLLTCPVILIHLSNLTGLHEEYSKRTMTILVTDIGNIVWGITAAFTKGPLKILFFMIGLFYGVTCFFQIAKVYIESYHTLPKGVCRKICKIMAYVFFCSWLMFPVMFIAGHEGLGLITPYTSGIGHLILDLISKNTWGFLGHHLRVKIHEHILIHGDIRKTTTINVAGENMEIETFVDEEEEGGVAAAKSRITSEGEYIPLDQIDINVFCYENEV

SEQ ID NO: 261 - protein, 258 aa, Artificial sequence
  Archaerhodopsin-3
MDPIALQAGYDLLGDGRPETLWLGIGTLLMLIGTFYFLVRGWGVTDKDAREYYAVTILVPGIASAAYLSMFFGIGLTEVTVGGEMLDIYYARYADWLFTTPLLLLDLALLAKVDRVTIGTLVGVDALMIVTGLIGALSHTAIARYSWWLFSTICMIVVLYFLATSLRSAAKERGPEVASTFNTLTALVLVLWTAYPILWIIGTEGAGVVGLGIETLLFMVLDVTAKVGFGFILLRSRAILGDTEAPEPSAGADVSAAD

SEQ ID NO: 262 - protein, 293 aa, Artificial sequence
  Arch3.0
MDPIALQAGYDLLGDGRPETLWLGIGTLLMLIGTFYFLVRGWGVTDKDAREYYAVTILVPGIASAAYLSMFFGIGLTEVTVGGEMLDIYYARYADWLFTTPLLLLDLALLAKVDRVTIGTLVGVDALMIVTGLIGALSHTAIARYSWWLFSTICMIVVLYFLATSLRSAAKERGPEVASTFNTLTALVLVLWTAYPILWIIGTEGAGVVGLGIETLLFMVLDVTAKVGFGFILLRSRAILGDTEAPEPSAGADVSAADRPVVAAAAKSRITSEGEYIPLDQIDINVFCYENEV

SEQ ID NO: 263 - protein, 248 aa, Artificial sequence
  ArchT
MDPIALQAGYDLLGDGRPETLWLGIGTLLMLIGTFYFIVKGWGVTDKEAREYYSITILVPGIASAAYLSMFFGIGLTEVTVAGEVLDIYYARYADWLFTTPLLLLDLALLAKVDRVSIGTLVGVDALMIVTGLIGALSHTPLARYSWWLFSTICMIVVLYFLATSLRAAAKERGPEVASTFNTLTALVLVLWTAYPILWIIGTEGAGVVGLGIETLLFMVLDVTAKVGFGFILLRSRAILGDTEAPEP

SEQ ID NO: 264 - protein, 278 aa, Artificial sequence
  ArchT with ER export and trafficking signal sequences
MDPIALQAGYDLLGDGRPETLWLGIGTLLMLIGTFYFIVKGWGVTDKEAREYYSITILVPGIASAAYLSMFFGIGLTEVTVAGEVLDIYYARYADWLFTTPLLLLDLALLAKVDRVSIGTLVGVDALMIVTGLIGALSHTPLARYSWWLFSTICMIVVLYFLATSLRAAAKERGPEVASTFNTLTALVLVLWTAYPILWIIGTEGAGVVGLGIETLLFMVLDVTAKVGFGFILLRSRAILGDTEAPEPAAAKSRITSEGEYIPLDQIDINVFCYENEV

SEQ ID NO: 265 - protein, 242 aa, Artificial sequence
  GtR3
MLVGEGAKLDVHGCKTVDMASSFGKALLEFVFIVFACITLLLGINAAKSKAASRVLFPATFVTGIASIAYFSMASGGGWVIAPDCRQLFVARYLDWLITTPLLLIDLGLVAGVSRWDIMALCLSDVLMIATGAFGSLTVGNVKWVWWFFGMCWFLHIIFALGKSWAEAAKAKGGDSASVYSKIAGITVITWFCYPVVWVFAEGFGNFSVTFEVLIYGVLDVISKAVFGLILMSGAATGYESI

SEQ ID NO: 266 - protein, 272 aa, Artificial sequence
  GtR3 with ER export and trafficking signal sequences
MLVGEGAKLDVHGCKTVDMASSFGKALLEFVFIVFACITLLLGINAAKSKAASRVLFPATFVTGIASIAYFSMASGGGWVIAPDCRQLFVARYLDWLITTPLLLIDLGLVAGVSRWDIMALCLSDVLMIATGAFGSLTVGNVKWVWWFFGMCWFLHIIFALGKSWAEAAKAKGGDSASVYSKIAGITVITWFCYPVVWVFAEGFGNFSVTFEVLIYGVLDVISKAVFGLILMSGAATGYESIAAAKSRITSEGEYIPLDQIDINVFCYENEV

SEQ ID NO: 267 - protein, 262 aa, Oxyrrhis marina
MAPLAQDWTYAEWSAVYNALSFGIAGMGSATIFFWLQLPNVTKNYRTALTITGIVTLIATYHYFRIFNSWVAAFNVGLGVNGAYEVTVSGTPFNDAYRYVDWLLTVPLLLVELILVMKLPAKETVCLAWTLGIASAVMVALGYPGEIQDDLSVRWFWWACAMVPFVYVVGTLVVGLGAATAKQPEGVVDLVSAARYLTVVSWLTYPFVYIVKNIGLAGSTATMYEQIGYSAADVTAKAVFGVLIWAIANAKSRLEEEGKLRA

SEQ ID NO: 268 - protein, 292 aa, Artificial sequence
  rhodopsin type II proton pump with ER export and trafficking signal sequences
MAPLAQDWTYAEWSAVYNALSFGIAGMGSATIFFWLQLPNVTKNYRTALTITGIVTLIATYHYFRIFNSWVAAFNVGLGVNGAYEVTVSGTPFNDAYRYVDWLLTVPLLLVELILVMKLPAKETVCLAWTLGIASAVMVALGYPGEIQDDLSVRWFWWACAMVPFVYVVGTLVVGLGAATAKQPEGVVDLVSAARYLTVVSWLTYPFVYIVKNIGLAGSTATMYEQIGYSAADVTAKAVFGVLIWAIANAKSRLEEEGKLRAAAAKSRITSEGEYIPLDQIDINVFCYENEV

SEQ ID NO: 269 - protein, 313 aa, Leptosphaeria maculans
MIVDQFEEVLMKTSQLFPLPTATQSAQPTHVAPVPTVLPDTPIYETVGDSGSKTLWVVFVLMLIASAAFTALSWKIPVNRRLYHVITTIITLTAALSYFAMATGHGVALNKIVIRTQHDHVPDTYETVYRQVYYARYIDWAITTPLLLLDLGLLAGMSGAHIFMAIVADLIMVLTGLFAAFGSEGTPQKWGWYTIACIAYIFVVWHLVLNGGANARVKGEKLRSFFVAIGAYTLILWTAYPIVWGLADGARKIGVDGEIIAYAVLDVLAKGVFGAWLLVTHANLRESDVELNGFWANGLNREGAIRIGEDDGA

SEQ ID NO: 270 - protein, 351 aa, Artificial sequence
  Mac3.0
MIVDQFEEVLMKTSQLFPLPTATQSAQPTHVAPVPTVLPDTPIYETVGDSGSKTLWVVFVLMLIASAAFTALSWKIPVNRRLYHVITTIITLTAALSYFAMATGHGVALNKIVIRTQHDHVPDTYETVYRQVYYARYIDWAITTPLLLLDLGLLAGMSGAHIFMAIVADLIMVLTGLFAAFGSEGTPQKWGWYTIACIAYIFVVWHLVLNGGANARVKGEKLRSFFVAIGAYTLILWTAYPIVWGLADGARKIGVDGEIIAYAVLDVLAKGVFGAWLLVTHANLRESDVELNGFWANGLNREGAIRIGEDDGARPVVAVSKAAAKSRITSEGEYIPLDQIDINVFCYENEV

SEQ ID NO: 271 - protein, 291 aa, Natronomonas Pharaonis
MTETLPPVTESAVALQAEVTQRELFEFVLNDPLLASSLYINIALAGLSILLFVFMTRGLDDPRAKLIAVSTILVPVVSIASYTGLASGLTISVLEMPAGHFAEGSSVMLGGEEVDGVVTMWGRYLTWALSTPMILLALGLLAGSNATKLFTAITFDIAMCVTGLAAALTTSSHLMRWFWYAISCACFLVVLYILLVEWAQDAKAAGTADMFNTLKLLTVVMWLGYPIVWALGVEGIAVLPVGVTSWGYSFLDIVAKYIFAFLLLNYLTSNESVVSGSILDVPSASGTPADD

SEQ ID NO: 272 - protein, 320 aa, Artificial sequence
  NpHR3.0
MTETLPPVTESAVALQAEVTQRELFEFVLNDPLLASSLYINIALAGLSILLFVFMTRGLDDPRAKLIAVSTILVPVVSIASYTGLASGLTISVLEMPAGHFAEGSSVMLGGEEVDGVVTMWGRYLTWALSTPMILLALGLLAGSNATKLFTAITFDIAMCVTGLAAALTTSSHLMRWFWYAISCACFLVVLYILLVEWAQDAKAAGTADMFNTLKLLTVVMWLGYPIVWALGVEGIAVLPVGVTSWGYSFLDIVAKYIFAFLLLNYLTSNESVVSGSILDVPSASGTPADDAAAKSRITSEGEYIPLDQIDINFCYENEV

SEQ ID NO: 273 - protein, 303 aa, Artificial sequence
  NOIR3.1
MVTQRELFEFVLNDPLLASSLYINIALAGLSILLFVFMTRGLDDPRAKLIAVSTILVPVVSIASYTGLASGLTISVLEMPAGHFAEGSSVMLGGEEVDGVVTMWGRYLTWALSTPMILLALGLLAGSNATKLFTAITFDIAMCVTGLAAALTTSSHLMRWFWYAISCACFLVVLYILLVEWAQDAKAAGTADMFNTLKLLTVVMWLGYPIVWALGVEGIAVLPVGVTSWGYSFLDIVAKYIFAFLLLNYLTSNESVVSGSILDVPSASGTPADDAAAKSRITSEGEYIPLDQIDINFCYENEV

SEQ ID NO: 274 - protein, 365 aa, Dunaliella saliva
MRRRESQLAYLCLFVLIAGWAPRLTESAPDLAERRPPSERNTPYANIKKVPNITEPNANVQLDGWALYQDFYYLAGSDKEWVVGPSDQCYCRAWSKSHGTDREGEAAVVWAYIVFAICIVQLVYFMFAAWKATVGWEEVYVNIIELVHIALVIWVEFDKPAMLYLNDGQMVPWLRYSAWLLSCPVILIHLSNLTGLKGDYSKRTMGLLVSDIGTIVFGTSAALAPPNHVKVILFTIGLLYGLFTFFTAAKVYIEAYHTVPKGQCRNLVRAMAWTYFVSWAMFPILFILGREGFGHITYFGSSIGHFILEIFSKNLWSLLGHGLRYRIRQHIIIHGNLTKKNKINIAGDNVEVEEYVDSNDKDSDV

SEQ ID NO: 275 - protein, 395 aa, Artificial sequence
  Dunaliella salina channel rhodopsin with ER export and trafficking signal sequences
MRRRESQLAYLCLFVLIAGWAPRLTESAPDLAERRPPSERNTPYANIKKVPNITEPNANVQLDGWALYQDFYYLAGSDKEWVVGPSDQCYCRAWSKSHGTDREGEAAVVWAYIVFAICIVQLVYFMFAAWKATVGWEEVYVNIIELVHIALVIWVEFDKPAMLYLNDGQMVPWLRYSAWLLSCPVILIHLSNLTGLKGDYSKRTMGLLVSDIGTIVFGTSAALAPPNHVKVILFTIGLLYGLFTFFTAAKVYIEAYHTVPKGQCRNLVRAMAWTYFVSWAMFPILFILGREGFGHITYFGSSIGHFILEIFSKNLWSLLGHGLRYRIRQHIIIHGNLTKKNKINIAGDNVEVEEYVDSNDKDSDVAAAKSRITSEGEYIPLDQIDINVFCYENEV

SEQ ID NO: 276 - protein, 348 aa, Artificial sequence
  iC1C2
MSRRPWLLALALAVALAAGSAGASTGSDATVPVATQDGPDYVFHRAHERMLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTCPVILIRLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSKYGSNVGHTIIDLMSKQCWGLLGHYLRVLIHEHILIHGDIRKTTKLNIGGTEIEVETLVEDEAEAGAV

SEQ ID NO: 277 - protein, 378 aa, Artificial sequence
  iC1C2 with ER export and trafficking signal sequences
MSRRPWLLALALAVALAAGSAGASTGSDATVPVATQDGPDYVFHRAHERMLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTCPVILIRLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSKYGSNVGHTIIDLMSKQCWGLLGHYLRVLIHEHILIHGDIRKTTKLNIGGTEIEVETLVEDEAEAGAVAAAKSRITSEGEYIPLDQIDINVFCYENEV

SEQ ID NO: 278 - protein, 348 aa, Artificial sequence
  SwiChR(iC1C2-C167AorTorS)
  misc_feature (167)..(167): Xaa can be any naturally occurring amino acid
MSRRPWLLALALAVALAAGSAGASTGSDATVPVATQDGPDYVFHRAHERMLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTXPVILIRLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSKYGSNVGHTIIDLMSKQCWGLLGHYLRVLIHEHILIHGDIRKTTKLNIGGTEIEVETLVEDEAEAGAV

SEQ ID NO: 279 - protein, 378 aa, Artificial sequence
  SwiChR(iC1C2-C167AorTorS) with ER export and trafficking signal sequences
  misc_feature (167)..(167): Xaa can be any naturally occurring amino acid
MSRRPWLLALALAVALAAGSAGASTGSDATVPVATQDGPDYVFHRAHERMLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTXPVILIRLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSKYGSNVGHTIIDLMSKQCWGLLGHYLRVLIHEHILIHGDIRKTTKLNIGGTEIEVETLVEDEAEAGAVAAAKSRITSEGEYIPLDQIDINVFCYENEV

SEQ ID NO: 280 - protein, 309 aa, Artificial sequence
  ibC1C2
MDYGGALSAVGLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTCPVILIRLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSKYGSNVGHTIIDLMSKQCWGLLGHYLRVLIHEHILIHGDIRKTTKLNIGGTEIEVETLVEDEAEAGAV

SEQ ID NO: 281 - protein, 339 aa, Artificial sequence
  ibC1C2 with ER export and trafficking signal sequences
MDYGGALSAVGLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTCPVILIRLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSKYGSNVGHTIIDLMSKQCWGLLGHYLRVLIHEHILIHGDIRKTTKLNIGGTEIEVETLVEDEAEAGAVAAAKSRITSEGEYIPLDQIDINVFCYENEV

SEQ ID NO: 282 - protein, 310 aa, Artificial sequence
  ChR2
MDYGGALSAVGRELLFVTNPVVVNGSVLVPEDQCYCAGWIESRGTNGAQTASNVLQWLSAGFSILLLMFYAYQTWKSTCGWEEIYVCAISMVKVILEFFFSFKNPSMLYLATGHRVKWLRYASWLLTCPVILIRLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSKYGSNVGHTIIDLMSKQCWGLLGHYLRVLIHEHILIHGDIRKTTKLNIGGTEIEVETLVEDEAEAGAVP

SEQ ID NO: 283 - protein, 340 aa, Artificial sequence
  ChR2 with ER export and trafficking signal sequences
MDYGGALSAVGRELLFVTNPVVVNGSVLVPEDQCYCAGWIESRGTNGAQTASNVLQWLSAGFSILLLMFYAYQTWKSTCGWEEIYVCAISMVKVILEFFFSFKNPSMLYLATGHRVKWLRYASWLLTCPVILIRLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSKYGSNVGHTIIDLMSKQCWGLLGHYLRVLIHEHILIHGDIRKTTKLNIGGTEIEVETLVEDEAEAGAVPAAAKSRITSEGEYIPLDQIDINVFCYENEV

SEQ ID NO: 284 - protein, 344 aa, Artificial sequence
  C1V1
MSRRPWLLALALAVALAAGSAGASTGSDATVPVATQDGPDYVFHRAHERMLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTCPVLLIRLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISKYGSNIGHSILDLIAKQMWGVLGNYLRVKIHEHILLYGDIRKKQKITIAGQEMEVETLVAEEED

SEQ ID NO: 285 - protein, 374 aa, Artificial sequence
  C1V1 with ER export and trafficking signal sequences
MSRRPWLLALALAVALAAGSAGASTGSDATVPVATQDGPDYVFHRAHERMLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTCPVLLIRLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISKYGSNIGHSILDLIAKQMWGVLGNYLRVKIHEHILLYGDIRKKQKITIAGQEMEVETLVAEEEDAAAKSRITSEGEYIPLDQIDINVFCYENEV

SEQ ID NO: 286 - protein, 305 aa, Artificial sequence
  ibC1V1
MDYGGALSAVGLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTCPVLLIRLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISKYGSNIGHSILDLIAKQMWGVLGNYLRVKIHEHILLYGDIRKKQKITIAGQEMEVETLVAEEED

SEQ ID NO: 287 - protein, 335 aa, Artificial sequence
  ibC1V1 with ER export and trafficking signal sequences
MDYGGALSAVGLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTCPVLLIRLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISKYGSNIGHSILDLIAKQMWGVLGNYLRVKIHEHILLYGDIRKKQKITIAGQEMEVETLVAEEEDAAAKSRITSEGEYIPLDQIDINVFCYENEV

SEQ ID NO: 288 - protein, 350 aa, Artificial sequence
  iReaChR
MVSRRPWLLALALAVALAAGSAGASTGSDATVPVATQDGPDYVFHRAHERMLFQTSYTLENNGSVICIPNNGQCFCLAWLKSNGTNAEKLAANILQWVSFALSVACLGWYAYQAWRATCGWEEVYVALISMMKSIIEAFHSFDSPATLWLSSGNGVKWMRYGSWLLTCPVILIRLSNLTGLKDDYSKRTMGLLVSDVGCIVWGATSAMCTGWTKILFFLISLSYGMYTYFHAAKVYIEAFHTVPKGLCRQLVRAMAWLFFVSWGMFPVLFLLGPEGFGHISKYGSNIGHSILDLIAKQMWGVLGNYLRVKIHEHILLYGDIRKKQKITIAGQEMEVETLVAEEEDKYESS

SEQ ID NO: 289 - protein, 380 aa, Artificial sequence
  iReaChR with ER export and trafficking signal sequences
MVSRRPWLLALALAVALAAGSAGASTGSDATVPVATQDGPDYVFHRAHERMLFQTSYTLENNGSVICIPNNGQCFCLAWLKSNGTNAEKLAANILQWVSFALSVACLGWYAYQAWRATCGWEEVYVALISMMKSIIEAFHSFDSPATLWLSSGNGVKWMRYGSWLLTCPVILIRLSNLTGLKDDYSKRTMGLLVSDVGCIVWGATSAMCTGWTKILFFLISLSYGMYTYFHAAKVYIEAFHTVPKGLCRQLVRAMAWLFFVSWGMFPVLFLLGPEGFGHISKYGSNIGHSILDLIAKQMWGVLGNYLRVKIHEHILLYGDIRKKQKITIAGQEMEVETLVAEEEDKYESSAAAKSRITSEGEYIPLDQIDINVFCYENEV

SEQ ID NO: 290 - protein, 310 aa, Artificial sequence
  ibReaChR
MDYGGALSAVGLFQTSYTLENNGSVICIPNNGQCFCLAWLKSNGTNAEKLAANILQWVSFALSVACLGWYAYQAWRATCGWEEVYVALISMMKSIIEAFHSFDSPATLWLSSGNGVKWMRYGSWLLTCPVILIRLSNLTGLKDDYSKRTMGLLVSDVGCIVWGATSAMCTGWTKILFFLISLSYGMYTYFHAAKVYIEAFHTVPKGLCRQLVRAMAWLFFVSWGMFPVLFLLGPEGFGHISKYGSNIGHSILDLIAKQMWGVLGNYLRVKIHEHILLYGDIRKKQKITIAGQEMEVETLVAEEEDKYESS

SEQ ID NO: 291 - protein, 340 aa, Artificial sequence
  ibReaChR with ER export and trafficking signal sequences
MDYGGALSAVGLFQTSYTLENNGSVICIPNNGQCFCLAWLKSNGTNAEKLAANILQWVSFALSVACLGWYAYQAWRATCGWEEVYVALISMMKSIIEAFHSFDSPATLWLSSGNGVKWMRYGSWLLTCPVILIRLSNLTGLKDDYSKRTMGLLVSDVGCIVWGATSAMCTGWTKILFFLISLSYGMYTYFHAAKVYIEAFHTVPKGLCRQLVRAMAWLFFVSWGMFPVLFLLGPEGFGHISKYGSNIGHSILDLIAKQMWGVLGNYLRVKIHEHILLYGDIRKKQKITIAGQEMEVETLVAEEEDKYESSAAAKSRITSEGEYIPLDQIDINVFCYENEV

SEQ ID NO: 292 - protein, 315 aa, Artificial Sequence
  MyoD-ERT2 fusion polypeptide
PSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATA

SEQ ID NO: 293 - protein, 955 aa, Artificial sequence
  CD4-Linker-MK2-eLOV-TEVcs-GAL4
METDTLLLWVLLLWVPGSTGDGAQPARSYPYDVPDYAYPYDVPDYALDFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRKGSGSTSGSGSGGSRGSGGSSGGMNGAIGGDLLLNFPDMSVLERQRAHLKYLNPTFDSPLAGFFADSSMITGGEMDSYLSTAGLNLPMMYGETTVEGDSRLSISPETTLGTGNFKAAKFDTETKDCNEAAKKMTMNRDDLVEEGEEEKSKITEQNNGSTKSIKKMKHKAKKEENNFSNDSSKVTKELEKTDYIHGGSGSFNARRKLAGAILFTMLATRNFSGSFNARRKLAGAILFTMLATRNFSELAEKLAGLDINGGASGSRATTLERIEKSFVITDPRLPDNPIIFVSDSFLQLTEYSREEILGRNCRFLQGPETDRATVRKIRDAIDNQTEVTVQLINYTKSGKKFWNLFHLQPMRDQKGDVQYFIGVQLDGTERVRDAAEREAVMLVKKTAEEIDEAAKENLYFQMGGGSDYKDDDDKKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSANFNQSGNIADSSLSFTFTNSSNGPNLITTQTNSQALSQPIASSNVHDNFMNNEITASKIDDGNNSKPLSPGWTDQTAYNAFGITTGMFNTTTMDDVYNYLFDDEDTPPNPKKE

SEQ ID NO: 294 - protein, 399 aa, Artificial sequence
  CaM-Linker-TEV(1-219)
MDQLTEEQIAEFKEAFSLLDKDGDGTITTKELGTGMRSLGQNPTEAELQDMINEVDADGDGTIDFPEFLTMMARKMKYTDSEEEIREAFRVFDKDGNGYISAAELRHVMTNLGEKLTDEEVDEMIREADIDGDGQVNYEEFVQMMTAKGKPIPNPLLGLDSTGGSGSGSGGSYGSHVDYAGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMV

SEQ ID NO: 295 - protein, 904 aa, Artificial sequence
  CD4-CIBN-eLOV-TEVcs-GAL4
METDTLLLWVLLLWVPGSTGDGAQPARSYPYDVPDYAYPYDVPDYALDFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRKGSGSTSGSGSGGSRGSGGSSGGMNGAIGGDLLLNFPDMSVLERQRAHLKYLNPTFDSPLAGFFADSSMITGGEMDSYLSTAGLNLPMMYGETTVEGDSRLSISPETTLGTGNFKAAKFDTETKDCNEAAKKMTMNRDDLVEEGEEEKSKITEQNNGSTKSIKKMKHKAKKEENNFSNDSSKVTKELEKTDYIHELAEKLAGLDINGGASGSRATTLERIEKSFVITDPRLPDNPIIFVSDSFLQLTEYSREEILGRNCRFLQGPETDRATVRKIRDAIDNQTEVTVQLINYTKSGKKFWNLFHLQPMRDQKGDVQYFIGVQLDGTERVRDAAEREAVMLVKKTAEEIDEAAKENLYFQYGGGSDYKDDDDKKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSANFNQSGNIADSSLSFTFTNSSNGPNLITTQTNSQALSQPIASSNVHDNFMNNEITASKIDDGNNSKPLSPGWTDQTAYNAFGITTGMFNTTTMDDVYNYLFDDEDTPPNPKKE

SEQ ID NO: 296 - protein, 756 aa, Artificial sequence
  CRY2-Linker-TEV(1-219)
MGKPIPNPLLGLDSTMKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTREAQIMIGAAPGGSGSGGSGSGSGGSYGSHVDYAGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMV

SEQ ID NO: 297 - protein, 865 aa, Artificial sequence
  Beta2-AR-Linker-eLOV-TEVcs-GAL4
MGQPGNGSAFLLAPNGSHAPDHDVTQERDEVWVVGMGIVMSLIVLAIVFGNVLVITAIAKFERLQTVTNYFITSLACADLVMGLAVVPFGAAHILMKMWTFGNFWCEFWTSIDVLCVTASIETLCVIAVDRYFAITSPFKYQSLLTKNKARVIILMVWIVSGLTSFLPIQMHWYRATHQEAINCYANETCCDFFTNQAYAIASSIVSFYVPLVIMVFVYSRVFQEAKRQLQKIDKSEGRFHVQNLSQVEQDGRTGHGLRRSSKFCLKEHKALKTLGIIMGTFTLCWLPFFIVNIVHVIQDNLIRKEVYILLNWIGYVNSGFNPLIYCRSPDFRIAFQELLCLRRSSLKAYGNGYSSNGNTGEQSGYHVEQEKENKLLCEDLPGTEDFVGHQGTVPSDNIDSQGRNCSTNDSLLELAEKLAGLDINGGASGSRATTLERIEKSFVITDPRLPDNPIIFVSDSFLQLTEYSREEILGRNCRFLQGPETDRATVRKIRDAIDNQTEVTVQLINYTKSGKKFWNLFHLQPMRDQKGDVQYFIGVQLDGTERVRDAAEREAVMLVKKTAEEIDEAAKENLYFQMGGGSDYKDDDDKKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSANFNQSGNIADSSLSFTFTNSSNGPNLITTQTNSQALSQPIASSNVHDNFMNNEITASKIDDGNNSKPLSPGWTDQTAYNAFGITTGMFNTTTMDDVYNYLFDDEDTPPNPKKEGKPIPNPLLGLDST

SEQ ID NO: 298 - protein, 657 aa, Artificial sequence
  Beta2-arrestin-Linker-TEV(1-219)
MGEKPGTRVFKKSSPNCKLTVYLGKRDFVDHLDKVDPVDGVVLVDPDYLKDRKVFVTLTCAFRYGREDLDVLGLSFRKDLFIATYQAFPPMPNPPRPPTRLQDRLLKKLGQHAHPFFFTIPQNLPCSVTLQPGPEDTGKACGVDFEIRAFCAKSIEEKSHKRNSVRLIIRKVQFAPETPGPQPSAETTRHFLMSDRRSLHLEASLDKELYYHGEPLNVNVHVTNNSAKTVKKIRVSVRQYADICLFSTAQYKCPVAQLEQDDQVSPSSTFCKVYTITPLLSDNREKRGLALDGQLKHEDTNLASSTIVKEGANKEVLGILVSYRVKVKLVVSRGGDVSVELPFVLMHPKPHDHITLPRPQSAPREIDIPVDTNLIEFDTNYATDDDIVFEDFARLRLKGMKDDDCDDQFCYPYDVPDYAYPYDVPDYAGGSGSGSGGSGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMV

SEQ ID NO: 299 - DNA, 1299 bp, Artificial sequence
  nucleic acid encoding PPI detection system
aggcctccaaggcggagtactgtcctccgggctggcggagtactgtcctccggcaaggtcggagtactgtcctccgacactagaggtcggagtactgtcctccgacgcaaggcggagtactgtcctccgggctgcggagtactgtcctccggcaaggtcggagtactgtcctccgacactagaggtcggagtactgtcctccgacgcaaggtcggagtactgtcctccgacactagaggtcggagtactgtcctccgacgcaaggtcggagtactgtcctccgacactagaggtcggagtactgtcctccgacgcaaggcggagtactgtcctccgggctggcggagtactgtcctccggcaagggtcgactctagagggtatataatggatcccatcgcgtctcagcctcactttgagctcctccacacgaattccccgataccgtcgattcaaggagcttgcttgttctttttgcagaagctcagaataaacgctcaactttggcagatctaccaagcttggtaccgagctcggatccactagtgatccccgggtaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaggacctttga

SEQ ID NO: 300 - protein, 997 aa, Artificial sequence
  CD4-Linker-FRB-eLOV-TEVcs-GAL4
METDTLLLWVLLLWVPGSTGDGAQPALDFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRKGSGSTSGSGSGGSRGSGGSSGGMNGAIGGDLLLNFPDMSVLERQRAHLKYLNPTFDSPLAGFFADSSMITGGEMDSYLSTAGLNLPMMYGETTVEGDSRLSISPETTLGTGNFKAAKFDTETKDCNEAAKKMTMNRDDLVEEGEEEKSKITEQNNGSTKSIKKMKHKAKKEENNFSNDSSKVTKELEKTDYIHGGSGSVAILWHEMWHEGLEEASRLYFGERNVKGMFEVLEPLHAMMERGPQTLKETSFNQAYGRDLMEAQEWCRKYMKSGNVKDLTQAWDLYYHVFRRISELAEKLAGLDINGGASGSRATTLERIEKSFVITDPRLPDNPIIFVSDSFLQLTEYSREEILGRNCRFLQGPETDRATVRKIRDAIDNQTEVTVQLINYTKSGKKFWNLFHLQPMRDQKGDVQYFIGVQLDGTERVRDAAEREAVMLVKKTAEEIDEAAKENLYFQMGGGSDYKDDDDKKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSANFNQSGNIADSSLSFTFTNSSNGPNLITTQTNSQALSQPIASSNVHDNFMNNEITASKIDDGNNSKPLSPGWTDQTAYNAFGITTGMFNTTTMDDVYNYLFDDEDTPPNPKKEGKPIPNPLLGLDST

SEQ ID NO: 301 - protein, 355 aa, Artificial sequence
  FKBP-Linker-TEV(1-219)
MGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEYPYDVPDYAYPYDVPDYAGGSGSGSGGSGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMV

SEQ ID NO: 302 - protein, 15 aa, Artificial Sequence
  biotin-acceptor tag (BAT)
GLNDIFEAQKIEWHE